(19) United States
(12) Patent Application Publication
(10) Pub. No.: US 2024/0376120 A1
(43) Pub. Date: Nov. 14, 2024

(54) CAMPTOTHECIN COMPOUND, PREPARATION METHOD THEREFOR AND USE THEREOF

(71) Applicant: SHANGHAI BEST-LINK BIOSCIENCE, LLC, Shanghai (CN)

(72) Inventors: Fuyao ZHANG, Shanghai (CN); Xianjie CHEN, Shanghai (CN)

(21) Appl. No.: 18/684,856

(22) PCT Filed: Aug. 31, 2022
(86) PCT No.: PCT/CN2022/116085

§ 371 Date: Feb. 20, 2024

(30) Foreign Application Priority Data
Sep. 1, 2021 (CN) 202111020912.1

(51) Int. Cl. C07D 491/22; A61K 31/4375; A61P 35/00

(52) U.S. Cl. CPC C07D 491/22; A61K 31/4375; A61P 35/00

(57) ABSTRACT

Disclosed are a camptothecin compound as shown in general formula (I), and a preparation method therefor and a use thereof, the definition of each group being as set forth in the description. The compound of the present invention has good antitumor activity and good application prospects.

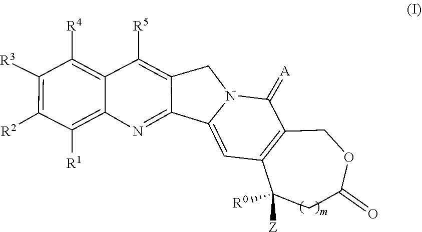

This application claims priority to Chinese Application No. 2021110209121, filed on Sep. 1, 2021. The contents of which are incorporated herein by its entirety.

### FIELD OF THE INVENTION

The present invention relates to the field of medicinal chemistry, and specifically to a camptothecin analogue compound, its preparation method and use.

### BACKGROUND OF THE INVENTION

Camptothecin (CPT) is a natural pentacyclic alkaloid, which was extracted from Camptotheca accuminata in China by Wall and other scientists in 1966 and got its name. It was used as traditional Chinese medicine to treat cancer in the early days. Although it has extensive anti-tumor activity, it has not received much attention due to its poor stability and solubility, especially its rapid hydrolysis of the lactone ring at physiological pH and loss of biological activity.

The structure and hydrolysis schematic diagram of camptothecin are shown below:

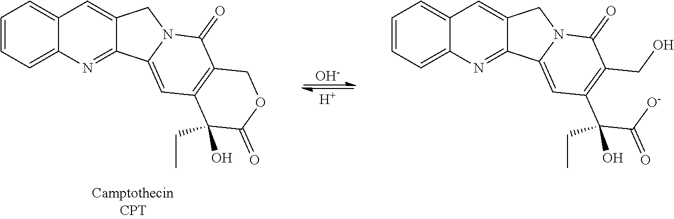

It was not until the 1980s that scientists revealed that its mechanism of action was topoisomerase I inhibitor that it attracted interest and was developed. In view of the pharmacological limitations of CPT, especially to improve the solubility and pharmacokinetics of CPT, various derivatives with different structures have been developed and used in clinical trials, among which irinotecan and topotecan are the the most successful of examples, especially Irinotecan, which has been proved to be widely used in the routine treatment of colorectal cancer. Recently, second-generation CPT analogs (such as lurtotecan, rubitecan, exatecan, and belotecan) have been developed to treat ovarian cancer and small cell lung cancer.

The structures of irinotecan and topotecan are shown in the figure below:

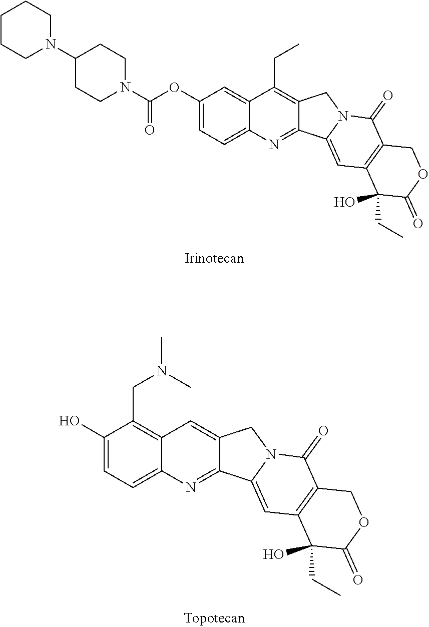 [SMILES: CCc1c2c(nc3ccc(OC(=O)N4CCC(N5CCCCC5)CC4)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1]

Recently, the development and progress of drug delivery technology, especially the development of the field of antibody drug conjugate (ADC), has brought great development to camptothecin drugs. For example, the success of IMMU-132 and DS-8201a in clinical research and their approval for marketing also indicate that camptothecin compounds may have their unique advantages in ADC drugs and will create greater medicinal value in the future.

Although irinotecan and other drugs are successful in clinic, they still have obvious shortcomings, such as the activity of prodrug is lower than mother drug, poor permeability, and poor tolerability. Camptothecin or its derivatives can be further modified and improved to meet clinical needs.

The present invention designs and synthesizes a series of new camptothecin derivatives with good anti-tumor activity.

### SUMMARY OF THE INVENTION

The invention aims to provide a novel camptothecin compound, its preparation method and application.

In one aspect, the present invention relates to a camptothecin compound represented by formula (I) or a pharmaceutically acceptable salt thereof:

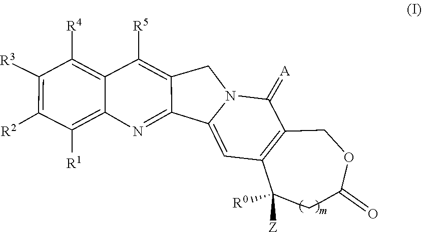

Wherein:
R⁰ is C1-C4 alkyl, C1-C4 haloalkyl, C1-C4 cycloalkyl, C1-C4 deuterated alkyl, C2-C4 alkynyl, or C2-C4 alkenyl;
R¹, R², R³ and R⁴ are each independently hydrogen, deuterium, halogen, cyano, hydroxyl, thiol, sulfone, sulfoxide, substituted or unsubstituted amino, nitro, alkynyl, alkenyl, alkyl, haloalkyl, cycloalkyl, alkoxy, alkylmercapto, heterocycloalkyl, deuterated alkyl, aryl, or heteroaryl;
Alternatively, R¹ and R², R² and R³, R³ and R⁴ each form a 5-7 membered cycloalkyl or heterocycloalkyl group together with their co-connected carbon atom;
R⁵ is

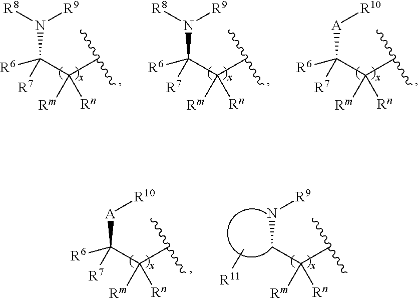

(the ring in

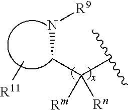

is 3-7 membered heterocycle),

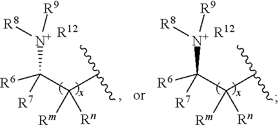

R⁶ is hydrogen, deuterium, cyano, alkynyl, alkenyl, alkyl, haloalkyl, cycloalkyl, heterocycloalkyl, deuterated alkyl, aryl or heteroaryl;
R⁷ is cyano, alkynyl, alkenyl, alkyl, haloalkyl, cycloalkyl, heterocycloalkyl, deuterated alkyl, aryl or heteroaryl;
Alternatively, R⁶ and R⁷ each form a 3-7 membered cycloalkyl or heterocycloalkyl group together with their co-connected carbon atom;
R⁸ and R⁹ are each independently hydrogen, deuterium, hydroxyl, substituted or unsubstituted amino, alkyl, haloalkyl, cycloalkyl, heterocycloalkyl, deuterated alkyl, aryl, heteroaryl,

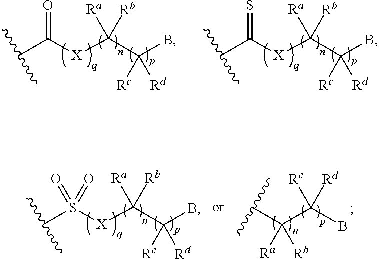

Alternatively, R⁸ and R⁹ form a 3-7 membered heterocycloalkyl group together with their co-connected nitrogen atoms.
R¹⁰ is hydrogen, alkynyl, alkenyl, alkyl, haloalkyl, cycloalkyl, alkylmercapto, heterocycloalkyl, deuterated alkyl, aryl, heteroaryl,

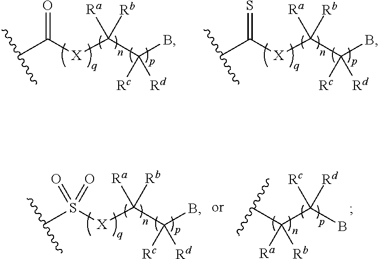

R¹¹ is one or more substituents on a 3-7 membered heterocyclic ring, which is selected from hydrogen, deuterium, halogen, cyano, hydroxyl, substituted or unsubstituted amino, alkynyl, alkenyl, alkyl, haloalkyl, ring Alkyl, alkoxy, alkylmercapto, heterocycloalkyl, deuterated alkyl, aryl, or heteroaryl;
R¹² is selected from alkyl;
R^{a} and R^{b} are each independently hydrogen, deuterium, halogen, cyano, hydroxyl, mercapto, substituted or unsubstituted amino, alkynyl, alkenyl, alkyl, haloalkyl, cycloalkyl, alkoxy, alkylmercapto, heterocycloalkyl, deuterated alkyl, aryl, or heteroaryl;
Alternatively, R^{a} and R^{b} form a 3-7 membered cycloalkyl or heterocycloalkyl group together with their co-connected carbon atom;
R^{c} and R^{d} are each independently hydrogen, deuterium, halogen, cyano, hydroxyl, mercapto, substituted or unsubstituted amino, alkynyl, alkenyl, alkyl, haloalkyl, cycloalkyl, alkoxy, alkylmercapto, heterocycloalkyl, deuterated alkyl, aryl, or heteroaryl;
Alternatively, Re and Rd each form a 3-7 membered cycloalkyl or heterocycloalkyl group together with their co-connected carbon atom;
Alternatively, R^{a} and R^{b}, R^{b} and R^{c}, R^{c} and R^{d} each form a cycloalkyl or heterocycloalkyl group together with their co-connected carbon atom;
B is hydrogen, deuterium, halogen, hydroxyl, mercapto, substituted or unsubstituted amino, alkynyl, alkenyl, alkoxy, alkylmercapto, aryl, or heteroaryl;
R^{m} and Rⁿ are each independently hydrogen, deuterium, halogen, cyano, hydroxyl, substituted or unsubstituted amino, alkynyl, alkenyl, alkyl, haloalkyl, cycloalkyl, alkoxy, alkylmercapto, heterocycloalkyl, deuterated alkyl, aryl, or heteroaryl;

Alternatively, R^{m} and Rⁿ form a cycloalkyl or heterocycloalkyl group together with co-connected carbon atom;
x, n and p are 0, 1, 2, 3 or 4;
q is 0 or 1;
m is 0 or 1;
X is O, S or NH;
A is O or S, and when A-R¹⁰ is a hydroxyl group, not all R¹, R², R³ and R⁴ are hydrogen (H);
Z is OH, SH or F.

In one embodiment of the present invention, R⁰ is preferably C1-C4 alkyl.

In one embodiment of the present invention, R¹, R², R³ and R⁴ are each independently preferably hydrogen, halogen, hydroxyl, alkyl, haloalkyl, alkoxy or deuterated alkyl; alternatively, R² and R³ form a heterocycloalkyl group together with their co-connected carbon atom;

In one embodiment of the invention, R² is preferably hydrogen or halogen; alternatively, R² and R³ form a heterocycloalkyl group together with their co-connected carbon atom;

In one embodiment of the present invention, R³ is preferably hydrogen, halogen, hydroxyl, alkyl, haloalkyl, alkoxy or deuterated alkyl; alternatively, R² and R³ form a heterocycloalkyl group together with their co-connected carbon atom.

In one embodiment of the invention, R⁴ is preferably hydrogen, halogen or alkyl.

In one embodiment of the invention, R⁶ is preferably hydrogen or alkyl.

In one embodiment of the present invention, R⁷ is preferably alkyl, haloalkyl or deuterated alkyl.

In one embodiment of the present invention, R⁸ and R⁹ are each independently preferably hydrogen, alkyl,

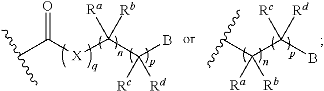

alternatively, R⁸ and R⁹ form a 3-7 membered heterocycloalkyl group together with their co-connected nitrogen atom.

In one embodiment of the invention, R¹⁰ is preferably hydrogen.

In one embodiment of the invention, R¹¹ is preferably hydrogen.

In one embodiment of the present invention, R¹² is preferably an alkyl group.

In one embodiment of the present invention, R^{a} and R^{b} are each independently preferably hydrogen, deuterium, alkyl, cycloalkyl or haloalkyl; alternatively, each of R^{a} and R^{b} form a cycloalkyl group together with their co-connected carbon atom; alternatively, R^{b} and R^{c} each form a cycloalkyl group together with their co-connected carbon atom.

In one embodiment of the present invention, R^{c} and R^{d} are each independently preferably hydrogen, deuterium, alkyl, cycloalkyl or haloalkyl; alternatively, each of R^{c} and R^{d} form a cycloalkyl group together with their co-connected carbon atom; alternatively, R^{b} and Re each form a cycloalkyl group together with their co-connected carbon atom.

In one embodiment of the present invention, B is preferably hydrogen, hydroxyl or substituted or unsubstituted amino group.

In a certain embodiment of the present invention, R^{m} and Rⁿ are each independently preferably hydrogen or a substituted or unsubstituted amino group.

In one embodiment of the present invention, R⁵ is preferably

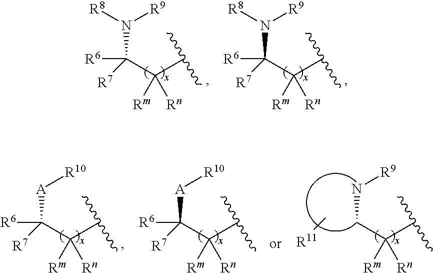

(the ring in

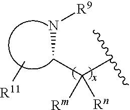

is a 3-7 membered heterocyclic ring), more preferably

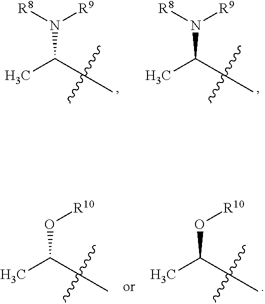

In one embodiment of the present invention, the alkynyl groups described in R¹, R², R³, R⁴, R⁶, R⁷, R¹⁰, R¹¹, R^{a}, R^{b}, R^{c}, R^{d}, B, R^{m} and Rⁿ are each independently preferably C2-C10 alkynyl group, more preferably C2-C₆ alkynyl group, most preferably C2-C4 alkynyl group.

In one embodiment of the present invention, the alkenyl groups described in R¹, R², R³, R⁴, R⁶, R⁷, R¹⁰, R¹¹, R^{a}, R^{b}, R^{c}, R^{d}, B, R^{m} and Rⁿ are each independently preferably C2-C10 alkenyl, more preferably C2-C₆ alkenyl, most preferably C2-C4 alkenyl.

In one embodiment of the present invention, the alkyl groups described in R¹, R², R³, R⁴, R⁶, R⁷, R⁸, R⁹, R¹⁰, R¹¹, R¹², R^{a}, R^{b}, R^{c}, R^{d}, R^{m} and Rⁿ are each independently preferably C1-C20 alkyl group, more preferably C1-C10 alkyl group, further preferably C1-C8 alkyl group, and particularly preferably methyl or ethyl group.

In one embodiment of the present invention, the haloalkyl groups described in R¹, R², R³, R⁴, R⁶, R⁷, R⁸, R⁹, R¹⁰, R¹¹, R¹², R^{b}, R^{c}, R^{d}, R^{m} and Rⁿ are each independently preferably halogenated C1-C20 alkyl group, more preferably halogenated C1-C10 alkyl group, further preferably halogenated C1-C8 alkyl group, and particularly preferably trifluoromethyl group.

In one embodiment of the present invention, the deuterated alkyl groups described in R¹, R², R³, R⁴, R⁶, R⁷, R⁸, R⁹, R¹⁰, R¹¹, R^{a}, R^{b}, R^{c}, R^{d}, R^{m} and Rⁿ are each independently deuterated C1-C20 alkyl group, more preferably deuterated C1-C10 alkyl group, further preferably deuterated C1-C8 alkyl group, particularly preferably methyl-d3 group.

In one embodiment of the present invention, the cycloalkyl groups described in R¹, R², R³, R⁴, R⁶, R⁷, R⁸, R⁹, R¹⁰, R¹¹, R^{a}, R^{b}, R^{c}, R^{d}, R^{m} and Rⁿ are each independently preferably C3-C20 cycloalkyl group, more preferably C3-C12 cycloalkyl group, further preferably C3-C10 cycloalkyl group, particularly preferably C3-C6 cycloalkyl group, and most preferably cyclopropyl or cyclohexyl group.

In one embodiment of the present invention, the heterocycloalkyl groups described in R¹, R², R³, R⁴, R⁶, R⁷, R⁸, R⁹, R¹⁰, R¹¹, R^{a}, R^{b}, R^{c}, R^{d}, R^{m} and Rⁿ are each independently preferably 3-20 membered heterocycloalkyl group, more preferably 3-12 membered heterocycloalkyl group, and further preferably dioxolane, dioxane or piperazine.

In one embodiment of the present invention, the alkoxy groups described in R¹, R², R³, R⁴, R¹¹, R^{a}, R^{b}, R^{c}, R^{d}, B, R^{m} and Rⁿ are each independently preferably —O—C1-C10 alkyl group or —O—C3-C10 cycloalkyl group, more preferably —O—C1-C8 alkyl group or —O—C3-C6 cycloalkyl group.

In one embodiment of the present invention, the alkyl mercapto groups described in R¹, R², R³, R⁴, R¹⁰, R¹¹, R^{a}, R^{b}, R^{c}, R^{d}, B, R^{m} and Rⁿ are each independently preferably —S—C1-C10 Alkyl or —S—C3-C10 cycloalkyl, more preferably —S—C1-C8 alkyl or —S—C3-C6 cycloalkyl.

In one embodiment of the present invention, the aryl groups described in R¹, R², R³, R⁴, R⁶, R⁷, R⁸, R⁹, R¹⁰, R¹¹, R^{a}, R^{b}, R^{c}, R^{d}, B, R^{m} and Rⁿ are each independently preferably C6-C18 aryl group, and more preferably C6-C10 aryl group.

In one embodiment of the present invention, the heteroaryl groups described in R¹, R², R³, R⁴, R⁶, R⁷, R⁸, R⁹, R¹⁰, R¹¹, R^{a}, R^{b}, R^{c}, R^{d}, B, R^{m} and Rⁿ are each independently preferably a 5-12 membered heteroaryl group, and more preferably 5-6 membered heteroaryl group.

In one embodiment of the present invention, the halogens described in R¹, R², R³, R⁴, R¹¹, R^{a}, R^{b}, R^{c}, R^{d}, B, R^{m} and Rⁿ are each independently preferably fluorine, chlorine, bromine or iodine, and more preferably fluorine.

In one embodiment of the present invention, the halogens in the haloalkyl groups described in R¹, R², R³, R⁴, R⁶, R⁷, R⁸, R⁹, R¹⁰, R¹¹, R^{a}, R^{b}, R^{c}, R^{d}, R^{m} and Rⁿ are each independently preferably fluorine, chlorine, bromine or iodine, and more preferably fluorine.

In one embodiment of the present invention, the substituted or unsubstituted amino groups described in R¹, R², R³, R⁴, R⁸, R⁹, R¹¹, R^{a}, R^{b}, R^{c}, R^{d}, B, R^{m} and Rⁿ are NH₂ group, monosubstituted NH₂ group or disubstituted NH₂ group. When the NH₂ group is substituted, the substituent is preferably independently C1-C20 alkyl group, more preferably C1-C10 alkyl group, further preferably C1-C8 alkyl group, and particularly preferably methyl group.

In one embodiment of the invention, R⁰ is preferably ethyl.

In one embodiment of the present invention, R¹ is preferably hydrogen.

In one embodiment of the present invention, R² is preferably hydrogen or fluorine; alternatively, R² and R³ preferably form

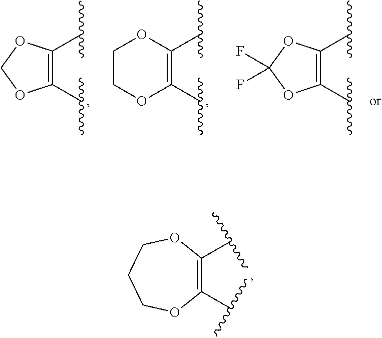

more preferably

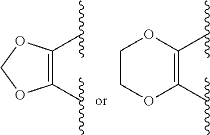

together with their co-connected carbon atoms.

In one embodiment of the present invention, R³ is preferably hydrogen, fluorine, hydroxyl, methyl, difluoromethyl, trifluoromethyl, methoxy or methyl-d3; alternatively, R² and R³ preferably form

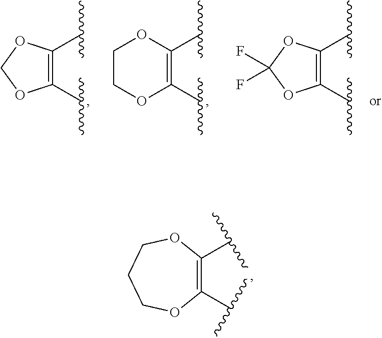

more preferably

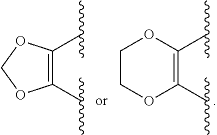

In one embodiment of the invention, R⁴ is preferably hydrogen or methyl.

In one embodiment of the present invention, R⁶ is preferably hydrogen or methyl, and more preferably hydrogen.

In one embodiment of the present invention, R⁷ is preferably methyl, ethyl, trifluoromethyl or methyl-d3.

In one embodiment of the present invention, R⁸ and R⁹ are each independently preferably hydrogen, methyl,

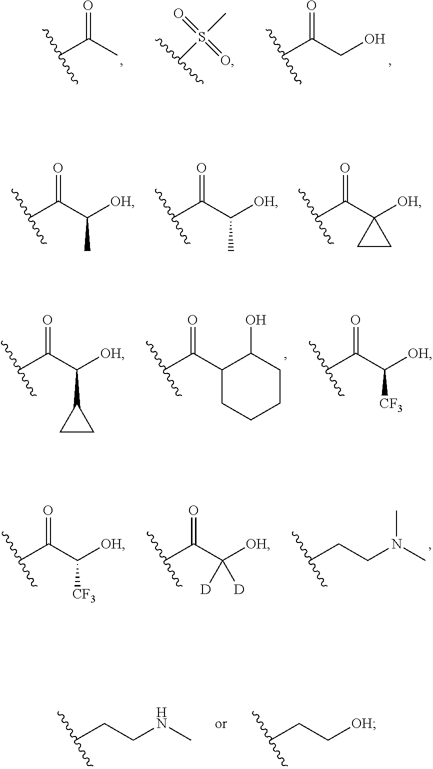

alternatively, R⁸ and R⁹ preferably form

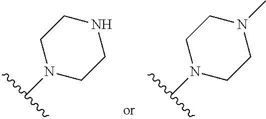

together with their co-connected nitrogen atom.

In one embodiment of the present invention, R⁵ is preferably

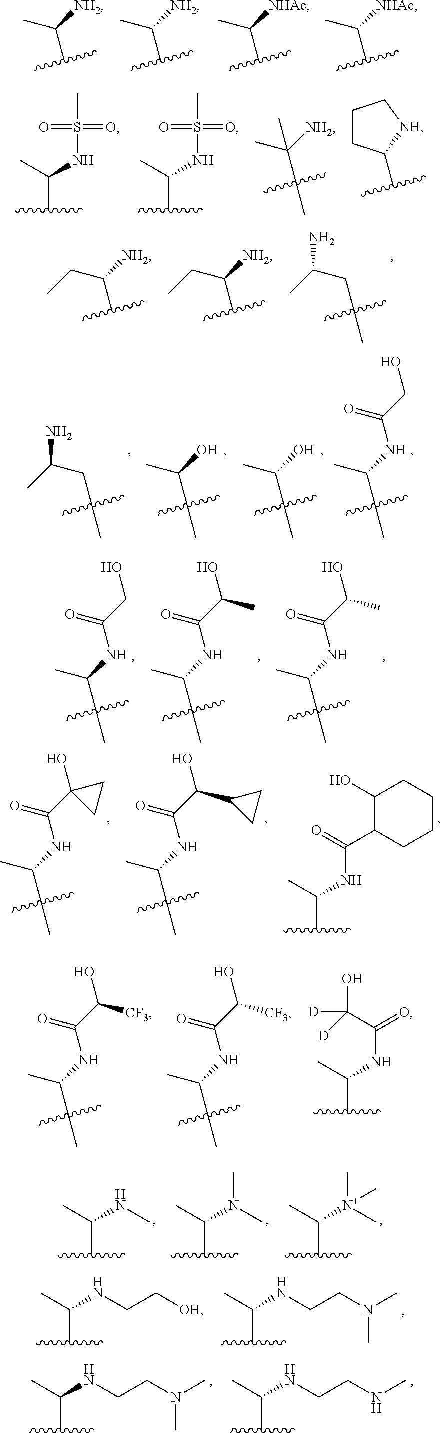

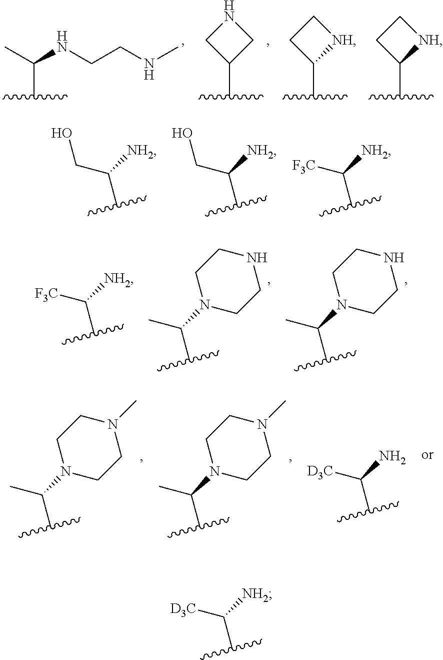

more preferably

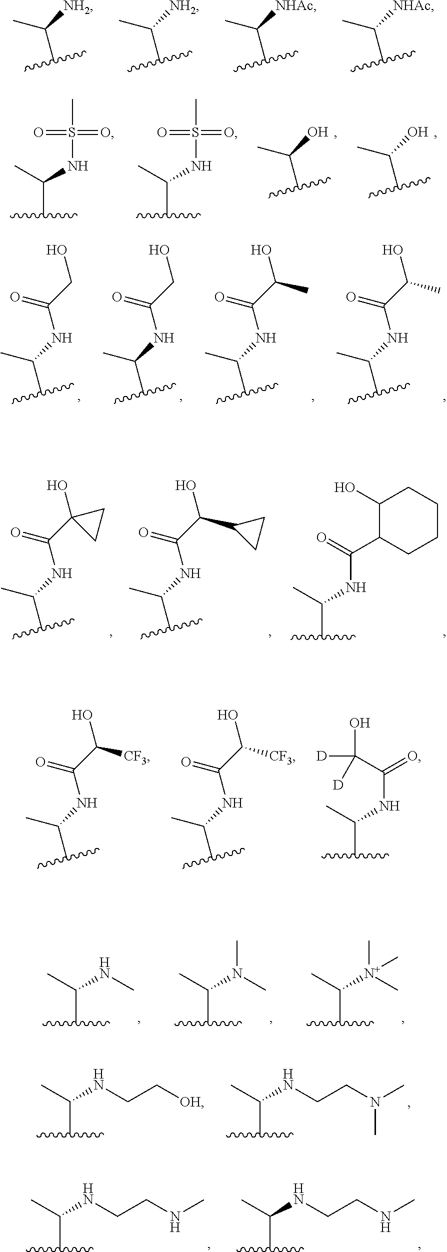

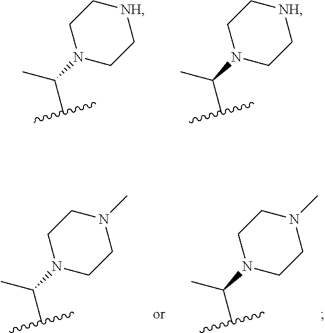

further preferably

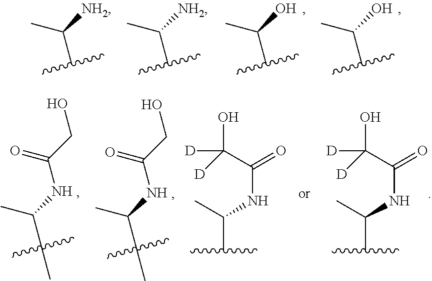

In one embodiment of the invention, A is preferably oxygen.

In one embodiment of the invention, m is preferably 0.

In one embodiment of the invention, Z is preferably hydroxyl.

In a preferred embodiment of the present invention, the camptothecin compound represented by general formula (I) or a pharmaceutically acceptable salt thereof is a camptothecin compound represented by general formula (II) or a pharmaceutically acceptable salt thereof:

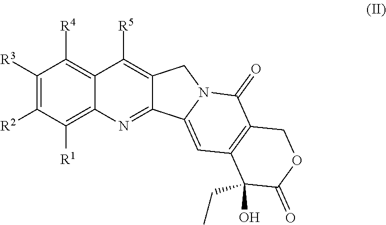

Wherein,
R¹, R², R³, R⁴ and R⁵ are as defined in the present invention.

In a preferred embodiment of the present invention, the camptothecin compound represented by general formula (I) or a pharmaceutically acceptable salt thereof is a camptothecin compound represented by general formula (III) or a pharmaceutically acceptable salt thereof:

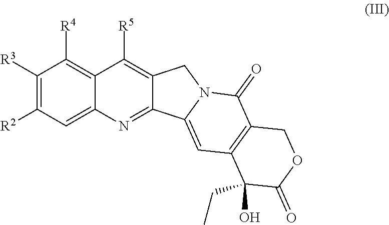

Wherein:
R², R³ and R⁴ are each independently hydrogen, deuterium, halogen, cyano, hydroxyl, mercapto, sulfonyl, sulfoxide, substituted or unsubstituted amino, alkenyl, alkynyl, alkyl, haloalkyl, cycloalkyl base, alkoxy, heterocycloalkyl or deuterated alkyl;
Alternatively, R² and R³, R³ and R⁴ each form a 5-7 membered cycloalkyl or heterocycloalkyl group together with their co-connected carbon atom;
R⁵ is

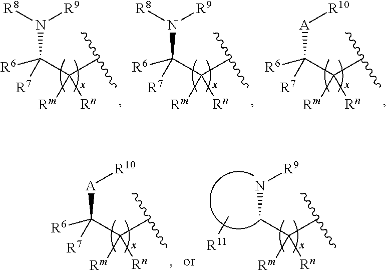

(the ring in

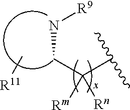

is 3-7 membered heterocycle);
R⁶ is hydrogen, deuterium, alkyl, haloalkyl, cycloalkyl, heterocycloalkyl, deuterated alkyl, aryl or heteroaryl;
R⁷ is alkyl, haloalkyl, cycloalkyl, heterocycloalkyl, deuterated alkyl, aryl or heteroaryl;
Alternatively, R⁶ and R⁷ form a 3-7 membered cycloalkyl or heterocycloalkyl group together with their co-connected carbon atom;
R⁸ and R⁹ are each independently hydrogen, deuterium, alkyl, haloalkyl, cycloalkyl, heterocycloalkyl, deuterated alkyl, aryl, heteroaryl,

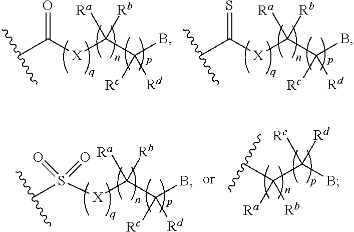

Alternatively, R⁸ and R⁹ form a 3-7 membered cycloalkyl or heterocycloalkyl group together with their co-connected nitrogen atom;
R¹⁰ is hydrogen, alkyl, haloalkyl, cycloalkyl, heterocycloalkyl, deuterated alkyl, aryl or heteroaryl,

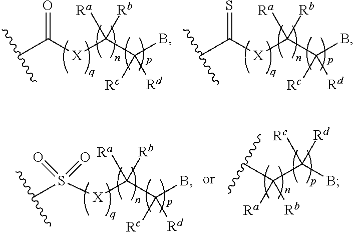

R¹¹ is one or more substituents of a 3-7 membered heterocycle, which is hydrogen, deuterium, halogen, cyano, hydroxyl, substituted or unsubstituted amino, alkynyl, alkenyl, alkyl, haloalkyl, cycloalkyl, alkoxy, alkylmercapto, heterocycloalkyl, deuterated alkyl, aryl or heteroaryl;
R^{a} and R^{b} are each independently hydrogen, deuterium, halogen, alkyl, haloalkyl, cycloalkyl, heterocycloalkyl or deuterated alkyl;
Alternatively, R^{a} and R^{b} form a 3-7 membered cycloalkyl or heterocycloalkyl group together with their co-connected carbon atom;
R^{c} and R^{d} are each independently hydrogen, deuterium, halogen, cyano, hydroxyl, substituted or unsubstituted amino, alkynyl, alkenyl, alkyl, haloalkyl, cycloalkyl, alkoxy, alkylmercapto, heterocycle alkyl, deuterated alkyl, aryl or heteroaryl;
Alternatively, R^{c} and R^{d} form a 3-7 membered cycloalkyl or heterocycloalkyl group together with their co-connected carbon atom;
Alternatively, R^{a} and R^{b}, R^{b} and Re, R^{c} and R^{d} each form a cycloalkyl or heterocycloalkyl group together with their co-connected carbon atom;
B is hydrogen, deuterium, hydroxyl, mercapto, substituted or unsubstituted amino;
R^{m} and Rⁿ are each independently hydrogen, deuterium, halogen, hydroxyl, alkyl, haloalkyl, cycloalkyl or deuterated alkyl;
Alternatively, R^{m} and Rⁿ form a cycloalkyl or heterocycloalkyl group together with their co-connected carbon atom;
x, n and p are 0, 1, 2, 3 or 4;
q is 0 or 1;
X is O, S or NH;
A is O or S, and when A-R¹⁰ is OH, R², not all R³ and R⁴ are hydrogen (H).

In a preferred embodiment of the present invention, the camptothecin compound represented by general formula (I) or a pharmaceutically acceptable salt thereof is a camptothecin compound represented by general formula (IV) or a pharmaceutically acceptable salt thereof:

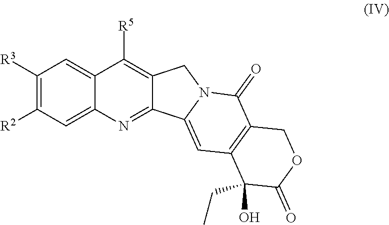

Wherein:
R² and R³ are each independently hydrogen, deuterium, halogen, hydroxyl, mercapto, C1-C4 alkyl sulfoxide, C1-C4 alkyl, C1-C4 haloalkyl, C1-C4 alkoxy, C1-C4 deuterium Alkyl, C2-C4 alkynyl or C2-C4 alkenyl;
Alternatively, R² and R³ together with their co-attached carbon atoms form the following cyclic structure:

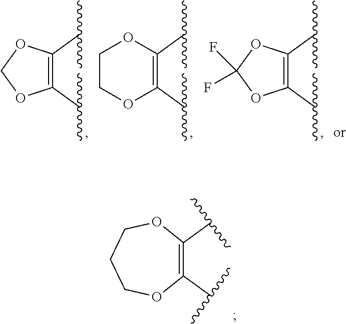

R⁵ is

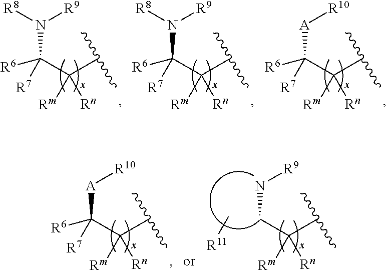

(the ring in

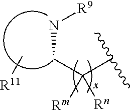

is 3-7 membered heterocycle);
R⁶ is hydrogen, deuterium, alkyl, haloalkyl, cycloalkyl, heterocycloalkyl or deuterated alkyl;
R⁷ is selected from alkyl, haloalkyl, cycloalkyl, heterocycloalkyl or deuterated alkyl;
Alternatively, R⁶ and R⁷ form a 3-7-membered cycloalkyl or heterocycloalkyl group together with their co-connected carbon atoms;
R⁸ and R⁹ are each independently hydrogen, deuterium, alkyl, haloalkyl, cycloalkyl, heterocycloalkyl, deuterated alkyl, aryl, heteroaryl,

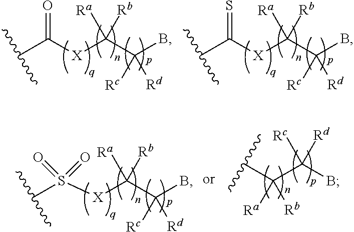

Alternatively, R⁸ and R⁹ form a 3-7-membered cycloalkyl or heterocycloalkyl group together with their co-connected nitrogen atoms;
R¹⁰ is hydrogen, alkyl, haloalkyl, cycloalkyl, heterocycloalkyl or deuterated alkyl,

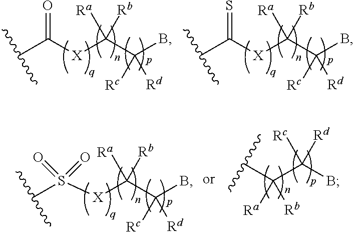

R¹¹ is one or more substituents of a 3-7 membered heterocycle, which is hydrogen, deuterium, halogen, cyano, hydroxyl, substituted or unsubstituted amino, alkyl, haloalkyl, cycloalkyl or heterocycloalkyl;
R^{a} and R^{b} are each independently hydrogen, deuterium, halogen, alkyl, haloalkyl or cycloalkyl;
Alternatively, R^{a} and R^{b} form a 3-7 membered cycloalkyl or heterocycloalkyl group together with their co-connected carbon atoms;
R^{c} and R^{d} are each independently hydrogen, deuterium, halogen, cyano, hydroxyl, substituted or unsubstituted amino, alkyl, haloalkyl, cycloalkyl, alkoxy, alkylmercapto, heterocycloalkyl or deuterated alkyl base;
Alternatively, R^{c} and R^{d} form a 3-7 membered cycloalkyl or heterocycloalkyl group together with their co-connected carbon atoms;
Alternatively, R^{a} and R^{b}, R^{b} and Re, R^{c} and R^{d} each form a 3-7 membered cycloalkyl or heterocycloalkyl group together with their carbon atoms;
B is hydroxyl, substituted or unsubstituted amino;
R^{m} and Rⁿ are each independently hydrogen, deuterium, halogen, hydroxyl, alkyl, haloalkyl, cycloalkyl or deuterated alkyl;
Alternatively, R^{m} and Rⁿ form a 3-7 membered cycloalkyl or heterocycloalkyl group together with their co-connected carbon atoms;
x is 0 or 1;
n and p are 0, 1 or 2;
q is 0 or 1;
X is O, S or NH;
A is O or S, and when A-R¹⁰ is OH, R² and R³ are not both hydrogen atoms (H).

In a preferred embodiment of the present invention, the camptothecin compound represented by general formula (I) or a pharmaceutically acceptable salt thereof is a camptothecin compound represented by general formula (I-1) or a pharmaceutically acceptable salt thereof:

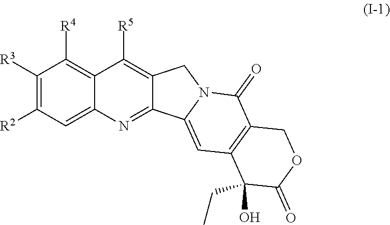

Wherein:
R² and R³ are each independently selected from H, D, F, Cl, CH₃, CD₃, CF₂H, CF₃, OH, SH, S(O)CH₃, S(O₂)CH₃, OCH₃ or SCH₃;
R⁴ is each independently selected from H, D, F, Cl, CH₃;
Alternatively, R² and R³ together with their co-connected carbon atom form a ring structure as follows:

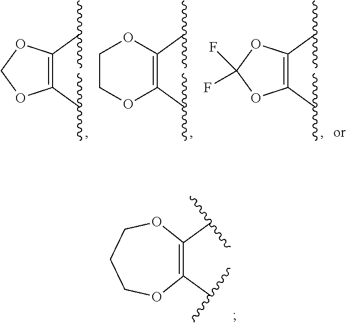

R⁵ is selected from

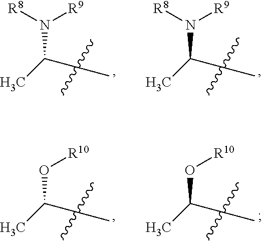

R⁸, R⁹ and R¹⁰ are as defined in any one of the invention.

In a preferred embodiment of the present invention, the camptothecin compound represented by general formula (I) or a pharmaceutically acceptable salt thereof is a camptothecin compound represented by general formula (V) or a pharmaceutically acceptable salt thereof:

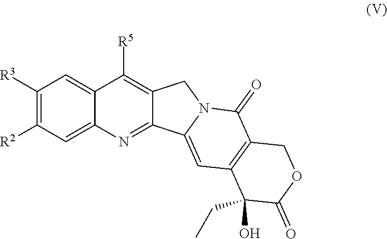

Wherein:
R² and R³ are each independently selected from H, D, F, Cl, CH₃, CD₃, CF₂H, CF₃, OH, SH, S(O)CH₃, S(O₂)CH₃, OCH₃ or SCH₃;
Alternatively, R² and R³ together with their co-connected carbon atom form a ring structure as follows:

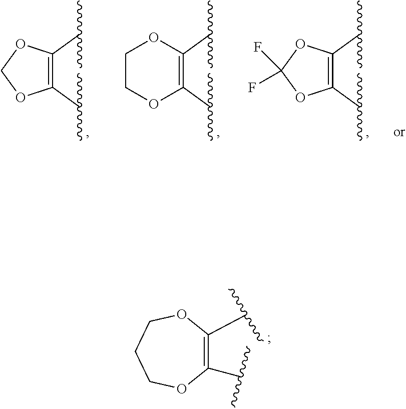

R⁵ is selected from the following structures:

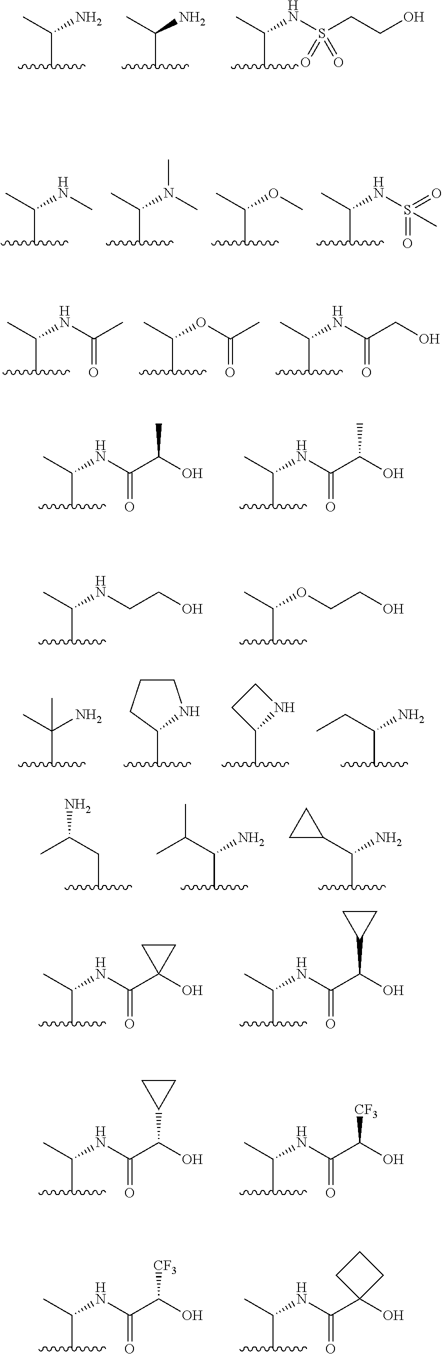

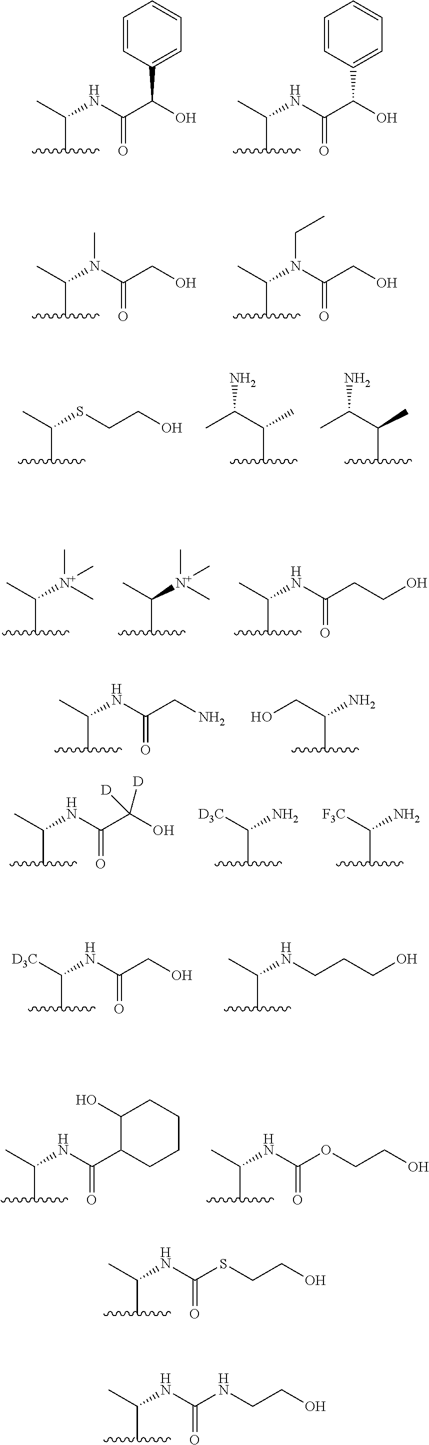

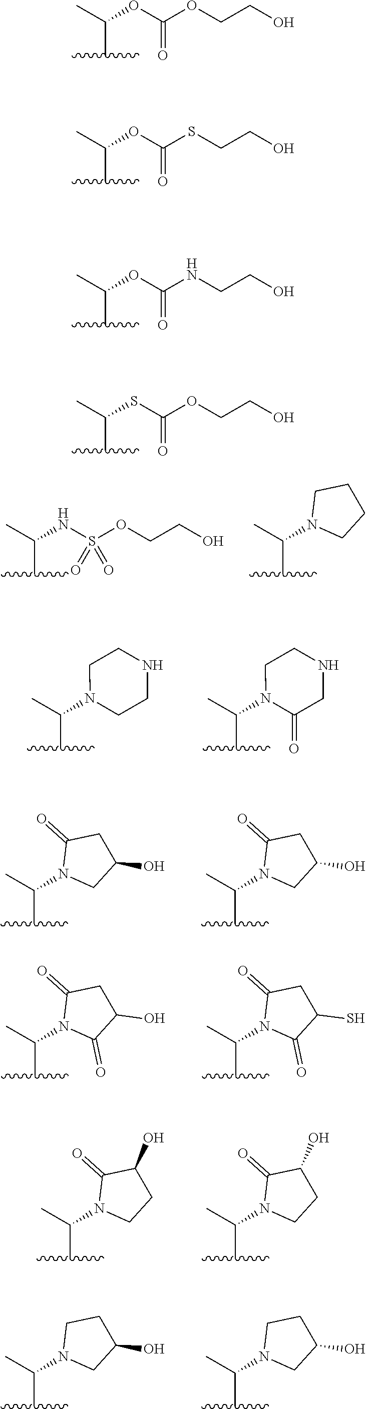

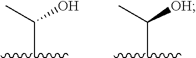

when R⁵ is selected from

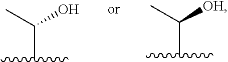

R² and R³ are not both hydrogen atoms (H).

In an embodiment of the present invention, R⁵ can also be the following structure:

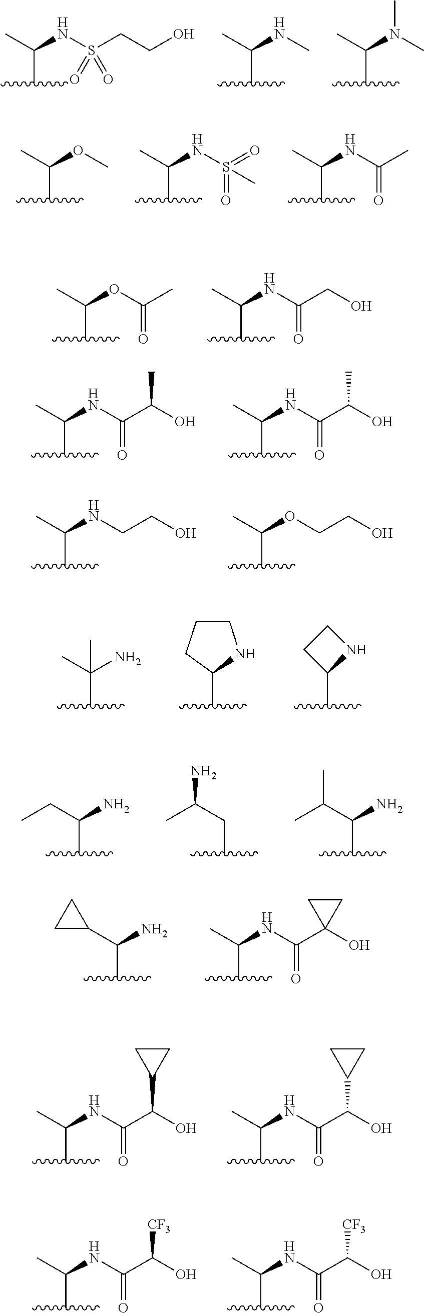

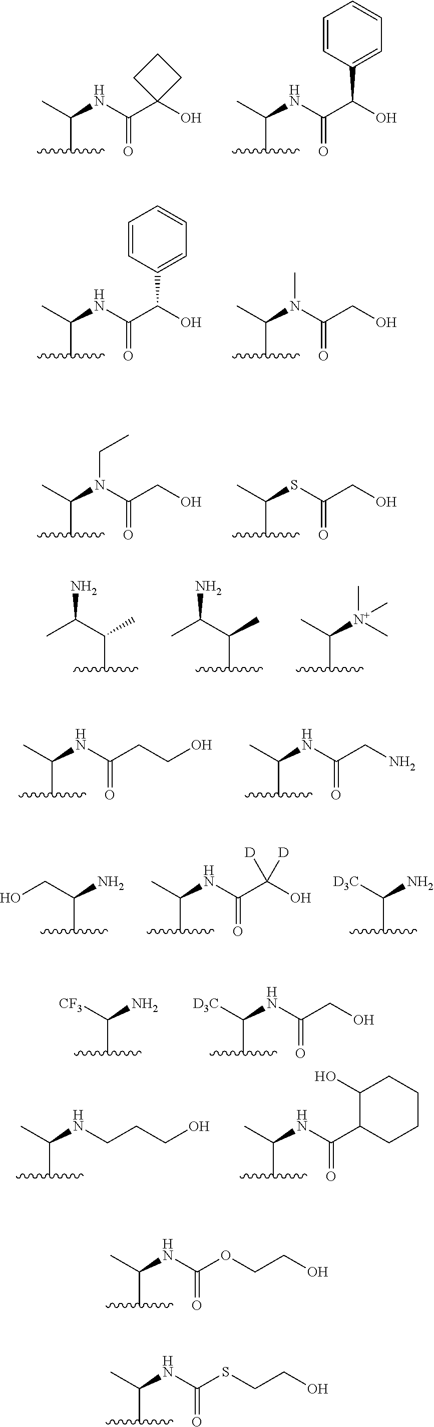

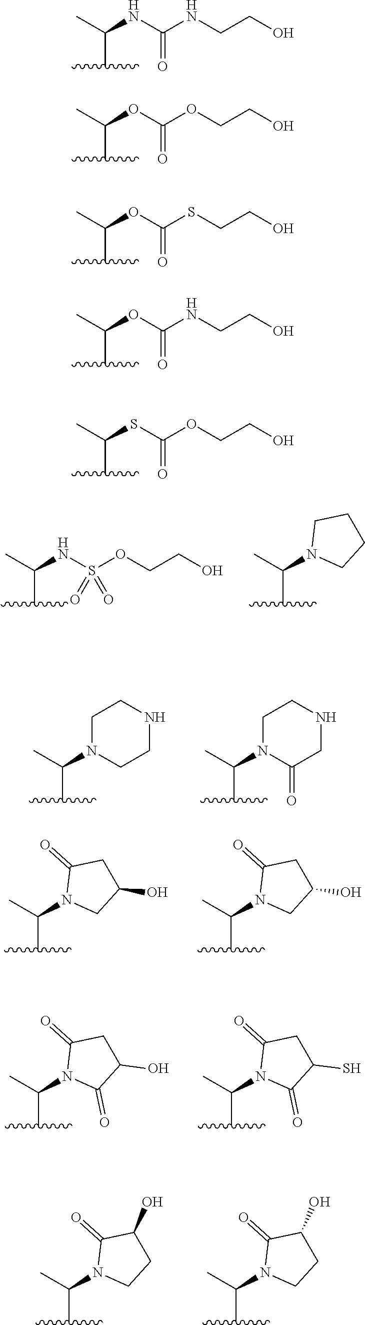

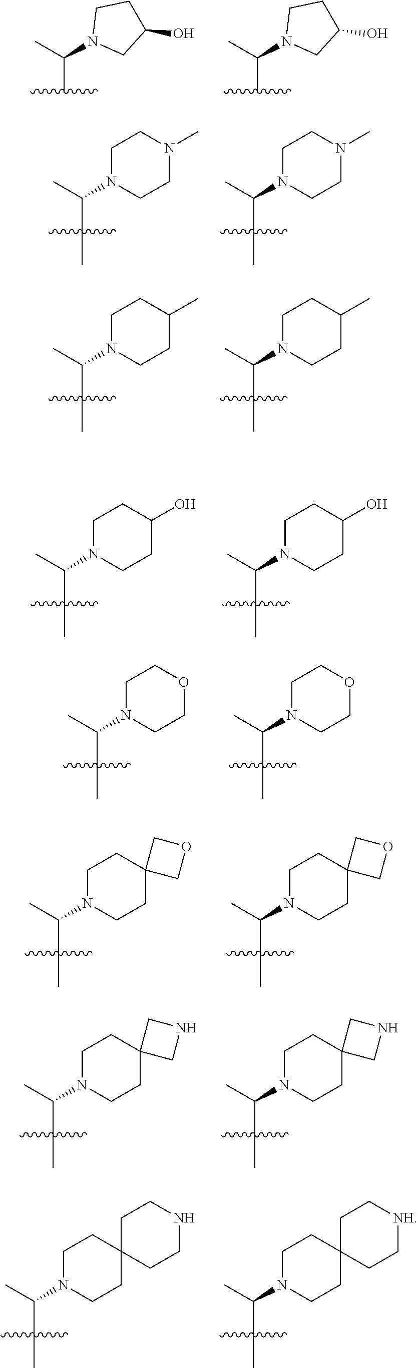

In a preferred embodiment of the present invention, the compound is any one of the following compounds:

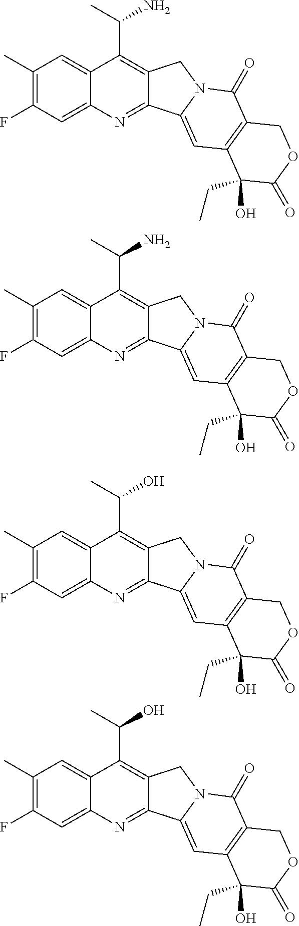 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@H](C)O ; CC[C@@]1(O)C(=O)OCc2c1cc1n(c2=O)Cc2c-1nc1cc(F)c(C)cc1c2[C@@H](C)O]

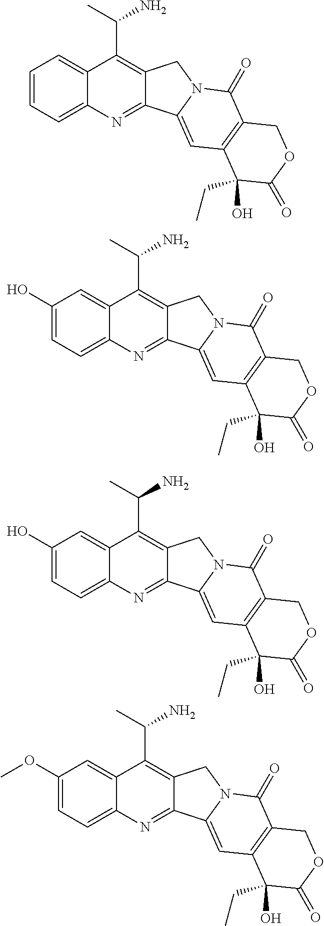

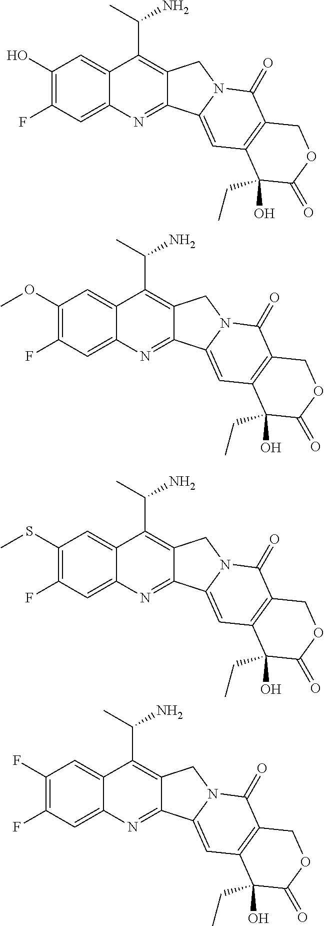 [SMILES: CC[C@@]1(O)C(=O)OCc2c1cc1n(c2=O)Cc2c-1nc1cc(F)c(O)cc1c2[C@H](C)N ; CC[C@@]1(O)C(=O)OCc2c1cc1n(c2=O)Cc2c-1nc1cc(F)c(OC)cc1c2[C@H](C)N ; CC[C@@]1(O)C(=O)OCc2c1cc1n(c2=O)Cc2c-1nc1cc(F)c(SC)cc1c2[C@H](C)N ; CC[C@@]1(O)C(=O)OCc2c1cc1n(c2=O)Cc2c-1nc1cc(F)c(F)cc1c2[C@H](C)N]

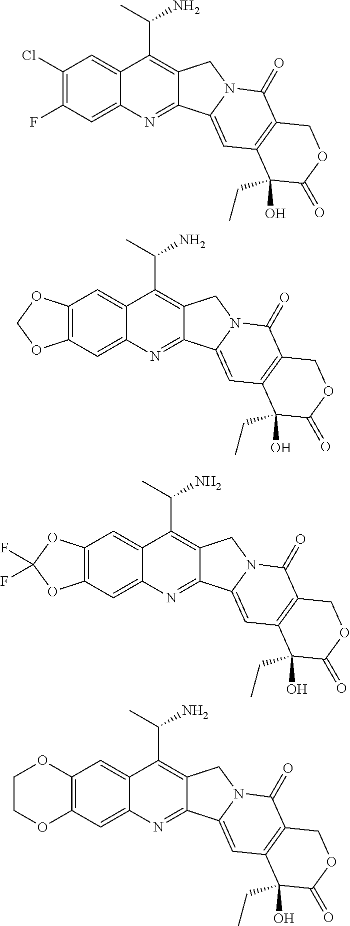

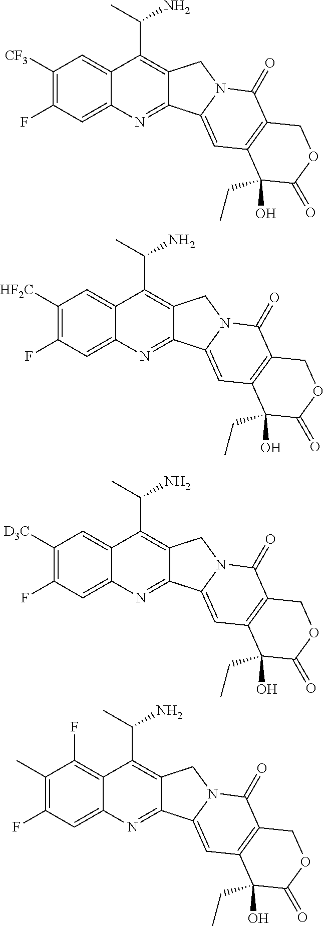

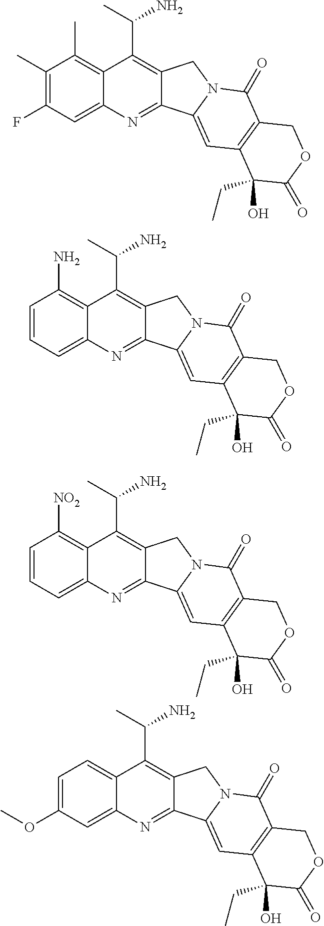

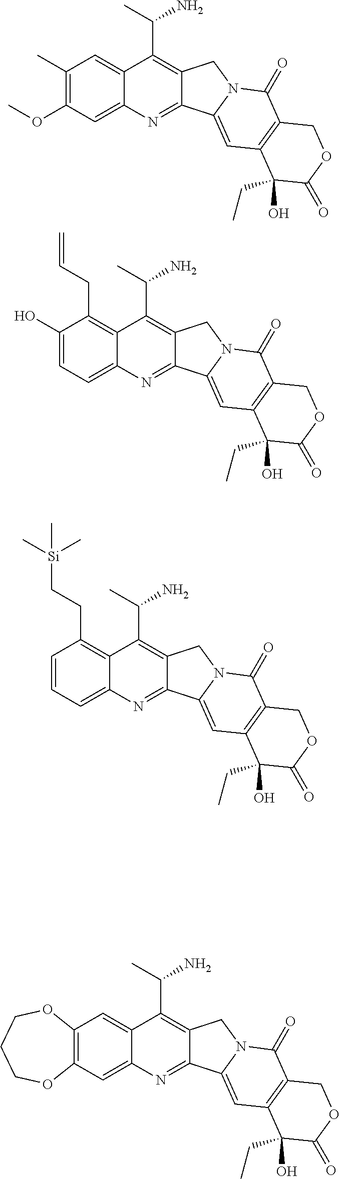 [SMILES: CC[C@@]1(O)C(=O)OCc2c1cc1n(c2=O)Cc2c-1nc1cc(OC)c(C)cc1c2[C@H](C)N ; C=CCc1c(O)ccc2nc3c(c([C@H](C)N)c12)Cn1c-3cc2c(c1=O)COC(=O)[C@]2(O)CC ; CC[C@@]1(O)C(=O)OCc2c1cc1n(c2=O)Cc2c-1nc1cccc(CC[Si](C)(C)C)c1c2[C@H](C)N ; CC[C@@]1(O)C(=O)OCc2c1cc1n(c2=O)Cc2c-1nc1cc3c(cc1c2[C@H](C)N)OCCCO3]

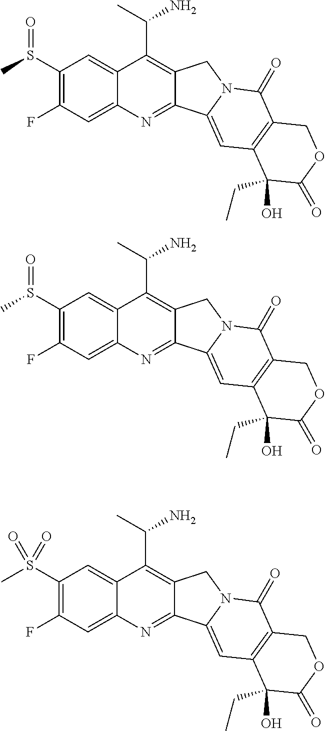 [SMILES: CC[C@@]1(O)C(=O)OCc2c1cc1n(c2=O)Cc2c-1nc1cc(F)c([S@@](C)=O)cc1c2[C@H](C)N ; CC[C@@]1(O)C(=O)OCc2c1cc1n(c2=O)Cc2c-1nc1cc(F)c([S@](C)=O)cc1c2[C@H](C)N ; CC[C@@]1(O)C(=O)OCc2c1cc1n(c2=O)Cc2c-1nc1cc(F)c(S(C)(=O)=O)cc1c2[C@H](C)N]

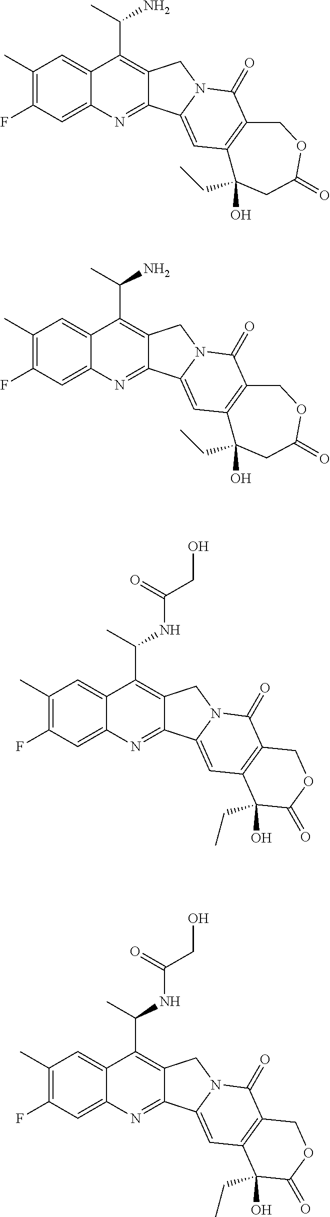 [SMILES: CC[C@@]1(O)CC(=O)OCc2c1cc1n(c2=O)Cc2c-1nc1cc(F)c(C)cc1c2[C@H](C)N ; CC[C@@]1(O)CC(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@H](C)NC(=O)CO ; CC[C@@]1(O)C(=O)OCc2c1cc1n(c2=O)Cc2c-1nc1cc(F)c(C)cc1c2[C@@H](C)NC(=O)CO]

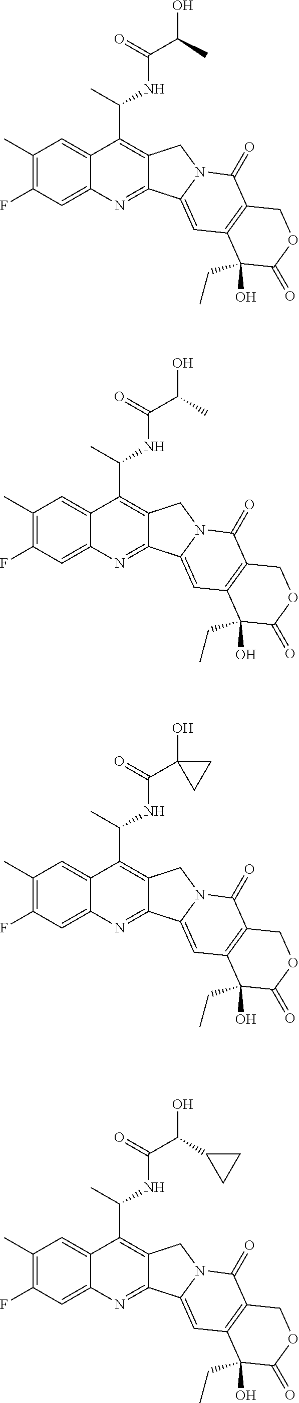 [SMILES: CC[C@@]1(O)C(=O)OCc2c1cc1n(c2=O)Cc2c-1nc1cc(F)c(C)cc1c2[C@H](C)NC(=O)[C@H](C)O ; CC[C@@]1(O)C(=O)OCc2c1cc1n(c2=O)Cc2c-1nc1cc(F)c(C)cc1c2[C@H](C)NC(=O)[C@@H](C)O ; CC[C@@]1(O)C(=O)OCc2c1cc1n(c2=O)Cc2c-1nc1cc(F)c(C)cc1c2[C@H](C)NC(=O)C1(O)CC1 ; CC[C@@]1(O)C(=O)OCc2c1cc1n(c2=O)Cc2c-1nc1cc(F)c(C)cc1c2[C@H](C)NC(=O)[C@H](O)C1CC1]

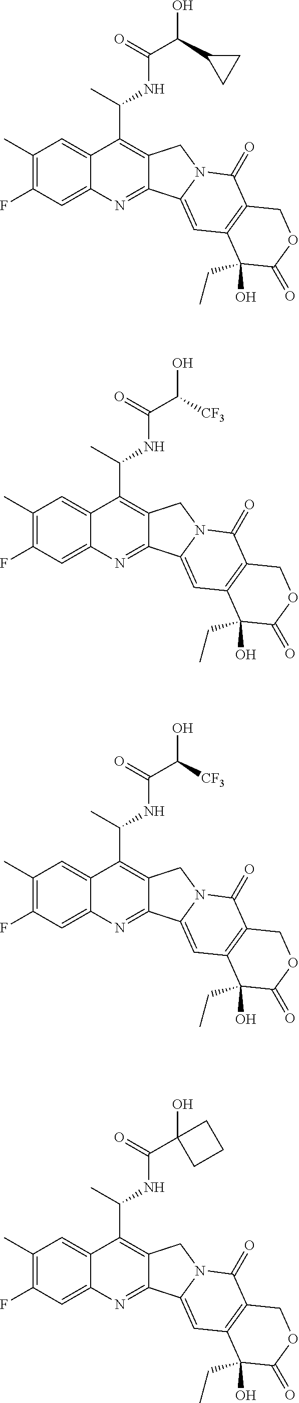 [SMILES: CC[C@@]1(O)C(=O)OCc2c1cc1n(c2=O)Cc2c-1nc1cc(F)c(C)cc1c2[C@H](C)NC(=O)[C@@H](O)C1CC1 ; CC[C@@]1(O)C(=O)OCc2c1cc1n(c2=O)Cc2c-1nc1cc(F)c(C)cc1c2[C@H](C)NC(=O)[C@H](O)C(F)(F)F ; CC[C@@]1(O)C(=O)OCc2c1cc1n(c2=O)Cc2c-1nc1cc(F)c(C)cc1c2[C@H](C)NC(=O)[C@@H](O)C(F)(F)F ; CC[C@@]1(O)C(=O)OCc2c1cc1n(c2=O)Cc2c-1nc1cc(F)c(C)cc1c2[C@H](C)NC(=O)C1(O)CCC1]

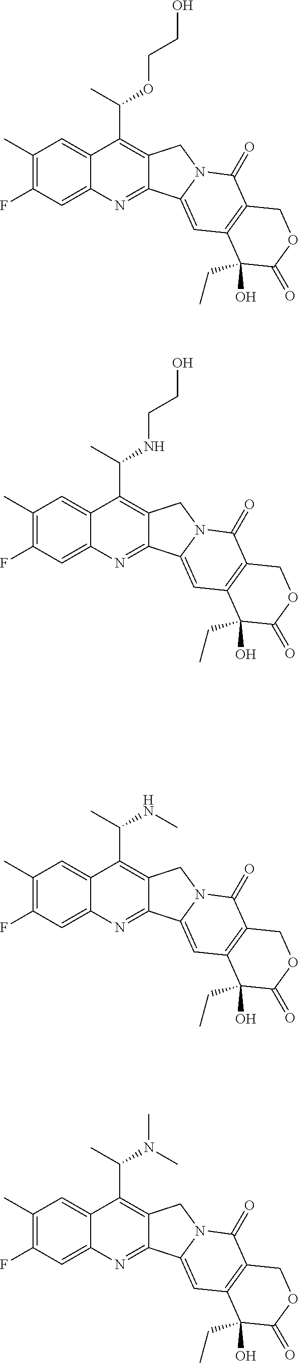 [SMILES: CC[C@@]1(O)C(=O)OCc2c1cc1n(c2=O)Cc2c-1nc1cc(F)c(C)cc1c2[C@H](C)OCCO ; CC[C@@]1(O)C(=O)OCc2c1cc1n(c2=O)Cc2c-1nc1cc(F)c(C)cc1c2[C@H](C)NCCO ; CC[C@@]1(O)C(=O)OCc2c1cc1n(c2=O)Cc2c-1nc1cc(F)c(C)cc1c2[C@H](C)NC ; CC[C@@]1(O)C(=O)OCc2c1cc1n(c2=O)Cc2c-1nc1cc(F)c(C)cc1c2[C@H](C)N(C)C]

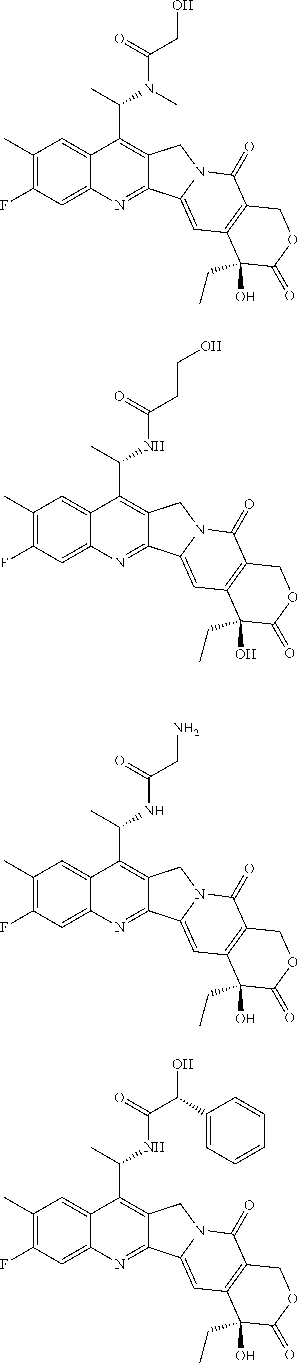

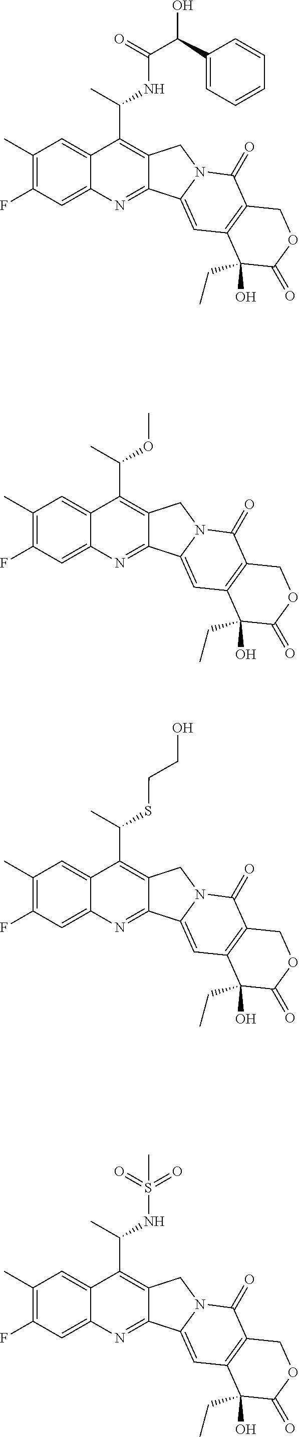 [SMILES: CC[C@@]1(O)C(=O)OCc2c1cc1n(c2=O)Cc2c-1nc1cc(F)c(C)cc1c2[C@H](C)NC(=O)[C@@H](O)c1ccccc1 ; CC[C@@]1(O)C(=O)OCc2c1cc1n(c2=O)Cc2c-1nc1cc(F)c(C)cc1c2[C@H](C)OC ; CC[C@@]1(O)C(=O)OCc2c1cc1n(c2=O)Cc2c-1nc1cc(F)c(C)cc1c2[C@H](C)SCCO ; CC[C@@]1(O)C(=O)OCc2c1cc1n(c2=O)Cc2c-1nc1cc(F)c(C)cc1c2[C@H](C)NS(C)(=O)=O]

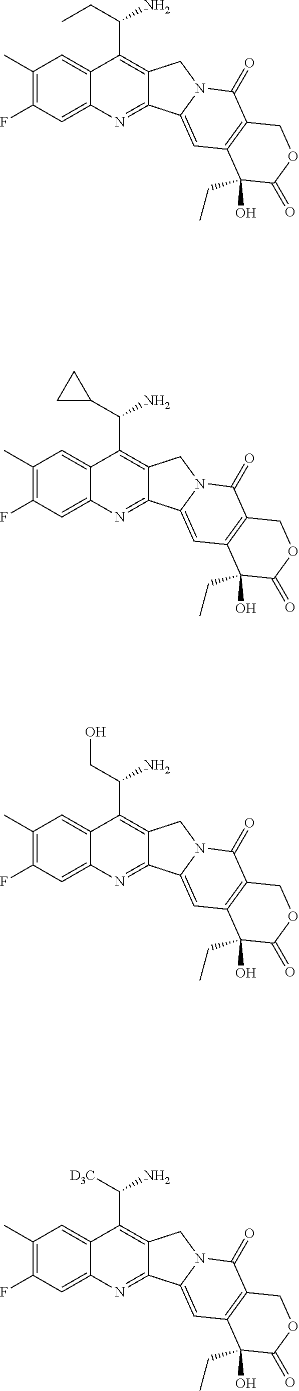 [SMILES: CC[C@H](N)c1c2c(nc3cc(F)c(C)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2[C@@H](N)C1CC1 ; CC[C@@]1(O)C(=O)OCc2c1cc1n(c2=O)Cc2c-1nc1cc(F)c(C)cc1c2[C@@H](N)CO ; [2H]C([2H])([2H])[C@H](N)c1c2c(nc3cc(F)c(C)cc13)-c1cc3c(c(=O)n1C2)COC(=O)[C@]3(O)CC]

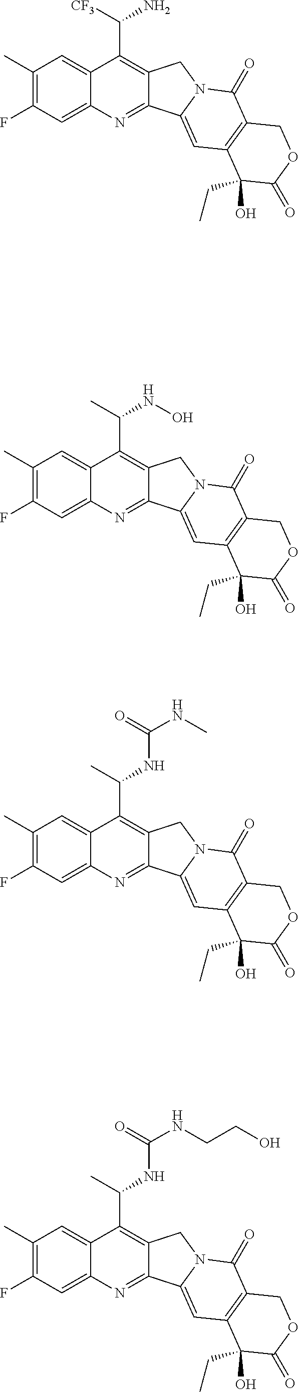 [SMILES: CC[C@@]1(O)C(=O)OCc2c1cc1n(c2=O)Cc2c-1nc1cc(F)c(C)cc1c2[C@@H](N)C(F)(F)F ; CC[C@@]1(O)C(=O)OCc2c1cc1n(c2=O)Cc2c-1nc1cc(F)c(C)cc1c2[C@H](C)NO ; CC[C@@]1(O)C(=O)OCc2c1cc1n(c2=O)Cc2c-1nc1cc(F)c(C)cc1c2[C@H](C)NC(=O)NC ; CC[C@@]1(O)C(=O)OCc2c1cc1n(c2=O)Cc2c-1nc1cc(F)c(C)cc1c2[C@H](C)NC(=O)NCCO]

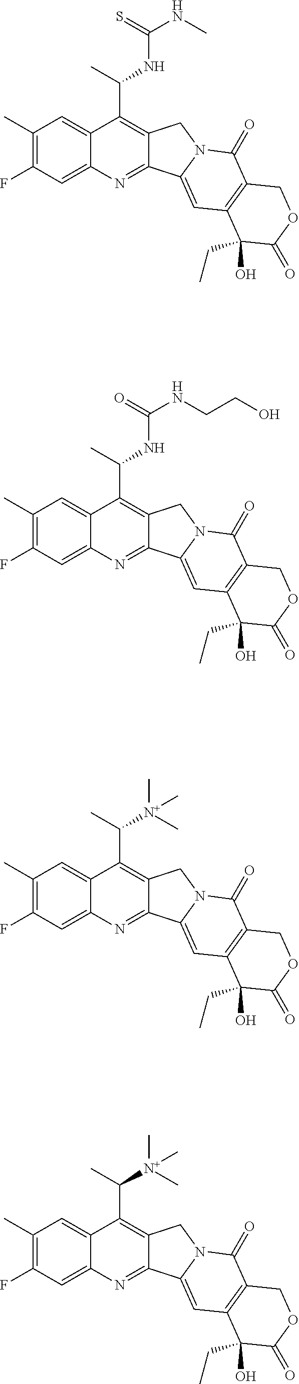 [SMILES: CC[C@@]1(O)C(=O)OCc2c1cc1n(c2=O)Cc2c-1nc1cc(F)c(C)cc1c2[C@H](C)NC(=S)NC ; CC[C@@]1(O)C(=O)OCc2c1cc1n(c2=O)Cc2c-1nc1cc(F)c(C)cc1c2[C@H](C)NC(=O)NCCO ; CC[C@@]1(O)C(=O)OCc2c1cc1n(c2=O)Cc2c-1nc1cc(F)c(C)cc1c2[C@H](C)[N+](C)(C)C ; CC[C@@]1(O)C(=O)OCc2c1cc1n(c2=O)Cc2c-1nc1cc(F)c(C)cc1c2[C@@H](C)[N+](C)(C)C]

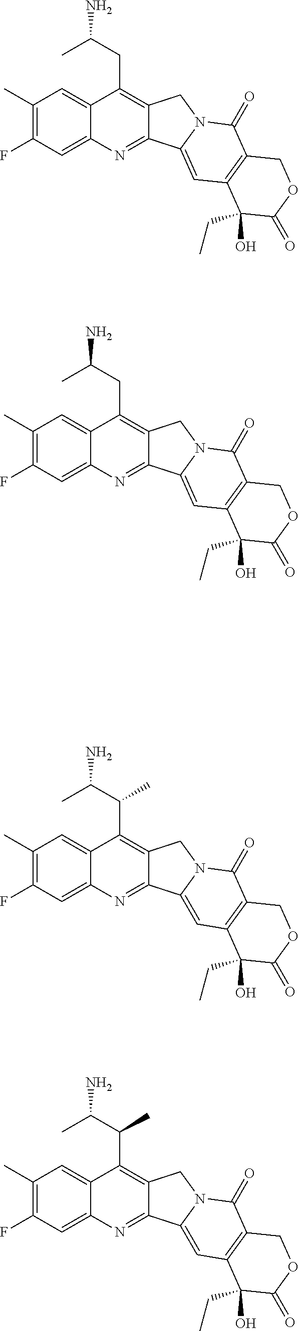 [SMILES: CC[C@@]1(O)C(=O)OCc2c1cc1n(c2=O)Cc2c-1nc1cc(F)c(C)cc1c2C[C@H](C)N ; CC[C@@]1(O)C(=O)OCc2c1cc1n(c2=O)Cc2c-1nc1cc(F)c(C)cc1c2C[C@@H](C)N ; CC[C@@]1(O)C(=O)OCc2c1cc1n(c2=O)Cc2c-1nc1cc(F)c(C)cc1c2[C@@H](C)[C@H](C)N ; CC[C@@]1(O)C(=O)OCc2c1cc1n(c2=O)Cc2c-1nc1cc(F)c(C)cc1c2[C@H](C)[C@H](C)N]

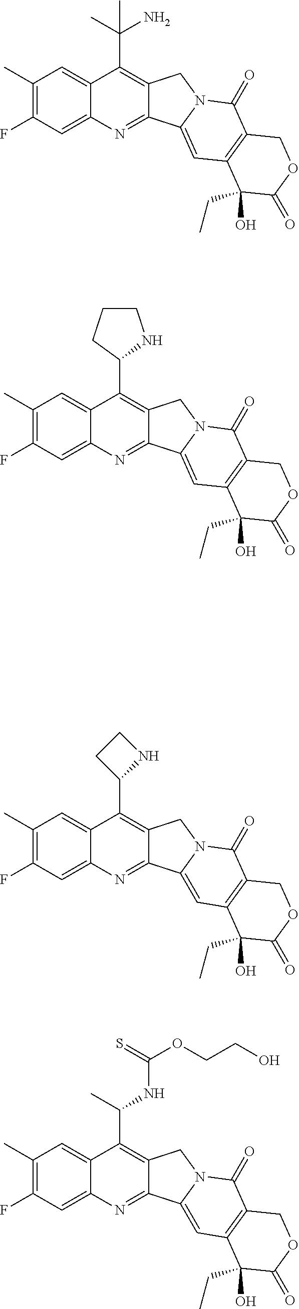 [SMILES: CC[C@@]1(O)C(=O)OCc2c1cc1n(c2=O)Cc2c-1nc1cc(F)c(C)cc1c2C(C)(C)N ; CC[C@@]1(O)C(=O)OCc2c1cc1n(c2=O)Cc2c-1nc1cc(F)c(C)cc1c2[C@@H]1CCCN1 ; CC[C@@]1(O)C(=O)OCc2c1cc1n(c2=O)Cc2c-1nc1cc(F)c(C)cc1c2[C@@H]1CCN1 ; CC[C@@]1(O)C(=O)OCc2c1cc1n(c2=O)Cc2c-1nc1cc(F)c(C)cc1c2[C@H](C)NC(=S)OCCO]

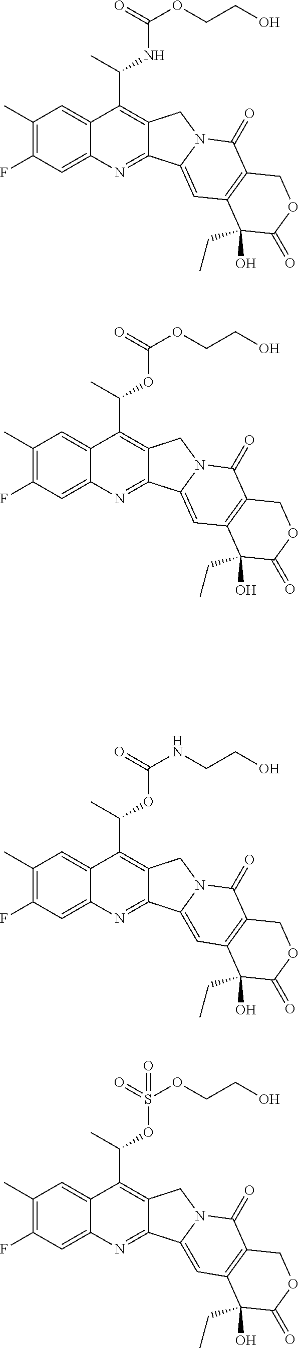 [SMILES: CC[C@@]1(O)C(=O)OCc2c1cc1n(c2=O)Cc2c-1nc1cc(F)c(C)cc1c2[C@H](C)NC(=O)OCCO ; CC[C@@]1(O)C(=O)OCc2c1cc1n(c2=O)Cc2c-1nc1cc(F)c(C)cc1c2[C@H](C)OC(=O)OCCO ; CC[C@@]1(O)C(=O)OCc2c1cc1n(c2=O)Cc2c-1nc1cc(F)c(C)cc1c2[C@H](C)OC(=O)NCCO ; CC[C@@]1(O)C(=O)OCc2c1cc1n(c2=O)Cc2c-1nc1cc(F)c(C)cc1c2[C@H](C)OS(=O)(=O)OCCO]

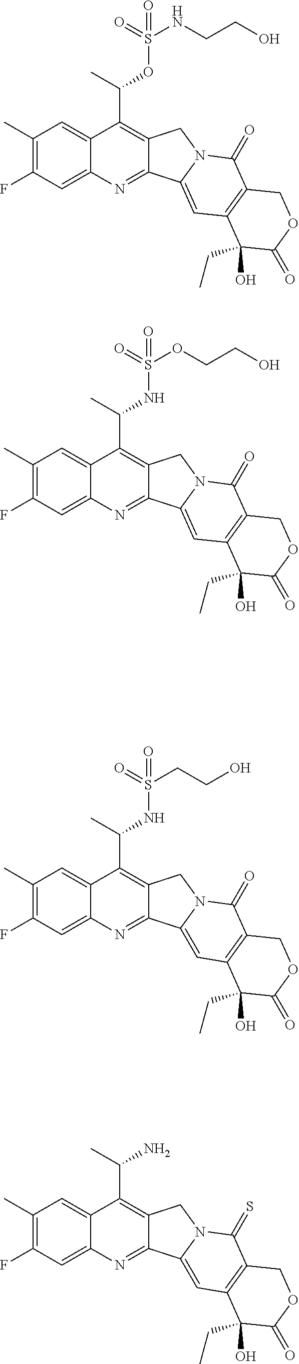 [SMILES: CC[C@@]1(O)C(=O)OCc2c1cc1n(c2=O)Cc2c-1nc1cc(F)c(C)cc1c2[C@H](C)OS(=O)(=O)NCCO ; CC[C@@]1(O)C(=O)OCc2c1cc1n(c2=O)Cc2c-1nc1cc(F)c(C)cc1c2[C@H](C)NS(=O)(=O)OCCO ; CC[C@@]1(O)C(=O)OCc2c1cc1n(c2=O)Cc2c-1nc1cc(F)c(C)cc1c2[C@H](C)NS(=O)(=O)CCO ; CC[C@@]1(O)C(=O)OCc2c1cc1n(c2=S)Cc2c-1nc1cc(F)c(C)cc1c2[C@H](C)N]

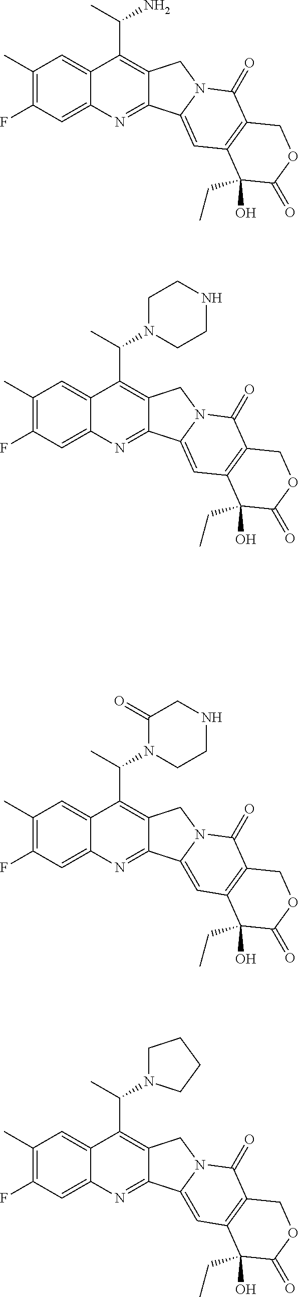 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@H](C)N1CCNCC1 ; CC[C@@]1(O)C(=O)OCc2c1cc1n(c2=O)Cc2c-1nc1cc(F)c(C)cc1c2[C@H](C)N1CCNCC1=O ; CC[C@@]1(O)C(=O)OCc2c1cc1n(c2=O)Cc2c-1nc1cc(F)c(C)cc1c2[C@H](C)N1CCCC1]

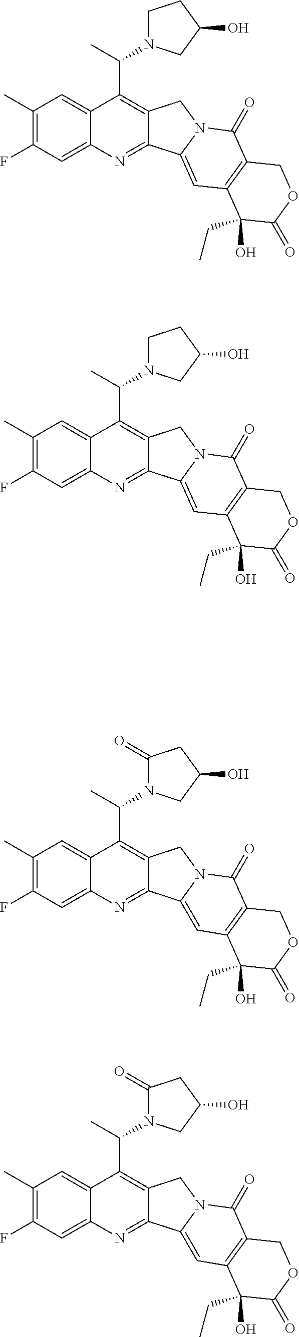 [SMILES: CC[C@@]1(O)C(=O)OCc2c1cc1n(c2=O)Cc2c-1nc1cc(F)c(C)cc1c2[C@H](C)N1CC[C@@H](O)C1 ; CC[C@@]1(O)C(=O)OCc2c1cc1n(c2=O)Cc2c-1nc1cc(F)c(C)cc1c2[C@H](C)N1CC[C@H](O)C1 ; CC[C@@]1(O)C(=O)OCc2c1cc1n(c2=O)Cc2c-1nc1cc(F)c(C)cc1c2[C@H](C)N1C[C@H](O)CC1=O ; CC[C@@]1(O)C(=O)OCc2c1cc1n(c2=O)Cc2c-1nc1cc(F)c(C)cc1c2[C@H](C)N1C[C@@H](O)CC1=O]

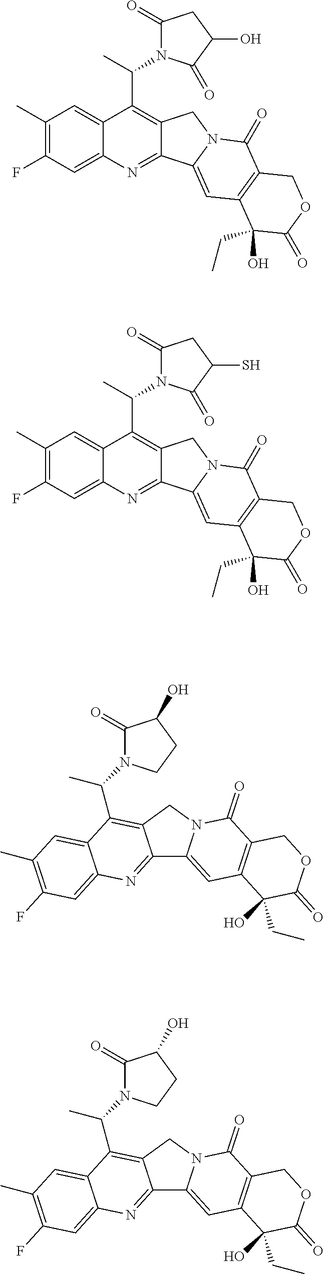 [SMILES: CC[C@@]1(O)C(=O)OCc2c1cc1n(c2=O)Cc2c-1nc1cc(F)c(C)cc1c2[C@H](C)N1C(=O)CC(O)C1=O ; CC[C@@]1(O)C(=O)OCc2c1cc1n(c2=O)Cc2c-1nc1cc(F)c(C)cc1c2[C@H](C)N1C(=O)CC(S)C1=O ; CC[C@@]1(O)C(=O)OCc2c1cc1n(c2=O)Cc2c-1nc1cc(F)c(C)cc1c2[C@H](C)N1CC[C@H](O)C1=O ; CC[C@@]1(O)C(=O)OCc2c1cc1n(c2=O)Cc2c-1nc1cc(F)c(C)cc1c2[C@H](C)N1CC[C@@H](O)C1=O]

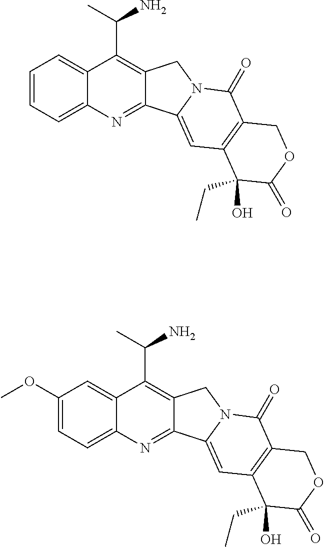 [SMILES: CC[C@@]1(O)C(=O)OCc2c1cc1n(c2=O)Cc2c-1nc1ccccc1c2[C@@H](C)N ; CC[C@@]1(O)C(=O)OCc2c1cc1n(c2=O)Cc2c-1nc1ccc(OC)cc1c2[C@@H](C)N]

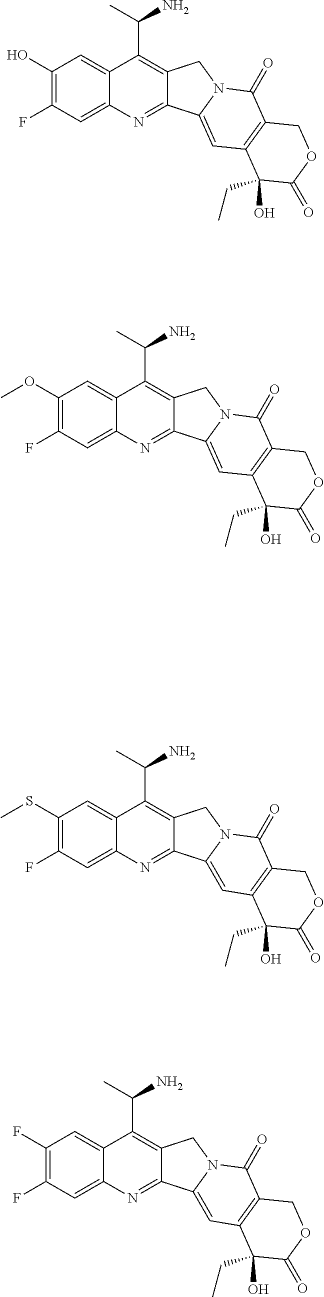 [SMILES: CC[C@@]1(O)C(=O)OCc2c1cc1n(c2=O)Cc2c-1nc1cc(F)c(O)cc1c2[C@@H](C)N ; CC[C@@]1(O)C(=O)OCc2c1cc1n(c2=O)Cc2c-1nc1cc(F)c(OC)cc1c2[C@@H](C)N ; CC[C@@]1(O)C(=O)OCc2c1cc1n(c2=O)Cc2c-1nc1cc(F)c(SC)cc1c2[C@@H](C)N ; CC[C@@]1(O)C(=O)OCc2c1cc1n(c2=O)Cc2c-1nc1cc(F)c(F)cc1c2[C@@H](C)N]

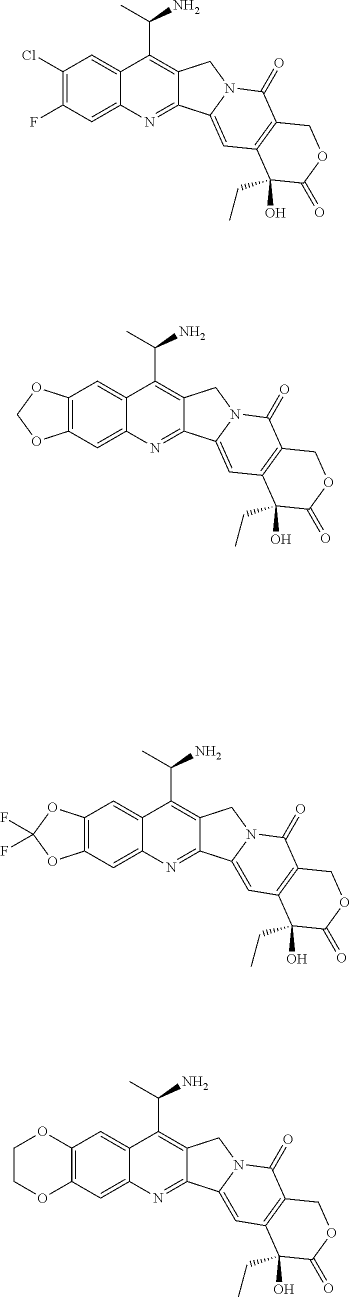 [SMILES: CC[C@@]1(O)C(=O)OCc2c1cc1n(c2=O)Cc2c-1nc1cc(F)c(Cl)cc1c2[C@@H](C)N ; CC[C@@]1(O)C(=O)OCc2c1cc1n(c2=O)Cc2c-1nc1cc3c(cc1c2[C@@H](C)N)OCO3 ; CC[C@@]1(O)C(=O)OCc2c1cc1n(c2=O)Cc2c-1nc1cc3c(cc1c2[C@@H](C)N)OC(F)(F)O3 ; CC[C@@]1(O)C(=O)OCc2c1cc1n(c2=O)Cc2c-1nc1cc3c(cc1c2[C@@H](C)N)OCCO3]

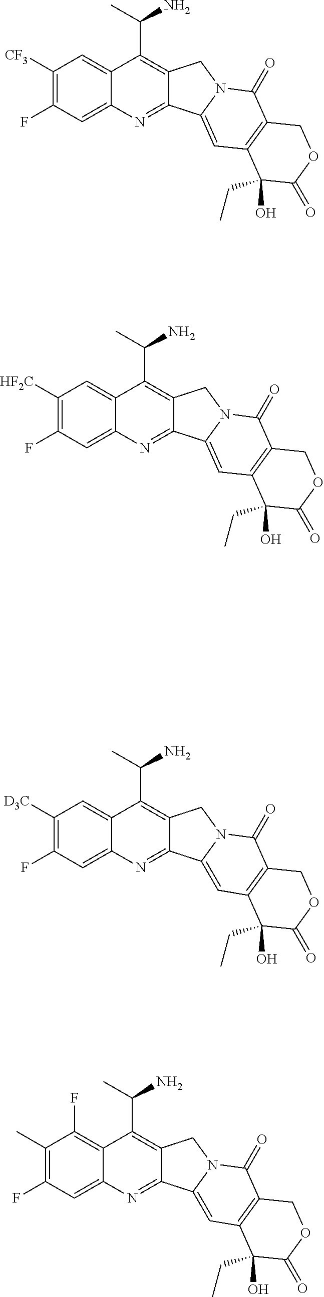

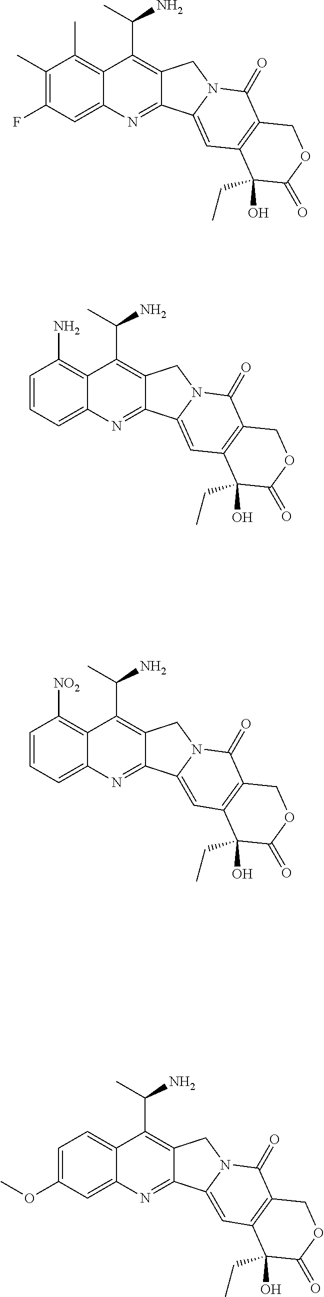 [SMILES: CC[C@@]1(O)C(=O)OCc2c1cc1n(c2=O)Cc2c-1nc1cc(F)c(C)c(C)c1c2[C@@H](C)N ; CC[C@@]1(O)C(=O)OCc2c1cc1n(c2=O)Cc2c-1nc1cccc(N)c1c2[C@@H](C)N ; CC[C@@]1(O)C(=O)OCc2c1cc1n(c2=O)Cc2c-1nc1cccc([N+](=O)[O-])c1c2[C@@H](C)N ; CC[C@@]1(O)C(=O)OCc2c1cc1n(c2=O)Cc2c-1nc1cc(OC)ccc1c2[C@@H](C)N]

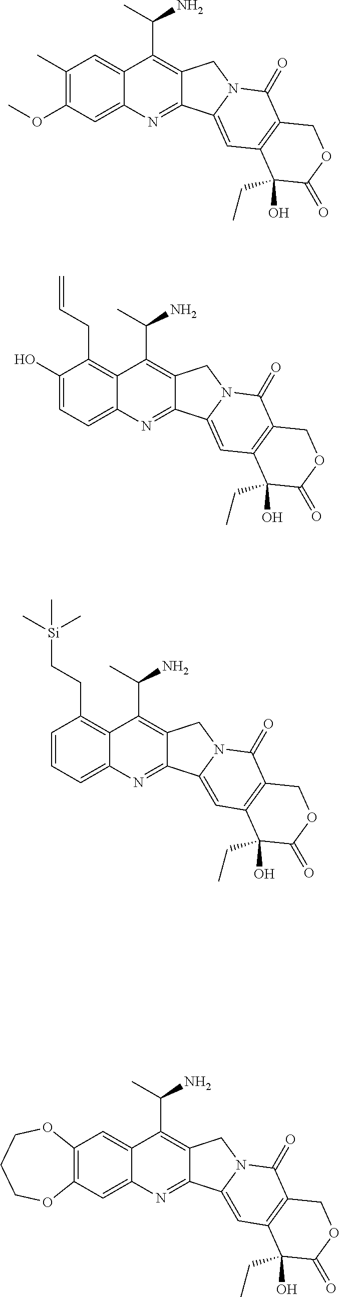 [SMILES: CC[C@@]1(O)C(=O)OCc2c1cc1n(c2=O)Cc2c-1nc1cc(OC)c(C)cc1c2[C@@H](C)N ; C=CCc1c(O)ccc2nc3c(c([C@@H](C)N)c12)Cn1c-3cc2c(c1=O)COC(=O)[C@]2(O)CC ; CC[C@@]1(O)C(=O)OCc2c1cc1n(c2=O)Cc2c-1nc1cccc(CC[Si](C)(C)C)c1c2[C@@H](C)N ; CC[C@@]1(O)C(=O)OCc2c1cc1n(c2=O)Cc2c-1nc1cc3c(cc1c2[C@@H](C)N)OCCCO3]

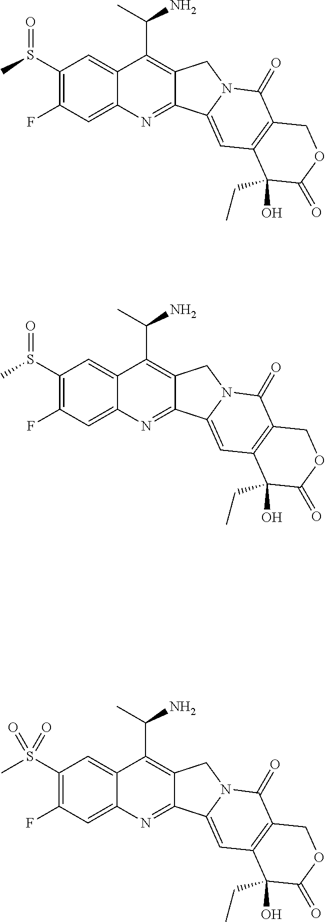 [SMILES: CC[C@@]1(O)C(=O)OCc2c1cc1n(c2=O)Cc2c-1nc1cc(F)c([S@@](C)=O)cc1c2[C@@H](C)N ; CC[C@@]1(O)C(=O)OCc2c1cc1n(c2=O)Cc2c-1nc1cc(F)c([S@](C)=O)cc1c2[C@@H](C)N ; CC[C@@]1(O)C(=O)OCc2c1cc1n(c2=O)Cc2c-1nc1cc(F)c(S(C)(=O)=O)cc1c2[C@@H](C)N]

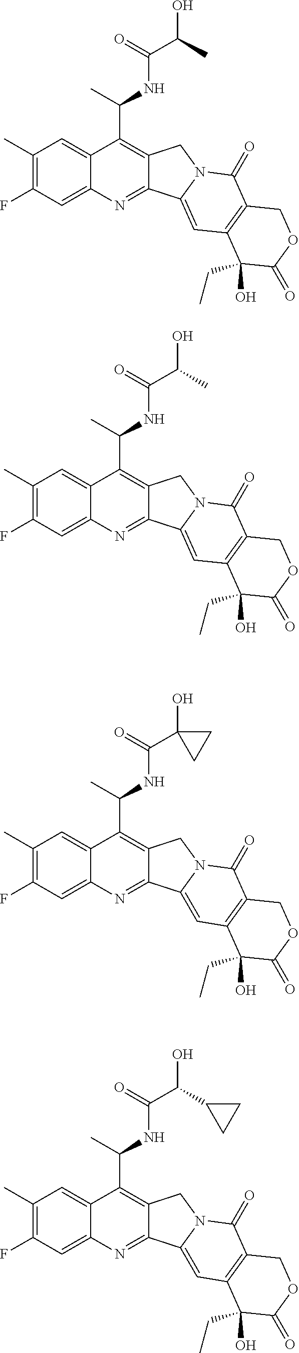

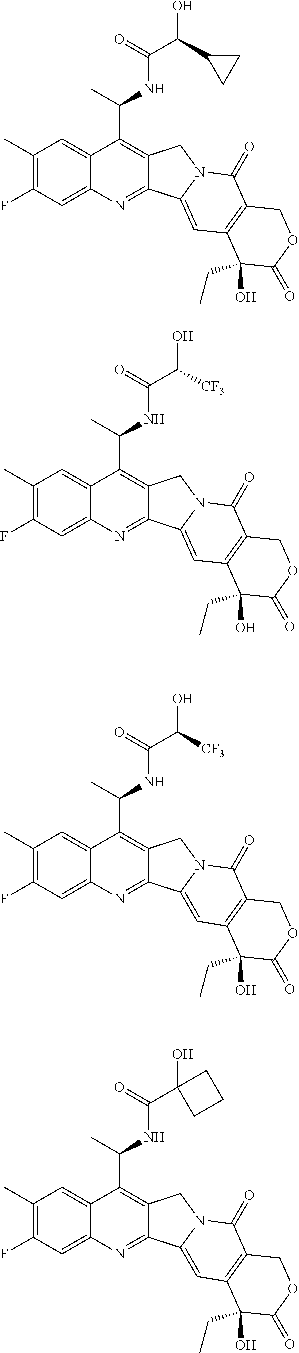

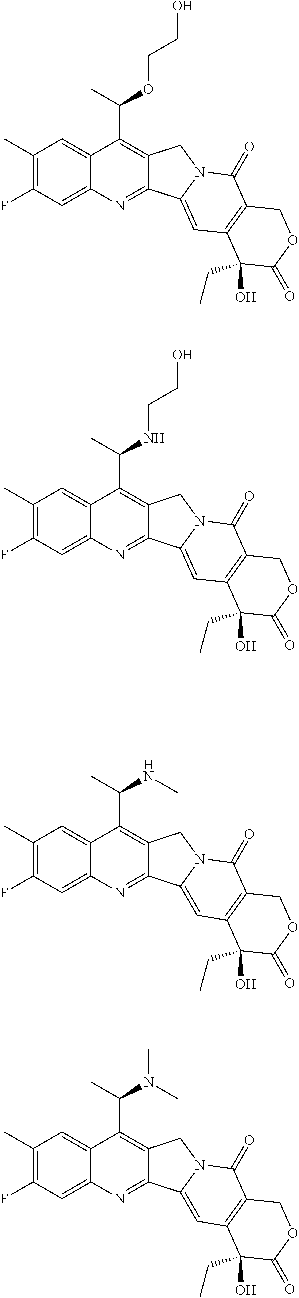 [SMILES: CC[C@@]1(O)C(=O)OCc2c1cc1n(c2=O)Cc2c-1nc1cc(F)c(C)cc1c2[C@@H](C)OCCO ; CC[C@@]1(O)C(=O)OCc2c1cc1n(c2=O)Cc2c-1nc1cc(F)c(C)cc1c2[C@@H](C)NCCO ; CC[C@@]1(O)C(=O)OCc2c1cc1n(c2=O)Cc2c-1nc1cc(F)c(C)cc1c2[C@@H](C)NC ; CC[C@@]1(O)C(=O)OCc2c1cc1n(c2=O)Cc2c-1nc1cc(F)c(C)cc1c2[C@@H](C)N(C)C]

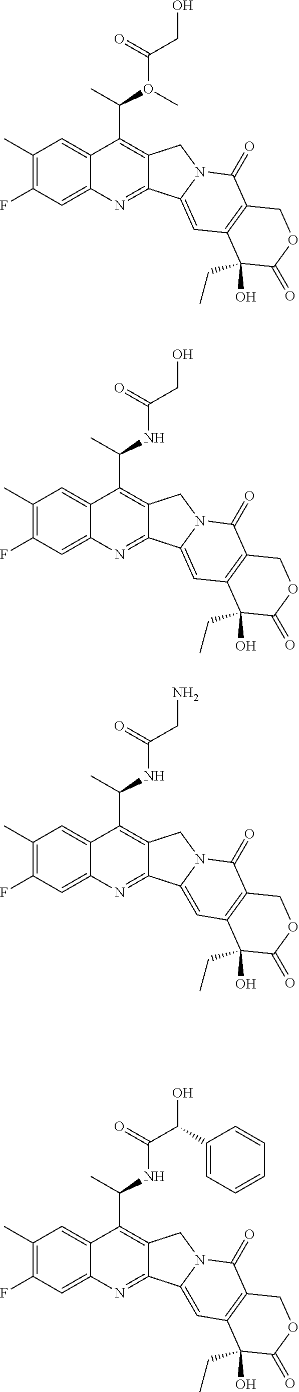

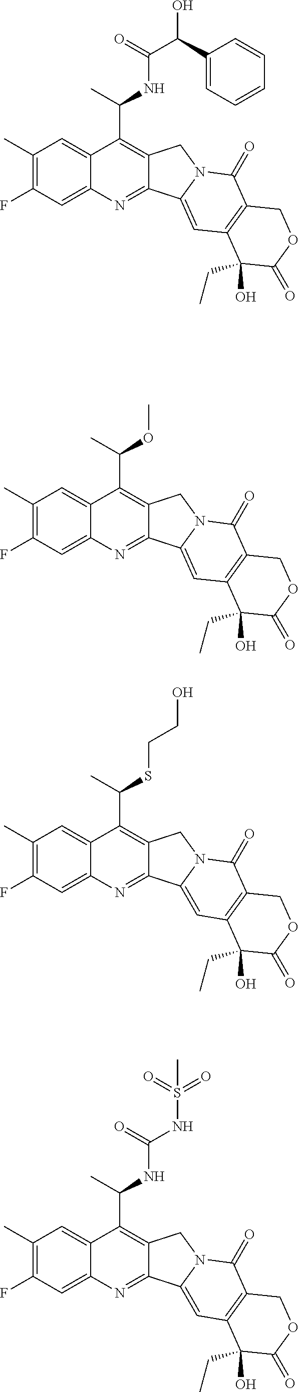

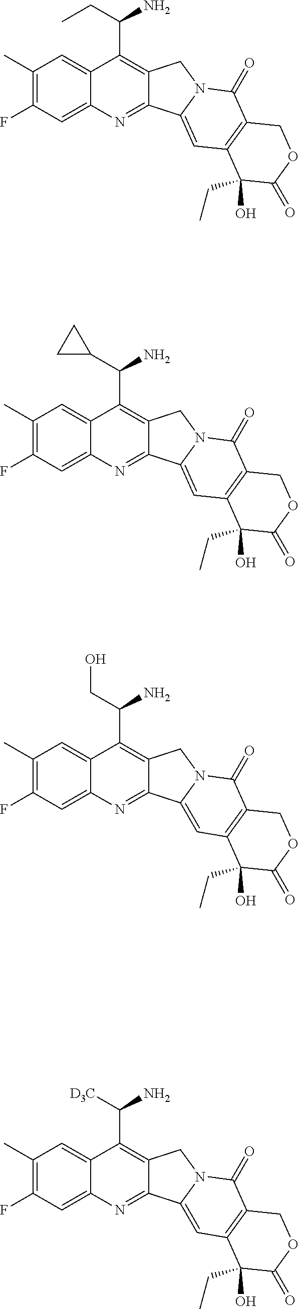 [SMILES: CC[C@@H](N)c1c2c(nc3cc(F)c(C)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2[C@H](N)C1CC1 ; CC[C@@]1(O)C(=O)OCc2c1cc1n(c2=O)Cc2c-1nc1cc(F)c(C)cc1c2[C@H](N)CO ; [2H]C([2H])([2H])[C@@H](N)c1c2c(nc3cc(F)c(C)cc13)-c1cc3c(c(=O)n1C2)COC(=O)[C@]3(O)CC]

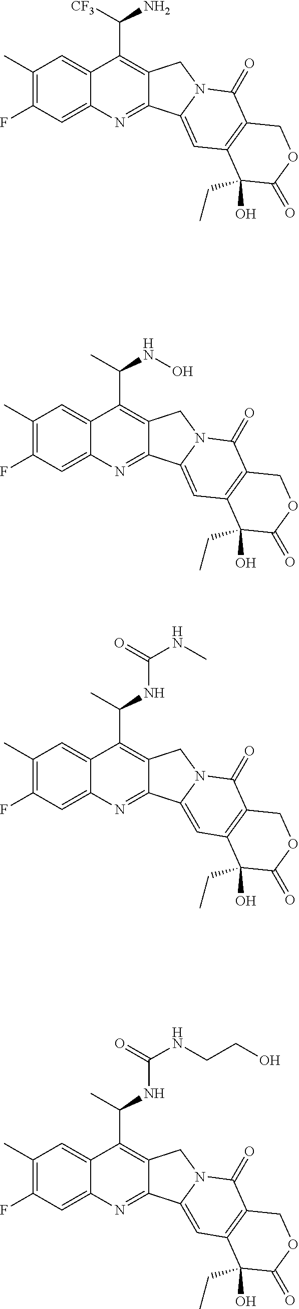 [SMILES: CC[C@@]1(O)C(=O)OCc2c1cc1n(c2=O)Cc2c-1nc1cc(F)c(C)cc1c2[C@H](N)C(F)(F)F ; CC[C@@]1(O)C(=O)OCc2c1cc1n(c2=O)Cc2c-1nc1cc(F)c(C)cc1c2[C@@H](C)NO ; CC[C@@]1(O)C(=O)OCc2c1cc1n(c2=O)Cc2c-1nc1cc(F)c(C)cc1c2[C@@H](C)NC(=O)NC ; CC[C@@]1(O)C(=O)OCc2c1cc1n(c2=O)Cc2c-1nc1cc(F)c(C)cc1c2[C@@H](C)NC(=O)NCCO]

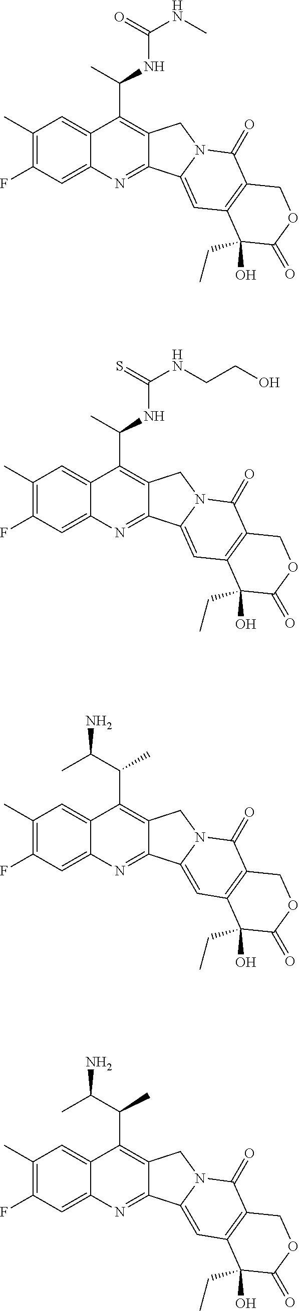 [SMILES: CC[C@@]1(O)C(=O)OCc2c1cc1n(c2=O)Cc2c-1nc1cc(F)c(C)cc1c2[C@@H](C)NC(=O)NC ; CC[C@@]1(O)C(=O)OCc2c1cc1n(c2=O)Cc2c-1nc1cc(F)c(C)cc1c2[C@@H](C)NC(=S)NCCO ; CC[C@@]1(O)C(=O)OCc2c1cc1n(c2=O)Cc2c-1nc1cc(F)c(C)cc1c2[C@@H](C)[C@@H](C)N ; CC[C@@]1(O)C(=O)OCc2c1cc1n(c2=O)Cc2c-1nc1cc(F)c(C)cc1c2[C@H](C)[C@@H](C)N]

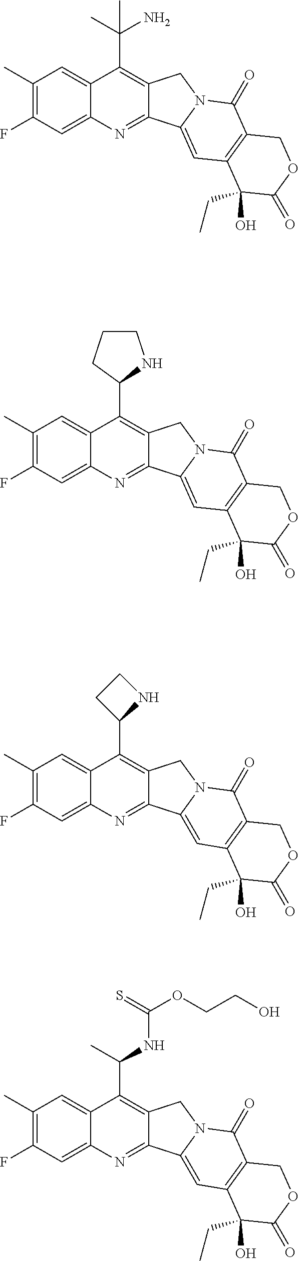 [SMILES: CC[C@@]1(O)C(=O)OCc2c1cc1n(c2=O)Cc2c-1nc1cc(F)c(C)cc1c2C(C)(C)N ; CC[C@@]1(O)C(=O)OCc2c1cc1n(c2=O)Cc2c-1nc1cc(F)c(C)cc1c2[C@H]1CCCN1 ; CC[C@@]1(O)C(=O)OCc2c1cc1n(c2=O)Cc2c-1nc1cc(F)c(C)cc1c2[C@H]1CCN1 ; CC[C@@]1(O)C(=O)OCc2c1cc1n(c2=O)Cc2c-1nc1cc(F)c(C)cc1c2[C@@H](C)NC(=S)OCCO]

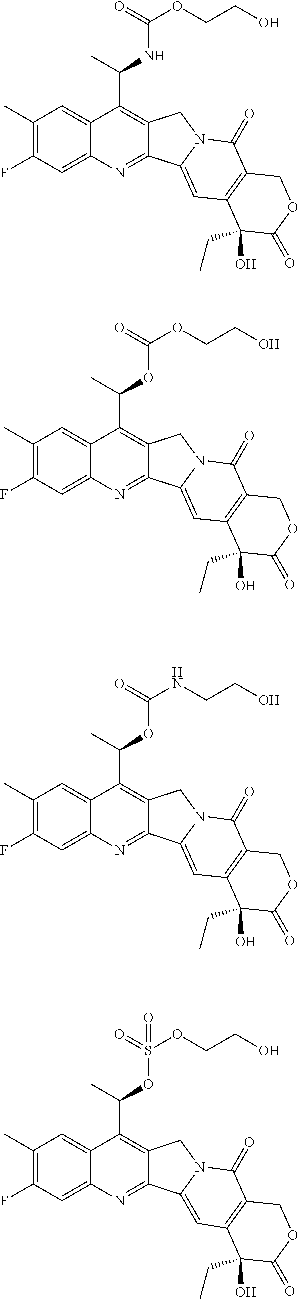 [SMILES: CC[C@@]1(O)C(=O)OCc2c1cc1n(c2=O)Cc2c-1nc1cc(F)c(C)cc1c2[C@@H](C)NC(=O)OCCO ; CC[C@@]1(O)C(=O)OCc2c1cc1n(c2=O)Cc2c-1nc1cc(F)c(C)cc1c2[C@@H](C)OC(=O)OCCO ; CC[C@@]1(O)C(=O)OCc2c1cc1n(c2=O)Cc2c-1nc1cc(F)c(C)cc1c2[C@@H](C)OC(=O)NCCO ; CC[C@@]1(O)C(=O)OCc2c1cc1n(c2=O)Cc2c-1nc1cc(F)c(C)cc1c2[C@@H](C)OS(=O)(=O)OCCO]

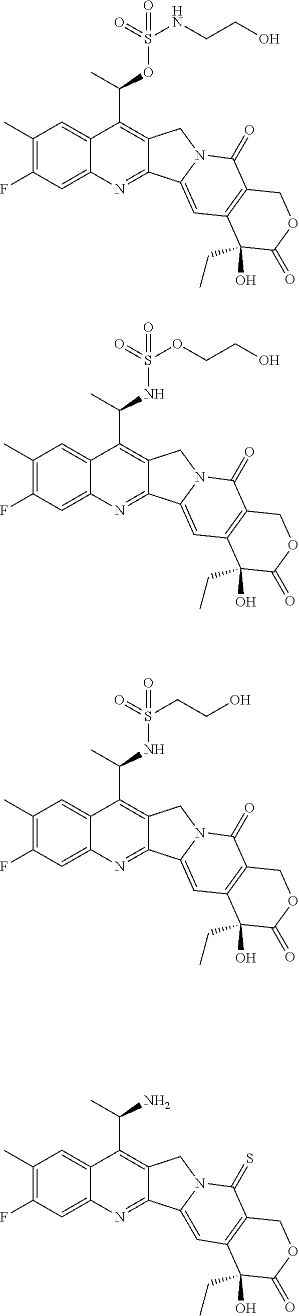 [SMILES: CC[C@@]1(O)C(=O)OCc2c1cc1n(c2=O)Cc2c-1nc1cc(F)c(C)cc1c2[C@@H](C)OS(=O)(=O)NCCO ; CC[C@@]1(O)C(=O)OCc2c1cc1n(c2=O)Cc2c-1nc1cc(F)c(C)cc1c2[C@@H](C)NS(=O)(=O)OCCO ; CC[C@@]1(O)C(=O)OCc2c1cc1n(c2=O)Cc2c-1nc1cc(F)c(C)cc1c2[C@@H](C)NS(=O)(=O)CCO ; CC[C@@]1(O)C(=O)OCc2c1cc1n(c2=S)Cc2c-1nc1cc(F)c(C)cc1c2[C@@H](C)N]

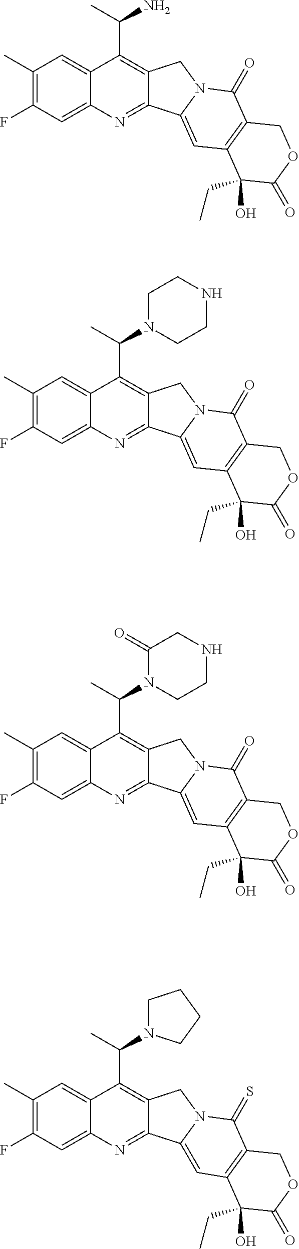 [SMILES: CC[C@@]1(O)C(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@@H](C)N1CCNCC1 ; CC[C@@]1(O)C(=O)OCc2c1cc1n(c2=O)Cc2c-1nc1cc(F)c(C)cc1c2[C@@H](C)N1CCNCC1=O ; CC[C@@]1(O)C(=O)OCc2c1cc1n(c2=S)Cc2c-1nc1cc(F)c(C)cc1c2[C@@H](C)N1CCCC1]

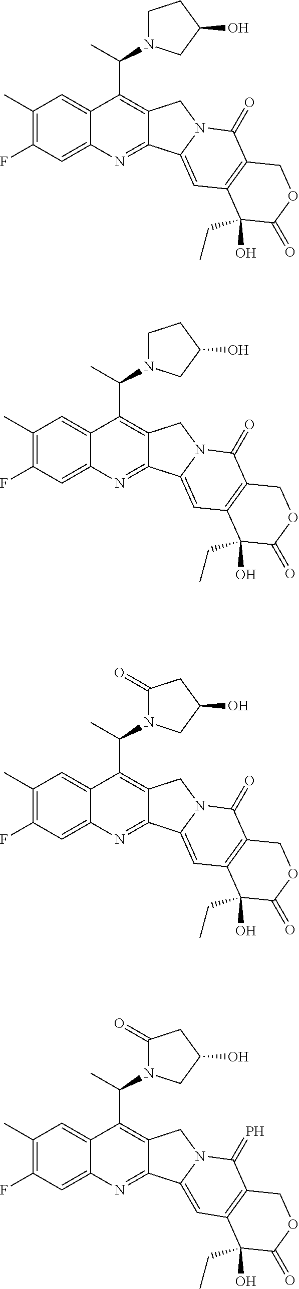

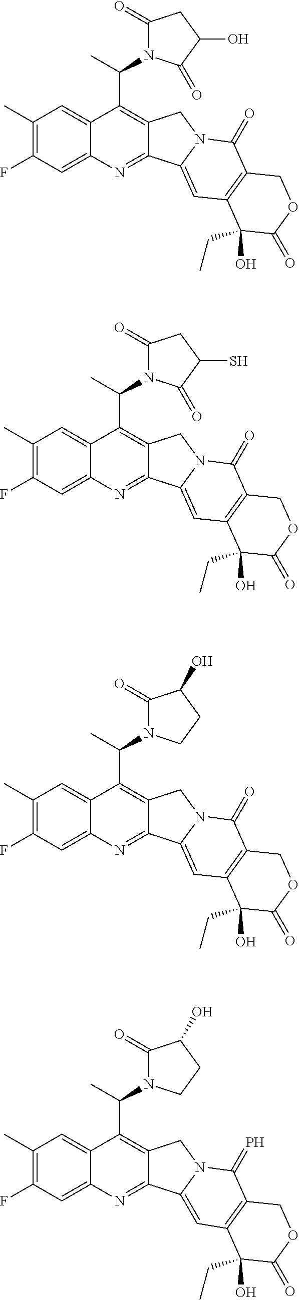

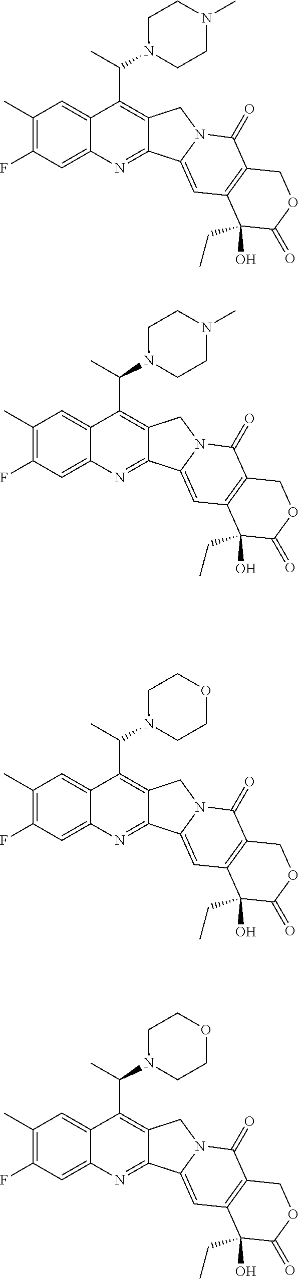

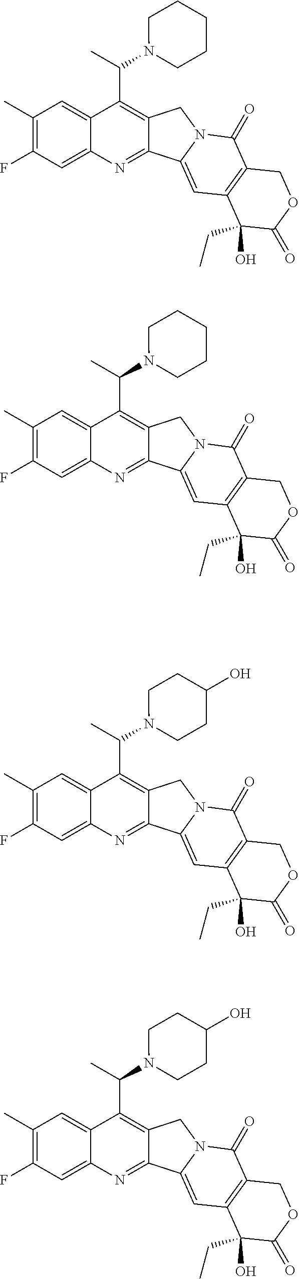

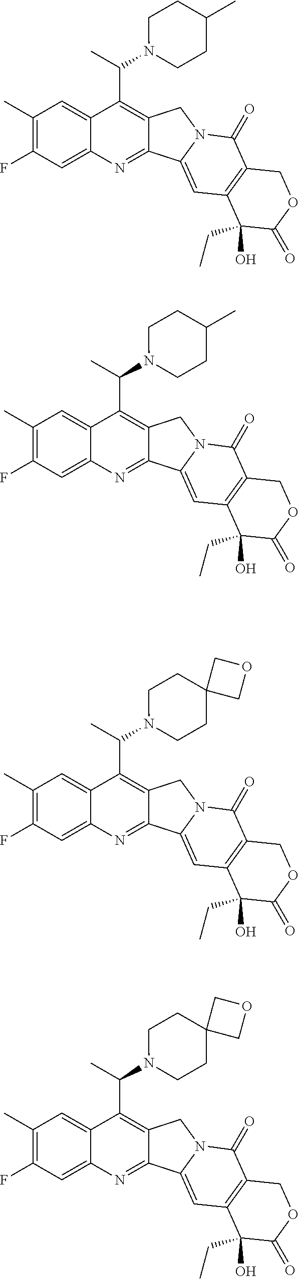

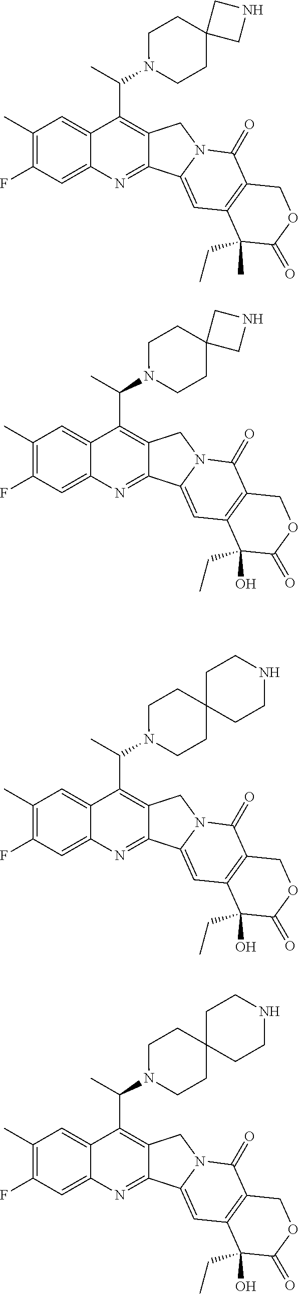

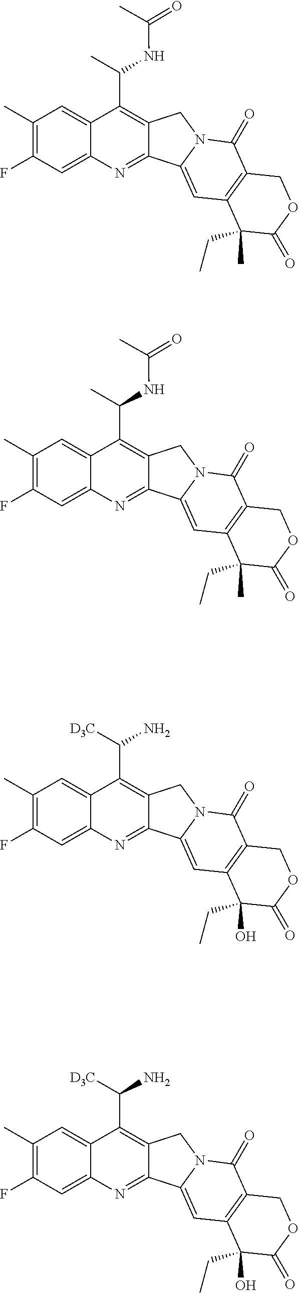

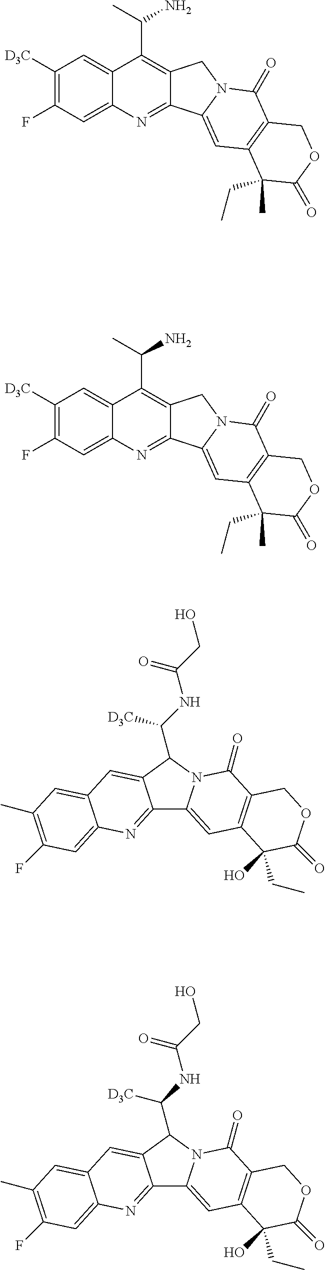

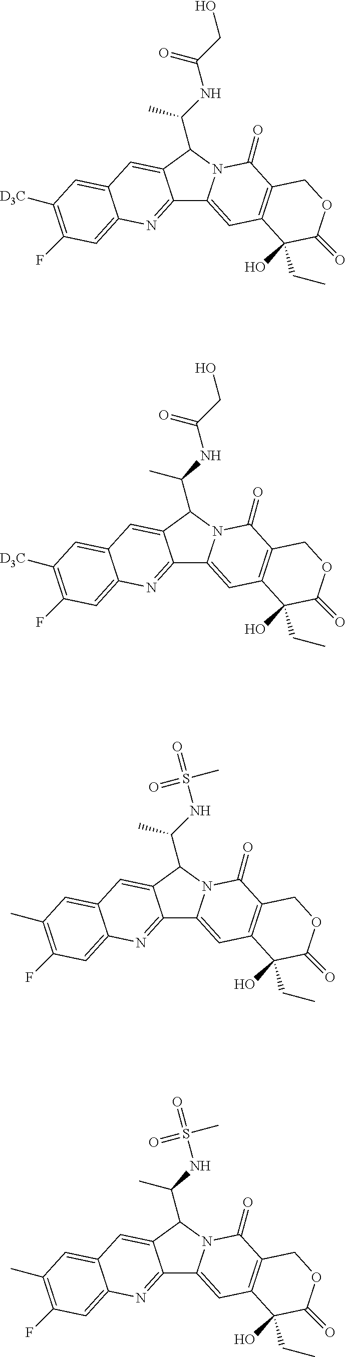 [SMILES: [2H]C([2H])([2H])c1cc2cc3c(nc2cc1F)-c1cc2c(c(=O)n1C3[C@H](C)NC(=O)CO)COC(=O)[C@]2(O)CC ; [2H]C([2H])([2H])c1cc2cc3c(nc2cc1F)-c1cc2c(c(=O)n1C3[C@@H](C)NC(=O)CO)COC(=O)[C@]2(O)CC ; CC[C@@]1(O)C(=O)OCc2c1cc1n(c2=O)C([C@H](C)NS(C)(=O)=O)c2cc3cc(C)c(F)cc3nc2-1 ; CC[C@@]1(O)C(=O)OCc2c1cc1n(c2=O)C([C@@H](C)NS(C)(=O)=O)c2cc3cc(C)c(F)cc3nc2-1]

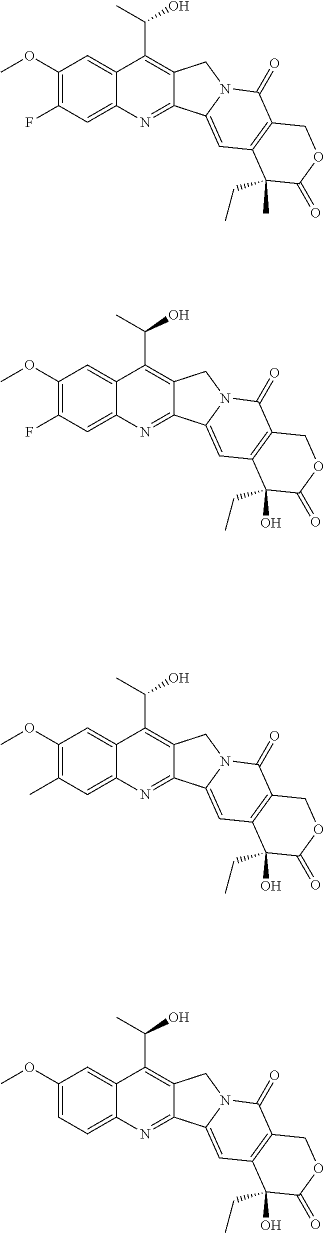 [SMILES: CC[C@@]1(C)C(=O)OCc2c1cc1n(c2=O)Cc2c-1nc1cc(F)c(OC)cc1c2[C@H](C)O ; CC[C@@]1(O)C(=O)OCc2c1cc1n(c2=O)Cc2c-1nc1cc(F)c(OC)cc1c2[C@@H](C)O ; CC[C@@]1(O)C(=O)OCc2c1cc1n(c2=O)Cc2c-1nc1cc(C)c(OC)cc1c2[C@H](C)O ; CC[C@@]1(O)C(=O)OCc2c1cc1n(c2=O)Cc2c-1nc1ccc(OC)cc1c2[C@@H](C)O]

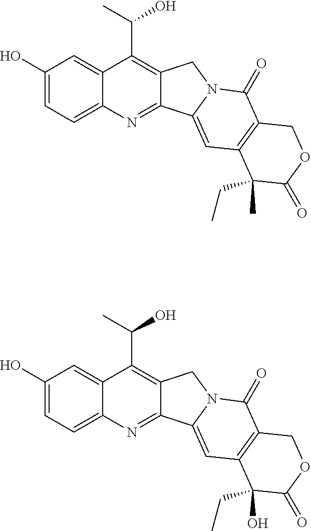 [SMILES: CC[C@@]1(C)C(=O)OCc2c1cc1n(c2=O)Cc2c-1nc1ccc(O)cc1c2[C@H](C)O ; CC[C@@]1(O)C(=O)OCc2c1cc1n(c2=O)Cc2c-1nc1ccc(O)cc1c2[C@@H](C)O]

In a preferred embodiment of the present invention, the compound is any one of the following compounds:

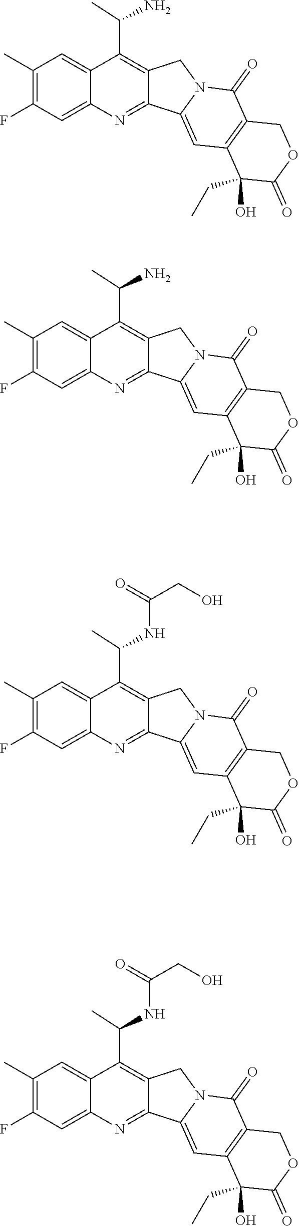 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@H](C)NC(=O)CO ; CC[C@@]1(O)C(=O)OCc2c1cc1n(c2=O)Cc2c-1nc1cc(F)c(C)cc1c2[C@@H](C)NC(=O)CO]

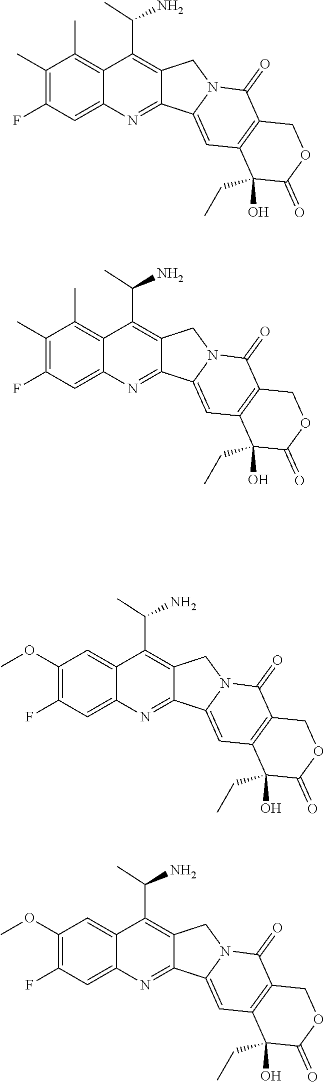 [SMILES: CC[C@@]1(O)C(=O)OCc2c1cc1n(c2=O)Cc2c-1nc1cc(F)c(C)c(C)c1c2[C@H](C)N ; CC[C@@]1(O)C(=O)OCc2c1cc1n(c2=O)Cc2c-1nc1cc(F)c(C)c(C)c1c2[C@@H](C)N ; CC[C@@]1(O)C(=O)OCc2c1cc1n(c2=O)Cc2c-1nc1cc(F)c(OC)cc1c2[C@H](C)N ; CC[C@@]1(O)C(=O)OCc2c1cc1n(c2=O)Cc2c-1nc1cc(F)c(OC)cc1c2[C@@H](C)N]

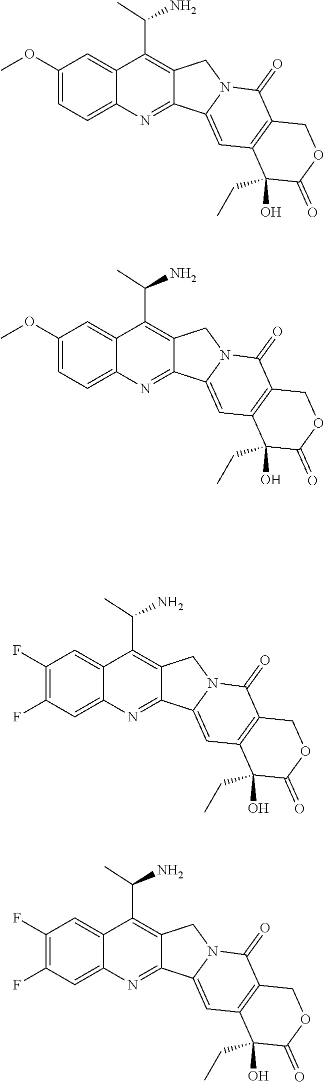 [SMILES: CC[C@@]1(O)C(=O)OCc2c1cc1n(c2=O)Cc2c-1nc1ccc(OC)cc1c2[C@H](C)N ; CC[C@@]1(O)C(=O)OCc2c1cc1n(c2=O)Cc2c-1nc1ccc(OC)cc1c2[C@@H](C)N ; CC[C@@]1(O)C(=O)OCc2c1cc1n(c2=O)Cc2c-1nc1cc(F)c(F)cc1c2[C@H](C)N ; CC[C@@]1(O)C(=O)OCc2c1cc1n(c2=O)Cc2c-1nc1cc(F)c(F)cc1c2[C@@H](C)N]

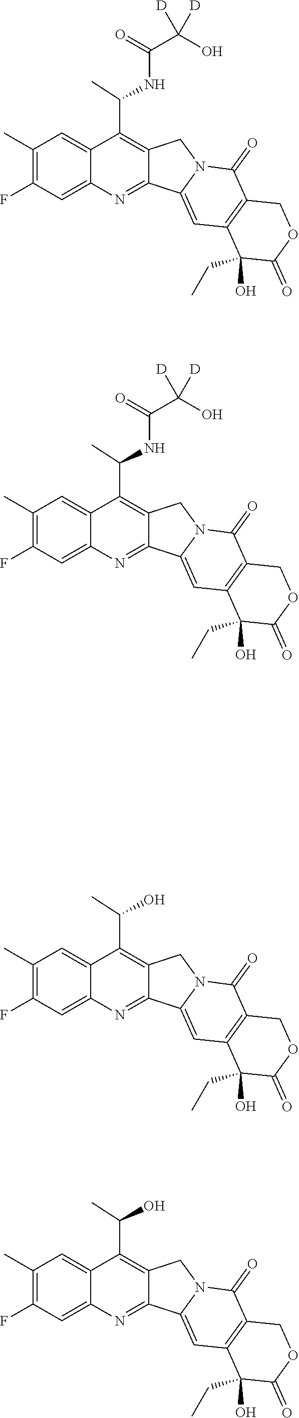 [SMILES: [2H]C([2H])(O)C(=O)N[C@@H](C)c1c2c(nc3cc(F)c(C)cc13)-c1cc3c(c(=O)n1C2)COC(=O)[C@]3(O)CC ; [2H]C([2H])(O)C(=O)N[C@H](C)c1c2c(nc3cc(F)c(C)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2[C@H](C)O ; CC[C@@]1(O)C(=O)OCc2c1cc1n(c2=O)Cc2c-1nc1cc(F)c(C)cc1c2[C@@H](C)O]

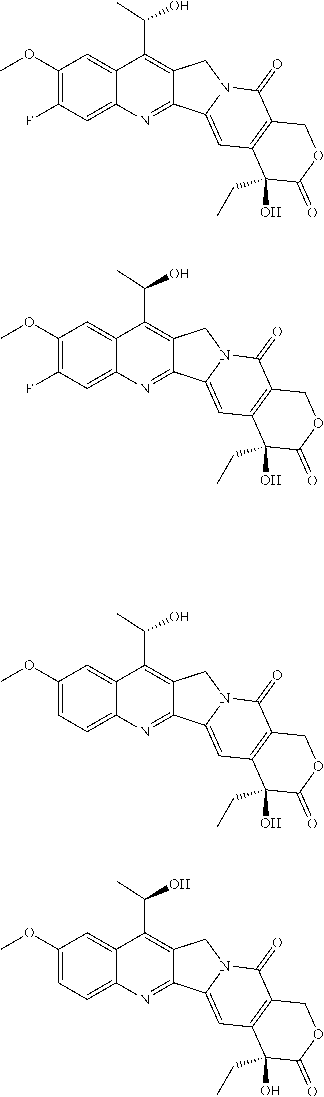 [SMILES: CC[C@@]1(O)C(=O)OCc2c1cc1n(c2=O)Cc2c-1nc1cc(F)c(OC)cc1c2[C@H](C)O ; CC[C@@]1(O)C(=O)OCc2c1cc1n(c2=O)Cc2c-1nc1cc(F)c(OC)cc1c2[C@@H](C)O ; CC[C@@]1(O)C(=O)OCc2c1cc1n(c2=O)Cc2c-1nc1ccc(OC)cc1c2[C@H](C)O ; CC[C@@]1(O)C(=O)OCc2c1cc1n(c2=O)Cc2c-1nc1ccc(OC)cc1c2[C@@H](C)O]

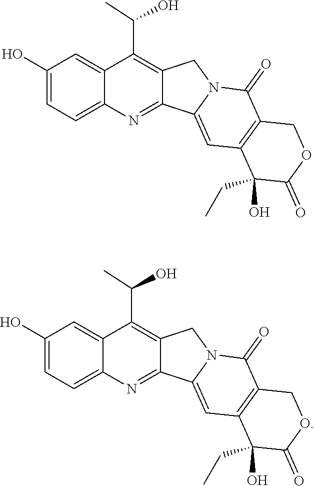 [SMILES: CC[C@@]1(O)C(=O)OCc2c1cc1n(c2=O)Cc2c-1nc1ccc(O)cc1c2[C@H](C)O ; CC[C@@]1(O)C(=O)OCc2c1cc1n(c2=O)Cc2c-1nc1ccc(O)cc1c2[C@@H](C)O]

In a second aspect, the present invention also provides a method for preparing the compound according to any one of general formulas (I)-(V) or (V-1) or a pharmaceutically acceptable salt thereof, which comprises the following steps:

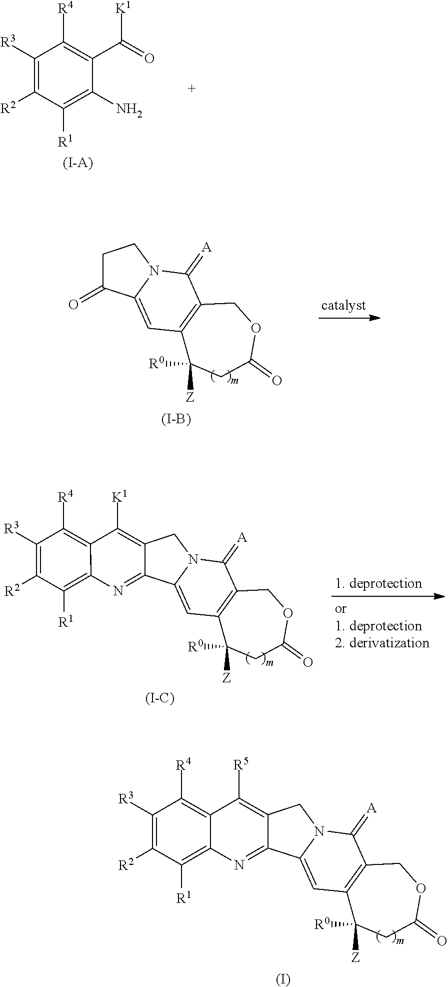

Intermediate (I-A) and intermediate (I-B) were heated for ring-closing under catalyst conditions to obtain intermediate formula (I-C), and then deprotected or derivatized after deprotecting to obtain compound formula (I);

Wherein the catalyst is p-toluenesulfonic acid monohydrate, pyridine p-toluenesulfonate complex (PPTS) or camphorsulfonic acid (CSA), the reaction temperature is 50-200° C., and the molar ratio of intermediate (I-A) to intermediate (I-B) is 5:1-1:5;

K¹ is selected from

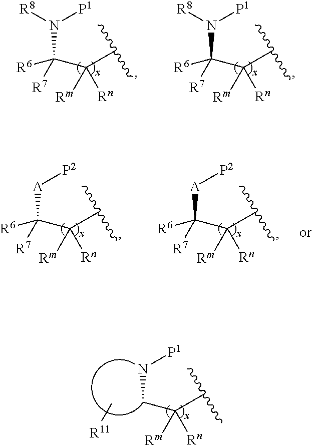

(the ring in

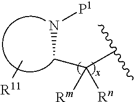

is 3-7 membered heterocycle);

P¹ and P² are protective groups, where P¹ is selected from benzyloxycarbonyl (Cbz), benzyl (Bn), p-methoxybenzyl (PMB), 9-fluorenylmethoxycarbonyl (Fmoc), allyloxy carbonyl (Alloc), 2-(trimethylsilyl) ethoxycarbonylation (Teoc), phthalyl (Pht), p-toluenesulfonyl (Ts), trifluoroacetyl, trityl (Trt), 2,4-dimethoxybenzyl (DMB), acetyl (Ac) or other ester protecting groups; where P² is selected from benzyloxycarbonyl (Cbz), benzyl (Bn), p-methoxy benzyl (PMB), tert-butyldiphenylsilyl (TBDPS), tert-butyldimethylsilyl (TBS/TBDMS), triisopropylsilyl (TIPS), allyloxycarbonyl (Alloc), 2-(trimethylsilyl) ethoxycarbonylation (Teoc), trityl (Trt), 2, 4-dimethoxybenzyl (DMB), methoxymethyl ether (MOM), acetyl (Ac) or other ester, ether, silicon ether protecting groups; R⁰, R¹, R², R³, R⁴, R⁵, R⁶, R⁷, R⁸, R¹¹, R^{m}, Rⁿ, x, A, and Z are as described in any one of the present invention.

In one aspect, the present invention relates to a pharmaceutical composition, which includes an effective amount of a compound according to any one of the present invention or a pharmaceutically acceptable salt thereof and a pharmaceutically acceptable carrier, diluent agents or excipients.

The present invention further relates to the use of a compound according to any one of the present invention or a pharmaceutically acceptable salt thereof or a pharmaceutical composition according to any one of the present inventions in the preparation of a medicament for treating or preventing tumors.

The present invention further relates to the use of a compound as described in any one of the present invention or a pharmaceutically acceptable salt thereof or a pharmaceutical composition as described in any one of the present invention in the preparation and treatment of cancer, wherein the cancer is selected from one or more of breast cancer, ovarian cancer, prostate cancer, melanoma cancer, brain cancer, nasopharyngeal cancer, esophageal cancer, gastric cancer, liver cancer, pancreatic cancer, colorectal cancer, lung cancer, renal cancer, skin cancer, glioblastoma, neuroblastoma, sarcoma, osteochondroma, bone cancer, seminomas, testicular tumors, uterine tumors, head and neck tumors, multiple myeloma, malignant lymphoma, polycythemia vera, leukemia, thyroid tumors, ureteral tumors, bladder tumors, gallbladder carcinoma, cholangiocarcinoma, or choriocarcinoma.

The present invention further relates to the use of the compound according to any one of the present invention or a pharmaceutically acceptable salt thereof for the preparation of drug conjugates.

In a preferred embodiment of the present invention, the drug conjugate is selected from: antibody-drug conjugate (ADC), peptide drug conjugate (PDC), small molecule drug conjugates (SMDC), polymer drug conjugates, lipid drug conjugates or protein drug conjugates.

The present invention further relates to the use of the compound according to any one of the present invention or a pharmaceutically acceptable salt thereof in a drug delivery system, including microspheres, micelles, liposomes, polymer nanoparticles, Liposome nanoparticles or small molecule nanoparticles.

### DETAILED DESCRIPTION OF THE INVENTION

Unless otherwise stated to the contrary, the terms used in the specification and claims have the following definitions.

The term “alkyl” refers to a saturated aliphatic hydrocarbon group, including linear or branched groups of 1 to 20 carbon atoms, preferably 1 to 10 carbon atoms, more preferably 1 to 8 carbon atoms, and non-limiting examples include, but are not limited to, methyl, ethyl, n-propyl, n-butyl, isobutyl, tert-butyl, sec-butyl, n-pentyl, 2-methylbutyl, 3-methylbutyl, 1, 1-dimethylpropyl, 1, 2-dimethylpropyl, 2, 2-dimethylpropyl, 1-ethylpropyl, n-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1-ethyl-2-methylpropyl, n-heptyl, 2-methylhexyl, 3-methylhexyl, 4-methylhexyl, 5-methylhexyl, 2,2-dimethylpentyl, 2,3-dimethylpentyl, 2,3-dimethylpentyl, 2, 4-dimethylpentyl, 3,3-dimethylpentyl, 3,4-dimethylpentyl, 2-ethylpentyl, 3-ethylpentyl, octyl, nonyl, decyl, undecyl, dodecyl, and various isomers thereof, and the like. The alkyl can be substituted or unsubstituted and can be substituted at any available junction, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, cycloalkylmercapto, heterocycloalkylmercapto, oxo, amino, haloalkyl, hydroxyalkyl, carboxyl or carboxylate group, etc. When “alkyl” and its prefix are used herein, both linear and branched saturated carbon bonds are included.

The term “cycloalkyl” refers to a saturated or partially unsaturated monocyclic or polycyclic group comprising from 3 to 20 carbon atoms, preferably 3 to 12 carbon atoms, more preferably 3 to 10 carbon atoms, and most preferably 3 to 6 carbon atoms. Non-limiting examples of monocyclic cycloalkyl include, but are not limited to, cyclopropyl, cyclobutyl, cyclopentyl, cyclopentenyl, cyclohexyl, cyclohexenyl, cyclohexadiene, cycloheptyl, cyclooctyl, and the like. Non-limiting examples of polycyclic cycloalkyl include, but are not limited to, spirocycloalkyl, fused cycloalkyl and bridged cycloalkyl. Cycloalkyl can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkoxy, cycloalkoxy, cycloalkoxy, cycloalkylmercapto, heterocycloalkylmercapto, oxo, amino, haloalkyl, hydroxyalkyl, carboxyl or carboxylate group, and the like.

The term “haloalkyl” refers to an alkyl can be substituted by one or more than one the same or different halogen atoms, wherein the definition of the alkyl is as defined herein.

The term “deuterated alkane” means that an alkyl group may be substituted by one or more deuterium atoms, wherein alkyl group is as defined herein.

The term “alkenyl” refers to an alkyl as defined in the present invention consisting of at least two carbon atoms and at least one carbon-carbon double bond, preferably C₂-C₁₀ alkenyl, more preferably C₂-C₆ alkenyl, most preferably C₂-C₄ alkenyl, such as vinyl, propenyl, 1-propenyl, and the like. The alkenyl group can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkoxy, cycloalkoxy, cycloalkoxy, cycloalkylmercapto, heterocycloalkylmercapto, oxo, amino, haloalkyl, hydroxyalkyl, carboxyl or carboxylate group, and the like.

The term “alkynyl” refers to an alkyl as defined in the present invention consisting of at least two carbon atoms and at least one carbon-carbon triple bond, preferably C₂-C₁₀ alkynyl, more preferably C₂-C₆ alkynyl, most preferably C₂-C₄ alkynyl, such as ethynyl, 1-propynyl, 2-propynyl, and the like. The alkynyl group can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkoxy, cycloalkoxy, cycloalkoxy, cycloalkylmercapto, heterocycloalkylmercapto, oxo, amino, haloalkyl, hydroxyalkyl, carboxyl or carboxylate group, and the like.

The term “heterocycloalkyl” refers to a saturated or partially unsaturated monocyclic or polycyclic hydrocarbon group comprising from 3 to 20 ring atoms, wherein one or more than one ring atoms are selected from heteroatoms of N, O, S(O)ₘ, P(O)ₘ (wherein m is an integer from 0 to 2), but excluding ring moiety of —O—O, —O—S— or —S—S— and the remaining ring atoms are carbon. Preferably 3 to 12 ring atoms containing 1 to 4 heteroatoms, and non-limiting examples of monocyclic heterocycloalkyl include pyrrolyl, piperidinyl, piperazinyl, morpholinyl, tetrahydrofuranyl, pyranyl, and the like. Polycyclic heterocycloalkyl include spiro heterocycloalkyl, fused heterocycloalkyl and bridged heterocycloalkyl. Heterocycloalkyl can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, cycloalkylmercapto, heterocycloalkylmercapto, oxo, amino, haloalkyl, hydroxyalkyl, carboxyl or carboxylate group, and the like.

The term “alkoxy” refers to —O-(alkyl) and —O-(cycloalkyl), wherein the definitions of the alkyl and the cycloalkyl are as described in the description. Non-limiting examples include, but are not limited to, methoxy, ethoxy, propoxy, butoxy, cyclopropoxy, cyclobutoxy, cyclopentyloxy, cyclohexyloxy, and the like. The alkoxy group can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, heterocycloalkyloxy, cycloalkylmercapto, heterocycloalkylmercapto, oxo, amino, haloalkyl, hydroxyalkyl, carboxyl or carboxylate group, and the like.

The term “alkylmercapto” refers to —S-(alkyl) and —S-(cycloalkyl), wherein the definitions of the alkyl and the cycloalkyl are as described in the description. Non-limiting examples include, but are not limited to, methylmercapto, ethylmercapto, propylmercapto, butylmercapto, cyclopropylmercapto, cyclobutylmercapto, cyclopentylmercapto, cyclohexylmercapto, and the like. Alkylmercapto can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkylmercapto, heterocycloalkylmercapto, oxo, amino, haloalkyl, hydroxyalkyl, carboxyl or carboxylate group, and the like.

The term “substituted or unsubstituted amino” refers to NH₂, monosubstituted NH₂ and disubstituted NH₂. When substituted, the monosubstituent or disubstituted group is preferably independently selected from alkyl, hydroxyl, mercapto, alkenyl, alkynyl, alkoxy, alkylmercapto, alkylamino, cycloalkyl, heterocycloalkyl, aryl, heteroaryl, cycloalkoxy, heterocycloalkoxy, cycloalkylmercapto, heterocycloalkylmercapto, oxo group, amino, haloalkyl, hydroxyalkyl, carboxyl or carboxylate group, etc. Disubstituted groups can form a nonaromatic cyclic structure together with the nitrogen atom to which they are attached.

The term “aryl” refers to any stable conjugated hydrocarbon ring system group with 6-18 carbon atoms, preferably 6-10 carbon atoms, which can be monocyclic, bicyclic, tricyclic or more cyclic aromatic groups, such as phenyl, naphthyl and anthracene, etc. The aryl ring can be fused to a heteroaryl, heterocycloalkyl or cycloalkyl ring. The aryl group may be substituted or unsubstituted, and when substituted, the substituent group is preferably one or more groups independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy and cycloalksulfhydryl.

The term “heteroaryl” refers to an aromatic ring system in which at least one carbon atom in the ring is replaced by a heteroatom selected from N, O or S, preferably a 5- to 7-membered monocyclic moiety or a 7- to 12-membered bicyclic moiety, more preferably a 5- to 6-membered heteroaryl, such as pyrrolyl, imidazolyl, pyridyl, pyrimidinyl, thiazolyl, thienyl, pyrazinyl, triazolyl, tetrazolyl, oxazolyl, indazolyl, and the like. The heteroayl ring can be fused to a ring of aryl, heterocycloalkyl or cycloalkyl. The heteroaryl can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, heterocycloalkyloxy, cycloalkylmercapto, heterocycloalkylmercapto, oxo, amino, haloalkyl, hydroxyalkyl, carboxyl or carboxylate group.

The term “sulfone group” refers to

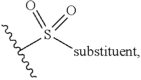

wherein the substituent is preferably alkyl, alkenyl, alkynyl, amino, alkoxy, alkylmercapto, alkylamino, cycloalkyl, haloalkyl, heterocycloalkyl, aryl, heteroaryl, cycloalkoxy, heterocycloalkoxy, cycloalkyl mercapto or heterocycloalkyl mercapto.

The term “sulfoxide” refers to

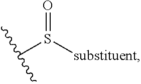

wherein the substituent is preferably alkyl, alkenyl, alkynyl, amino, alkoxy, alkylmercapto, alkylamino, cycloalkyl, haloalkyl, heterocycloalkyl, aromatic radical, heteroaryl, cycloalkoxy, heterocycloalkoxy, cycloalkylmercapto or heterocycloalkylmercapto.

The term “alkyl sulfoxide” refers

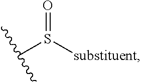

wherein the substituent is preferably an alkyl group, as defined above.

The term “hydroxyl” refers to —OH.

The term “halogen” refers to fluorine, chlorine, bromine or iodine.

The term “nitro” refers to —NO₂.

The term “amino” refers to —NH₂.

The term “cyano” refers to —CN.

The term “carboxylic acid” refers to —C(O)OH.

The term “mercapto” refers to —SH.

The term “carboxylate group” refers to —C(O)O-alkyl, —C(O)O-aryl, or —C(O)O— cycloalkyl, wherein the definitions of alkyl, the aryl, and the cycloalkyl are as defined above.

The term “substituted” means that one or more than one hydrogen or deuterium atoms in the group, preferably 1 to 5 hydrogens or deuterium atoms, are independently substituted by a corresponding number of substituents.

The term “pharmaceutically acceptable salt” refers to a salt that can retain the biological effectiveness of the free base without other toxic and side effects, and can be an acidic salt, a basic salt or an amphoteric salt. Non-limiting examples include, but are not limited to, acidic salts including hydrochloride, hydrobromide, sulfate, pyrosulfate, bisulfate, sulfite, bisulfite, phosphate, monohydrogen phosphate, dihydrogen phosphate, metaphosphate, pyrophosphate, nitrate, acetate, propionate, caprate, octanoate, formate, acrylate, isobutyrate, hexanoate, heptanoate, oxalate, malonate, succinate, suberate, benzoate, methyl benzoate, phthalate, maleate, methanesulfonate, p-toluenesulfonate, benzenesulfonate, (D, L)-tartrate, citrate, maleate, (D, L-) malate, fumarate, stearate, oleate, cinnamate, laurate, glutamate, aspartate, triflate, mandelate, ascorbate, salicylate, and the like. When the compound of the present invention contains acidic groups, pharmaceutically acceptable salts thereof can further include alkali metal salts (e.g., sodium salt or potassium salt), alkaline earth metal salts (e.g., calcium salt or magnesium salt), organic base salts (e.g., alkyl aromatics, amino acids, etc.).

The term “pharmaceutical composition” refers to a mixture of one or more than one compounds described herein or pharmaceutically acceptable salts or prodrugs thereof and other chemical components, as well as other components such as physiologically acceptable carriers and excipients. The purpose of the pharmaceutical composition is to promote the administration of the organism and to facilitate the absorption of the active ingredient and exert biological activity.

Abbreviations for any protecting groups, amino acids, and other compounds are commonly used and recognized abbreviations, unless otherwise specified, or refer to IUPAC-IUBC Commission on Biochemical Nomenclature (See Biochem. 1972, 11, 942-944).

### BRIEF DESCRIPTION OF THE DRAWINGS

FIG. 1 illustrates tumor growth curves of the Example 27 and Example 28 in BxPC-3 mouse tumor xenograft model.

FIG. 2 illustrates the body weight change curves of the Example 27 and Example 28 in BxPC-3 mouse tumor xenograft model.

### EXAMPLES

The following examples further describe the present invention, but these examples should not limit the scope of the present invention.

In the examples of the invention, the experimental methods without specifying specific conditions are generally in accordance with conventional methods and conditions, or in accordance with the conditions recommended by the manufacturers of raw materials or commodities. The reagents without specific sources are conventional reagents purchased from the market.

All compounds of the present invention can be determined by nuclear magnetic resonance (NMR) or mass spectrometry (MS). The NMR shift (d) is recorded in units of 10⁻⁶ (ppm). The NMR measuring instrument is Bruker AVANCE-400 spectrometer. The deuterated solvents are deuterated chloroform (CDCl₃), deuterated methanol (CD₃OD), deuterium oxide (D₂O) or deuterated dimethyl sulfoxide (DMSO-d₆), and the internal standard is tetramethylsilane (TMS).

Low-resolution mass spectrometry (MS) is determined by Agilent 6120 quadruple LCMS mass spectrometer.

The HPLC purity is determined by Agilent HPLC Agilent 1260/1220 chromatograph (Agilent Zorb Ax BonusRP 3.5 μm×4.6 mm×150 mm or Boston pHlex ODS 4.6 mm×150 mm×3 μm).

The compounds of the present invention and their intermediates can be isolated and purified by conventional preparative HPLC, silica gel plate, column chromatography, or flash column chromatography.

The thin-layer chromatography silica gel plate uses Yantai Huanghai, Yantai Xinnuo Chemical Industry HSGF254, or Qingdao GF254 silica gel plate. The silica gel plate used for thin-layer chromatography (TLC) is 2.5×5 cm, 0.2 mm-0.25 mm, and the thin layer chromatography separation (Prep-TLC) used for purifying products is 1 mm or 0.4 mm-0.5 mm, 20×20 cm.

Column chromatography (silica gel column chromatography) is generally used in sizes of 100-200 mesh or 200-300 mesh or 300-400 mesh.

The flash separator is Agela Technologies MP200, and the column is generally Flash column silica-CS (12 g-330 g).

The preparative HPLC (Prep-HPLC) is Gilson GX-281, and the column model is Welch Ultimate XB-C18 21.2 mm×250 mm×10 μm.

The chiral columns are CHIRALCEL OD-H, OJ-H or CHIRALPAK AD-H, AS-H 4.6 mm×250 mm×5 μm, and the preparation column types are CHIRALCEL OD-H, OJ-H or CHIRALPAK AD-H, AS-H 10 mm×250 mm×5 μm.

The known starting materials of the present invention can be synthesized by methods known in the art, or purchased from suppliers such as Sigma-Aldrich, ACROS, Alaf, TCI, J&K Scientific, energy-chemical, Accela ChemBio, Macklin, Siyanbio chemical companies and the like.

Anhydrous solvents such as anhydrous tetrahydrofuran, anhydrous dichloromethane or anhydrous N, N-dimethylacetamide are commercially available from the above chemical companies.

Unless otherwise specified in the examples, the reaction is generally carried out under a nitrogen or argon atmosphere. The nitrogen or argon atmosphere refers to that the reaction flask is connected to a balloon of nitrogen or argon having a volume of about 1 L and subjected to three pumping displacements.

The hydrogen atmosphere means that the reaction flask is connected to a hydrogen balloon having a volume of about 1 L and subjected to three pumping displacements.

The pressurized hydrogenation reaction uses a pressure-resistant sealed glass reaction vessel and is connected to a hydrogen pressure gauge.

In the examples, unless otherwise specified, the reaction temperature is room temperature, and the temperature is 15-25° C.

The reactions in the examples are generally monitored by LCMS or TLC, wherein the LCMS is as described above. The developing solvent system used for TLC is generally: dichloromethane and methanol, petroleum ether and ethyl acetate, dichloromethane and ethyl acetate, petroleum ether and dichloromethane, ethyl acetate and methanol, etc. The volume ratio of the solvent is adjusted according to the polarity of the compound, and a small amount (0.1%-10%) of base (such as triethylamine or 37% ammonia water, etc.) or acid (such as acetic acid, etc.) can also be added for adjustment.

The compounds can be purified by Prep-TLC, column chromatography or Agela preparation system. The elution solvent system is generally dichloromethane and methanol, petroleum ether and ethyl acetate, dichloromethane and ethyl acetate, petroleum ether and dichloromethane, ethyl acetate and methanol, etc. The volume ratio of the solvent is adjusted according to the polarity of the compound. A small amount (0.1%-10%) of base (such as triethylamine or 37% ammonia water, etc.) or acid (such as acetic acid, etc.) can also be added for adjustment.

The following abbreviations are used throughout the present invention:
CPT: Camptothecin
DMF: N, N-dimethylformamide
THF: Tetrahydrofuran
DCM: Dichloromethane
MeOH: Methanol
HCl: Hydrochloric acid
Boc₂O: Di-tert-butyl dicarbonate
EDC·HCl: 1-(3-Dimethylaminopropyl)-3-ethylcarbodiimide hydrochloride
DMAP: 4-Dimethylaminopyridine
TBHP: Tert-butyl hydroperoxide
CAN: Acetonitrile
NBS: N-bromosuccinimide
CDI: N,N′-carbonyldiimidazole
TFA: Trifluoroacetic acid
DMTMM: 4-(4, 6-Dimethoxytriazin-2-yl)-4-methylmorpholine hydrochloride
DMSO: Dimethyl sulfoxide
K₂CO₃: Potassium carbonate
Na₂S₂O₃: Sodium thiosulfate
NaHCO₃: Sodium bicarbonate
NaBH(OAc)₃: Sodium triacetoxyborohydride
Dess-martinperiodinane: 1,1,1-Tris (acetyloxy)-1,1-dihydro-1,2-benziodoxol-3-(1H)-one
n-BuLi: n-Butyllithium
NH₃·H₂O: Ammonium hydroxide
CDCl₃: Deuterated chloroform
H₂: Hydrogen
H₂O: Water
Pd₂(dba)₃: Tris (dibenzylideneacetone) dipalladium
RT: Retention time
Dxd:

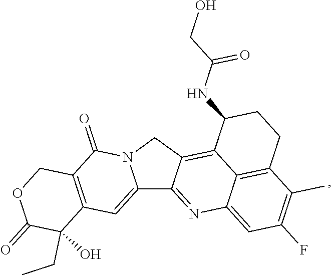 [SMILES: CC[C@@]1(O)C(=O)OCc2c1cc1n(c2=O)Cc2c-1nc1cc(F)c(C)c3c1c2[C@@H](NC(=O)CO)CC3]

### CAS is 1599440-33-1.

### Examples 1 and 2 (Synthesis Method I)

### Synthesis Method I

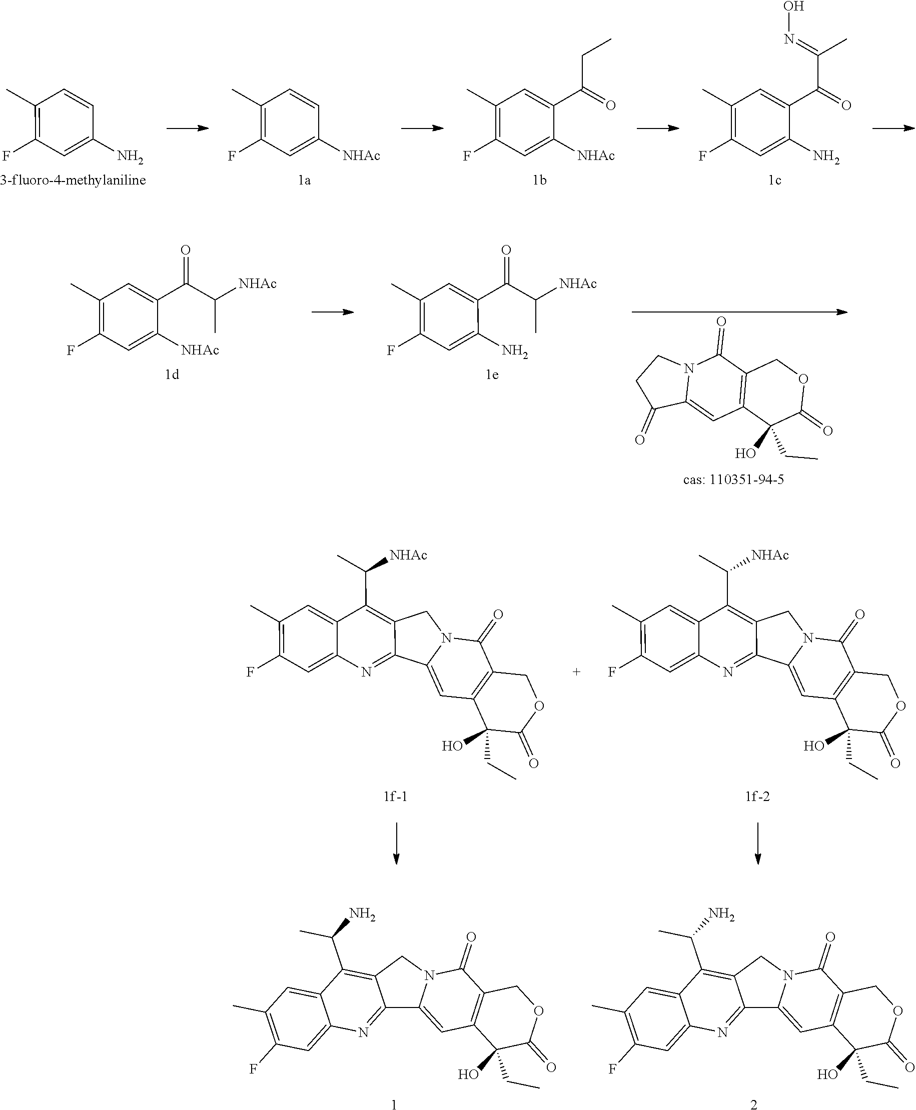

### Synthesis of Compound 1a

Under cooling in an ice-water bath, acetic anhydride (12.7 g, 0.12 mol) was added dropwise to a solution of 3-fluoro-4-methylaniline (7.8 g, 0.06 mol) in dichloromethane (50 mL), followed by triethylamine (18.9 g, 0.19 mol). The reaction was stirred for 2 hours, quenched with water, and extracted with dichloromethane (100 mL×3). The combined organic phase was washed with birne, dried over anhydrous sodium sulfate, filtered, concentrated, and sulrried with petroleum ether to obtain the compound 1a (10 g, 96% yield).

MS (ESI), m/z, 168.1 [M+1]⁺.

### Synthesis of Compound 1b

The compound 1a (16 g, 0.10 mol) was dissolved in toluene (100 mL) at 10° C., and propionaldehyde (10.2 g, 0.2 mol) and palladium acetate (0.9 g, 4 mmol) were added to it, and trifluoroacetic acid (11.2 g, 0.1 mol) and TBHP (25 g, 4 mmol) were added under nitrogen atmosphere. Then the reaction was heated to 40-50° C. for 20 h, and then extracted with water. The water phase was extracted with dichloromethane (200 mL×3). The combined organic phase was washed with brine, dried over anhydrous sodium sulfate, filtered, concentrated and purified by column chromatography to obtain compound 1b (1.5 g).

¹H NMR (400 MHZ, CDCl³) δ 11.89 (s, 1H), 8.49-8.52 (m, 1H), 7.74-7.76 (m, 1H), 3.00-3.06 (m, 2H), 2.22-2.27 (m, 6H), 1.20-1.23 (m, 3H).

### Synthesis of Compound 1c

At 10° C., the compound 1b (2.2 g, 10 mmol) was dissolved in dichloromethane (16 mL), and TMSCl (2.2 g, 20 mmol) and sodium nitrite (1.4 g, 20 mmol) were added to above mixture, and the reaction was stirred at 10° C. for 24 h. The reaction was monitored by LCMS, quenched by water, and the water phase was extracted with dichloromethane (50 mL×3). The organic phases were combined, washed with brine, dried over anhydrous sodium sulfate, filtered, concentrated and purified by column chromatography to obtain compound 1c (1.5 g).

¹H NMR (400 MHZ, CDCl₃) δ 12.19 (s, 1H), 10.21 (s, 1H), 7.36-7.41 (m, 3H), 5.76 (s, 1H), 5.76 (s, 3H), 1.95-1.97 (m, 6H).

### Synthesis of Compound 1d

The compound 1c (1.26 g, 6.0 mmol) was dissolved in acetic acid (10.0 mL) at 20° C., and acetic anhydride (10.0 mL) and zinc powder (3.2 g, 60 mmol) were added. The reaction was stirred for 16 h at 80° C. The product was monitored by LCMS, quenched by water, and the water phase was extracted with dichloromethane (50 mL×3). The organic phases were combined, washed with brine, dried over anhydrous sodium sulfate, filtered, concentrated and purified by column chromatography to obtain compound 1d (1.2 g).

¹H NMR (400 MHZ, CDCl₃) δ 11.50 (s, 1H), 8.50-8.54 (m, 1H), 7.77-7.79 (m, 2H), 6.43-6.45 (m, 1H), 5.77-5.61 (m, 1H), 2.23-2.28 (m, 6H), 2.09 (s, 3H), 1.41-1.43 (m, 3H).

### Synthesis of Compound 1e

The compound 1d (1.18 g, 4.2 mmol) was dissolved in ethanol (10 mL) at 20° C., and sodium hydroxide aqueous solution (20%, 5.0 mL) was added. The reaction was stirred at 20° C. for 16 h. LCMS showed that the product was formed, quenched with water, and extracted with dichloromethane (50 mL×3). The organic phase was washed with brine, dried over anhydrous sodium sulfate, filtered, concentrated and purified by column chromatography to obtain compound 1e (0.5 g).

¹H NMR (400 MHZ, CDCl₃) δ ppm 7.44-7.46 (m, 1H), 6.44-6.46 (m, 2H), 5.88-6.00 (m, 2H), 2.18 (s, 3H), 1.98 (s, 3H), 2.09 (s, 3H), 1.61 (m, 3H).

### Synthesis of Compound 1f-1 and 1f-2

At 20° C., compound 1e (0.48 g, 2.0 mmol) was added to toluene (5 mL), followed by ketone (CAS is 110351-94-5) (0.63 g, 2.42 mmol) and p-toluenesulfonic acid monohydrate (38 mg, 0.20 mmol), and the mixture was stirred for 16 h at 120° C. and was monitored by LCMS. After the reaction was completed, the crude product was directly purified by HPLC to obtain compounds 1f-1 (40 mg, retention time was 2.507 min in HPLC) and 1f-2 (40 mg, retention time was 2.547 min in HPLC). The stereoconfiguration of compound 1f-1, Example 1, compound 1f-2 and Example 2 was determined by synthesis method II (asymmetric synthesis).

MS (ESI), m/z, 466.1 [M+H]⁺.

HPLC conditions: Instrument: Agilent 1260 HPLC; Column: Agilent Zorbax Bonus RP (3.5 μm*4.6 mm*150 mm); Column temperature: 40° C.; Gradient: 1.0 mL/min (buffer 0.1% TFA), 5 to 100% ACN/water (1-10 min); 100% ACN (10-15 min); 100 to 5% ACN/water (15-20 min).

### Synthesis of Example 1

At 20° C., 1f-1 (40 mg, 0.086 mmol) was dissolved in 6 N hydrochloric acid aqueous solution (1.0 mL), heated to 100-105° C., stirred for 16 h and monitored by LCMS. After the reaction was completed, the mixture was concentrated to obtain the Example 1 (20 mg, hydrochloride form).

MS (ESI), m/z, 424.2 [M+H]⁺.

¹H NMR (400 MHZ, CDCl₃) δ ppm 8.86 (d, J=5.6 Hz, 1H), 8.42 (d, J=8.0 Hz, 1H), 7.88 (d, J=10.8 Hz, 1H), 7.36 (s, 1H), 5.54-5.56 (m, 2H), 5.39 (s, 2H), 5.38-5.19 (m, 1H), 2.51 (s, 3H), 1.89-1.98 (m, 5H), 1.52 (t, J=7.2 Hz, 3H), 0.86 (t, J=7.2 Hz, 3H).

### Synthesis of Example 2

At 20° C., 1f-2 (40 mg, 0.086 mmol) was dissolved in 6 N hydrochloric acid aqueous solution (1.0 mL), heated to 100-105° C., stirred for 16 h and monitored by LCMS. After the reaction was completed, the mixture was concentrated to obtain the Example 2 (18 mg, hydrochloride form).

MS (ESI), m/z, 424.2 [M+H]⁺.

¹H NMR (400 MHz, CDCl₃) δ ppm 8.86 (d, J=5.6 Hz, 1H), 8.42 (d, J=8.0 Hz, 1H), 7.88 (d, J=10.8 Hz, 1H), 7.31 (s, 1H), 5.71-5.68 (m, 2H), 5.44 (s, 2H), 5.42-5.19 (m, 1H), 2.54 (s, 3H), 1.91-1.83 (m, 5H), 1.51 (t, J=7.2 Hz, 3H), 0.86 (t, J=7.2 Hz, 3H).

### Example 1 (Synthesis Method II)

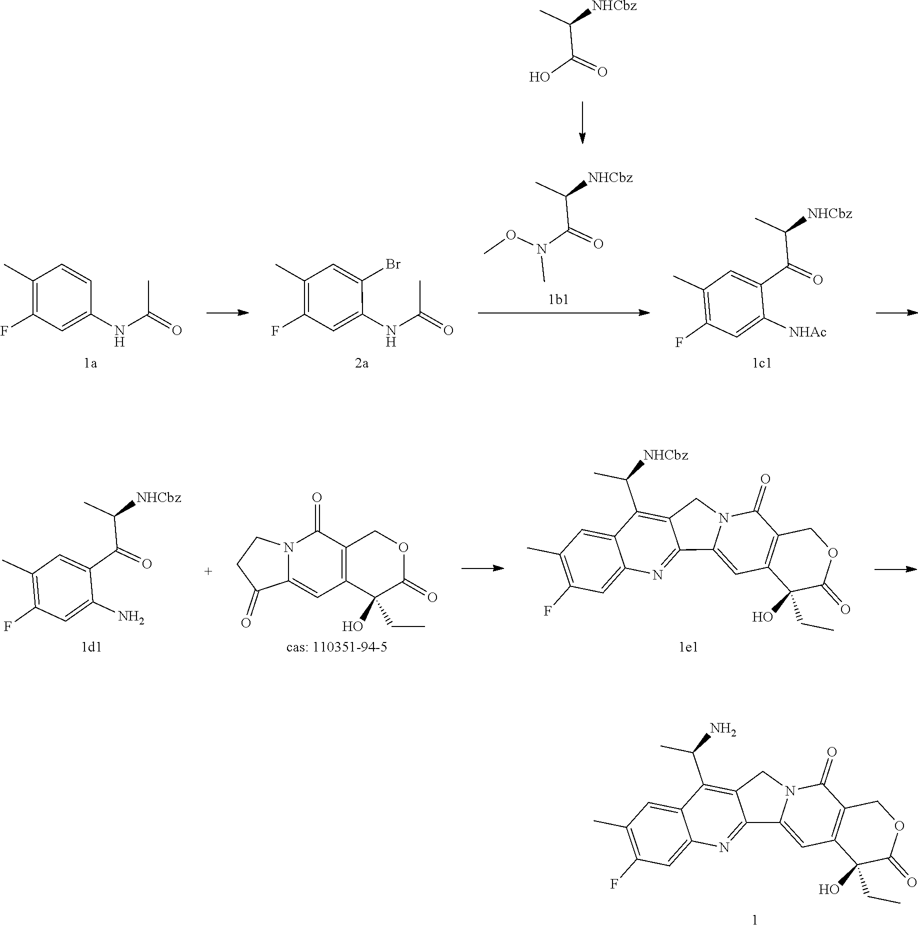

### Synthesis of Compound 2a

At 5-10° C., NBS (12.8 g, 0.07 mol) was added into DMF solution (80 mL) of 1a (8.0 g, 0.05 mol) in batches, stirred for 12 h, and poured into ice water (300 mL). The mixture was stirred for 0.5 h, filtered, washed with saturated sodium bicarbonate and brine, filtered, and then dried under vacuum to obtain compound 2a (10 g, yield 85%).

MS (ESI), m/z, 246.1 [M+H]⁺.

¹H NMR (400 MHZ, Chloroform-d) δ ppm 8.14 (d, J=11.7 Hz, 1H), 7.54 (s, 1H), 7.33 (d, J=7.6 Hz, 1H), 2.22 (s, 3H), 2.21 (d, J=2.0 Hz, 3H).

### Synthesis of Compound 1b1

To a solution of N-Cbz-L-alanine (10.0 g, 0.045 mol) in dichloromethane (100 mL) was added CDI (9.5 g, 0.058 mol, 1.3 eq) at 5° C. and stirred for 1 h, followed by dimethylhydroxylamine hydrochloride (6.5 g, 0.067 mol, 1.5 eq) and triethylamine (13.6 g, 0.13 mol, 2.0 eq) were added to the above mixture and continued to stir for 1 h. The mixture was quenched with water, extracted with dichloromethane (100 mL×3). The combined organic phase was washed with 1 N HCl, saturated sodium bicarbonate and brine, dried over anhydrous sodium sulfate, filtered and concentrated to obtain crude product 1b1 (11 g, yield 92%, ee 99.7%).

MS (ESI), m/z, 266.90 [M+H]⁺.

### Synthesis of Compound 1c1

To a solution of 2a (2.0 g, 8.13 mmol) in dry tetrahydrofuran (40 mL) was added dropwise n-butyllithium solution (2.5 M in hexane, 7.1 mL, 17.9 mmol, 2.2 eq) at −60° C. and continued to stir for 1 h. And then 1b1 (3.2 g, 12.2 mmol, 1.5 eq) in tetrahydrofuran solution (10 mL) was added dropwise into above solution and stirred for 1 h (−60-−20° C.). The mixture was quenched with ammonium chloride aqueous solution, and the water phase was extracted with ethyl acetate (80 mL×3), combined the organic phases, wash with brine, dried over anhydrous sodium sulfate, filtered, concentrated, and purified by column chromatography to obtain compound 1c1 (330 mg, ee 80%).

MS (ESI), m/z, 372.80 [M+H]⁺.

### Synthesis of Compound 1d1

3M hydrochloric acid (5 mL) was added to 1c1 (131 mg, 0.35 mmol) in methanol and tetrahydrofuran solution (5/5 mL) in an ice-water bath at 5° C., then warmed to room temperature 25° C. and stirred for 6 h, diluted with water and extracted with ethyl acetate (20 mL×3), the organic phases were combined, washed with brine, dried over anhydrous sodium sulfate, filtered, and purified by column chromatography to obtain compound 1d1 (95 mg, ee 80%).

MS (ESI), m/z, 331.1 [M+H]⁺.

### Synthesis of Compound 1e1

To a solution of 1d1 (95 mg, 0.29 mmol, 1.0 eq) in toluene (5 mL) was added ketone 110351-94-5 (90 mg, 0.34 mmol, 1.2 eq) together with p-toluenesulfonic acid monohydrate (11 mg, 0.06 mmol, 0.2 eq) and stirred for 2 h at 110° C. After the reaction was completed, the mixture was concentrated under reduced pressure. The residue was diluted with water, stirred, filtered and the collected solid was purified by slurrying with ethanol and water (10/2 mL) to obtain compound 1e1 (90 mg, ee 99%).

MS (ESI), m/z, 558.2 [M+H]⁺.

### Synthesis of Chiral Example 1

10% Pd/C (55% wet, 20 mg) was added to the mixed suspension solution of 1e1 (70 mg, 0.13 mmol) in 6M HCl (1 mL) and methanol (5 mL). The flash was evacuated and backfilled with H₂ (3×) and stirred under an atmosphere of H₂ for 1 h at 25° C. The mixture was filtered, and the crude product was purified by Prep-HPLC (CH₃CN/H₂O, 0.05% TFA) to obtain Example 1 (8 mg, ee 99%).

MS (ESI), m/z, 424.10 [M+1]⁺.

The characterization data of LCMS and ¹H NMR were consistent with the synthesis method I.

### Example 2 (Synthesis Method II)

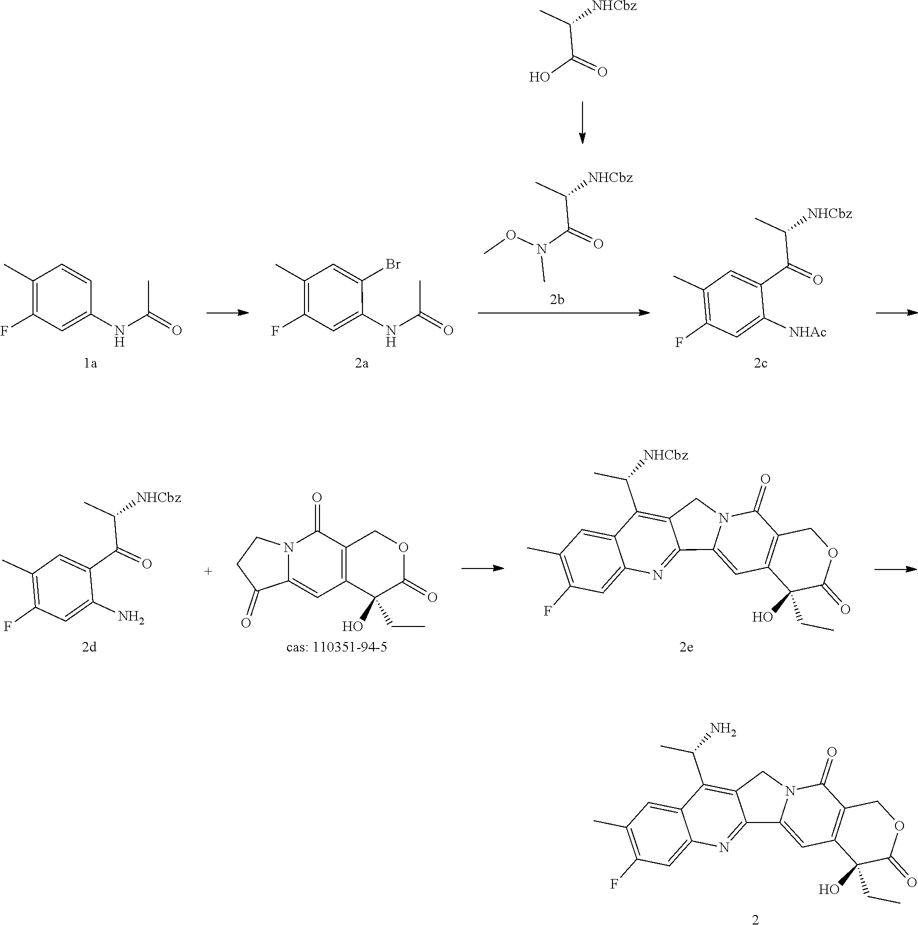

### Synthesis of Compound 2b

To a solution of N-Cbz-L-alanine (10.0 g, 0.045 mol) in dichloromethane (100 mL) was added CDI (9.5 g, 0.058 mol) at 5° C. and stirred for 1 h, followed by dimethylhydroxylamine hydrochloride (6.5 g, 0.067 mol) and triethylamine (13.6 g, 0.13 mol) were added to the above mixture and continued to stir for 1 h. The mixture was quenched with water, and the aqueous phase was extracted with dichloromethane (100 mL×3). The organic phases were combined, washed with 1N HCl, saturated sodium bicarbonate, brine, dried over anhydrous sodium sulfate, filtered and concentrated to obtain crude product 2b (11 g, yield 92%).

MS (ESI), m/z, 267.0 [M+H]⁺.

### Synthesis of Compound 2c

To a 2a (6.0 g, 0.024 mol) solution of dry tetrahydrofuran solution (80 mL) was added dropwise n-butyllithium solution (2.5 M in hexane, 21 mL, 0.054 mol) at −60° C., and stirred for 1 h. To the above mixture, 2b (7.8 g, 0.029 mol) in tetrahydrofuran (50 mL) was added dropwise, and the reaction was stirred for 2 h (−60-−20° C.). The mixture was quenched with ammonium chloride aqueous and extracted with ethyl acetate (80 mL×3). The organic phases were combined, washed with brine, dried over anhydrous sodium sulfate, filtered, concentrated, and purified by column chromatography to obtain compound 2c (1.1 g).

MS (ESI), m/z, 373.2 [M+H]⁺.

¹H NMR (400 MHZ, Chloroform-d) δ ppm 11.50 (s, 1H), 8.52 (d, J=12.5 Hz, 1H), 7.75 (d, J=8.2 Hz, 1H), 7.44-7.28 (m, 4H), 5.70 (d, J=7.8 Hz, 1H), 5.37 (t, J=7.4 Hz, 1H), 5.13 (s, 2H), 2.28 (s, 3H), 2.22 (s, 2H), 1.43 (d, J=7.1 Hz, 3H).

### Synthesis of Compound 2d

To a solution of 2c (300 mg, 0.80 mmol) in methanol and tetrahydrofuran (3/5 mL) was added concentrated hydrochloric acid (3 mL) and stirred for 12 h. The mixture was concentrated, basified with sodium bicarbonate to pH>7, diluted with water and extracted with dichloromethane (50 mL×3). The combined organic phase was washed with brine, dried over anhydrous sodium sulfate, filtered, and purified by column chromatography to obtain compound 2d (250 mg).

MS (ESI), m/z, 331.1 [M+H]⁺.

### Synthesis of Compound 2e

To a solution of 2d (400 mg, 1.21 mmol) in toluene (5 mL) was added ketone (CAS is 110351-94-5) (0.35 g, 1.33 mmol) and PPTS (60 mg, 0.24 mmol), and the reaction was stirred at 120° C. for 12 h. After completion, the mixture was concentrated and purified by column chromatography to obtain compound 2e (430 mg).

MS (ESI), m/z, 558.2 [M+H]⁺.

### Synthesis of Example 2

To a solution of 2e (400 mg, 0.72 mmol) and 6 N hydrochloric acid solution (100 mL/100 mL) was added 10% Pd/C (water content 55%, 100 mg) at room temperature 25° C. The flash was evacuated and backfilled with nitrogen and then H₂ (3×) and stirred under an atmosphere of H₂ for 12 h. The mixture was filtered with celite, washed with dichloromethane/methanol, concentrated, and slurried with ethanol to obtain Example 2 (hydrochloride), 300 mg.

The characterization data of LCMS and ¹H NMR were consistent with the synthesis method I.

### Examples 3 and 4

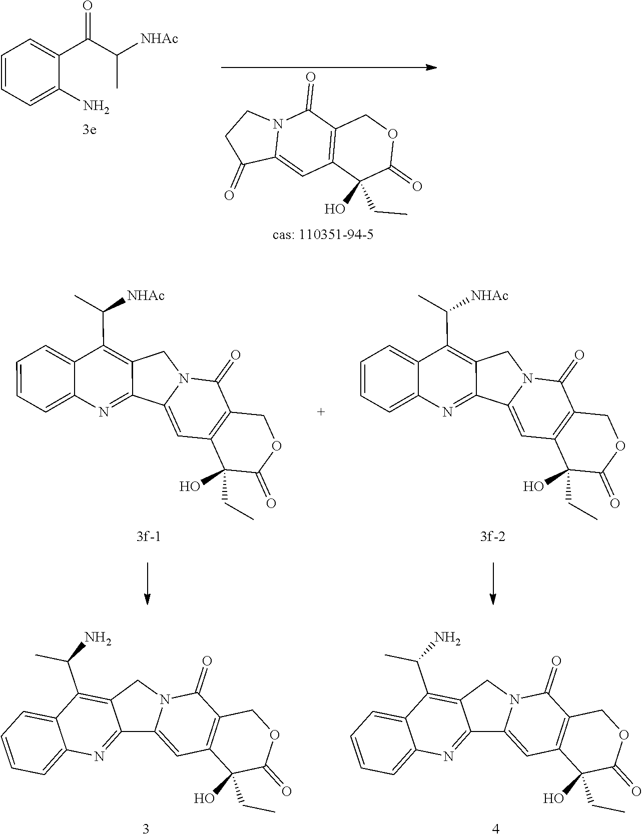

### Synthesis of Compound 3e

Compound 3e was synthesized according to a similar method to compound 1e in Examples 1 and 2 (synthesis method I).

### Synthesis of Compounds 3f-1 and 3f-2

Compound 3e (0.41 g, 2.0 mmol) was added to toluene (5 mL) at 20° C. To the above solution were added ketone (CAS is 110351-94-5) (0.63 g, 2.42 mmol) and p-toluenesulfonic acid monohydrate (38 mg, 0.20 mmol). The reaction was stirred at 120° C. for 16 h. LCMS showed that the product was formed. After the reaction was completed, the crude product was concentrated and purified by Prep-HPLC to obtain compounds 3f-1 (48 mg) and 3f-2 (45 mg).

MS (ESI), m/z, 434.1 [M+H]⁺.

### Synthesis of Example 3

3f-1 (48 mg, 0.11 mmol) was dissolved in 6N hydrochloric acid aqueous solution (1.0 mL) at 20° C. The mixture was heated to 100-105° C., and stirred for 16 h. LCMS showed that the product was formed. After the reaction was completed, the mixture was concentrated to obtain the Example 3 (25 mg, hydrochloride).

MS (ESI), m/z, 392.2 [M+H]⁺.

¹H NMR (400 MHZ, DMSO) δ 8.89 (s, 3H), 8.45 (d, J=8.6 Hz, 1H), 8.26 (dd, J=8.4, 1.0 Hz, 1H), 7.94 (dd, J=11.3, 4.0 Hz, 1H), 7.88-7.77 (m, 1H), 7.38 (s, 1H), 6.59 (s, 1H), 5.79-5.36 (m, 5H), 2.01-1.81 (m, 2H), 1.77 (d, J=7.0 Hz, 3H), 0.88 (t, J=7.3 Hz, 3H).

### Synthesis of Example 4

3f-2 (45 mg, 0.10 mmol) was dissolved in 6N hydrochloric acid aqueous solution (1.0 mL) at 20° C. The mixture was heated to 100-105° C. and stirred for 16 h. LCMS showed that the product was formed. After the reaction was completed, the mixture was concentrated to obtain the Example 4 (23 mg, hydrochloride).

MS (ESI), m/z, 392.2 [M+H]⁺.

¹H NMR (400 MHZ, DMSO) δ 8.89 (s, 3H), 8.46 (d, J=8.5 Hz, 1H), 8.26 (dd, J=8.4, 1.0 Hz, 1H), 7.94 (dd, J=11.3, 4.0 Hz, 1H), 7.87-7.77 (m, 1H), 7.38 (s, 1H), 6.59 (s, 1H), 5.83-5.36 (m, 5H), 1.95-1.82 (m, 2H), 1.79 (d, J=7.0 Hz, 3H), 0.88 (t, J=7.3 Hz, 3H).

The chiral configurations of Examples 3 and 4 were determined by synthesis method II (chiral synthesis).

### Example 3 (Synthesis Method II)

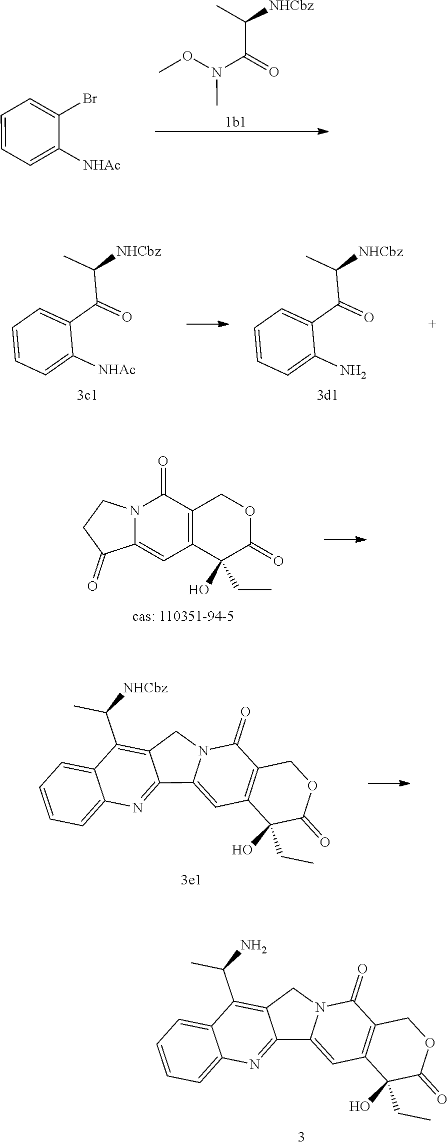

### Synthesis of Compound 3c1

To a solution of N-(2-bromophenyl) acetamide (2.0 g, 9.34 mmol) in dry tetrahydrofuran (50 mL) was added dropwise n-butyllithium solution (2.5 M in hexane, 8.2 mL, 17.9 mmol, 2.2 eq) at −60° C. and stirred for 1 h. A solution of 1b1 (3.7 g, 14.0 mmol, 1.5 eq) in tetrahydrofuran (10 mL) was added dropwise to the above solution, and stirred for 1 h (−60-−20° C.). The reaction solution was quenched with ammonium chloride aqueous solution, the aqueous phase was extracted with ethyl acetate (100 mL×3), the organic phases were combined, washed with brine, dried over anhydrous sodium sulfate, filtered, concentrated, and purified by column chromatography to obtain compound 3c1. 550 mg.

MS (ESI), m/z, 341.00 [M+H]⁺.

### Synthesis of Compound 3d1

To a solution of 3c1 (300 mg, 0.88 mmol) in methanol and tetrahydrofuran (5/5 mL) was added 3M hydrochloric acid (5 mL) in an ice-water bath at 5° C., then warmed to room temperature 25° C. and stirred for 12 h. The reaction solution was diluted with water and extracted with ethyl acetate (50 mL×3). The organic phases were combined, washed with brine, dried over anhydrous sodium sulfate, filtered, and purified by column chromatography to obtain compound 3d1, 180 mg.

MS (ESI), m/z, 299.1 [M+H]⁺.

### Synthesis of Compound 3e1

To a solution of 3d1 (150 mg, 0.50 mmol, 1.0 eq) in toluene (5 mL) was added ketone (CAS 110351-94-5) (158 mg, 0.60 mmol, 1.2 eq) and p-toluenesulfonic acid monohydrate (19 mg, 0.1 mmol, 0.2 eq), the reaction solution was heated to 110° C. and stirred for 3 h. After the reaction was completed, the reaction solution was concentrated under vacuo, added with water, stirred and filtered. The solid was purified by slurring with ethanol and water (10/2 mL) to obtain compound 3e1, 120 mg (ee, 95%).

MS (ESI), m/z, 526.1 [M+H]⁺.

### Asymmetric Synthesis of Example 3

To the mixed suspension solution of 3e1 (80 mg, 0.15 mmol) in 6M HCl (1 mL) and methanol (5 mL) was added 10% Pd/C (55% wet, 25 mg), backfilled with hydrogen, and stirred under an atmosphere of H₂ for 1 h at 25° C. The reaction solution was filtered, and the crude product was purified by Prep-HPLC (CH₃CN/H₂O, 0.05% TFA) and concentrated to obtain Example 3 (20 mg, ee, 95%).

MS (ESI), m/z, 392.10 [M+1]⁺.

The characterization data of LCMS and ¹H NMR were consistent with the synthesis method I.

### Example 4 (Synthesis Method II)

Synthesis of Example 4 (Synthesis Method II): It was synthesized according to the similar method in Example 3 (Synthesis Method II) and chiral intermediate 2b. The characterization data of LCMS and ¹H NMR were consistent with the synthesis method I.

### Examples 5 and 6

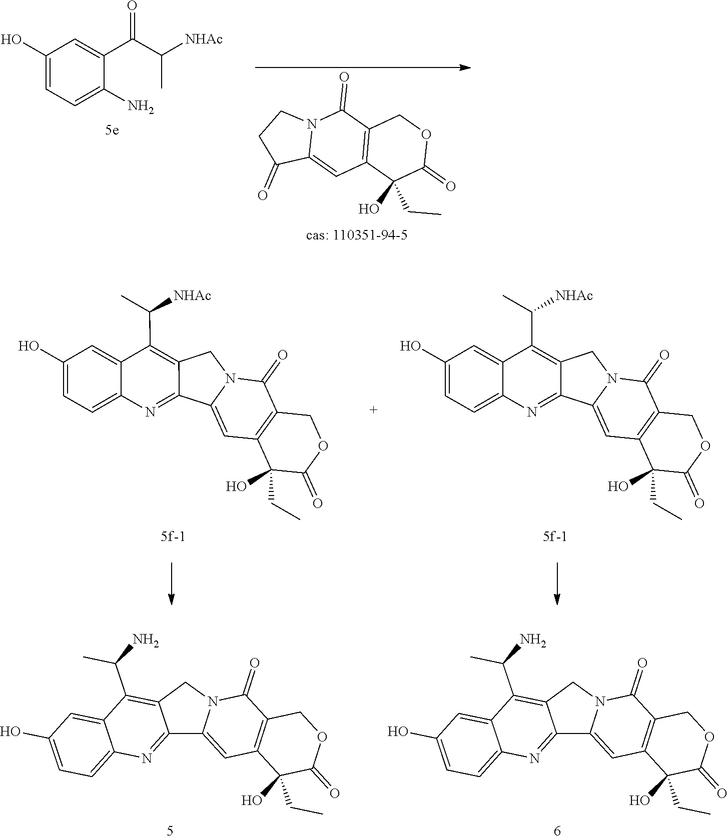

### Synthesis of Compound 5e

Compound 5e was synthesized according to a similar synthesis method to compound 1e in Examples 1 and 2 (synthesis Method 1).

### Synthesis of Compounds 5f-1 and 5f-2

5e (0.44 g, 2.0 mmol) was added to toluene (5 mL) at 20° C., and then ketone (CAS is 110351-94-5) (0.63 g, 2.42 mmol) and p-toluenesulfonic acid monohydrate (38 mg, 0.20 mmol) was added. The reaction was stirred at 120° C. for 16 h, and LCMS showed that the product was formed. After the reaction was completed, the mixture was concentrated to obtain crude product, which was directly purified by Prep-HPLC to obtain compounds 5f-1 (55 mg) and 5f-2 (58 mg).

MS (ESI), m/z, 450.1 [M+H]⁺.

### Synthesis of Example 5

5f-1 (55 mg, 0.12 mmol) was dissolved in 6N hydrochloric acid aqueous solution (1.0 mL) at 20° C. The mixture was heated to 100-105° C. and stirred for 16 h. LCMS showed that the product was formed. After the reaction was completed, the mixture was concentrated to obtain the Example 5 (30 mg, hydrochloride).

MS (ESI), m/z, 408.2 [M+H]⁺.

### Synthesis of Example 6

5f-2 (58 mg, 0.13 mmol) was dissolved in 6N hydrochloric acid aqueous solution (1.0 mL) at 20° C. The mixture was heated to 100-105° C. and stirred for 16 h. LCMS showed that the product was formed. After the reaction was completed, the mixture was concentrated to obtain the Example 6 (32 mg, hydrochloride).

The stereoconfiguration of Examples 5 and 6 were determined by their synthesis method II (asymmetric synthesis).

### Examples 5 and 6 (Synthesis Method II)

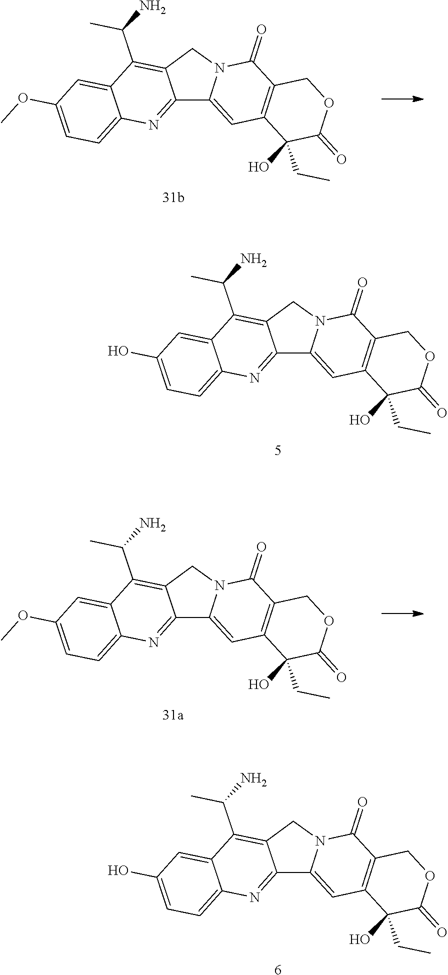

### Asymmetric Synthesis of Example 5 (Synthesis Method II)

To the suspension solution of compound 31b (15 mg, 0.03 mmol) in dry dichloromethane (2 mL) was added a solution of boron tribromide in dichloromethane (2.0 M, 0.1 mL, 0.20 mmol). The mixture was slowly warmed to room temperature 25° C. and stirred for 12 h. The reaction solution was quenched with saturated sodium bicarbonate solution, extracted with dichloromethane (10 mL×3), the organic phases were combined, washed with brine, dried over anhydrous sodium sulfate, concentrated, and purified by Prep-HPLC (CH₃CN/H₂O, 0.05% TFA) to obtain Example 5 (6.5 mg).

MS (ESI), m/z, 408.2 [M+H]⁺.

The characterization data of LCMS and ¹H NMR were consistent with Example 5 in synthesis method I.

### Asymmetric Synthesis of Example 6 (Synthesis Method II)

To the suspension solution of compound 31a (18 mg, 0.03 mmol) in dry dichloromethane (2 mL) was added a solution of boron tribromide in dichloromethane (2.0 M, 0.1 mL, 0.20 mmol). The mixture was slowly warmed to room temperature 25° C. and stirred for 12 h. The reaction solution was quenched with saturated sodium bicarbonate solution, extracted with dichloromethane (10 mL×3), the organic phases were combined, washed with brine, dried over anhydrous sodium sulfate, concentrated, and purified by Prep-HPLC (CH₃CN/H₂O, 0.05% TFA) to obtain Example 6 (5.6 mg).

MS (ESI), m/z, 408.2 [M+H]⁺.

The characterization data of LCMS and ¹H NMR were consistent with Example 6 in synthesis method I.

### Example 7

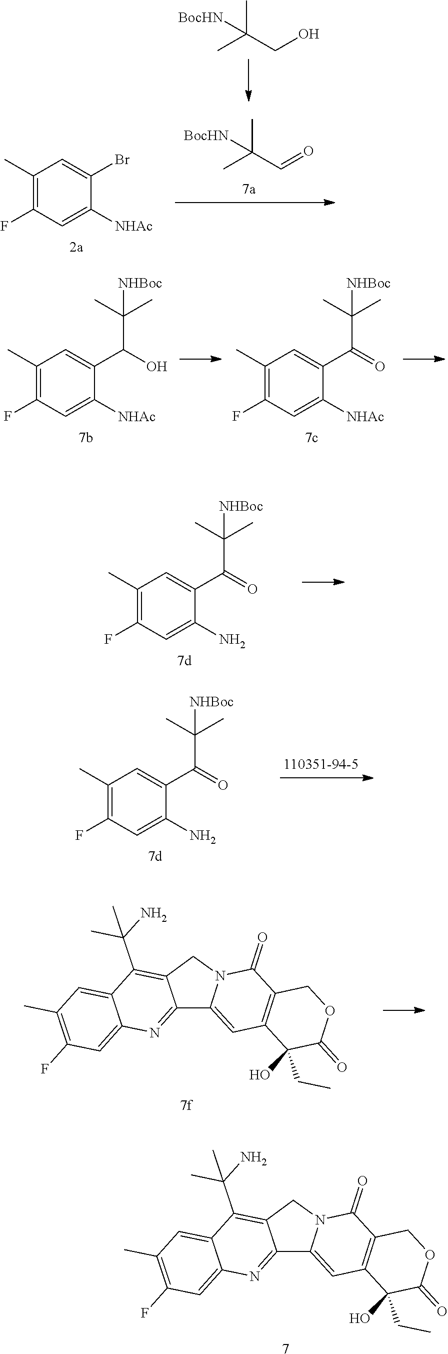

### Synthesis of Compound 7a

To a solution of N-Boc-2-amino-2-methyl-1-propanol (2.0 g, 0.011 mol) in dichloromethane (20 mL) at 5° C. was added Dess-Martin oxidizing agent (5.4 g, 8.94 mmol) and stirred for 1 h. The mixture was quenched with saturated Na₂S₂O₃ (10 mL) and NaHCO₃ (10 mL), the aqueous phase was extracted with dichloromethane (10 mL×3). The organic phases were combined, washed with brine, dried over anhydrous sodium sulfate, filtered, concentrated, and purified by column chromatography (petroleum ether/ethyl acetate) to give compound 7a (900 mg, yield 46%).

MS (ESI), m/z, 132.1 [M-55]⁺.

### Synthesis of Compound 7b

To a solution of 2a (1.0 g, 4.07 mmol) in dry tetrahydrofuran solution (10 mL) was added dropwise n-butyllithium solution (2.5 M in hexane, 3.6 mL, 8.9 mmol) at −60° C., and stirred for 1 h. A solution of 7a (0.76 g, 4.07 mmol) in tetrahydrofuran (2 mL) was added dropwise to the above solution, and the reaction was stirred at this temperature for 1 h. The mixture was quenched with aqueous ammonium chloride solution, and the aqueous phase was extracted with ethyl acetate (10 mL×3). The organic phases were combined, washed with brine, dried over anhydrous sodium sulfate, filtered, concentrated, and purified by column chromatography (petroleum ether/ethyl acetate) to obtain compound 7b (200 mg).

MS (ESI), m/z, 355.0 [M+H]⁺.

### Synthesis of Compound 7c

To a solution of 7b (200 mg, 0.57 mmol) in dichloromethane (20 mL) was added Dess-Martin oxidizing agent (360 mg, 0.85 mmol) at 5° C. and the reaction was stirred for 1 h (5-20° C.). The mixture was quenched with saturated Na₂S₂O₃ (10 mL) and NaHCO₃ (10 mL), and the aqueous phase was extracted with dichloromethane (10 mL×3). The organic phases were combined, washed with brine, dried over anhydrous sodium sulfate, filtered, concentrated, and the crude product was purified by column chromatography (petroleum ether/ethyl acetate) to obtain compound 7c (200 mg, yield 99%).

MS (ESI), m/z, 353.0 [M-55]⁺.

### Synthesis of Compound 7d

To a solution of 7c (200 mg, 0.57 mmol) in methanol (5 mL) was added 6 N hydrochloric acid (3 mL) at room temperature 25° C. and stirred for 48 h. The reaction solution was concentrated, basified with 2N NaOH to pH>7, and the aqueous phase was extracted with dichloromethane (10 mL×3). The organic phases were combined, washed with brine, dried over anhydrous sodium sulfate, filtered, and concentrated to obtain a crude product 7d (80 mg), which was directly used in the next reaction.

MS (ESI), m/z, 211.0 [M+H]⁺.

### Synthesis of Compound 7e

To a solution of 7d (80 mg, 0.38 mmol) in ethyl acetate/dichloromethane (5/5 mL) was added acetic anhydride (46 mg, 0.46 mmol) at 5° C. and stirred for 4 h (5-20° C.). Water (10 mL) was added to the above solution, and the aqueous phase was extracted with dichloromethane (10 mL×3). The organic phases were combined, washed with brine, dried over anhydrous sodium sulfate, filtered, and concentrated to obtain compound 7e (90 mg).

MS (ESI), m/z, 253.0 [M+H]⁺.

### Synthesis of Compound 7f

To a solution of 7e (90 mg, 0.32 mmol) in toluene (2 mL) was added ketone (CAS is 110351-94-5) (113 mg, 0.38 mmol) and p-toluenesulfonic acid monohydrate (30 mg, 0.16 mmol), and heated to 120° C. down and stirred for 12 h. After the reaction was completed, the reaction solution was concentrated and directly purified by column chromatography (dichloromethane/methanol) to obtain compound 7f (80 mg).

MS (ESI), m/z, 479.9 [M+H]⁺.

### Synthesis of Example 7

A mixture of 7f (80 mg, 0.17 mmol) and concentrated hydrochloric acid (5 mL) was heated to 130° C. for 12 h. After the reaction was completed, the reaction solution was concentrated and directly purified by Prep-HPLC to obtain Example 7 (23 mg, TFA salt).

MS (ESI), m/z, 437.8 [M+H]⁺.

¹H NMR (400 MHZ, DMSO-d₆) δ (ppm) 11.86 (d, J=57.6 Hz, 1H), 9.87 (s, 2H), 8.53-8.11 (m, 2H), 7.99 (d, J=10.8 Hz, 1H), 6.96-6.34 (m, 1H), 5.68-4.53 (m, 4H), 2.54 (d, J=1.7 Hz, 3H), 2.06-1.92 (m, 6H), 1.85 (d, J=8.0 Hz, 2H), 0.86 (q, J=7.3, 5.1 Hz, 3H).

### Example 8

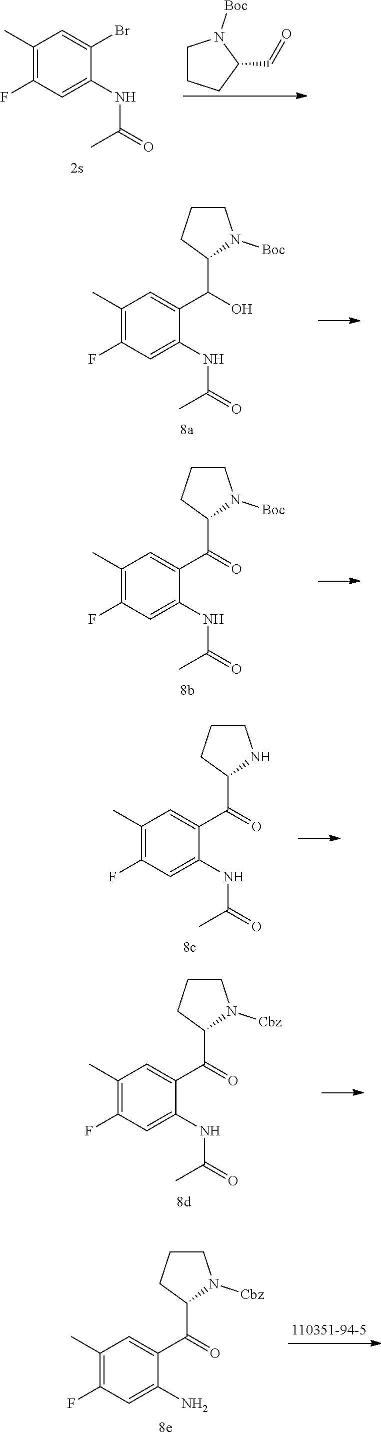

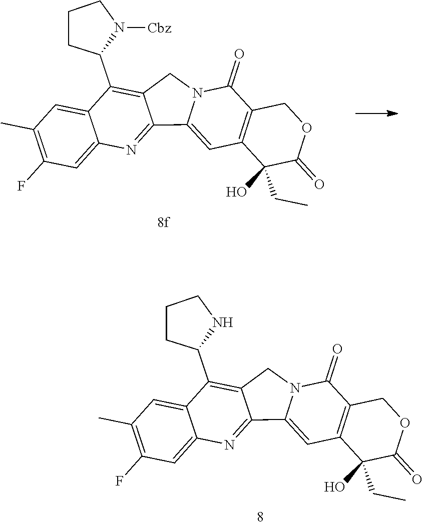 [SMILES: CC[C@@]1(O)C(=O)OCc2c1cc1n(c2=O)Cc2c-1nc1cc(F)c(C)cc1c2[C@@H]1CCCN1C(=O)OCc1ccccc1 ; "8f" ; CC[C@@]1(O)C(=O)OCc2c1cc1n(c2=O)Cc2c-1nc1cc(F)c(C)cc1c2[C@@H]1CCCN1 ; "8"]

### Synthesis of Compound 8a

To a solution of 2a (2.0 g, 8.13 mmol) in dry tetrahydrofuran solution (20 mL) was added dropwise n-butyllithium solution (2.5 M in hexane, 7.2 mL, 17.9 mmol) at −60° C. and stirred for 1 h. A solution of N-Boc-L-prolinaldehyde (2.4 g, 12.2 mmol) in tetrahydrofuran (5 mL) was added dropwise to the above solution and stirred for 1 h at this temperature. The mixture was quenched with aqueous ammonium chloride solution, and the aqueous phase was extracted with ethyl acetate (30 mL×3). The organic phases were combined, washed with brine, dried over anhydrous sodium sulfate, filtered, concentrated, and purified by column chromatography (petroleum ether/ethyl acetate) to obtain compound 8a (1.0 g, containing isomers).

MS (ESI), m/z, 367.0 [M+H]⁺.

### Synthesis of Compound 8b

To a solution of 8a (1.8 g, 4.92 mmol) in dichloromethane (20 mL) was added Dess-Martin oxidizing agent (3.1 g, 7.38 mmol) at 5° C. and the reaction was stirred for 1 h (5-20° C.). The mixture was quenched with saturated Na₂S₂O₃ (20 mL) and NaHCO₃ (20 mL), the aqueous phase was extracted with dichloromethane (50 mL×3), the organic phases were combined, washed with brine, dried over anhydrous sodium sulfate, filtered, concentrated, and purified by column chromatography (petroleum ether/ethyl acetate) to give compound 8b (1.0 g).

MS (ESI), m/z, 365.0 [M+H]⁺.

### Synthesis of Compound 8c

To a solution of 8b (300 mg, 0.82 mmol) in dichloromethane (5 mL) was added TFA (1 mL) at 5° C. and stirred for 1 h. The reaction solution was concentrated, diluted with dichloromethane, alkalized with sodium bicarbonate to pH>7, diluted with water and extracted with dichloromethane (20 mL×3). The organic phases were combined, washed with salt, dried over anhydrous sodium sulfate, filtered, and concentrated to obtain compound 8c (200 mg, yield 99%).

MS (ESI), m/z, 265.0 [M+H]⁺.

### Synthesis of Compound 8d

To a solution of 8c (200 mg, 0.55 mmol) in dichloromethane (10 mL) was add benzyl chloroformate (112 mg, 0.66 mmol) and triethylamine (111 mg, 1.10 mmol) at 5° C. and stirred for 1 h. The reaction solution was quenched with water, and the aqueous phase was extracted with dichloromethane (20 mL×3). The organic phases were combined, washed with brine, dried over anhydrous sodium sulfate, filtered, concentrated, and purified by column chromatography (petroleum ether/ethyl acetate) to obtain compound 8d (170 mg, yield 57%).

MS (ESI), m/z,399.0 [M+H]⁺.

### Synthesis of Compound 8e

To a solution of 8d (170 mg, 0.43 mmol) in methanol (3 mL) was added 6 N hydrochloric acid (3 mL) solution at room temperature 25° C. and stirred for 12 h. The reaction solution was concentrated, basified with 2N sodium hydroxide to pH>7, and the aqueous phase was extracted with dichloromethane (20 mL×3). The organic phases were combined, washed with salt, dried over anhydrous sodium sulfate, filtered, and concentrated to obtain crude product 8e (152 mg, yield 99%), which was directly used in the next step.

MS (ESI), m/z, 357.1 [M+H]⁺.

### Synthesis of Compound 8f

To a solution of 8e (150 mg, 0.42 mmol) in toluene (3 mL) was added ketone (CAS is 110351-94-5) (133 mg, 0.51 mmol) and p-toluenesulfonic acid monohydrate (40 mg, 0.21 mmol) and then heated to 120° C. and stirred for 6 h. The reaction solution was concentrated and directly purified by column chromatography (dichloromethane/methanol) to obtain compound 8f (150 mg, yield 61%).

MS (ESI), m/z, 584.1 [M+H]⁺.

### Synthesis of Example 8

To a solution of 8f (150 mg, 0.26 mmol) in methanol (10 mL) at room temperature 25° C. was added 10% Pd/C (water content 55%, 50 mg), backfilled with nitrogen and backfilled with hydrogen, and stirred under an atmosphere of H₂ for 1 h. The reaction solution was filtered with celite, the filtrate was concentrated, and the crude product was purified by Prep-HPLC to obtain Example 8 (55 mg).

MS (ESI), m/z, 449.9 [M+H]⁺.

¹H NMR (400 MHZ, DMSO-d₆) δ (ppm) 9.78 (s, 1H), 8.91 (s, 1H), 8.34 (d, J=7.9 Hz, 1H), 8.02 (d, J=10.4 Hz, 1H), 7.37 (s, 1H), 6.58 (s, 1H), 5.79-5.36 (m, 5H), 3.54 (ddd, J=14.2, 9.4, 3.8 Hz, 2H), 2.56 (d, J=1.7 Hz, 3H), 2.39-2.29 (m, 1H), 2.26-2.12 (m, 1H), 1.95-1.78 (m, 2H), 0.88 (t, J=7.3 Hz, 3H).

### Example 9

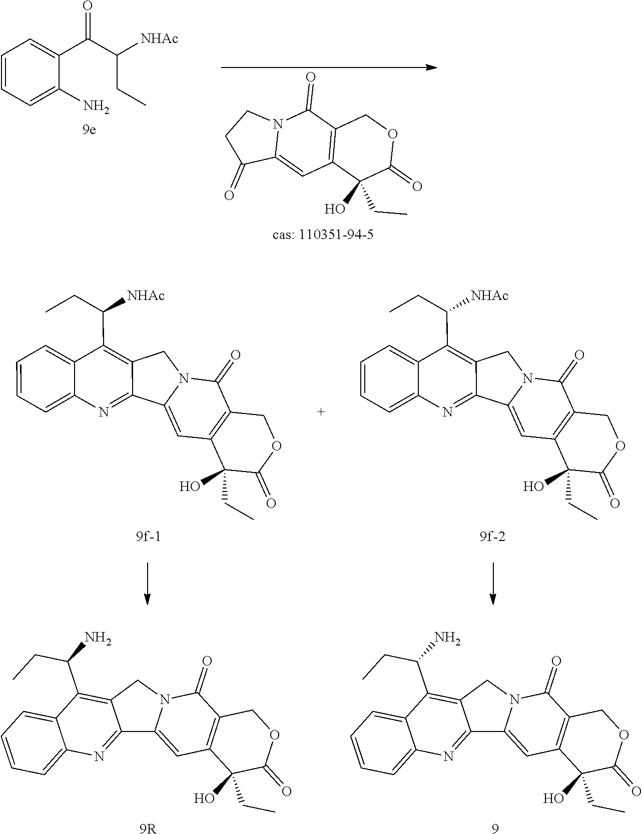

### Synthesis of Compound 9e

Compound 9e was synthesized according to a similar synthesis method to compound 1e in Examples 1 and 2 (synthesis method I).

### Synthesis of Compound 9f-2

9e (0.42 g, 2.0 mmol) was added to toluene (5 mL) at 20° C. Ketone (CAS is 110351-94-5) (0.63 g, 2.42 mmol) and p-toluenesulfonic acid monohydrate (38 mg, 0.20 mmol) were added, and the reaction was stirred at 120° C. for 16 h. LCMS showed that the product was formed. After the reaction was completed, the crude product was concentrated, and the crude product was directly purified by Prep-HPLC to obtain compounds 9f-1 (48 mg) and 9f-2 (51 mg).

### Synthesis of Example 9R

9f-1 (48 mg, 0.107 mmol) was added to 6 N hydrochloric acid aqueous solution (1.0 mL) at 20° C. and then heated to 100-105° C., stirred for 16 h. LCMS showed that the product was formed. After the reaction was completed, the reaction was concentrated to obtain Example 9R (23 mg, hydrochloride).

MS (ESI), m/z, 406.1 [M+H]⁺.

### Synthesis of Example 9

9f-2 (51 mg, 0.11 mmol) was added to 6N aqueous hydrochloric acid solution (1.0 mL) at 20° C. and then heated to 100-105° C. and stirred for 16 h. LCMS showed that the product was formed. After the reaction was completed, the mixture was concentrated to obtain Example 9 (28 mg, hydrochloride).

MS (ESI), m/z, 406.1 [M+H]⁺.

¹H NMR (400 MHZ, DMSO) δ ppm 8.31 (d, J=8.5 Hz, 1H), 7.88 (dd, J=8.4, 1.0 Hz, 1H), 7.73 (dd, J=11.3, 4.0 Hz, 1H), 7.69 (m, 1H), 7.35 (s, 1H), 5.63 (s, 1H), 5.51 (s, 1H), 5.46-5.33 (m, 5H), 1.91-1.80 (m, 4H), 1.00 (t, J=7.3 Hz, 3H), 0.88 (t, J=7.3 Hz, 3H).

The stereoconfigurations of Example 9 and Example 9R were determined by their synthesis method II (asymmetric synthesis).

### Example 9 (Synthesis Method II)

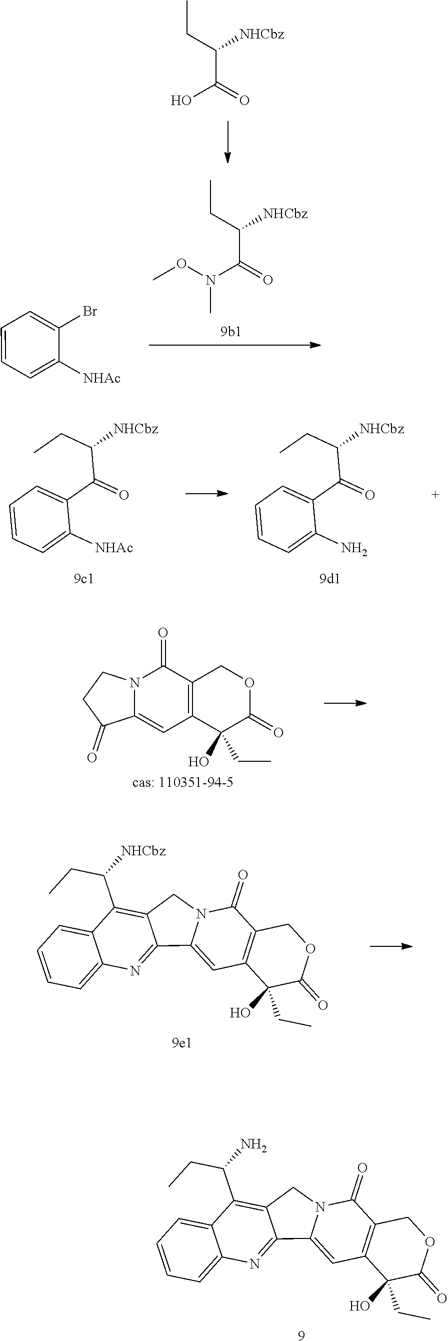

### Synthesis of Compound 9b1

To a solution of(S)-2-(benzyloxycarbonylamino) butyric acid (3.0 g, 12.6 mmol) in dichloromethane (60 mL) at 5° C. was added CDI (3.1 g, 19.0 mmol, 1.5 eq) and stirred for 1 h, then N,O-Dimethylhydroxylamine HCl (1.84 g, 19.0 mmol, 1.5 eq) and triethylamine (3.2 g, 31.6 mmol, 2.5 eq) were added to the above mixture and stirred for 1 h. The reaction solution was quenched with water, and the aqueous phase was extracted with dichloromethane (100 mL×3). The organic phases were combined, washed with 1 N HCl, washed with saturated sodium bicarbonate, washed with brine, dried over anhydrous sodium sulfate, filtered and concentrated to obtain crude product 9b1 (3.0 g, yield 85%).

MS (ESI), m/z, 280.9 [M+H]⁺.

### Synthesis of Compound 9c1

To a solution of N-(2-bromophenyl) acetamide (2.0 g, 9.34 mmol) in dry tetrahydrofuran (50 mL) at −60° C. was added dropwise n-butyllithium solution (2.5 M in hexane, 8.2 mL, 17.9 mmol, 2.2 eq) and stirred for 1 h. 9b1 (3.9 g, 14.0 mmol, 1.5 eq) in tetrahydrofuran (10 mL) was added dropwise to the above solution, and the reaction was stirred for 1 h (−60-−20° C.). The reaction solution was quenched with ammonium chloride aqueous solution, the aqueous phase was extracted with ethyl acetate (100 mL×3). The organic phases were combined, washed with brine, dried over anhydrous sodium sulfate, filtered, concentrated, and purified by column chromatography to obtain compound 9c1 (350 mg).

MS (ESI), m/z, 355.0 [M+H]⁺.

### Synthesis of Compound 9d1

To a solution of 9c1 (320 mg, 0.90 mmol) in methanol and tetrahydrofuran (5/5 mL) was added 3M hydrochloric acid (5 mL) in an ice-water bath at 5° C., then warmed to room temperature 25° C. and stirred for 12 h. The reaction solution was diluted with water and extracted with ethyl acetate (50 mL×3). The organic phases were combined, washed with brine, dried over anhydrous sodium sulfate, filtered, and separated and purified by column chromatography to obtain compound 9d1 (220 mg).

MS (ESI), m/z, 313.1 [M+H]⁺.

### Synthesis of Compound 9e1

To a solution (6 mL) of 9d1 (150 mg, 0.48 mmol, 1.0 eq) in toluene was added ketone (CAS is 110351-94-5) (190 mg, 0.72 mmol, 1.2 eq) and p-toluenesulfonic acid monohydrate (19 mg, 0.1 mmol, 0.2 eq). The reaction was heated to 110° C. and stirred for 5 h. After the reaction was completed, the reaction solution was concentrated under vacuo, added with water, stirred and filtered, and the solid was purified by slurring with ethanol and water (10/2 mL) to obtain compound 9e1 (180 mg, ee 92%).

MS (ESI), m/z, 540.1 [M+H]⁺.

### Asymmetric Synthesis of Example 9

To a suspension solution of 9e1 (100 mg, 0.19 mmol) in 6M HCl (1 mL) and methanol (5 mL) was added 10% Pd/C (55% wet, 30 mg), backfilled with hydrogen, and stirred under hydrogen atmosphere at 25° C. for 1 h. After the reaction was completed, filtered, and the crude product was purified and concentrated, purified with Prep-HPLC (CH₃CN/H₂O, 0.05% TFA) to obtain Example 9 (46 mg, ee 94%).

MS (ESI), m/z, 406.1 [M+1]⁺.

The characterization data of LCMS and ¹H NMR were consistent with the synthesis method I.

### Example 9R (Synthesis Method II)

Synthesis of Example 9R (Synthesis Method II): synthesized according to a method similar to that in Example 9 (Synthesis Method II). The characterization data of LCMS and ¹H NMR were consistent with the synthesis method I.

### Example 10

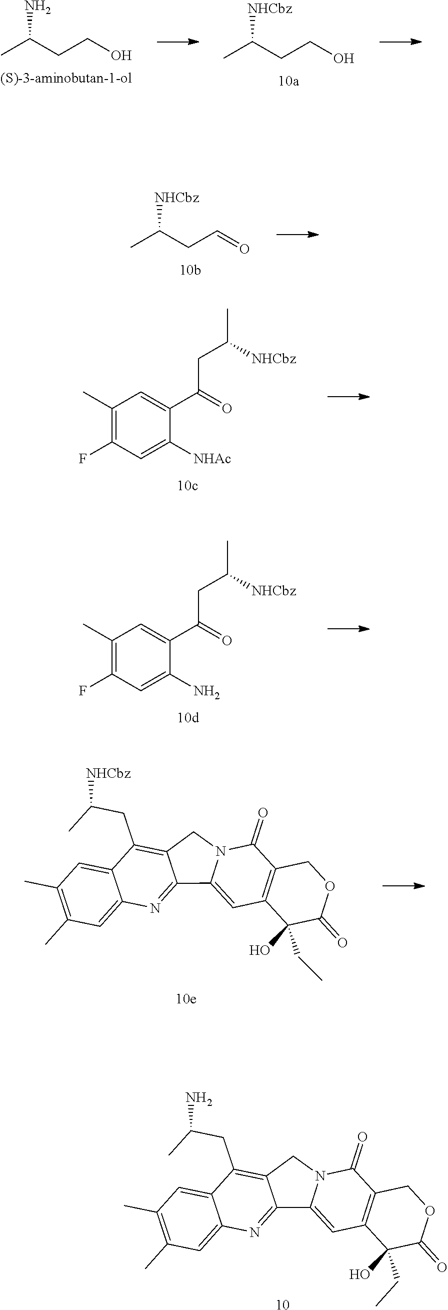

### Synthesis of Compound 10a

To a solution of(S)-3-aminobutanol (4.5 g, 0.051 mol) in tetrahydrofuran (50 mL) was added K₂CO3 (14 g, 0.1 mol) and water (10 mL) at 5° C., followed by chloroformic acid Benzyl ester (9.5 g, 0.055 mol) was added dropwise and stirred for 1 h. The mixture was quenched with water and the aqueous phase was extracted with dichloromethane (50 mL×3). The organic phases were combined, washed with brine, dried over anhydrous sodium sulfate, filtered, and compound 10a was concentrated (11.0 g, yield 97%).

MS (ESI), m/z, 224.0 [M+H]⁺.

### Synthesis of Compound 10b

To a solution of 10a (11.0 g, 0.05 mol) in dichloromethane (100 mL) was added Dess-Martin oxidizing agent (41.8 g, 0.1 mol) at 5° C. and stirred for 24 h (5-20° C.). The reaction solution was quenched with saturated Na₂S₂O₃ (50 mL) and NaHCO₃ (50 mL), and the aqueous phase was extracted with dichloromethane (100 mL×3). The organic phases were combined, washed with brine, dried over anhydrous sodium sulfate, filtered, concentrated, and the crude product was purified by column chromatography (petroleum ether/ethyl acetate) to obtain compound 10b (4.5 g, yield 41%).

MS (ESI), m/z, 222.1 [M+H]⁺.

### Synthesis of Compound 10c

10b (4.5 g, 0.021 mol) was dissolved in toluene (45 mL) at 10° C., and then 5a (1.75 g, 0.011 mol) and palladium acetate (90 mg, 0.4 mmol) were added. Under nitrogen atmosphere, trifluoroacetic acid (1.2 g, 0.01 mol) and TBHP (1.9 g, 0.02 mol) was added to the above mixture. The reaction was heated to 40-50° C. for 20 h. The reaction solution was quenched with water, and the aqueous phase was extracted with dichloromethane (50 mL×3). The organic phases were combined, washed with brine, dried over anhydrous sodium sulfate, filtered, concentrated, and the crude product was purified by column chromatography to obtain compound 10c (1.0 g).

MS (ESI), m/z, 387.2 [M+H]⁺.

### Synthesis of Compound 10d

Compound 10c (1.0 g, 2.6 mmol) was dissolved in ethanol (5 mL) at 20° C., then concentrated hydrochloric acid (1 mL) was added. The reaction solution was heated to 40-50° C. and stirred for 12 h. After the reaction was completed, the mixture was directly concentrated to obtain crude product 10d (0.89 g, 99% yield), which was directly used in the next step.

MS (ESI), m/z, 345.1 [M+H]⁺.

### Synthesis of Compound 10e

To a solution of 10d (crude product 800 mg, 2.32 mmol) in toluene (5 mL) was added ketone (CAS is 110351-94-5) (730 mg, 2.79 mmol) and p-toluenesulfonic acid monohydrate (44 mg, 0.23 mmol). The reaction was heated to 120° C. and stirred for 16 h. The reaction solution was concentrated and purified by column chromatography (dichloromethane/methanol) to obtain compound 10e (300 mg, yield 22%).

MS (ESI), m/z, 572.1 [M+H]⁺.

### Synthesis of Example 10

Compound 10e (300 mg, 0.52 mmol) was disssovled in tetrahydrofuran (20 mL) at 20° C., and Pd/C (20 mg) was added. The flash was evacuated and backfilled with H₂ (3×) and stirred under an atmosphere of H₂ for 16 h. The reaction solution was filtered and concentrated, and the crude product was purified by Prep-HPLC to obtain Example 10 (20 mg, TFA salt, purity 93%).

MS (ESI), m/z, 438.1 [M+H]⁺.

¹H NMR (400 MHZ, CDCl₃) δ ppm 8.23 (d, J=7.9 Hz, 1H), 7.94 (d, J=10.4 Hz, 1H), 7.87 (br, 2H), 7.34 (s, 1H), 6.58 (s, 1H), 5.47-5.23 (m, 4H), 3.73-3.69 (m, 2H), 2.52 (s, 3H), 2.01-1.87 (m, 2H), 1.26 (t, J=7.3 Hz, 3H), 0.89 (t, J=7.3 Hz, 3H).

### Example 11

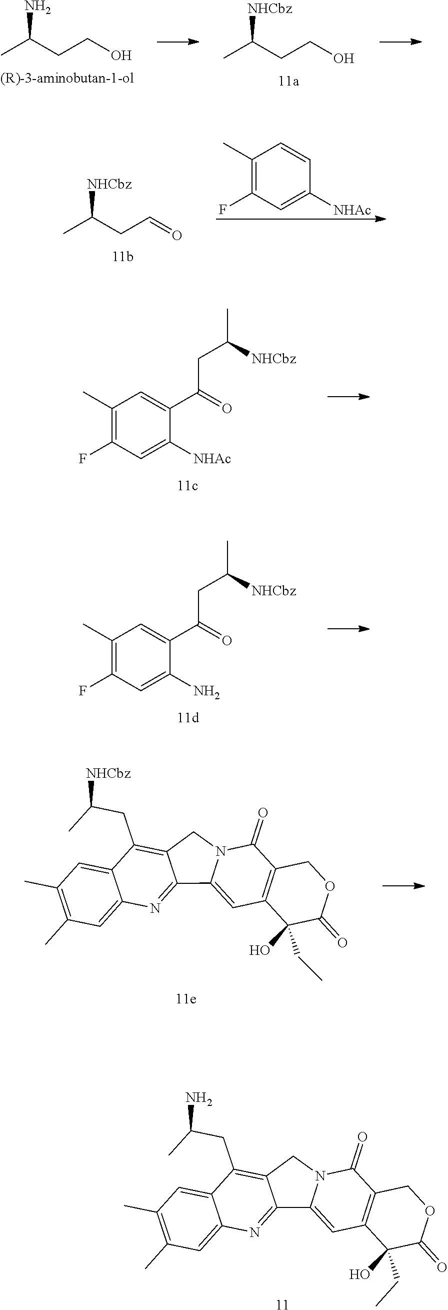

### Synthesis of Compound 11a

To a solution of (R)-3-aminobutanol (4.5 g, 0.051 mol) in tetrahydrofuran (50 mL) was added K₂CO₃ (14 g, 0.1 mol) and water (10 mL) at 5° C. Then benzyl chloroformate (9.5 g, 0.055 mol) was added dropwise to the above solution and continued to stir for 1 h. The reaction solution was quenched with water, and the aqueous phase was extracted with dichloromethane (50 mL×3). The organic phases were combined, washed with brine, dried over anhydrous sodium sulfate, filtered, concentrated to give compound 11a (11.2 g, yield 99%).

MS (ESI), m/z, 224.0 [M+H]⁺.

### Synthesis of Compound 11b

To a solution of 11a (11.0 g, 0.05 mol) in dichloromethane (100 mL) was added Dess-Martin oxidizing agent (41.8 g, 0.1 mol) at 5° C. and the reaction was stirred for 12 h (5-20° C.). The mixture was quenched with saturated Na₂S₂O₃ (50 mL) and NaHCO₃ (50 mL) and extracted the aqueous phase with dichloromethane (100 mL×3). The organic phases were combined, washed with brine, dried over anhydrous sodium sulfate, filtered, concentrated, and purified by column chromatography (petroleum ether/ethyl acetate) to obtain compound 11b (6.0 g, yield 55%).

MS (ESI), m/z, 222.1 [M+H]⁺.

### Synthesis of Compound 11c

11b (4.5 g, 0.021 mol) was dissolved in toluene (45 mL) at 10° C., and then 5a (1.75 g, 0.011 mol) and palladium acetate (90 mg, 0.4 mmol) was added. Trifluoroacetic acid (1.2 g, 0.01 mol) and TBHP (1.9 g, 0.02 mol) were added to the mixture under nitrogen atmosphere. The reaction was then heated to 40-50° C. for 24 h. The mixture was quenched with water and the aqueous phase was extracted with dichloromethane (50 mL×3). The organic phases were combined, washed with brine, dried over anhydrous sodium sulfate, filtered, concentrated, and purified by column chromatography to obtain compound 11c (1.2 g).

MS (ESI), m/z, 387.2 [M+H]⁺.

### Synthesis of Compound 11d

Compound 11c (1.0 g, 2.6 mmol) was dissolved in ethanol (5 mL) at 20° C., and then concentrated hydrochloric acid (1 mL) was added. The reaction was heated to 40-50° C. and stirred for 12 h. After the reaction was completed, the reaction solution was directly concentrated to obtain crude product 11d (0.8 g, 97% yield), which was directly used in the next step.

MS (ESI), m/z, 345.1 [M+H]⁺.

### Synthesis of Compound 11e

To a solution of 11d (crude 800 mg, 2.32 mmol) in toluene (5 mL) was added ketone (CAS is 110351-94-5) (730 mg, 2.79 mmol) and p-toluenesulfonic acid monohydrate (44 mg, 0.23 mmol). The reaction was heated to 120° C. and stirred for 16 h. The mixture was concentrated and purified by column chromatography (dichloromethane/methanol) to obtain compound 11e (500 mg, yield 40%).

MS (ESI), m/z, 572.1 [M+H]⁺.

### Synthesis of Example 11

Compound 11e (400 mg, 0.70 mmol) was dissolved in tetrahydrofuran (20 mL) at 20° C., then Pd/C (20 mg) was added. The flash was evacuated and backfilled with H₂ (3×) and stirred under an atmosphere of H₂ for 16 h. The reaction solution was filtered, concentrated, and purified by Prep-HPLC to obtain Example 11 (60 mg, TFA salt, purity 97%).

MS (ESI), m/z, 438.1 [M+H]⁺.

¹H NMR (400 MHZ, CDCl₃) δ ppm 8.24 (d, J=7.9 Hz, 1H), 8.02-7.98 (m, 2H), 7.40 (d, J=10.3 Hz, 1H), 6.63 (br, 2H), 5.51-5.08 (m, 3H), 3.73-3.69 (m, 1H), 2.52 (s, 3H), 2.01-1.87 (m, 2H), 1.26 (t, J=7.3 Hz, 3H), 0.89 (t, J=7.3 Hz, 3H).

### Example 12

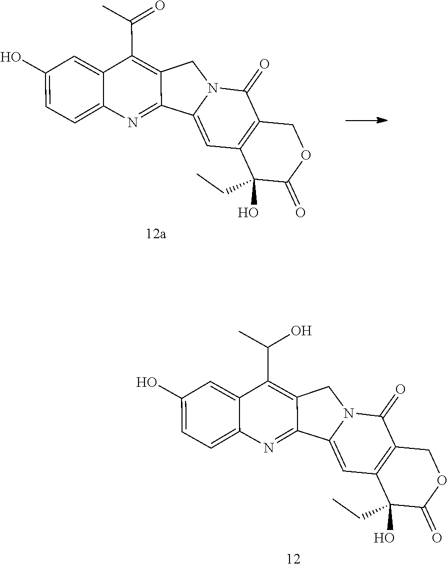 [SMILES: CC[C@@]1(O)C(=O)OCc2c1cc1n(c2=O)Cc2c-1nc1ccc(O)cc1c2C(C)=O ; "12a" ; CC[C@@]1(O)C(=O)OCc2c1cc1n(c2=O)Cc2c-1nc1ccc(O)cc1c2C(C)O ; "12"]

### Synthesis of Example 12

To a solution of compound 12a (200 mg, 0.49 mmol) in methanol (10 mL) was added sodium borohydride (37 mg, 0.98 mmol) at 0-5° C. and stirred for 0.5 h. The reaction solution was quenched with water, concentrated, and purified by silica gel column chromatography (DCM/MeOH=0-5%) to obtain Example 12 (100 mg, yield 50%).

MS (ESI), m/z, 409.1 [M+H]⁺.

### Example 12-1

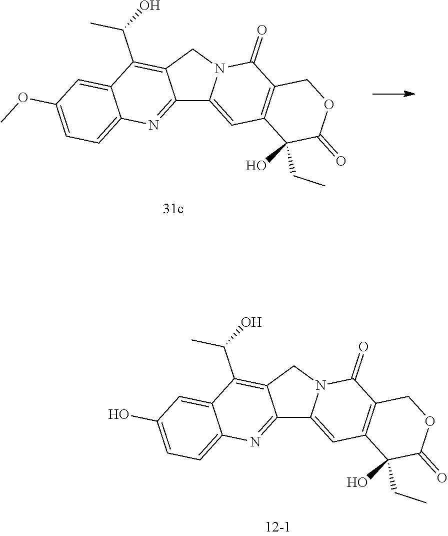 [SMILES: CC[C@@]1(O)C(=O)OCc2c1cc1n(c2=O)Cc2c-1nc1ccc(OC)cc1c2[C@H](C)O ; CC[C@@]1(O)C(=O)OCc2c1cc1n(c2=O)Cc2c-1nc1ccc(O)cc1c2[C@H](C)O]

### Synthesis of Example 12-1

The mixture of compound 31c (10 mg, 0.02 mmol) and 48% HBr acid aqueous solution was heated to reflux for 1 h. The mixture was concentrated and purified by Prep-HPLC to obtain Example 12-1 (2.2 mg).

MS (ESI), m/z, 409.1 [M+H]⁺.

### Example 12-2

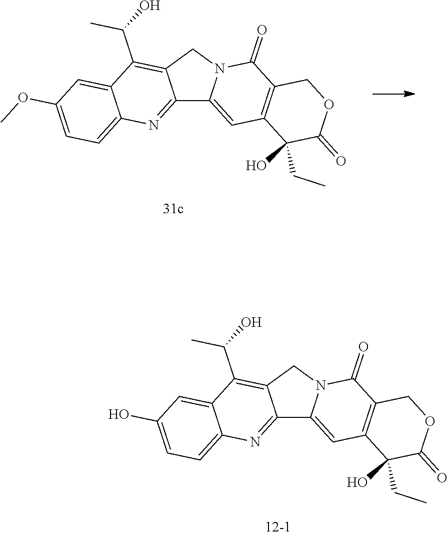 [SMILES: CC[C@@]1(O)C(=O)OCc2c1cc1n(c2=O)Cc2c-1nc1ccc(OC)cc1c2[C@H](C)O ; CC[C@@]1(O)C(=O)OCc2c1cc1n(c2=O)Cc2c-1nc1ccc(O)cc1c2[C@H](C)O]

### Synthesis of Example 12-2

The mixture of compound 31d (10 mg, 0.02 mmol) and 48% HBr acid aqueous solution was heated to reflux for 1 h. The mixture was concentrated and purified by Prep-HPLC (CH₃CN/H₂O, 0.05% TFA) to obtain Example 12-2 (3.2 mg).

MS (ESI), m/z, 409.1 [M+H]⁺.

### Example 12b

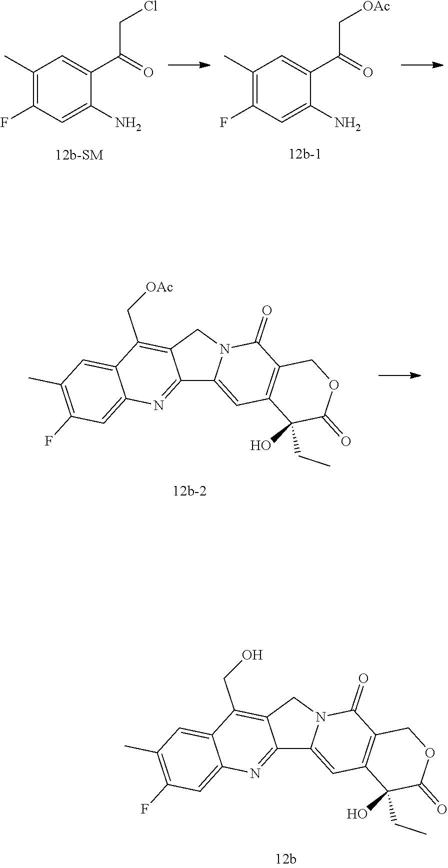 [SMILES: Cc1cc(C(=O)CCl)c(N)cc1F ; CC(=O)OCC(=O)c1cc(C)c(F)cc1N ; CC[C@@]1(O)C(=O)OCc2c1cc1n(c2=O)Cc2c-1nc1cc(F)c(C)cc1c2COC(C)=O ; CC[C@@]1(O)C(=O)OCc2c1cc1n(c2=O)Cc2c-1nc1cc(F)c(C)cc1c2CO]

### Synthesis of Compound 12b-1

To a solution of 12b-SM (synthesized according to the method of WO2020219287A1 compound 2a, 100 mg, 0.5 mmol, 1.0 eq) in dry DMF was added sodium acetate (82 mg, 1.0 mmol, 2.0 eq) at 25° C. under nitrogen atmosphere. The reaction was stirred 12 h and LCMS showed that the reaction was complete. The mixture was quenched with water, diluted with ethyl acetate, and the aqueous phase was extracted with ethyl acetate (30 mL×3). The organic phases were combined, washed with brine, dried over anhydrous sodium sulfate, filtered, and concentrated to obtain crude product 12b as light yellow solid (yield >99%), which was used directly in the next step.

MS (ESI), m/z, 226.0 [M+1]⁺.

### Synthesis of Compound 12b-2

To a solution of 12b-1 (110.0 mg, 0.49 mmol, 1.0 eq) in toluene (5 mL) was added ketone (CAS is 110351-94-5) (154.3 mg, 0.59 mmol, 1.2 eq) and p-toluenesulfonic acid monohydrate (18.6 mg, 0.10 mmol, 0.2 eq) at room temperature 25° C. under nitrogen atmosphere. The reaction was heated to 110° C. for 3 h, and LCMS showed that the reaction was complete. The mixture was quenched with water, diluted with dichloromethane, and the aqueous phase was extracted with dichloromethane (30 mL×3). The organic phases were combined, washed with brine, dried over anhydrous sodium sulfate, filtered, and concentrated to obtain a crude product (12b-2, 220 mg, colorless oil, yield 99%), which was used directly in the next reaction.

MS (ESI), m/z, 453.0 [M+1]⁺.

### Synthesis of Example 12b

To a solution of 12b-2 (crude 220 mg, 0.49 mmol, 1.0 eq) in methanol (5 mL) was added 2 N NaOH solution (1 mL) at room temperature 25° C. under nitrogen atmosphere, and the reaction was stirred for 1 h. The reaction solution was adjusted to pH<7 with 2 N HCl solution, concentrated, and purified by Pre-HPLC (CH₃CN/H₂O, 0.05% TFA) to obtain product 12b (40 mg, light yellow solid, yield 20%, purity 98.93%).

MS (ESI), m/z, 411.00 [M+1]⁺.

¹H NMR (400 MHZ, DMSO-d₆) δ 8.13 (d, J=8.2 Hz, 1H), 7.88 (d, J=10.9 Hz, 1H), 6.53 (d, J=1.4 Hz, 1H), 5.95-5.79 (m, 1H), 5.57-5.32 (m, 4H), 5.25 (d, J=5.5 Hz, 2H), 2.51 (p, J=1.9 Hz, 35H), 1.87 (hept, J=7.0 Hz, 2H), 0.88 (t, J=7.3 Hz, 3H).

### Examples 12c and 12d

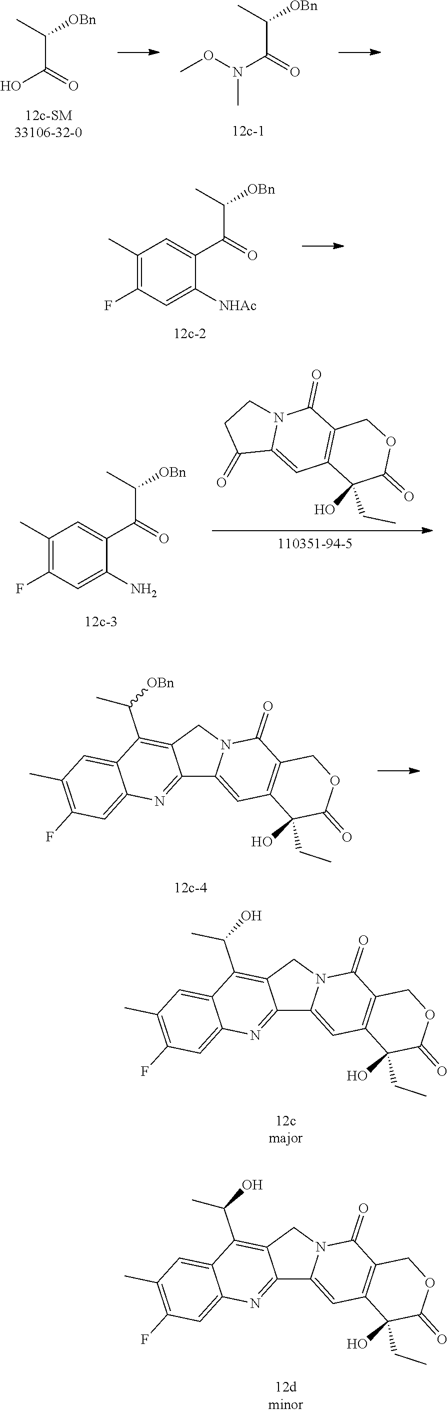

### Synthesis of Compound 12c-1

Compound 12c-SM was dissolved in DCM (100 mL) and cooled to 5° C. Then oxalyl chloride (21 g, 166.6 mmol) and 1 drop of DMF was added, and the reaction solution is stirred at room temperature for 2 h. After LCMS showed that the reaction was completed, the reaction solution was concentrated to obtain 15 g as a light-yellow oil with a yield of 91%, which was used directly in the next step.

To a solution of dimethylhydroxylamine hydrochloride (14.77 g, 150.8 mmol) in DCM (100 mL) was added DIPEA (39 g, 301.6 mmol) at 5° C., and stirred until the solution was clear. Then the above acid chloride (15 g, 75.4 mmol) in DCM (100 mL) was slowly added dropwise. After the dropwise addition was completed, stirred at 5° C. for 1 h. After LCMS showed that the raw material reaction was consumed, the reaction solution was quenched with water and extracted with DCM (100 mL×2). The organic phase was combined, washed with saturated sodium bicarbonate aqueous solution, washed with brine, dried over anhydrous sodium sulfate, filterred, concentrated, and purified by column chromatography (10˜25% DCM/PE) to obtain product as colorless oil (9 g, yield 54%).

MS (ESI), m/z, 224 [M+1]⁺.

### Synthesis of Compound 12c-2

Compound 2a (2 g, 8.13 mmol) was dissolved in anhydrous THF (30 mL) and the temperature was cooled to −70° C., and then 2.5 M butyllithium (7.2 mL, 17.9 mmol) was added dropwise. The reaction solution was continued to stir at −70° C. for 1 h. A solution of 12c-1 (1.63 g, 7.32 mmol) in anhydrous THF (10 mL) was added dropwise. After the dropwise addition, the reaction solution was continued to stir at −70° C. for 2 h. LCMS showed that the raw material was consumed, then quenched with saturated NH₄Cl, extracted with ethyl acetate. The organic phase was washed with brine, dried over anhydrous sodium sulfate, filtered, the filtrate was concentrated, and purified by column chromatography (DCM/PE=1:5) to obtain a colorless oil (1.1 g, yield, 41%).

MS (ESI), m/z, 514.7 [M+1]⁺.

### Synthesis of Compound 12c-3

12c-2 (1.1 g, 3.34 mmol) was dissolved in ethanol, and 6 M dilute hydrochloric acid (10 mL) was added. The reaction solution was heated to 60° C. and stirred for 3 h. After LCMS showed that the reaction was complete, the reaction solution was concentrated to obtain 0.9 g as colorless oil (94%, yield), which was used directly in the next step.

MS (ESI), m/z, 288 [M+1]⁺.

### Synthesis of Compound 12c-4

To a solution of 12c-4 (109 mg, 0.38 mmol) and ketone (CAS is 110351-94-5) (100 mg, 0.38 mmol) in toluene (5 mL) was added p-toluenesulfonic acid monohydrate (14 mg, 0.076 mmol). The mixture was heated to 80° C. and stirred for 8 hours. After LCMS showed that the reaction was complete (partial racemization occurred), the mixture was concentrated. The residue was diluted with ethyl acetate and saturated aqueous sodium bicarbonate solution and extracted with ethyl acetate (50 ml×3). The organic phases were combined, washed with brine, dried over anhydrous sodium sulfate, filtered, and concentrated to obtain 200 mg of light brown solid (100% yield).

MS (ESI), m/z, 515 [M+1]⁺.

### Synthesis of Examples 12c and 12d

12c-4 (180 mg, 0.35 mmol) was dissolved in ethyl acetate (20 mL), then methanol (1 mL) and wet 10% palladium on carbon (100 mg) were added. The flash was evacuated and backfilled with H₂ (3×) and stirred under an atmosphere of H₂ (H₂ balloon) for 2 h at room temperature. After LCMS showed that the reaction was completed, the mixture was filtered with celite and concentrated. The crude product was purified with Prep-TLC (MeOH/DCM=1:20) to obtain 90 mg as yellow solid, which was further purified by Prep-HPLC to obtain 12c (50 mg) and 12d (5 mg).

LCMS (ESI), m/z, 425 [M+1]⁺.

12c: ¹H NMR (400 MHZ, DMSO) δ 8.20 (d, J=8.3 Hz, 1H), 7.80 (d, J=10.8 Hz, 1H), 7.25 (s, 1H), 6.43 (s, 1H), 5.82 (d, J=3.1 Hz, 1H), 5.77-5.71 (m, 1H), 5.41-5.27 (m, 4H), 2.43-2.40 (m, 3H), 1.87-1.71 (m, 2H), 1.45 (d, J=6.5 Hz, 3H), 0.81 (t, J=7.3 Hz, 3H).

12d: ¹H NMR (400 MHZ, DMSO) δ 8.26 (d, J=8.2 Hz, 1H), 7.88 (d, J=10.8 Hz, 1H), 7.32 (s, 1H), 6.50 (s, 1H), 5.89 (s, 1H), 5.82 (q, J=6.3 Hz, 1H), 5.49-5.34 (m, 4H), 2.50-2.48 (m, 3H), 1.86 (td, J=14.4, 7.0 Hz, 2H), 1.52 (d, J=6.5 Hz, 3H), 0.88 (t, J=7.3 Hz, 3H).

### Example 13

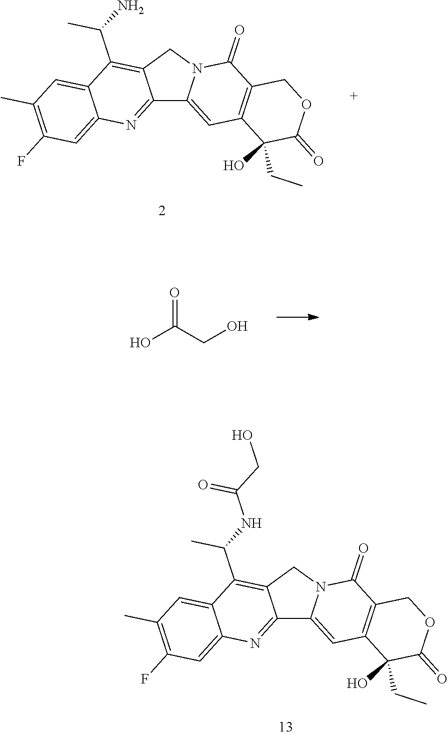 [SMILES: CC[C@@]1(O)C(=O)OCc2c1cc1n(c2=O)Cc2c-1nc1cc(F)c(C)cc1c2[C@H](C)N ; O=C(O)CO ; CC[C@@]1(O)C(=O)OCc2c1cc1n(c2=O)Cc2c-1nc1cc(F)c(C)cc1c2[C@H](C)NC(=O)CO]

### Synthesis of Example 13

To a solution of Example 2 (HCl salt, 100 mg, 0.22 mmol) in DMF (5 mL) was added glycolic acid (33 mg, 0.43 mmol), DMTMM (129 mg, 0.43 mmol) and triethylamine (110 mg, 1.09 mmol) at 0-5° C. The reaction was stirred for 1 h at this temperature. The mixture was quenched with water, concentrated, and purified by Prep-HPLC to obtain Example 13 (55 mg, yield 52%).

MS (ESI), m/z, 482.1 [M+H]⁺.

### Example 13b

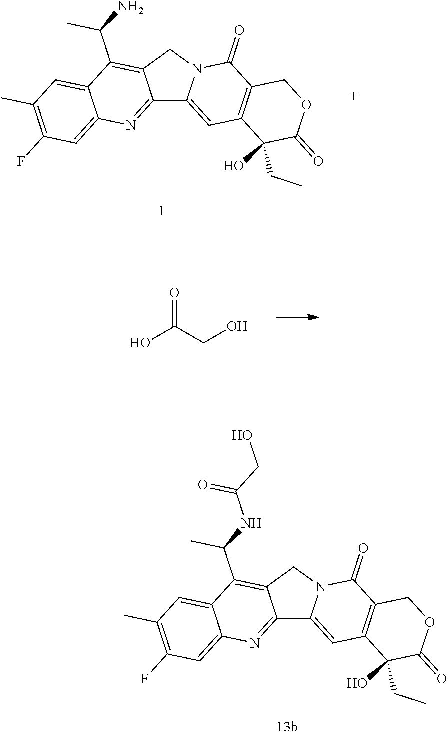 [SMILES: CC[C@@]1(O)C(=O)OCc2c1cc1n(c2=O)Cc2c-1nc1cc(F)c(C)cc1c2[C@@H](C)N ; O=C(O)CO ; CC[C@@]1(O)C(=O)OCc2c1cc1n(c2=O)Cc2c-1nc1cc(F)c(C)cc1c2[C@@H](C)NC(=O)CO]

### Synthesis of Example 13b

To a solution of Example 1 (HCl salt, 100 mg, 0.22 mmol) in DMF (5 mL) was added glycolic acid (33 mg, 0.43 mmol), HATU (134 mg, 0.43 mmol) and triethylamine (110 mg, 1.09 mmol) at 0-5° C. The reaction was stirred for 1 h at this temperature. The mixture was quenched with water, concentrated, and purified by Prep-HPLC to obtain Example 13b (50 mg, yield 50%).

MS (ESI), m/z, 482.1 [M+H]⁺.

### Example 14

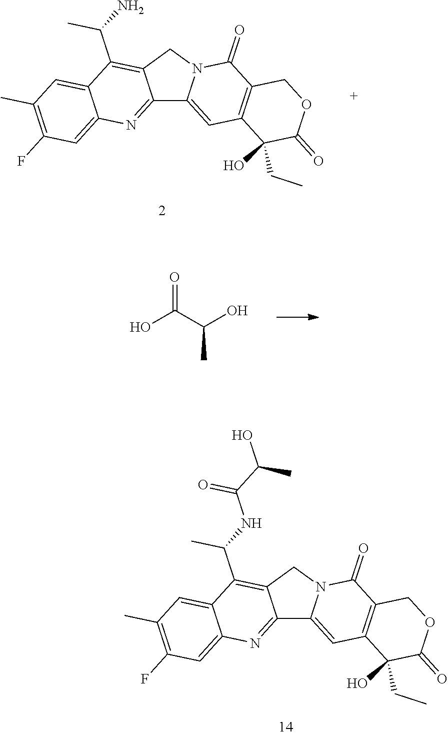

### Synthesis of Example 14

To a solution of Example 2 (HCl salt, 100 mg, 0.22 mmol) in DMF (5 mL) was added L-lactic acid (39 mg, 0.43 mmol) and DMTMM (129 mg, 0.43 mmol) and triethylamine (110 mg, 1.09 mmol) at 0-5° C. The reaction was stirred for 1 h at this temperature. The mixture was quenched with water, concentrated, and purified by Prep-HPLC to obtain Example 14 (60 mg, yield 55%).

MS (ESI), m/z, 496.1 [M+H]⁺.

### Example 15

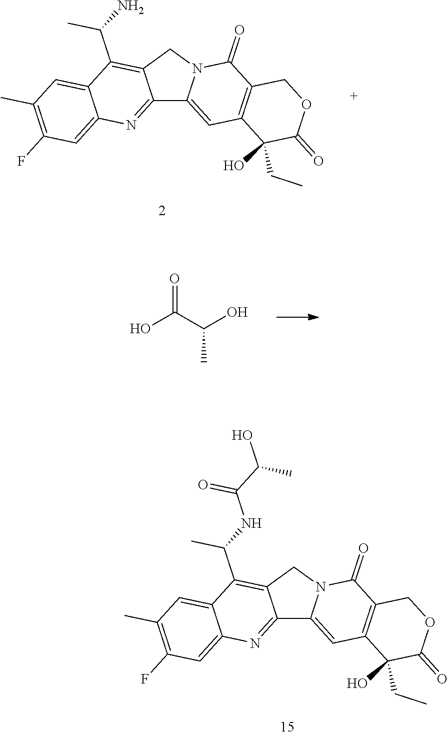

### Synthesis of Example 15

To a solution of Example 2 (HCl salt, 100 mg, 0.22 mmol) in DMF (5 mL) was added D-lactic acid (39 mg, 0.43 mmol), DMTMM (129 mg, 0.43 mmol) and triethylamine (110 mg, 1.09 mmol) at 0-5° C. The reaction was stirred for 1 h at this temperature. The mixture was quenched with water, concentrated, and purified by Prep-HPLC to obtain Example 15 (65 mg, yield 60%).

MS (ESI), m/z, 496.1 [M+H]⁺.

### Example 16

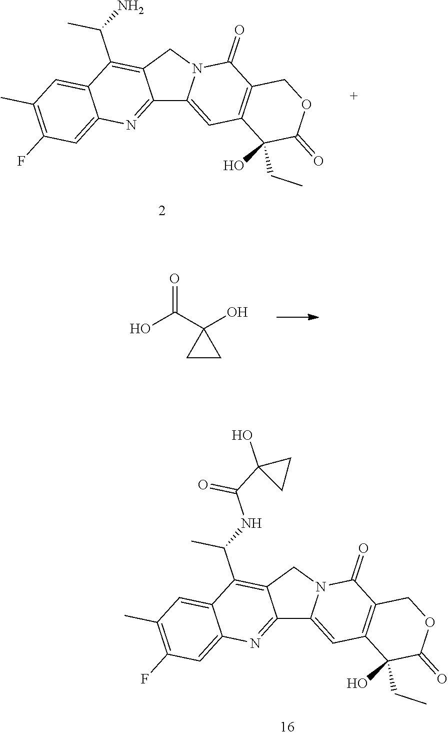 [SMILES: CC[C@@]1(O)C(=O)OCc2c1cc1n(c2=O)Cc2c-1nc1cc(F)c(C)cc1c2[C@H](C)N ; O=C(O)C1(O)CC1 ; CC[C@@]1(O)C(=O)OCc2c1cc1n(c2=O)Cc2c-1nc1cc(F)c(C)cc1c2[C@H](C)NC(=O)C1(O)CC1]

### Synthesis of Example 16

To a solution of Example 2 (hydrochloride, 100 mg, 0.22 mmol) in DMF (5 mL) was added 1-hydroxycyclopropanecarboxylic acid (44 mg, 0.43 mmol), DMTMM (129 mg, 0.43 mmol) and triethylamine (110 mg, 1.09 mmol) at 0-5° C. The reaction was stirred for 12 h at this temperature. The reaction solution was quenched with water, concentrated, and purified by Prep-HPLC to obtain Example 16 (40 mg, yield 36%).

MS (ESI), m/z, 508.1 [M+H]⁺.

### Example 17

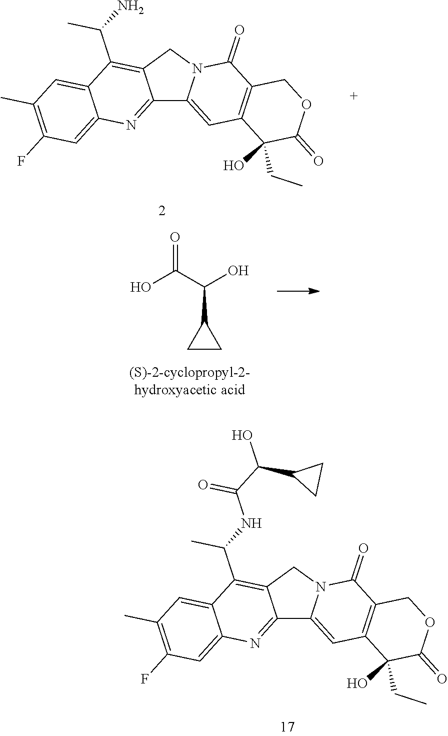 [SMILES: CC[C@@]1(O)C(=O)OCc2c1cc1n(c2=O)Cc2c-1nc1cc(F)c(C)cc1c2[C@H](C)N ; O=C(O)[C@@H](O)C1CC1 ; CC[C@@]1(O)C(=O)OCc2c1cc1n(c2=O)Cc2c-1nc1cc(F)c(C)cc1c2[C@H](C)NC(=O)[C@@H](O)C1CC1]

### Synthesis of Example 17

To a solution of Example 2 (HCl salt, 100 mg, 0.22 mmol) in DMF (5 mL) was added(S)-2-cyclopropyl-2-hydroxyacetic acid (50 mg, 0.43 mmol) (The synthesis method refers to compound 93d in patent WO2013185093A1), DMTMM (129 mg, 0.43 mmol) and triethylamine (110 mg, 1.09 mmol) at 0-5° C. The reaction was stirred at this temperature for 12 h. The reaction solution was quenched with water, concentrated, and purified by HPLC to obtain Example 17 (48 mg, yield 42%).

MS (ESI), m/z, 496.1 [M+H]⁺.

### Example 18

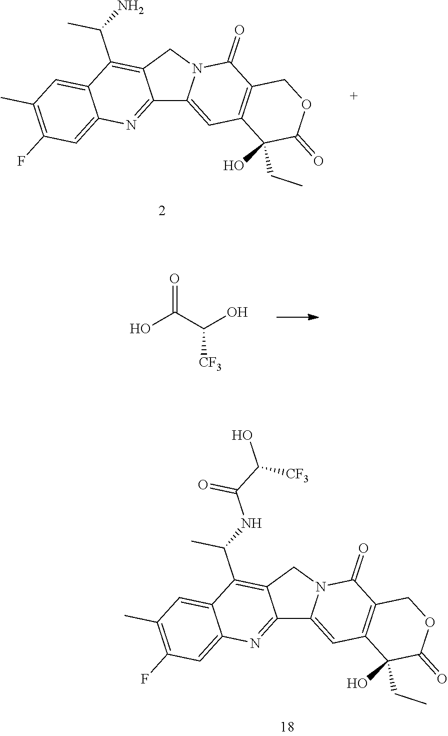 [SMILES: CC[C@@]1(O)C(=O)OCc2c1cc1n(c2=O)Cc2c-1nc1cc(F)c(C)cc1c2[C@H](C)N ; O=C(O)[C@H](O)C(F)(F)F ; CC[C@@]1(O)C(=O)OCc2c1cc1n(c2=O)Cc2c-1nc1cc(F)c(C)cc1c2[C@H](C)NC(=O)[C@H](O)C(F)(F)F]

### Synthesis of Example 18

To a solution of Example 2 (HCl salt, 100 mg, 0.22 mmol) in DMF (5 mL) was added(S)-(−)-trifluorolactic acid (63 mg, 0.43 mmol), DMTMM (129 mg, 0.43 mmol) and triethylamine (110 mg, 1.09 mmol) at 0-5° C. The reaction was stirred for 2 h at this temperature. The reaction solution was quenched with water, concentrated, and purified by Prep-HPLC to obtain Example 18 (65 mg, yield 54%).

MS (ESI), m/z, 550.1 [M+H]⁺.

### Example 19

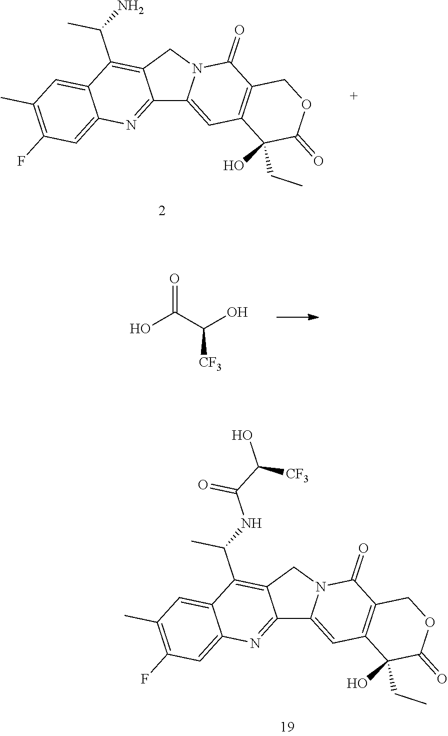 [SMILES: CC[C@@]1(O)C(=O)OCc2c1cc1n(c2=O)Cc2c-1nc1cc(F)c(C)cc1c2[C@H](C)N ; O=C(O)[C@@H](O)C(F)(F)F ; CC[C@@]1(O)C(=O)OCc2c1cc1n(c2=O)Cc2c-1nc1cc(F)c(C)cc1c2[C@H](C)NC(=O)[C@@H](O)C(F)(F)F]

### Synthesis of Example 19

To a solution of Example 2 (HCl salt, 100 mg, 0.22 mmol) in DMF (5 mL) was added (R)-(−)-trifluorolactic acid (63 mg, 0.43 mmol) and DMTMM (129 mg, 0.43 mmol) and triethylamine (110 mg, 1.09 mmol) at 0-5° C. The reaction was stirred for 2 h at this temperature. The reaction solution was quenched with water, concentrated, and purified by Prep-HPLC to obtain Example 19 (60 mg, yield 50%).

MS (ESI), m/z, 550.1 [M+H]⁺.

### Example 20

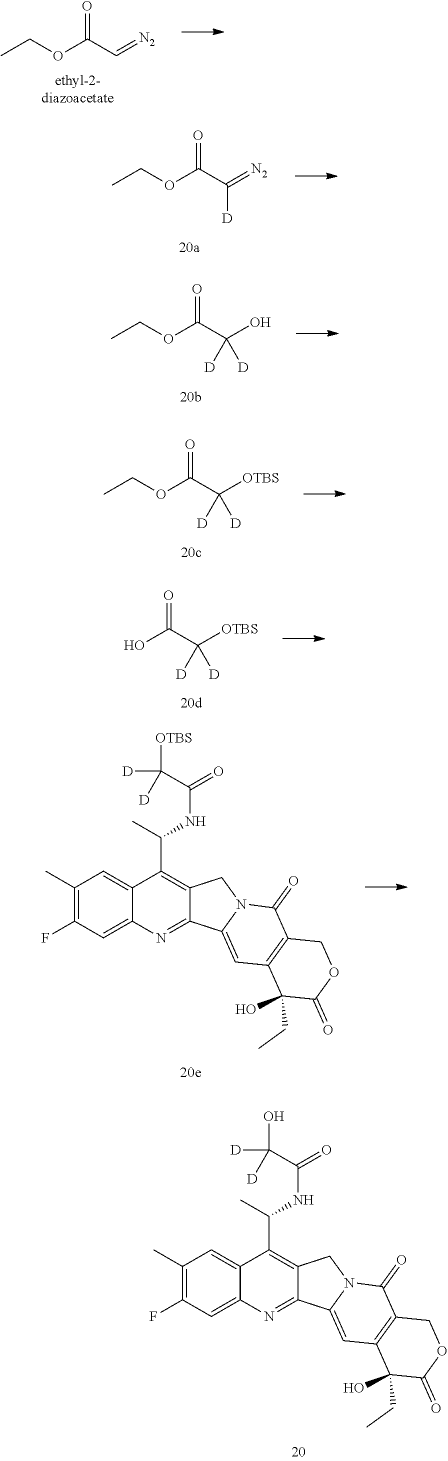

### Synthesis of Compound 20a

To a solution of ethyl diazoacetate (1.0 g, 8.77 mmol) in acetonitrile (20 mL) was added potassium carbonate (12 mg, 0.087 mmol) and deuterium oxide (10 mL) at 0-5° C. under nitrogen atmosphere. The reaction was stirred vigorously for 2 h. The reaction solution was extracted with dichloromethane (50 mL×3), and the organic phases were combined, dried over anhydrous sodium sulfate, filtered, and concentrated to obtain compound 20a (1.0 g), which was used directly in the next step.

### Synthesis of Compound 20b

To a solution of 20a (1.0 g, 8.70 mmol) in deuterium oxide (10 mL) was added deuterated hydrochloric acid (5 mL) at 25° C. under nitrogen atomsphere, and stirred for 12 h. The reaction was extracted with dichloromethane (50 mL×3), the organic phases were combined, dried over anhydrous sodium sulfate, filtered, and concentrated to obtain compound 20b (500 mg), which was used directly in the next step.

### Synthesis of Compound 20c

To a solution of 20b (500 mg, 4.72 mmol) in dichloromethane (10 mL) was added imidazole (481 mg, 7.08 mmol) and tert-butyldimethylchlorosilane (850 mg, 5.66 mmol) at 0-5° C. under nitrogen atomsphere. The reaction was stirred for 1 h, quenched by water, and extracted with dichloromethane (50 mL×3). The organic phases were combined, dried over anhydrous sodium sulfate, filtered, concentrated, and purified by column chromatography to obtain compound 20c (0.2 g).

¹H NMR (400 MHZ, Chloroform-d) δ ppm 4.19 (q, J=7.1 Hz, 2H), 1.27 (t, J=7.1 Hz, 3H), 0.92 (s, 9H), 0.10 (s, 6H).

### Synthesis of Compound 20d

To a solution of 20c (200 mg, 0.91 mmol) in methanol-d4 (10 mL) was added sodium deuteroxide (72 mg, 1.82 mmol) at 0-5° C. under nitrogen atmosphere. The reaction was stirred for 1 h, then adjusted to pH<7 with IN dilute deuterated hydrochloric acid. The mixture was extracted by dichloromethane (50 mL×3), and the organic phases were combined, dried over anhydrous sodium sulfate, filtered, and concentrated to obtain compound 20d (150 mg), which was used directly for the next step.

### Synthesis of Compound 20e

To a solution of Example 6 (HCl salt, 100 mg, 0.22 mmol) in DMF (5 mL) was added 20d (83 mg, 0.44 mmol), DMTMM (129 mg, 0.43 mmol) and triethylamine (110 mg, 1.09 mmol) at 0-5° C. The reaction was stirred for 2 h at this temperature. The reaction solution was quenched with water and extracted with dichloromethane (50 mL×3). The organic phases were combined, dried over anhydrous sodium sulfate, filtered, and concentrated to obtain compound 20e. The crude product was used directly in the next step. MS (ESI), m/z, 598.1 [M+H]⁺.

### Synthesis of Example 20

To a solution of the above crude compound 20e (130 mg, 0.22 mmol) in methanol (5 mL) was added 2 N hydrochloric acid (1 mL) at room temperature 25° C. The reaction was stirred for 1 h, concentrated, and purified by HPLC to obtain Example 20 (20 mg, purity 97%).

MS (ESI), m/z, 484.1 [M+H]⁺.

### Example 21

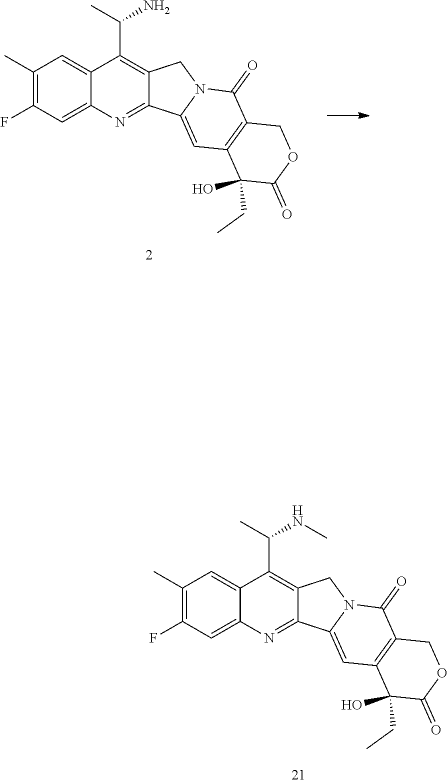 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@H](C)NC]

### Synthesis of Example 21

To a solution of Example 2 (HCl salt, 100 mg, 0.22 mmol) in dichloromethane (10 mL) was added triethylamine (66 mg, 0.65 mmol) and 37% formaldehyde solution (17 mg, 0.22 mmol) at 0-5° C. under nitrogen atmosphere. The reaction was stirred for 0.5 h, and then sodium triacetoxyborohydride (92 mg, 0.44 mmol) was added. The mixture was warmed to room temperature and stirred for 12 h. The reaction solution was diluted with dichloromethane and sodium bicarbonate solution. The organic phase was washed with water and brine, dried over anhydrous sodium sulfate, concentrated, and purified by Prep-HPLC to obtain Example 21 (TFA salt, 10 mg, purity 92%).

MS (ESI), m/z, 438.1 [M+H]⁺.

### Example 22

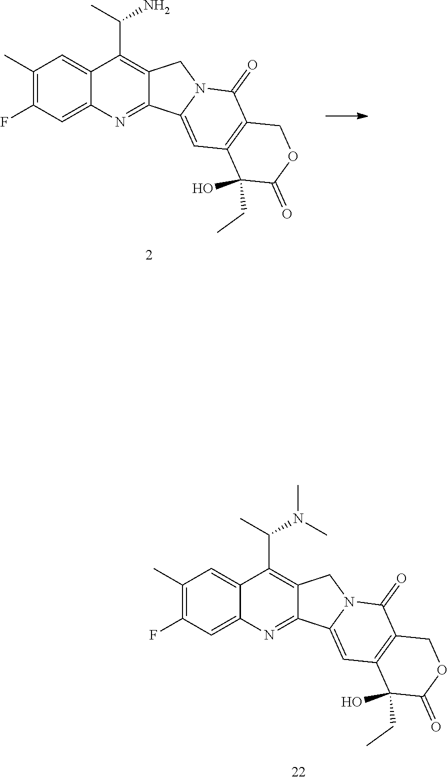 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@H](C)N(C)C]

### Synthesis of Example 22

To a solution of Example 2 (HCl salt, 100 mg, 0.22 mmol) in dichloromethane (10 mL) was added triethylamine (66 mg, 0.65 mmol) and 37% formaldehyde solution (175 mg, 2.18 mmol) at 0-5° C. under nitrogen atomsphere. The reaction was stirred for 0.5 h, and then sodium triacetoxyborohydride (138 mg, 0.65 mmol) was added. The reaction solution was warmed to room temperature and stirred for 12 h. The reaction solution was diluted with dichloromethane and sodium bicarbonate solution. The organic phase was washed with water and brine, dried over anhydrous sodium sulfate, concentrated, and purified by Prep-HPLC to obtain Example 22 (TFA salt, 60 mg, purity 95%).

MS (ESI), m/z, 452.1 [M+H]⁺.

### Example 23

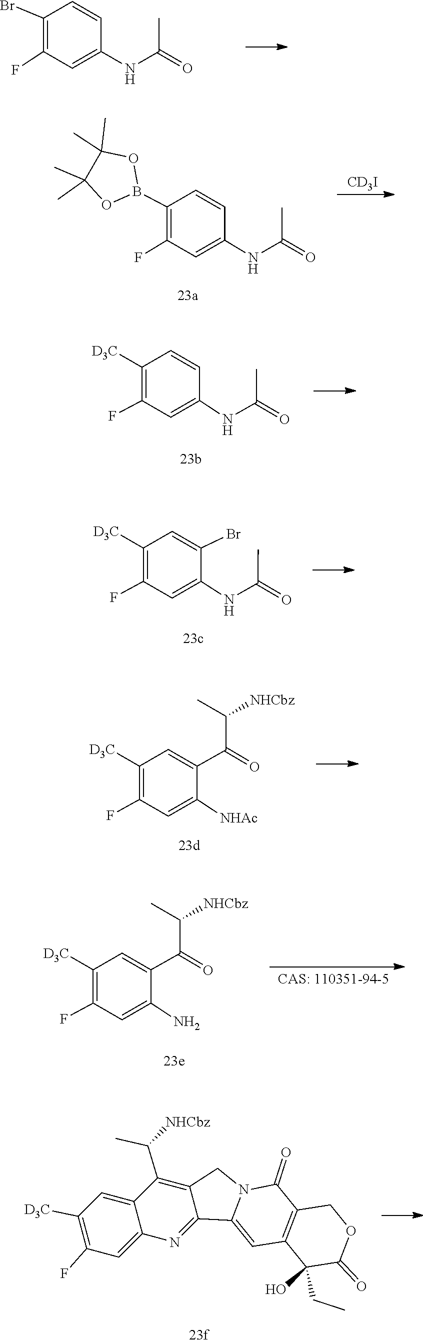

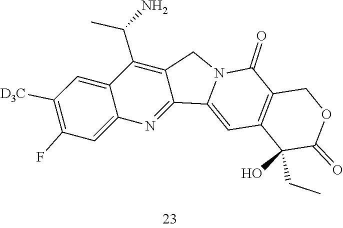 [SMILES: [2H]C([2H])([2H])c1cc2c([C@H](C)N)c3c(nc2cc1F)-c1cc2c(c(=O)n1C3)COC(=O)[C@]2(O)CC]

### Synthesis of Compound 23a

To a solution of 4′-bromo-3′-fluoroacetanilide (8.0 g, 0.035 mol) in DMSO (100 mL) was added pinacol diborate (26.5 g, 0.10 mol) and Pd₂(dba)₃ (4.8 g, 5.2 mmol), tricyclohexylphosphine (2.9 g, 0.01 mmol) and potassium acetate (10.2 g, 0.10 mmol). The reaction was heated at 90° C. for 18 h under nitrogen atmosphere. The reaction was monitored by LCMS. After the reaction was complete, the mixture was cooled to room temperature. The reaction solution was filtered, the filtrate was diluted with water, and the aqueous phase was extracted with ethyl acetate (100 mL×3). The organic phases were combined, washed with brine, dried over anhydrous sodium sulfate, filtered, concentrated, and purified by silica gel column chromatography (petroleum ether/ethyl acetate) to obtain product 23a (5.5 g, yield 57%).

MS (ESI), m/z, 280.2 [M+1]⁺.

### Synthesis of Compound 23b

To a solution of compound 23a (5.5 g, 0.02 mol) in DMF/H₂O (100/10 mL) was added deuterated methyl iodide (14.3 g, 0.1 mol), Pd₂(dba)₃ (1.8 g, 2.0 mmol), tris (o-methylphenyl) phosphorus (1.2 g, 4.0 mmol) and potassium carbonate (8.2 g, 0.06 mmol). The reaction was heated to 70° C. and stirred for 16 h under nitrogen atmosphere. The reaction was monitored by LCMS. The reaction solution was filtered, water was added to the filtrate and extracted with ethyl acetate (100 mL×3). The organic phases were combined, washed with brine, dried over anhydrous sodium sulfate, filtered, concentrated, and purified by silica gel column chromatography (petroleum ether/ethyl acetate) to obtain product 23b (3.0 g, yield 89%).

MS (ESI), m/z, 171.1 [M+1]⁺.

### Synthesis of Compound 23c

To a solution (50 mL) of 23b (3.0 g, 0.02 mol) in DMF was added NBS (4.7 g, 0.03 mol) in batches at 5-10° C., and the reaction was stirred for 12 h. The reaction solution was poured into ice water (200 mL), stirred for 0.5 h, filtered, and cake was washed with saturated sodium bicarbonate, water, and dried in vacuo to obtain compound 23c (3.8 g, yield 87%).

MS (ESI), m/z, 249.0 [M+H]⁺.

### Synthesis of Compound 23d

To a solution of 23c (3.0 g, 0.024 mol) in dry tetrahydrofuran (80 mL) was added dropwise n-butyllithium solution (2.5 M in hexane, 11 mL, 0.027 mol)−60° C. The reaction solution continued to stir for 1 h, and a solution of 6b (4.8 g, 0.018 mol) in tetrahydrofuran solution (20 mL) was added dropwise, and the reaction was stirred for 2 h (−60-−20° C.). The reaction solution was quenched with ammonium chloride aqueous solution, the aqueous phase was extracted with ethyl acetate (50 mL×3). The organic phases were combined, washed with brine, dried over anhydrous sodium sulfate, filtered, concentrated, and purified by column chromatography to obtain compound 23d (400 mg).

MS (ESI), m/z, 376.2 [M+H]⁺.

¹H NMR (400 MHZ, Chloroform-d) δ ppm 11.50 (s, 1H), 8.52 (d, J=12.5 Hz, 1H), 7.75 (d, J=8.2 Hz, 1H), 7.44-7.28 (m, 4H), 5.70 (d, J=7.8 Hz, 1H), 5.37 (t, J=7.4 Hz, 1H), 5.13 (s, 2H), 2.22 (s, 2H), 1.43 (d, J=7.1 Hz, 3H).

### Synthesis of Compound 23e

To a solution of 23d (300 mg, 0.81 mmol) in methanol and tetrahydrofuran (3/5 mL) was added concentrated hydrochloric acid (3 mL) and stirred for 12 h. The reaction solution was concentrated, basified with sodium bicarbonate to pH>7, diluted with water and extracted with dichloromethane (50 mL×3). The organic phases were combined, washed with brine, dried over anhydrous sodium sulfate, filtered, and purified by column chromatography to obtain compound 23e (200 mg).

MS (ESI), m/z, 331.1 [M+H]⁺.

### Synthesis of Compound 23f

To a solution of 23e (200 mg, 0.60 mmol) in toluene (5 mL) was added ketone (CAS is 110351-94-5) (0.19 g, 0.72 mmol) and p-toluenesulfonic acid monohydrate (12 mg, 0.24 mmol). The reaction solution was heated to 120° C. and stirred for 12 h. After the reaction was completed, the reaction solution was concentrated and purified by column chromatography to obtain compound 23f (150 mg).

MS (ESI), m/z, 561.1 [M+H]⁺.

### Synthesis of Example 23

To a solution of 23f (150 mg, 0.27 mmol) in methanol and 6 N hydrochloric acid solution (30 mL/30 mL) was added 10% Pd/C (water content 55%, 50 mg) at room temperature 25° C. The flash was evacuated and backfilled with N₂, and then H₂. The mixture was stirred for 6 h under H₂ atmosphere. The reaction mixture was filtered with celite, washed with dichloromethane/methanol, concentrated, and slurried with ethanol to obtain Example 23 (hydrochloride, 50 mg).

MS (ESI), m/z, 427.1 [M+H]⁺.

### Example 24

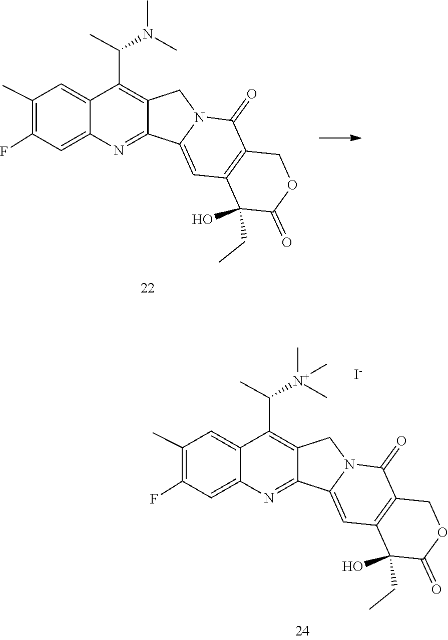 [SMILES: CC[C@@]1(O)C(=O)OCc2c1cc1n(c2=O)Cc2c-1nc1cc(F)c(C)cc1c2[C@H](C)N(C)C ; CC[C@@]1(O)C(=O)OCc2c1cc1n(c2=O)Cc2c-1nc1cc(F)c(C)cc1c2[C@H](C)[N+](C)(C)C ; [I-]]

### Synthesis of Example 24

To a solution of Example 22 (TFA salt, 20 mg, 0.04 mmol) in DMF (2 mL) was added methyl iodide (10 mg, 0.07 mmol) and triethylamine (11 mg, 0.10 mmol) at 0-5° C. under nitrogen atmosphere. The reaction solution was stirred for 0.5 h, concentrated, and purified by Prep-HPLC to obtain Example 24 (8 mg, purity 95%).

MS (ESI), m/z, 466.1 [M+H]⁺.

### Example 25

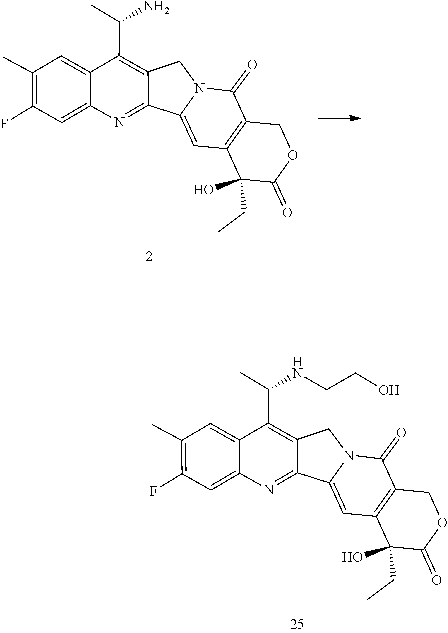 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@H](C)NCCO]

### Synthesis of Example 25

To a solution of Example 2 (HCl salt, 100 mg, 0.22 mmol) in dichloromethane (10 mL) was added triethylamine (66 mg, 0.65 mmol) and glycolaldehyde dimer (48 mg, 0.40 mmol) at 0-5° C. under nitrogen atmosphere. The reaction was stirred for 0.5 h, then sodium triacetoxyborohydride (92 mg, 0.44 mmol) was added. The mixture was slowly warmed to room temperature and stirred for 12 h. The reaction solution was diluted with dichloromethane and then sodium bicarbonate solution was added. The organic layer was washed with water and brine, dried over anhydrous sodium sulfate, concentrated, and purified by Prep-HPLC to obtain Example 25 (TFA salt, 35 mg).

MS (ESI), m/z, 468.1 [M+H]⁺.

### Example 25a

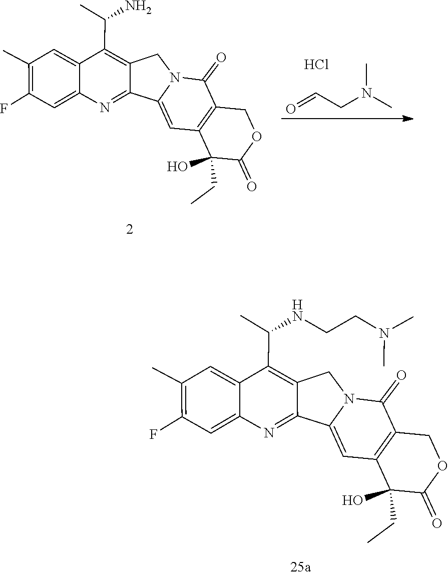

### Synthesis of Example 25a

To a solution of Example 2 (TFA salt, 100 mg, 0.22 mmol) in dichloromethane (10 mL) was added triethylamine (66 mg, 0.65 mmol) and dimethylaminoacetaldehyde hydrochloride (48 mg, 0.44 mmol) at 0-5° C. under nitrogen atmosphere. The reaction was stirred for 0.5 h, and then sodium cyanoborohydride (30 mg, 0.44 mmol) was added. The mixture was slowly warmed to room temperature and stirred for 16 h. LCMS showed that 25% product was formed. The reaction solution was diluted with dichloromethane and sodium bicarbonate solution was added. The organic layer was washed with water and brine, dried over anhydrous sodium sulfate, concentrated, and separated and purified by Prep-HPLC (CH₃CN/H₂O, 0.05% TFA) to obtain Example 25a (12 mg).

MS (ESI), m/z, 495.1 [M+H]⁺.

### Example 25b

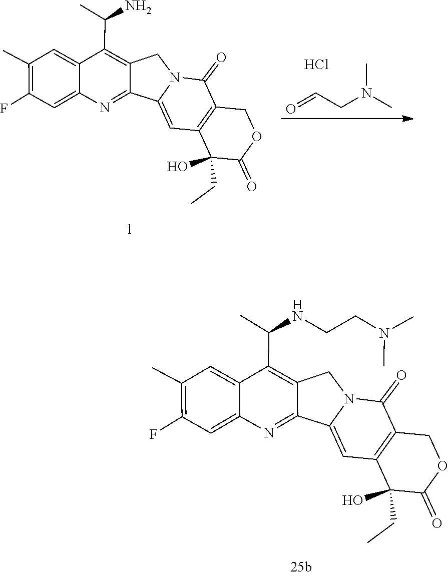

### Synthesis of Example 25b

According to the synthesis method of 25a, the crude product was purified by Prep-HPLC (CH₃CN/H₂O, 0.05% TFA) to obtain Example 25b (18 mg).

MS (ESI), m/z, 495.1 [M+H]⁺.

### Example 25c

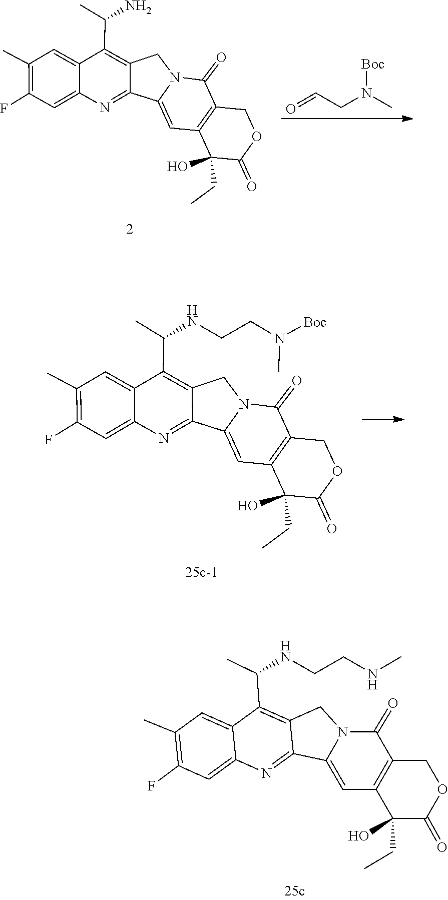

### Synthesis of Compound 25c-1

To a solution of Example 2 (TFA salt, 100 mg, 0.22 mmol) in dichloromethane (10 mL) was added triethylamine (66 mg, 0.65 mmol) and N-BOC-(methylamino) acetaldehyde (82 mg, 0.44 mmol) at 0-5° C. under nitrogen atmosphere. The reaction was stirred for 0.5 h, then sodium cyanoborohydride (30 mg, 0.44 mmol) was added. The mixture was warmed slowly to room temperature and stirred for 16 h. The reaction solution was quenched with water and extracted with dichloromethane (50 mL×3). The organic phases were combined, washed with salt, dried over anhydrous sodium sulfate, concentrated, and purified by Prep-HPLC (CH₃CN/H₂O, 0.05% TFA) to obtain compound 25c-1 (30 mg).

MS (ESI), m/z, 581.1 [M+H]⁺.

### Synthesis of Example 25c

To a solution of compound 25c-1 (30 mg) in dichloromethane (10 mL) was added trifluoroacetic acid (2 mL) at 0-5° C. under nitrogen atmosphere. The reaction solution was warmed slowly to room temperature and stirred for 2 h. LCMS showed that the reaction was complete. The reaction solution was concentrated and purified by Prep-HPLC (CH₃CN/H₂O, 0.05% TFA) to obtain Example 25c (7.3 mg).

MS (ESI), m/z, 481.0 [M+H]⁺.

### Example 25d

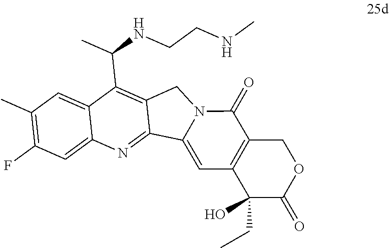 [SMILES: CC[C@@]1(O)C(=O)OCc2c1cc1n(c2=O)Cc2c-1nc1cc(F)c(C)cc1c2[C@@H](C)NCCNC]

### Synthesis of Example 25d

According to the synthesis method of 25c, the crude product was purified by Prep-HPLC (CH₃CN/H₂O, 0.05% TFA) to obtain Example 25d (11 mg).

MS (ESI), m/z, 481.0 [M+H]⁺.

### Example 26

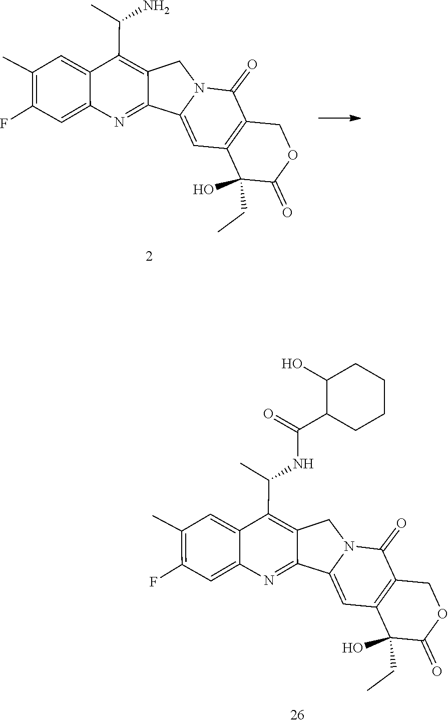 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@H](C)NC(=O)C1CCCCC1O]

### Synthesis of Example 26

To a solution of Example 2 (HCl salt, 100 mg, 0.22 mmol) in DMF (5 mL) was added 2-hydroxycyclohexylcarboxylic acid (58 mg, 0.43 mmol), DMTMM (129 mg, 0.43 mmol) and triethylamine (110 mg, 1.09 mmol) 0-5° C. The reaction was stirred for 2 h at this temperature. The reaction solution was quenched with water, concentrated, and purified by Prep-HPLC to obtain Example 26 (70 mg, yield 62%).

MS (ESI), m/z, 550.1 [M+H]⁺.

### Example 26a

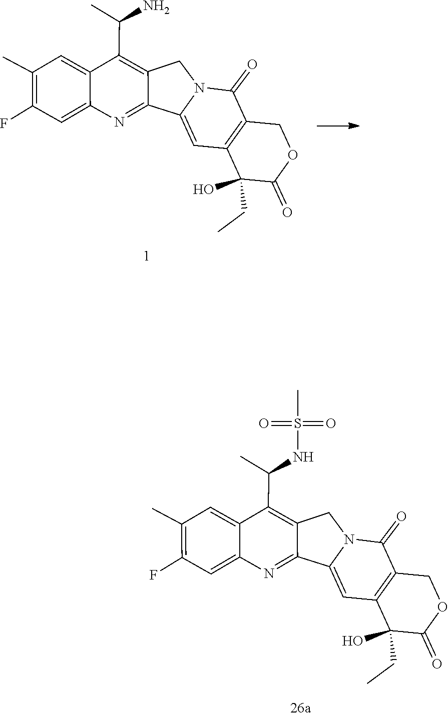 [SMILES: CC[C@@]1(O)C(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@@H](C)NS(C)(=O)=O]

### Synthesis of Example 26a

To a suspension solution of Example 1 HCl (50 mg, 0.11 mmol) in dry dichloromethane (2 mL) was added pyridine (43 mg, 0.54 mmol) and methyl sulfonate anhydride (38 mg, 0.22 mmol) at 5° C. of an ice-water bath and under nitrogen atmosphere. The reaction was warmed to room temperature and stirred for 2 h. The reaction solution was quenched with water, concentrated directly, and purified by Prep-HPLC (CH₃CN/H₂O, 0.05% TFA) to obtain Example 26a (22 mg).

MS (ESI), m/z, 502.1 [M+H]⁺.

### Example 26b

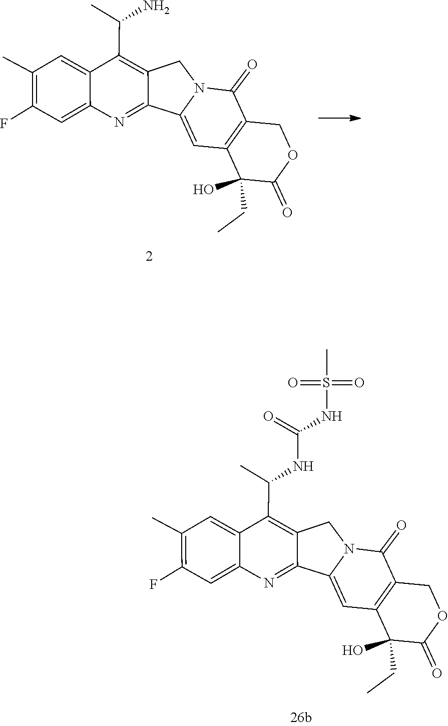

### Synthesis of Example 26b

To a suspension solution of Example 2 HCl (50 mg, 0.11 mmol) in dry dichloromethane (2 mL) was added pyridine (43 mg, 0.54 mmol) and methyl sulfonate anhydride (38 mg, 0.22 mmol) at 5° C. of an ice-water bath and under nitrogen atmosphere. The reaction was warmed to room temperature and stirred for 2 h. The reaction solution was quenched with water, concentrated directly, and purified by Prep-HPLC (CH₃CN/H₂O, 0.05% TFA) to obtain Example 26b (15.6 mg).

MS (ESI), m/z, 502.1 [M+H]⁺.

### Compound A

The synthesis method of compound A refers to the preparation method in patent WO2020/219287A1 (Example 5, compound 6a).

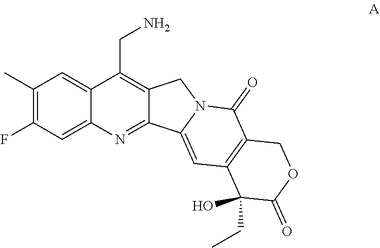 [SMILES: CC[C@@]1(O)C(=O)OCc2c1cc1n(c2=O)Cc2c-1nc1cc(F)c(C)cc1c2CN]

### Compound B (Exatecan)

The synthesis method of compound B (mesylate salt) refers to the preparation method in patent US2001/0034446A1 (Example 1).

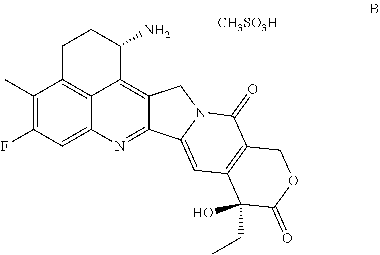 [SMILES: CC[C@@]1(O)C(=O)OCc2c1cc1n(c2=O)Cc2c-1nc1cc(F)c(C)c3c1c2[C@@H](N)CC3 ; "CH3SO3H"]

### Polymer Conjugate Example 27

Synthesis of compound 27a: Synthesis of BHALys [Lys]₃₂ [a-NH₂TFA]₃₂[e-PEG˜2000]₃₂

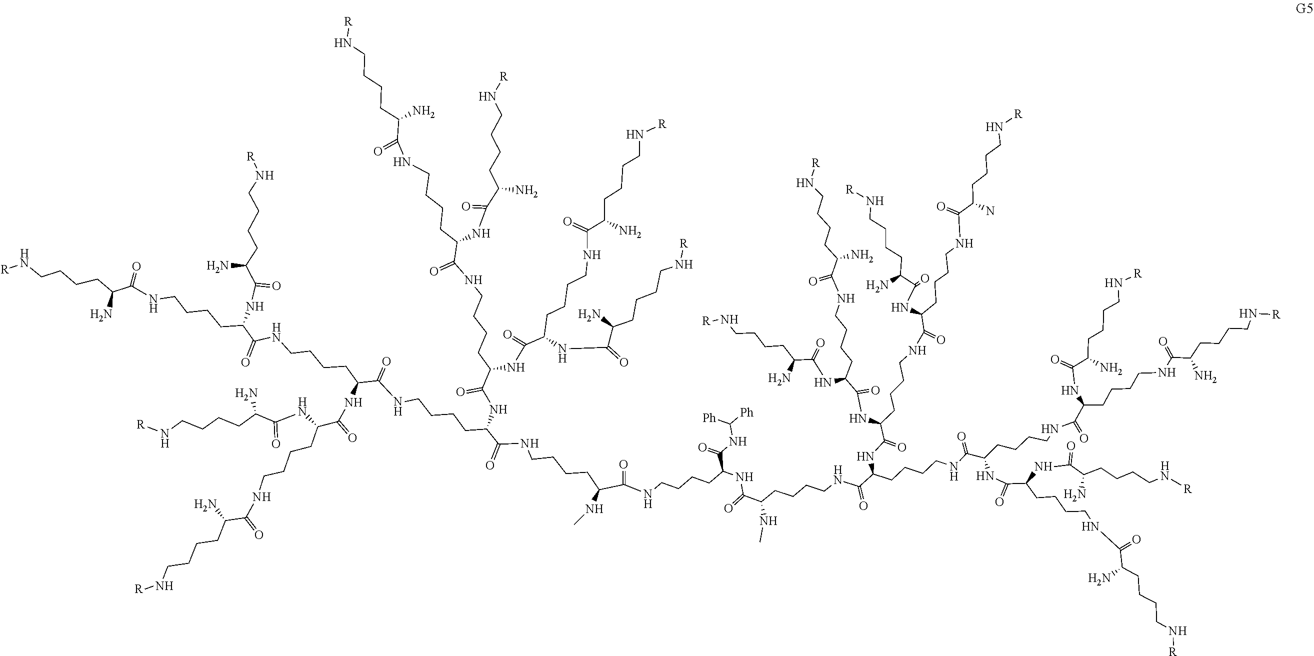

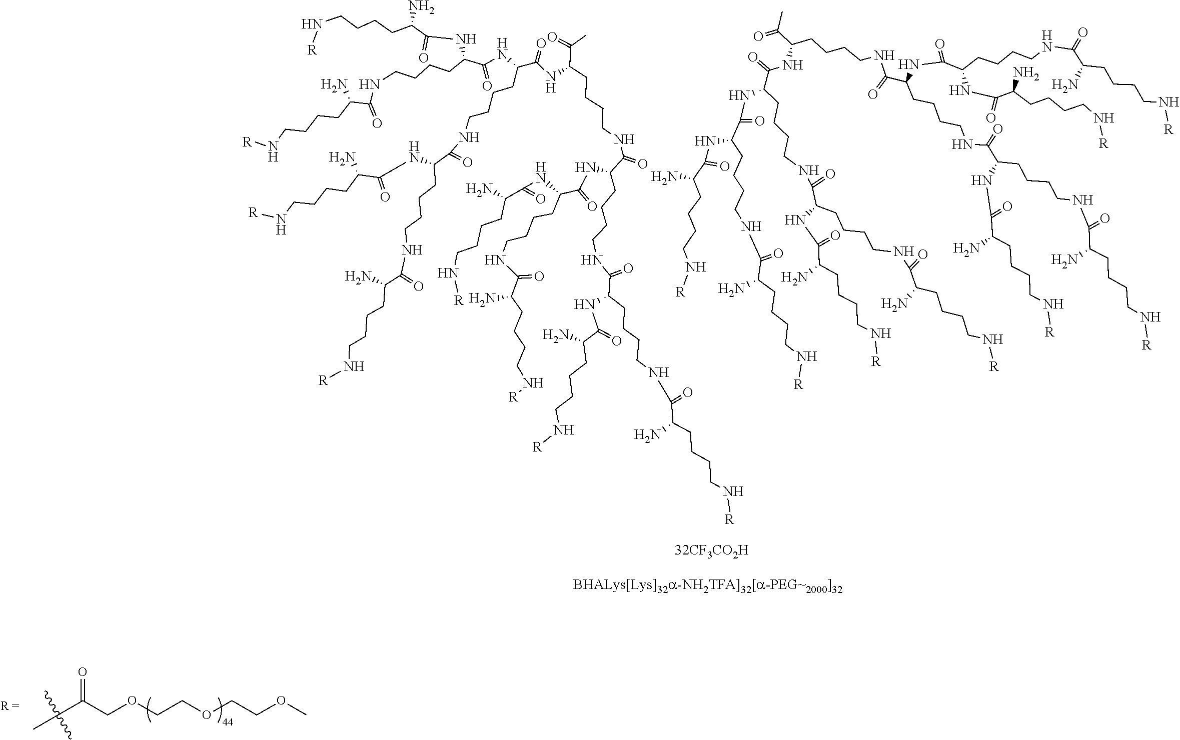

Dendrimer G5 compound BHALys[Lys]₃₂[a-NH₂TFA]₃₂[e-PEG˜2000]₃₂ was synthesized according to literature (Pharmaceutics 2018, 15, 10, 4568-4576) or patent (WO2020102852A1).

### Synthesis of Compound 27b

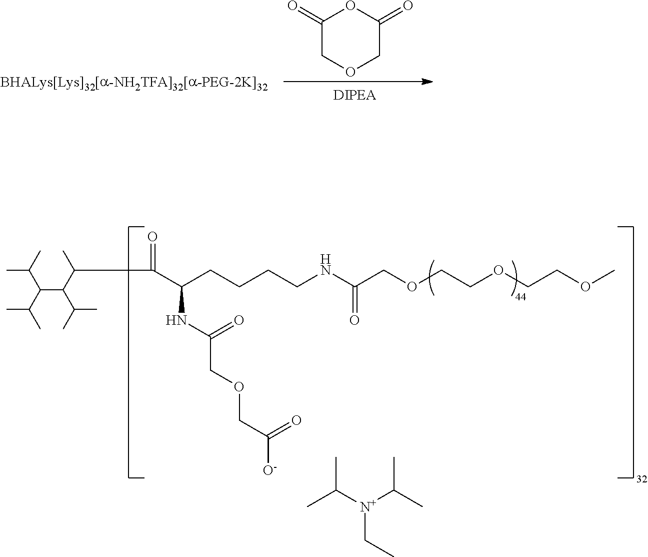

To a solution of 27a (800 mg, 0.01 mmol, 1.0 eq) in dry DMF (3 mL) was added diethylene glycol anhydride (76 mg, 0.66 mmol, 64.0 eq) and DIPEA (170 mg, 1.32 mmol, 128.0 eq) at room temperature 25° C. under nitrogen atmosphere. The reaction was stirred for 12 h. HPLC showed that the reaction was complete. MTBE (30 mL) was added to the above reaction solution and stirred for 1 h. The supernatant was discarded, and the residue was dried under vacuum to obtain crude product 27b (838 mg), which was used directly in the next step.

### Synthesis of Example 27

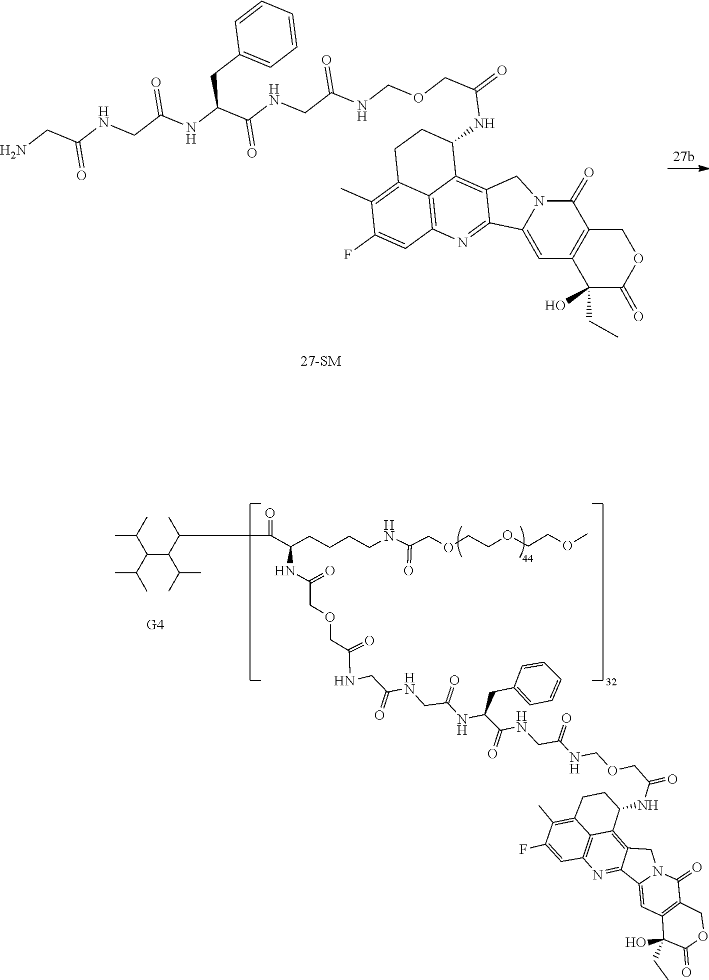

To a solution of 27b (150 mg, 0.0018 mmol, 1.0 eq) in DMF (5 mL) was added 27-SM (synthesized according to the synthesis method of Example 1 in patent WO2015155998A1, 74 mg, 0.089 mmol, 48.0 eq), PyBOP (77 mg, 0.15 mmol, 80.0 eq) and DIPEA (30 mg, 0.24 mmol, 128.0 eq) at 25° C. under nitrogen atmosphere. The reaction was stirred for 12 h, and HPLC showed that the reaction was complete. The reaction solution was concentrated, and the residue was dissolved in MeOH/H2O (50/50 mL), filtered, and purified by ultrafiltration with a 30K MWCO ultrafiltration membrane. The final product was freeze-dried to obtain 170 mg (off-white solid, HPLC purity >98%).

Drug content test: Accurately weigh 10 mg of the sample, add 0.5 mL of methanol and 0.5 mL of 6 N HCl solution and stir for 12 hours (decomposed into Dxd and a small amount of exatecan). An appropriate amount was detected by HPLC. The DXd content in the polymer was 13.5%.

### Polymer Conjugate Example 28

### Synthesis of Compound 28a

7

To a solution (5 mL) of 28-SM (synthesized according to the synthesis method of Example 1 in patent WO2019195665A1, 110 mg, 0.17 mmol), Example 13 (86 mg, 0.19 mmol, 1.1 eq) and DMTMM (75 mg, 0.26 mmol, 1.5 eq) and DIPEA (44 mg, 0.34 mmol, 2.0 eq) in DMF (5 mL) at 5° C. under nitrogen atomsphere. The reaction solution was stirred for 1 h, quenched with water, concentrated, purified by slurring with CH₂Cl₂/MTBE (3/30 mL), and filtered to obtain product 28a (160 mg, white solid, yield 89%).

MS (ESI), m/z, 1052.0 [M+1]⁺.

### Synthesis of Compound 28b

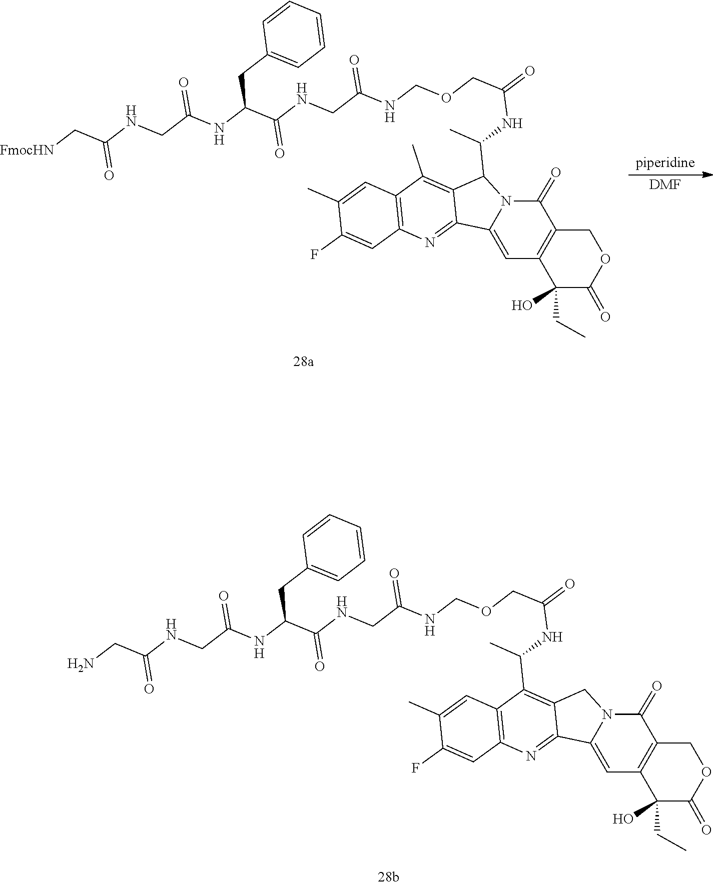

To a solution of 28a (160 mg, 0.15 mmol) was added piperidine (0.5 mL) in DMF (2 mL) at 5° C. and under nitrogen atmosphere. The reaction solution was stirred for 1 h, concentrated, purified by slurring with CH₂Cl₂/MTBE (3/30 mL), and filtered to obtain product 28b (100 mg, white solid, yield 79%).

MS (ESI), m/z, 828.6 [M+1]⁺.

### Synthesis of Example 28

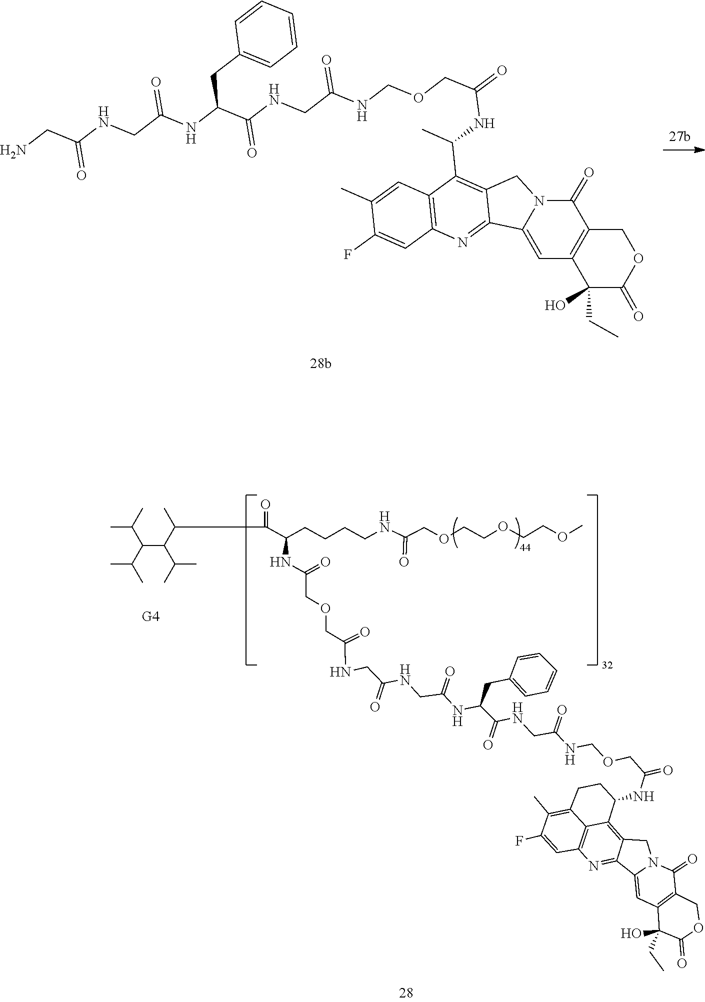

To a solution of 27b (150 mg, 0.0018 mmol) in DMF (5 mL) was added 28a (64 mg, 0.078 mmol, 42 eq), PyBOP (77 mg, 0.15 mmol, 80 eq), and DIPEA (30 mg, 0.24 mmol, 128 eq) at 25° C. under nitrogen atmosphere. The reaction solution was stirred for 12 h, and HPLC showed that the reaction was complete. The reaction solution was concentrated, and the residue was directly dissolved in MeOH/H₂O (50/50 mL), filtered, and purified by ultrafiltration with a 30K MWCO ultrafiltration membrane (H₂O/MeOH=3/2). The final product was freeze-dried to obtain 125 mg (off-white solid, HPLC purity >98%).

Drug content test: Accurately weigh 10 mg of the sample, add 0.5 mL of methanol and 0.5 mL of 6 N HCl solution, stir for 12 hours (decomposed into Example 1 and a small amount of Example 13). An appropriate amount was detected by HPLC. The Example 1 content in the polymer was 8.92%.

### Example 29a

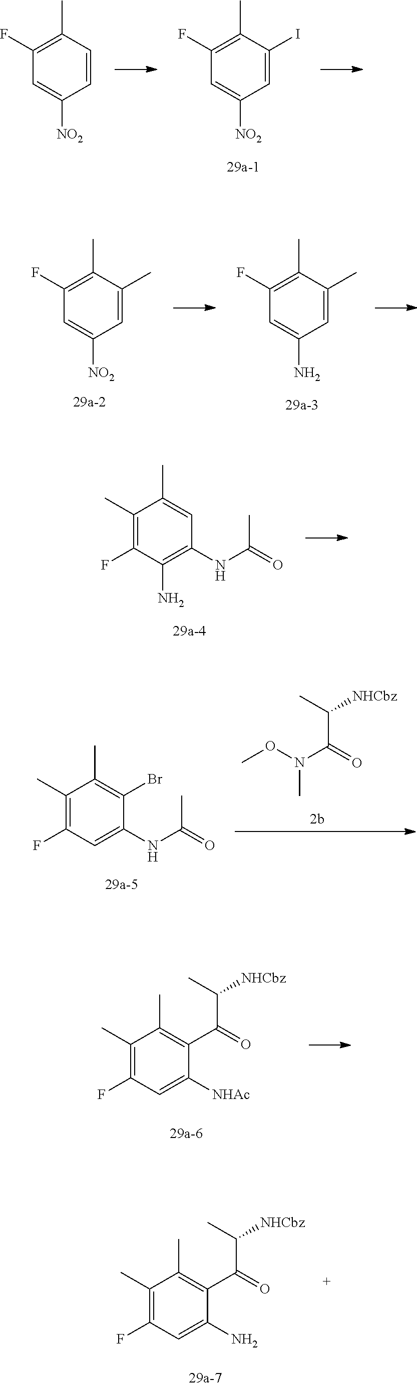

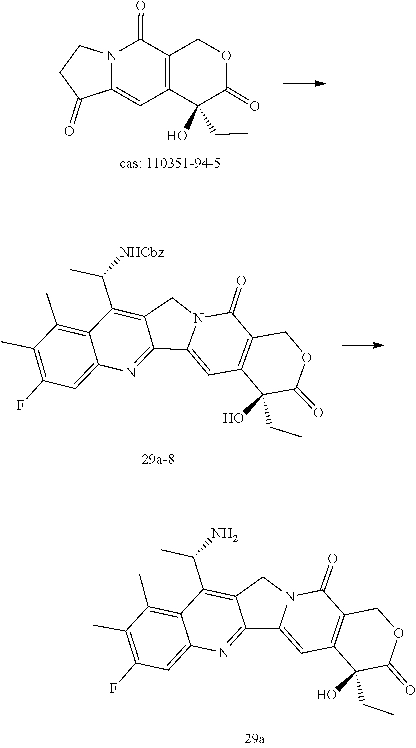 [SMILES: CC[C@@]1(O)C(=O)OCc2c1cc1n(c2=O)CCC1=O ; CC[C@@]1(O)C(=O)OCc2c1cc1n(c2=O)Cc2c-1nc1cc(F)c(C)c(C)c1c2[C@H](C)NC(=O)OCc1ccccc1 ; CC[C@@]1(O)C(=O)OCc2c1cc1n(c2=O)Cc2c-1nc1cc(F)c(C)c(C)c1c2[C@H](C)N]

### Synthesis of Compound 29a-1

2-fluoro-4-nitrotoluene (20 g, 129 mmol) was dissolved in concentrated sulfuric acid (200 mL) and cooled to 5° C. iodine (13.4 g, 52.9 mmol) and sodium iodate (10.5 g, 52.9 mmol) were slowly added under nitrogen atmosphere and stirred for 10 minutes. The reaction solution was stirred overnight at room temperature, and LCMS showed that the reaction was complete. A mixed solution of sodium sulfite (40 g), H₂O (400 mL) and methanol (160 mL) were slowly added to the reaction solution under an ice-water bath, and continue stirred for 1 h. A large amount of solid precipitate was formed. The precipitate was filtered, washed with ethanol, and dried to obtain 18 g of off-white solid.

MS (ESI), m/z, 282 [M+1]⁺.

¹H NMR (400 MHZ, CDCl₃) δ 8.42 (s, 1H), 7.83 (q, 1H), 2.39 (t, J=2.4 Hz, 3H).

### Synthesis of Compound 29a-2

29a-1 (3 g, 46.3 mmol), CH₃B(OH)₂ (8.33 g, 138.8 mmol), cesium fluoride (21 g, 138.8 mmol) and PdCl₂(dppf) (2.71 g, 3.7 mmol) were added to 1, 4-dioxane (150 mL) and then heated to 80° C. for 4 h under nitrogen atmosphere. After LCMS showed that the reaction of the raw materials was complete, the reaction solution was cooled to room temperature and filtered. The filtrate was concentrated and purified by column chromatography (DCM/PE=1:10) to obtain product (7 g, brown solid, 89% yield).

MS (ESI), m/z, 170 [M+1]⁺.

### Synthesis of Compound 29a-3

29a-2 (5 g, 29.6 mmol) was dissolved in mixture solution of THF (50 mL), EtOH (50 mL) and water (10 mL), followed by addition of ammonium chloride (784 mg, 14.8 mmol) and reduced iron powder (33 g, 592 mmol). The mixture was heated to 80° C. and stirred for 3 h. After LCMS showed that the reaction was complete, the reaction solution was cooled to room temperature and filtered. The filtrate was concentrated and dissolved in ethyl acetate. The organic phase was separated, washed with water and brine, dried over anhydrous sodium sulfate, filtered and concentrated to obtain 4.2 g of brown oil.

MS (ESI), m/z, 140 [M+1]⁺.

### Synthesis of Compound 29a-4

29a-3 (4.2 g, 30.22 mmol) was dissolved in DCM (100 mL) under an ice-water bath, and then triethylamine (9.16 g, 90.66 mmol) was added. A solution of acetyl chloride (2.85 g, 36.26 mmol) in DCM (20 mL) was slowly added dropwise, and after completing of addition, the mixture was stirred at 0° C. for 2 h. After LCMS showed that the reaction was complete, the reaction solution was quenched with saturated aqueous ammonium chloride solution (100 mL) and extracted with DCM (100 mL×2). The organic phases were combined, washed with saturated sodium bicarbonate and brine, dried over anhydrous sodium sulfate, filtered and concentrated to obtain 4.5 g of brown solid.

MS (ESI), m/z, 182 [M+1]⁺.

### Synthesis of Compound 29a-5

29a-4 (4.5 g, 24.9 mmol) was dissolved in DMF (100 mL), and NBS (6.64 g, 37.3 mmol) was added portionwise at 0° C. After the addition was completed, the mixture was stirred at room temperature overnight. After LCMS showed that the reaction was complete, the mixture was quenched with saturated aqueous sodium bicarbonate solution and extracted twice with MTBE. The organic phases were combined, washed with brine, dried over anhydrous sodium sulfate, and concentrated. The residue was slurried in a mixed solvent of petroleum ether and ethyl acetate (1:1) for 10 minutes and filtered to obtain 3 g of pure off-white solid.

MS (ESI), m/z, 260 [M]⁺, 262 [M+2]⁺.

### Synthesis of Compound 29a-6

2a (2 g, 7.69 mmol) was dissolved in anhydrous THF (50 mL), and then n-butyllithium (7.7 mL, 19.23 mmol) was added dropwise at −70° C. After stirring for 1 hour, a solution of 29a-5 (1.43 g, 5.38 mmol) in THE solution (20 mL) was slowly added dropwise. After addition, the temperature was warmed to 0° C. slowly and stirred for 1 h. After LCMS showed that the reaction was complete, the reaction was quenched with saturated aqueous ammonium chloride solution and extracted with MTBE. The organic phases were combined, washed with brine, dried over anhydrous sodium sulfate, filtered and concentrated. The crude product was purified by Prep-TLC to obtain compound 29a-6 as a light yellow oil (200 mg).

MS (ESI), m/z, 387 [M+1]⁺.

### Synthesis of Compound 29a-7

29a-6 (200 mg) was dissolved in ethanol (10 mL) and 6 M hydrochloric acid (10 mL) was added. The reaction was stirred for 3 h at room temperature. After LCMS showed that the reaction was complete, the reaction solution was concentrated several times to obtain a colorless oil, which was purified by Prep-HPLC to obtain compound 29a-7 (30 mg).

MS (ESI), m/z, 345 [M+1]⁺.

### Synthesis of Compound 29a-8

To a solution of 29a-7 (30 mg, 0.09 mmol, 1.0 eq) in toluene (2 mL) was added ketone (CAS is 110351-94-5) (28 mg, 0.10 mmol, 1.2 eq) and p-toluenesulfonic acid monohydrate (3.3 mg, 0.02 mmol, 0.2 eq), heated to 110° C. and stirred for 3 h. After the reaction was completed, the mixture was concentrated under vacuum, water was added, stirred and filtered, the solid was purified by slurring with ethanol and water (10/2 mL) to obtain compound 29a-8 (22 mg).

MS (ESI), m/z, 572.2 [M+H]⁺.

### Synthesis of Example 29a

To the mixed suspension solution of 29a-8 (20 mg, 0.03 mmol) in 6M HCl (0.5 mL) and methanol (0.5 mL) was added 10% Pd/C (55% wet, 10 mg). The flash was evacuated and backfilled with H₂ and stirred for 1 h under H₂ atmosphere at 25° C. The reaction was filtered with celite, and the crude product was purified by Prep-HPLC (CH₃CN/H₂O, 0.05% TFA) to obtain Example 29a (5 mg).

MS (ESI), m/z, 438.20 [M+1]⁺.

### Example 29b

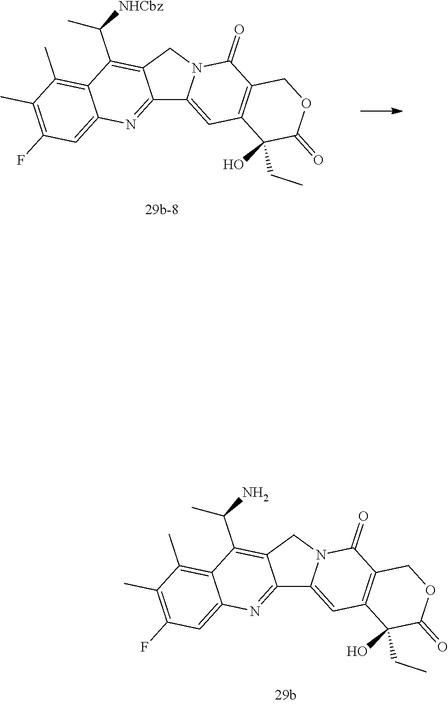 [SMILES: CC[C@@]1(O)C(=O)OCc2c1cc1n(c2=O)Cc2c-1nc1cc(F)c(C)c(C)c1c2[C@@H](C)NC(=O)OCc1ccccc1 ; CC[C@@]1(O)C(=O)OCc2c1cc1n(c2=O)Cc2c-1nc1cc(F)c(C)c(C)c1c2[C@@H](C)N]

### Synthesis of compounds 29b-1˜8

According to the synthesis method of compounds 29a-1˜8.

### Synthesis of Example 29b

To the mixed suspension solution of 29b-8 (50 mg, 0.09 mmol) in 6M HCl (1 mL) and methanol (1 mL) was added 10% Pd/C (55% wet, 20 mg). The flash was evacuated and backfilled with H₂ and stirred for 1 h under H₂ atmosphere at 25° C. The reaction was filtered with celite, and the crude product was purified by Prep-HPLC (CH₃CN/H₂O, 0.05% TFA) to obtain Example 29b (15 mg).

MS (ESI), m/z, 438.20 [M+1]⁺.

### Example 30a

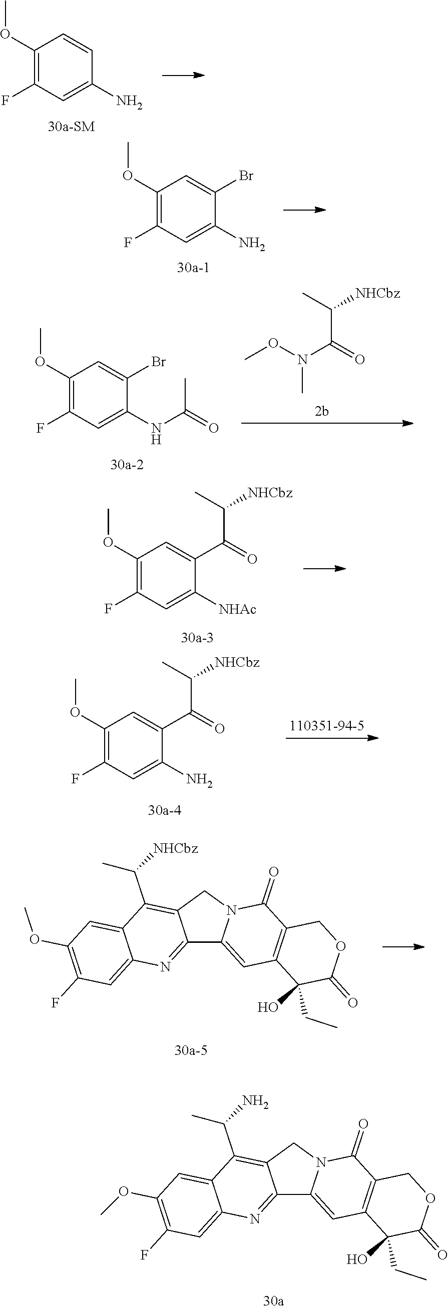

### Synthesis of Compound 30a-1

To a solution of 3-fluoro-4-methoxyaniline (10 g, 70.9 mmol, 1.0 eq) in DMF (50 mL) was added NBS (13.5 g, 80 mmol) at 25° C. under nitrogen atomsphere. The reaction solution was stirred overnight. After the reaction was completed, the mixture was quenched with water (200 mL), extracted with ethyl acetate (200 mL×3). The organic phases were combined, washed with brine, dried over anhydrous sodium sulfate, and purified by column chromatography with PE: EA (10:1) to obtain 5.0 g of light yellow solid (32.2% yield).

MS (ESI), m/z, 222.2 [M+H]⁺.

### Synthesis of Compound 30a-2

To a solution of 30a-1 (5 g, 22.8 mmol) in dichloromethane (150 mL) was add triethylamine (4.9 g, 48 mmol) and acetyl chloride (1.9 g, 24 mmol) at 25° C. under nitrogen atmosphere, and stirred for 3 h. After the reaction was completed, the mixture was quench with water (100 mL) and extracted with dichloromethane (50 mL×3). The organic phases were combined, washed with brine, dried over anhydrous sodium sulfate, and purified by column chromatography (PE/EA) to obtain product 30a-2 (5.5 g, 93.2% yield).

MS (ESI), m/z, 262.2 [M+H]⁺.

### Synthesis of Compound 30a-3

To a solution (10 0 mL) of 30a-2 (3 g, 11.5 mmol, 1.0 eq) in THF was added i-PrMgCl (2M, 15 mL, 30 mmol, 2.0 eq) and 2b (4.2 g, 16 mmol, 1.5 eq) at −25° C. under nitrogen atmosphere. The reaction solution was gradually warmed to room temperature and stirred for 5 h. After the reaction was completed, the reaction was quenched by aqueous ammonium chloride solution, extracted with ethyl acetate (50 mL×3), dried over anhydrous sodium sulfate, purified by column chromatography, and then Pre-TLC to obtain 150 mg (purity 80%, yield 3.5%).

MS (ESI), m/z, 389.2 [M+H]⁺.

### Synthesis of Compound 30a-4

To a solution of 30a-3 (150 mg, 0.38 mmol) in THF/MeOH (10/10 mL) was added hydrochloric acid (6M, 8 mL) at room temperature 25° C. under nitrogen atmosphere and stirred overnight. After the reaction was completed, the reaction solution was directly concentrated, neutralized with sodium bicarbonate, and extracted with ethyl acetate, concentrated. The residue was purified by Prep-TLC to give product 30a-4 as a light-yellow oil (85 mg, 64% yield).

MS (ESI), m/z, 347.2 [M+H]⁺.

### Synthesis of Compound 30a-5

To a solution of 30a-4 (85 mg, 0.24 mmol) in toluene (5 mL) was added ketone (CAS is 110351-94-5) (70 mg, 0.26 mmol) and p-toluenesulfonic acid monohydrate (50 mg, 4.12 mmol) under nitrogen atmosphere. The reaction was heated to 120° C. and stirred for 5 h. The reaction solution was concentrated and purified by column chromatography to obtain product 30a-5 as a light yellow solid (40 mg, purity 86%, yield 65%).

MS (ESI), m/z, 574.2 [M+H]⁺.

### Synthesis of Example 30a

To a suspension of 30a-5 (40 mg, 0.069 mmol) in MeOH/THF (5/5 mL) was added 10% palladium on carbon (5 mg) at 25° C. The flask was backfilled three times with hydrogen. The reaction solution was stirred under hydrogen atmosphere (hydrogen balloon) for 2 h. After the reaction was completed, filtered, concentrated, and purified by Prep-HPLC (CH₃CN/H₂O, 0.05% TFA) to obtain product 30a as a light yellow solid (4 mg, purity 95.2%).

MS (ESI), m/z, 440.2 [M+H]⁺.

¹H NMR (400 MHZ, DMSO) δ 8.54 (s, 2H), 8.09 (d, J=11.8 Hz, 1H), 7.80 (d, J=8.8 Hz, 1H), 7.34 (s, 1H), 6.87 (s, 1H), 6.54 (s, 1H), 5.69-5.33 (m, 5H), 4.14 (s, 3H), 1.99-1.82 (m, 2H), 1.79 (d, J=6.8 Hz, 3H), 0.89 (t, J=7.2 Hz, 3H).

### Example 30b

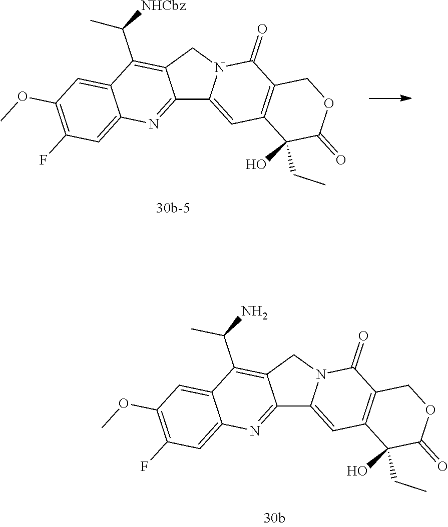 [SMILES: CC[C@@]1(O)C(=O)OCc2c1cc1n(c2=O)Cc2c-1nc1cc(F)c(OC)cc1c2[C@@H](C)NC(=O)OCc1ccccc1 ; CC[C@@]1(O)C(=O)OCc2c1cc1n(c2=O)Cc2c-1nc1cc(F)c(OC)cc1c2[C@@H](C)N]

### Synthesis of Compounds 30b-1˜5

According to the synthesis method of compounds 30a-1˜5.

### Synthesis of Example 30b

To the mixed suspension solution of 30b-5 (80 mg, 0.14 mmol) in MeOH/THF (8/8 mL) was added 10% Pd/C (10 mg). The flash was evacuated and backfilled with H₂ (3×) and stirred for 2 h under H₂ atmosphere. After the reaction was completed, the mixture was filtered, concentrated and purified by Prep-HPLC (CH₃CN/H₂O, 0.05% TFA) to obtain Example 30b as a light yellow solid (15 mg, purity 93.9%).

MS (ESI), m/z, 440.2 [M+H]⁺.

¹H NMR (400 MHZ, DMSO) δ 8.56 (s, 2H), 8.09 (d, J=11.8 Hz, 1H), 7.80 (d, J=8.8 Hz, 1H), 7.34 (s, 1H), 6.56 (s, 1H), 5.59 (q, J=18.8 Hz, 3H), 5.47 (s, 2H), 4.15 (s, 3H), 1.88 (dt, J=22.8, 7.2 Hz, 2H), 1.78 (d, J=6.8 Hz, 3H), 0.89 (t, J=7.2 Hz, 3H).

### Examples 30c and 30d

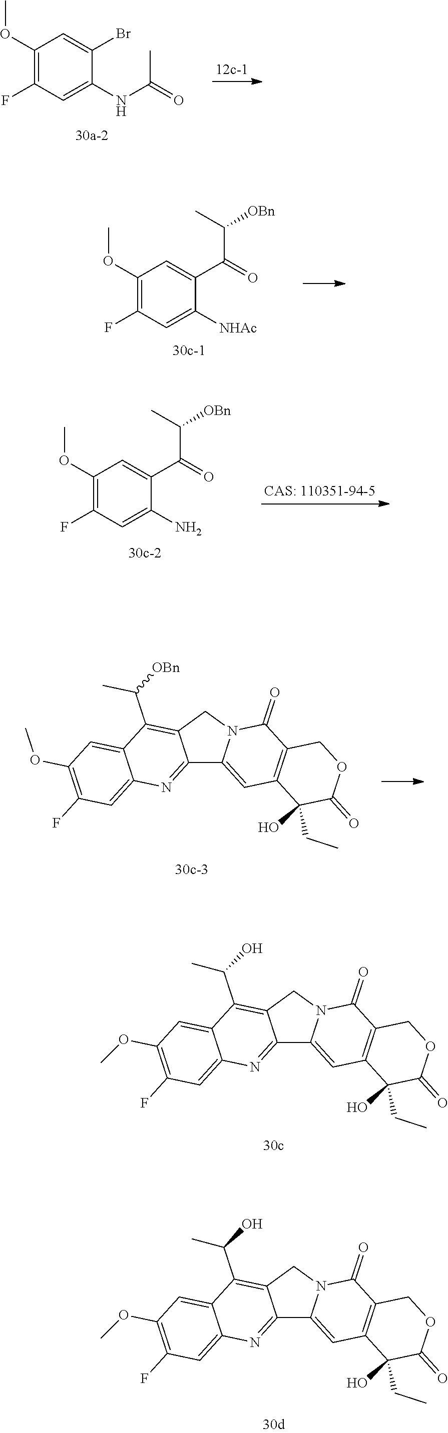 [SMILES: COc1cc(Br)c(NC(C)=O)cc1F ; COc1cc(C(=O)[C@H](C)OCc2ccccc2)c(NC(C)=O)cc1F ; COc1cc(C(=O)[C@H](C)OCc2ccccc2)c(N)cc1F ; CC[C@@]1(O)C(=O)OCc2c1cc1n(c2=O)Cc2c-1nc1cc(F)c(OC)cc1c2C(C)OCc1ccccc1 ; CC[C@@]1(O)C(=O)OCc2c1cc1n(c2=O)Cc2c-1nc1cc(F)c(OC)cc1c2[C@H](C)O ; CC[C@@]1(O)C(=O)OCc2c1cc1n(c2=O)Cc2c-1nc1cc(F)c(OC)cc1c2[C@@H](C)O]

### Synthesis of Compound 30c-1

Compound 30a-2 (2.5 g, 9.54 mmol, 1.0 eq) was dissolved in anhydrous THF (40 mL) and cooled to −70° C., then 2.5M butyllithium (8.4 mL, 21.0 mmol, 2.2 eq) was added dropwise. After addition, the mixture was continued to stir for 1 h. A solution of 12c-1 (3.2 g, 14.3 mmol, 1.5 eq) in anhydrous THF (10 mL) was added dropwise. After addition, the reaction solution was continued to stir at −70° C. for 2 h. The mixture was quenched with saturated NH₄Cl and extracted with ethyl acetate (100 mL×3). The organic phases were combined, washed with brine, dried over anhydrous sodium sulfate, filtered, concentrated, and purified by column chromatography (PE/EtOAc) to obtain 1.2 g.

MS (ESI), m/z, 346.1 [M+1]⁺.

### Synthesis of Compound 30c-2

30c-1 (1.2 g, 3.48 mmol) was dissolved in methanol and tetrahydrofuran (10/10 mL), then 6 M dilute hydrochloric acid (10 mL) was added and stirred for 12 h at room temperature. After LCMS showed almostly complete, the mixture was concentrated, diluted with water, and extracted with ethyl acetate (50 mL×3). The organic phases were combined, washed with brine, dried over anhydrous sodium sulfate, filtered, concentrated, and purified by column chromatography to obtain 0.7 g as colorless oil.

MS (ESI), m/z, 304.0 [M+1]⁺.

### Synthesis of Compound 30c-3

To a solution of 30c-2 (700 mg, 2.31 mmol, 1.0 eq) in toluene (10 mL) was added ketone (CAS is 110351-94-5) (668 mg, 2.54 mmol, 1.1 eq) and p-toluenesulfonic acid monohydrate (88 mg, 0.46 mmol, 0.2 eq) and the mixture was heated to 110° C. and stirred for 3 h. After the completion of the reaction by LCMS (partial racemization occurred), the reaction was concentrated, stirred with water, and filtered to obtain 800 mg of solid residue (yield, 65%).

MS (ESI), m/z, 531.0 [M+1]⁺.

### Synthesis of Examples 30c and 30d

30c-3 (400 mg, 0.75 mmol) was dissolved in ethyl acetate (20 mL), then methanol (2 mL) and wet 10% palladium on carbon (100 mg) were added. The reaction was stirred at room temperature under H₂ atmosphere (H₂ balloon) for 2 h. The mixture was filtered by celite, concentrated, and purified by Prep-HPLC (CH₃CN/H₂O, 0.05% TFA) to obtain 30c (55 mg) and 30d (23 mg) and other unseparated mixture (100 mg).

LCMS (ESI), m/z, 441.0 [M+1]⁺.

### Example 31a

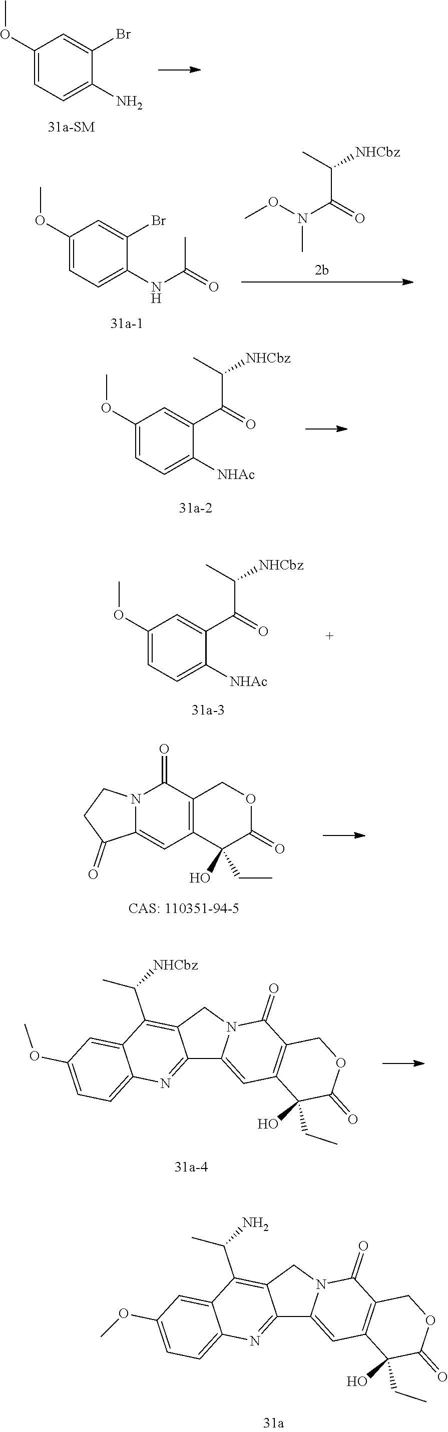

### Synthesis of Compound 31a-1

To a solution of 2-bromo-4-methoxyaniline (31a-SM) (10 g, 48 mmol, 1.0 eq) in CH₂Cl₂ (150 mL) was added triethylamine (4.9 g, 48 mmol) and acetyl chloride (3.8 g, 48 mmol) at room temperature 25° C. under nitrogen atmosphere, and then stirred for 3 h. After the reaction was completed, the reaction solution was quenched with water (100 mL) and extracted with dichloromethane (100 mL×3). The organic phases were combined, washed with brine, dried over anhydrous sodium sulfate, and purified by column chromatography to obtain compound 31a-1 (8 g, 67% yield).

MS (ESI), m/z, 250.2 [M+H]⁺.

### Synthesis of Compound 31a-2

To a solution of 31a-1 (4 g, 16 mmol, 1.0 eq) in THF (100 mL) was added i-PrMgCl (2 M, 16 mL, 32 mmol, 2 eq) and 2b (4.2 g, 16 mmol, 1.5 eq) at −25° C. under nitrogen atmosphere, the reaction solution was gradually warmed to room temperature and stirred for 5 h. After the reaction was completed, the reaction was quenched with ammonium chloride aqueous solution, extracted with ethyl acetate (50 mL×3), dried over anhydrous sodium sulfate, purified by column chromatography, and then further purified by Prep-TLC to obtain 300 mg (purity 70%, yield 4%).

MS (ESI), m/z, 377.2 [M+H]⁺.

### Synthesis of Compound 31a-3

To a solution of 31a-2 (300 mg, 0.53 mmol, 1.0 eq) in THF/MeOH (10/10 mL) was added hydrochloric acid (6M, 10 mL) under nitrogen atmosphere at room temperature 25° C. and stirred overnight. After the reaction was completed, the mixture was concentrated, neutralized with sodium bicarbonate, and extracted with ethyl acetate (50 mL×3). The organic phases were combined, washed with brine, dried over anhydrous sodium sulfate, filtered, concentrated, and purified by Prep-TLC to obtain product 31a-3 (180 mg, 53% yield).

MS (ESI), m/z, 335.2 [M+H]⁺.

### Synthesis of Compound 31a-4

To a solution of compound 31a-3 (180 mg, 0.53 mmol) in toluene (5 mL) was added ketone (CAS is 110351-94-5) (70 mg, 0.26 mmol) and p-toluenesulfonic acid monohydrate (50 mg, 4.12 mmol), heated to 120° C. and stirred for 5 h. The reaction solution was cooled to room temperature, concentrated, and purified by column chromatography to obtain product 31a-4 as a light yellow solid (95 mg, 65% yield).

MS (ESI), m/z, 562.2 [M+H]⁺.

### Synthesis of Example 31a

To a suspension solution of 31a-4 (18 mg, 0.032 mmol, 1.0 eq) in MeOH/THF (3/3 mL) was added 10% palladium on carbon (5 mg) at room temperature 25° C., backfilled with H₂ (3×), and stirred overnight under H₂ atmosphere (H₂ balloon). After the reaction was completed, the reaction was filtered, concentrated, and purified to obtain product 31a as a light yellow solid (2.2 mg, purity 92%).

MS (ESI), m/z, 422.2 [M+H]⁺.

¹H NMR (400 MHZ, DMSO) δ 8.47 (s, 2H), 8.19 (d, J=9.8 Hz, 1H), 7.69-7.59 (m, 2H), 7.33 (s, 1H), 6.56 (s, 1H), 5.68-5.28 (m, 5H), 4.04 (s, 3H), 1.88 (td, J=14.4, 7.2 Hz, 2H), 1.78 (d, J=6.8 Hz, 3H), 0.89 (t, J=7.2 Hz, 3H).

### Example 31b

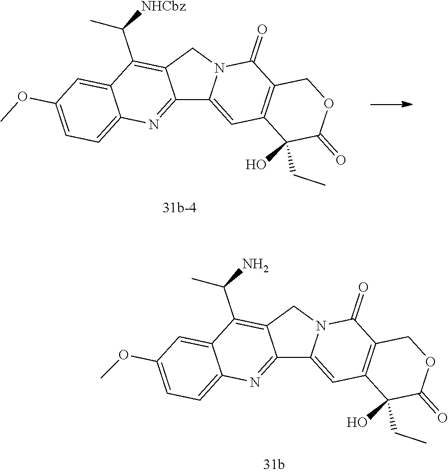 [SMILES: CC[C@@]1(O)C(=O)OCc2c1cc1n(c2=O)Cc2c-1nc1ccc(OC)cc1c2[C@@H](C)NC(=O)OCc1ccccc1 ; CC[C@@]1(O)C(=O)OCc2c1cc1n(c2=O)Cc2c-1nc1ccc(OC)cc1c2[C@@H](C)N]

### Synthesis of Compounds 31b-1˜4

According to the synthesis method of compounds 31a-1˜4.

### Synthesis of Example 31b

To a suspension solution of 31b-4 (50 mg, 0.09 mmol) in MeOH/THF (3/3 mL) was added 10% palladium on carbon (10 mg) at room temperature 25° C., backfilled with H₂ (3×), and stirred overnight under H₂ atmosphere (H₂ balloon). After the reaction was completed, the reaction was filtered, concentrated, and purified by Prep-HPLC (CH₃CN/H₂O, 0.05% TFA) to obtain product 31b as a light yellow solid (5.5 mg, purity 98%).

MS (ESI), m/z, 422.2 [M+H]⁺.

¹H NMR (400 MHZ, DMSO) δ 8.59 (s, 2H), 8.32-8.13 (m, 1H), 7.64 (dd, J=6.0, 2.6 Hz, 2H), 7.33 (s, 1H), 6.55 (s, 1H), 5.71-5.32 (m, 5H), 4.04 (s, 3H), 1.87 (td, J=14.2, 7.0 Hz, 2H), 1.78 (d, J=6.8 Hz, 3H), 0.89 (t, J=7.2 Hz, 3H).

### Examples 31c and 31d

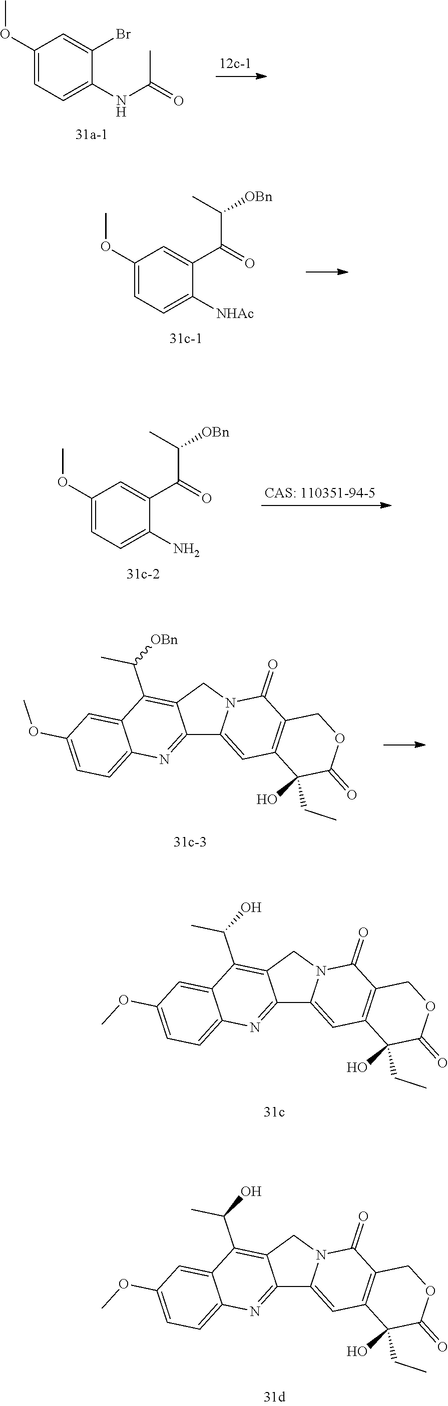 [SMILES: COc1ccc(NC(C)=O)c(Br)c1 ; COc1ccc(NC(C)=O)c(C(=O)[C@H](C)OCc2ccccc2)c1 ; COc1ccc(N)c(C(=O)[C@H](C)OCc2ccccc2)c1 ; CC[C@@]1(O)C(=O)OCc2c1cc1n(c2=O)Cc2c-1nc1ccc(OC)cc1c2C(C)OCc1ccccc1 ; CC[C@@]1(O)C(=O)OCc2c1cc1n(c2=O)Cc2c-1nc1ccc(OC)cc1c2[C@H](C)O ; CC[C@@]1(O)C(=O)OCc2c1cc1n(c2=O)Cc2c-1nc1ccc(OC)cc1c2[C@@H](C)O]

### Synthesis of Compound 31c-1

Compound 31a-1 (1.5 g, 6.20 mmol, 1.0 eq) was dissolved in anhydrous THF (30 mL) and cooled to −70° C., then 2.5M butyllithium (5.5 mL, 13.6 mmol, 2.2 eq) was added dropwise. After addition, the mixture was continued to stir for 1 h. A solution of 12c-1 (1.6 g, 7.44 mmol, 1.2 eq) in anhydrous THF (10 mL) was added dropwise. After addition, the reaction solution was continued to stir at −70° C. for 2 h. The mixture was quenched with saturated NH₄Cl and extracted with ethyl acetate (80 mL×3). The organic phases were combined, washed with brine, dried over anhydrous sodium sulfate, filtered, concentrated, and purified by column chromatography (PE/EtOAc) to obtain 0.6 g.

MS (ESI), m/z, 328.1 [M+1]⁺.

### Synthesis of Compound 31c-2

31c-1 (0.5 g, 1.5 mmol) was dissolved in methanol and tetrahydrofuran (10/10 mL), then 6 M dilute hydrochloric acid (10 mL) was added, and the reaction was stirred at room temperature for 12 hours. After LCMS showed that reaction was almost completed, the reaction solution was concentrated, diluted with water, and extracted with ethyl acetate (50 mL×3). The organic phases were combined, washed with brine, dried over anhydrous sodium sulfate, filtered, concentrated and purified by column chromatography to obtain 0.35 g as colorless oil. MS (ESI), m/z, 286.0 [M+1]⁺.

### Synthesis of Compound 31c-3

To a solution of 31c-2 (350 mg, 1.05 mmol, 1.0 eq) and ketone (CAS is 110351-94-5) (332 mg, 1.26 mmol, 1.2 eq) in toluene (10 mL) was added p-toluenesulfonic acid monohydrate (40 mg, 0.21 mmol, 0.2 eq) and the mixture was heated to 110° C. and stirred for 3 h. After LCMS showed that the reaction was completed (partial racemization occurred), the reaction solution was directly concentrated and purified by column chromatography to obtain 380 mg (yield, 70%).

MS (ESI), m/z, 513.0 [M+1]⁺.

### Synthesis of Examples 31c and 31d

31c-3 (350 mg, 0.68 mmol) was dissolved in ethyl acetate (20 mL), then methanol (2 mL) and wet 10% palladium on carbon (100 mg) were added, and the mixture was stirred at room temperature under H₂ atmosphere (H₂ balloon) for 1 h. The reaction solution was filtered through celite, concentrated, and purified by Prep-HPLC (CH₃CN/H₂O, 0.05% TFA) to obtain 31c (58 mg) and 31d (32 mg) and other unseparated mixtures (89 mg).

LCMS (ESI), m/z, 441.0 [M+1]⁺.

### Example 32a

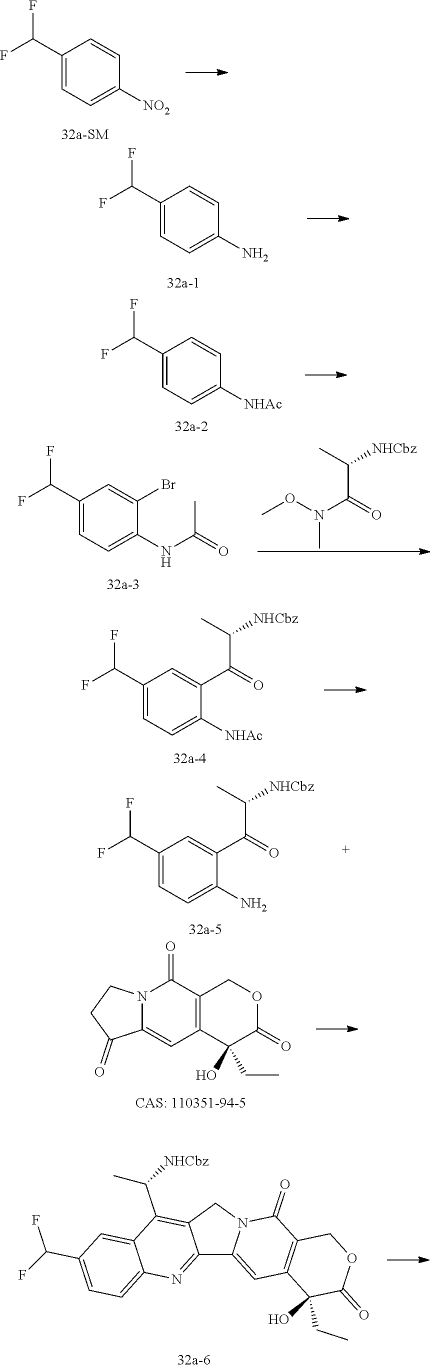

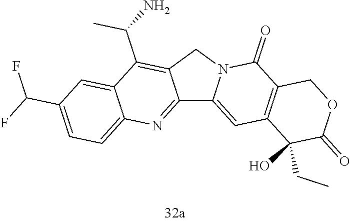 [SMILES: CC[C@@]1(O)C(=O)OCc2c1cc1n(c2=O)Cc2c-1nc1ccc(C(F)F)cc1c2[C@H](C)N]

### Synthesis of Compound 32a-1

To a solution of 4-difluoromethylnitrobenzene (5 g, 28 mmol, 1.0 eq) in EtOH (50 mL) was added reduced iron powder (2.4 g, 43 mmol, 1.5 eq) and ammonium chloride (2.3 g, 43 mmol, 1.5 eq) under nitrogen atmosphere. The reaction solution was heated to reflux and stirred overnight. After the reaction was completed, the reaction solution was cooled to room temperature, filtered, and the filtrate was diluted with water and ethyl acetate, and extracted with ethyl acetate (100 mL×3). The organic phases were combined, washed with salt, dried over anhydrous sodium sulfate, concentrated, and purified by column chromatography to obtain 3.6 g of light yellow solid (90% yield).

MS (ESI), m/z, 144.2 [M+H]⁺.

### Synthesis of Compound 32a-2

To a solution of 32a-1 (3.6 g, 25 mmol, 1.0 eq) in CH₂Cl₂ (150 mL) was added triethylamine (2.4 g, 24 mmol) and acetyl chloride (1.9 g, 24 mmol) at room temperature 25° C. under nitrogen atmosphere, and stirrd for 3 h. After the reaction was completed, the reaction solution was quenched with water (100 mL) and extracted with dichloromethane (100 mL×3). The organic phases were combined, washed with brine, dried over anhydrous sodium sulfate, concentrated, and purified by column chromatography to obtain compound 32a-2 (4.0 g, colorless oil, 87% yield).

MS (ESI), m/z, 186.2 [M+H]⁺.

### Synthesis of Compound 32a-3

To a solution of 32a-2 (3.8 g, 19 mmol) in DMF (50 mL) was added NBS (3.5 g, 19 mmol) and then stirred overnight at room temperature 25° C. under nitrogen atmosphere. After the reaction was completed, the mixture was quenched with water (200 mL), and extracted with ethyl acetate (100 mL×3). The organic phases were combined, washed with brine, dried over anhydrous sodium sulfate, concentrated, and purified by column chromatography to obtain compound 32a-3 (3.5 g, 80.6% yield).

MS (ESI), m/z, 264.2,266.2 [M+H]⁺.

### Synthesis of Compound 32a-4

To a solution of 32a-3 (3.5 g, 13.3 mmol, 1.0 eq) in THF (10 0 mL) was added i-PrMgCl (2M, 15 mL, 16 mmol, 2.0 eq) and 2b (3.0 g, 13.3 mmol, 1.5 eq) at −25° C. under nitrogen atmosphere. The reaction solution was gradually warmed to room temperature and stirred for 5 h. After the reaction was completed, the mixture was quenched with ammonium chloride aqueous solution, and extracted with ethyl acetate (50 mL×3). The organic phases were combined, washed with salt, dried over anhydrous sodium sulfate, filtered and concentrated, purified by column chromatography, and Prep-TLC to obtain 45 mg (purity 80%, yield 1%).

MS (ESI), m/z, 391.2 [M+H]⁺.

### Synthesis of Compound 32a-5

To a solution of 32a-4 (30 mg, purity 80%, 0.53 mmol, 1.0 eq) in THF/MeOH (5/5 mL) was added hydrochloric acid (6M, 5 mL) at room temperature 25° C. under nitrogen atmosphere, and stirred overnight. After the reaction was completed, the mixture was concentrated and purified by Prep-HPLC (CH₃CN/H₂O, 0.05% TFA) to obtain the target product 32a-5 (28 mg, purity 80%, yield 45%).

MS (ESI), m/z, 349.2 [M+H]⁺.

### Synthesis of Compound 32a-6

To a solution of 32a-5 (28 mg, 0.08 mmol) in toluene (5 mL) was added ketone (CAS is 110351-94-5) (16 mg, 0.08 mmol) and p-toluenesulfonic acid monohydrate (10 mg, 1.0 mmol, 1.5 eq) under nitrogen atmosphere. The reaction solution was heated to 120° C. and stirred for 5 h. The reaction solution was cooled to room temperature, directly concentrated and purified by column chromatography to obtain product 32a-6 (15 mg, 32% yield).

MS (ESI), m/z, 576.2 [M+H]⁺.

### Synthesis of Example 32a

To a suspension solution of 32a-6 (15 mg, 0.16 mmol) in MeOH/THF (5/5 mL) was added Pd/C (5 mg) at room temperature 25° C., backfilled with H₂ (3×), and stir overnight under H₂ atmosphere (H₂ balloon). After the reaction was completed, the reaction solution was filtered through celite, concentrated, and purified by Prep-HPLC (CH₃CN/H₂O, 0.05% TFA) to obtain product 32a as a light yellow solid (2 mg, purity 94%).

MS (ESI), m/z, 442.2 [M+H]⁺.

### Example 32b

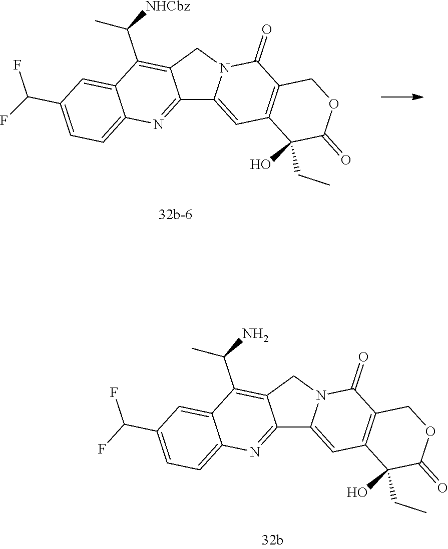 [SMILES: CC[C@@]1(O)C(=O)OCc2c1cc1n(c2=O)Cc2c-1nc1ccc(C(F)F)cc1c2[C@@H](C)NC(=O)OCc1ccccc1 ; CC[C@@]1(O)C(=O)OCc2c1cc1n(c2=O)Cc2c-1nc1ccc(C(F)F)cc1c2[C@@H](C)N]

### Synthesis of Compounds 32b-1˜6

According to the synthesis method of compounds 32a-1˜6.

### Synthesis of Example 32b

To a suspension solution of 32b-6 (30 mg, 0.05 mmol) in MeOH/THF (3/3 mL) was added 10% Pd/C (10 mg) at room temperature 25° C., backfilled with H₂ (3×), and stirred overnight under H₂ atmosphere (H₂ balloon). After the reaction was completed, the reaction was filtered, concentrated, and purified by Prep-HPLC (CH₃CN/H₂O, 0.05% TFA) to obtain product 32b as a light yellow solid (1.5 mg, purity 92%).

MS (ESI), m/z, 442.2 [M+H]⁺.

### Example 33a

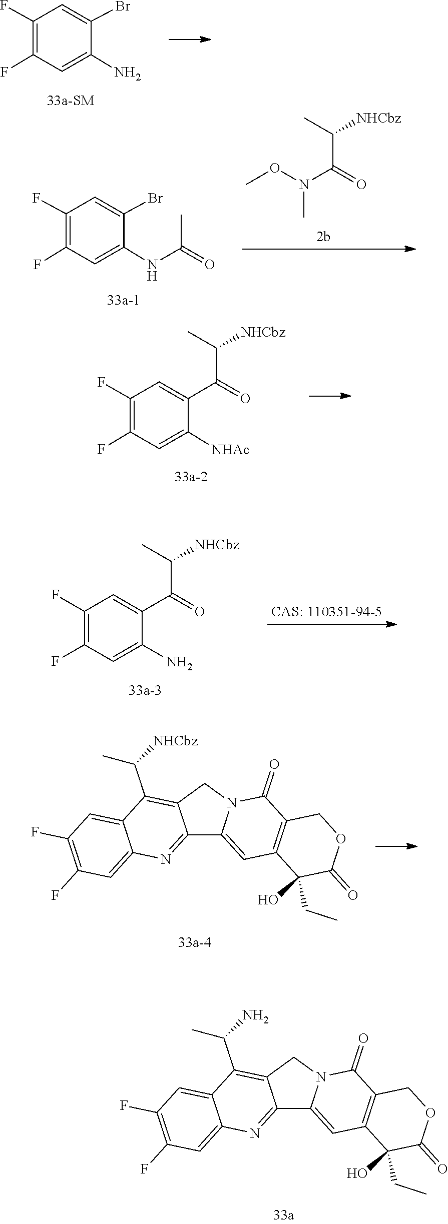

### Synthesis of Compound 33a-1

To a solution of 2-bromo-4,5-difluoroaniline (10 g, 48 mmol, 1.0 eq) in CH₂Cl₂ (150 mL) was added triethylamine (4.9 g, 48 mmol) and acetyl chloride (3.8 g, 48 mmol) at room temperature 25° C. under nitrogen atmosphere, and then stirred for 3 h. After the reaction was completed, quenched with water (100 mL), extracted with dichloromethane (300 mL×3). The organic phases were combined, washed with brine, dried over anhydrous sodium sulfate, concentrated, and purified by column chromatography to obtain compound 33a-1 (8 g, 67% yield).

MS (ESI), m/z, 250.2 [M+H]⁺.

### Synthesis of Compound 33a-2

To a solution of 33a-1 (4 g, 16 mmol, 1.0 eq) in THF (100 mL) was added i-PrMgCl (2 M, 16 mL, 32 mmol, 2.0 eq) and 2b (4.2 g, 16 mmol, 1.5 eq) at −25° C. under nitrogen atmosphere. The reaction solution was gradually warmed to room temperature and stirred for 5 h. After the reaction was completed, the reaction was quenched with ammonium chloride aqueous and extracted with ethyl acetate (50 mL×3). The organic phases were combined, washed with brine, dried over anhydrous sodium sulfate, filtered and concentrated, and purified by column chromatography to obtain compound 33a-2 (300 mg, purity 70%, yield 4%). MS (ESI), m/z, 377.2 [M+H]⁺.

### Synthesis of Compound 33a-3

To a solution of 33a-2 (300 mg, 0.53 mmol) in THF/MeOH (10/10 mL) was added hydrochloric acid (6 M, 10 mL) at room temperature 25° C. under nitrogen atmosphere and stirred overnight. After the reaction was completed, it was concentrated directly, neutralized with sodium bicarbonate, and extracted with ethyl acetate (50 mL×3). The organic phases were combined, washed with brine, dried over anhydrous sodium sulfate, filtered and concentrated, purified by Prep-TLC to give product 33a-3 (180 mg, 53% yield).

MS (ESI), m/z, 335.2 [M+H]⁺.

### Synthesis of compound 33a-4

To a solution of 33a-3 (180 mg, 0.53 mmol) in toluene (15 mL) was added ketone (CAS is 110351-94-5) (70 mg, 0.26 mmol) and p-toluenesulfonic acid monohydrate (50 mg, 4.12 mmol, 1.5 eq) under nitrogen atmosphere, and heated to 120° C. for 5 h. The reaction solution was cooled to room temperature, concentrated, and purified by column chromatography to obtain compound 33a-4 (95 mg, 65% yield).

MS (ESI), m/z, 562.2 [M+H]⁺.

### Synthesis of Example 33a

To a suspension solution of 33a-4 (95 mg, 0.16 mmol, 1.0 eq) in MeOH/THF (10/10 mL) was added 10% Pd/C (10 mg) at room temperature 25° C., backfilled with H₂ (3×), and stirred overnight under H₂ atmosphere (H₂ balloon). After the reaction was completed, the reaction was filtered with celite, concentrated, and purified by Prep-HPLC (CH₃CN/H₂O, 0.05% TFA) to obtain product 33a as a light yellow solid (35 mg).

MS (ESI), m/z, 428.2 [M+H]⁺.

33a: ¹H NMR (400 MHZ, DMSO) δ 8.61 (dd, J=12.8, 8.4 Hz, 3H), 8.34 (dd, J=11.2, 8.2 Hz, 1H), 7.38 (s, 1H), 6.58 (s, 1H), 5.60 (q, J=18.8 Hz, 2H), 5.46 (d, J=10.8 Hz, 3H), 1.87 (td, J=14.2, 7.0 Hz, 2H), 1.74 (d, J=6.8 Hz, 3H), 0.88 (dd, J=9.4, 5.4 Hz, 3H).

### Example 33b

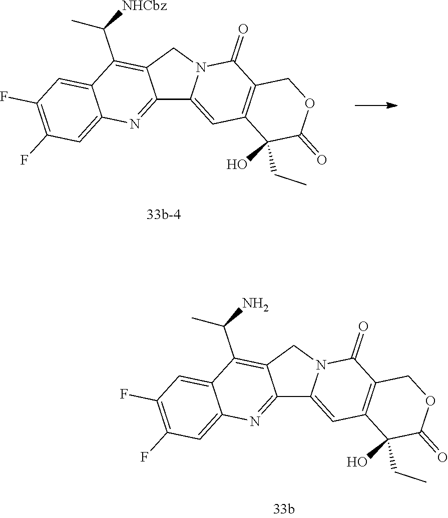 [SMILES: CC[C@@]1(O)C(=O)OCc2c1cc1n(c2=O)Cc2c-1nc1cc(F)c(F)cc1c2[C@@H](C)NC(=O)OCc1ccccc1 ; CC[C@@]1(O)C(=O)OCc2c1cc1n(c2=O)Cc2c-1nc1cc(F)c(F)cc1c2[C@@H](C)N]

### Synthesis of Compounds 33b-1˜4

According to the synthesis method of compounds 33a-1˜4.

### Synthesis of Example 33b

To a suspension solution of 33b-4 (50 mg, 0.09 mmol, 1.0 eq) in MeOH/THF (10/10 mL) was added 10% Pd/C (10 mg) at room temperature 25° C., backfilled with H₂ (3×), and stirred overnight under H₂ atmosphere (H₂ balloon). After the reaction was completed, the reaction was filtered with celite, concentrated, and purified by Prep-HPLC (CH₃CN/H₂O, 0.05% TFA) to obtain product 33b as a light yellow solid (13 mg).

MS (ESI), m/z, 428.2 [M+H]⁺.

33b: ¹H NMR (400 MHZ, DMSO) δ 8.61 (dd, J=12.8, 8.4 Hz, 3H), 8.34 (dd, J=11.2, 8.4 Hz, 1H), 7.38 (s, 1H), 6.49 (dd, J=57.0, 29.2 Hz, 2H), 5.60 (q, J=19.0 Hz, 3H), 5.49-5.33 (m, 2H), 1.89 (dd, J=15.0, 7.3 Hz, 2H), 1.81-1.69 (m, 2H), 0.92-0.82 (m, 3H).

### Example 34a

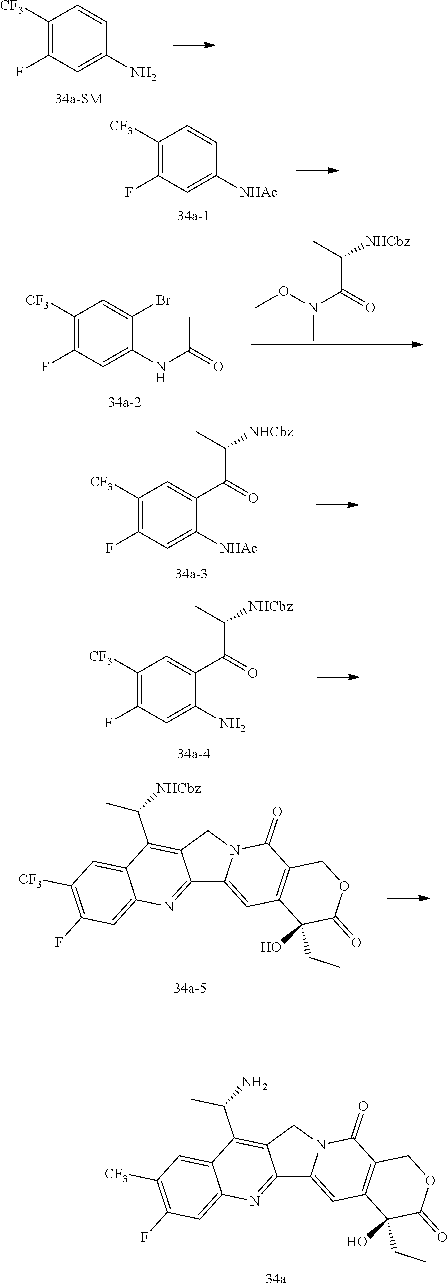

### Synthesis of Compound 34a-1

To a solution of 4-amino-2-fluorotrifluorotoluene (8 g, 44 mmol, 1.0 eq) in CH₂Cl₂ (150 mL) was added triethylamine (4.2 g, 44 mmol) and acetyl chloride (3.6 g, 44 mmol) at room temperature 25° C. under nitrogen atmosphere, and then stirred for 3 h. After the reaction was completed, quenched with water (100 mL), extracted with dichloromethane (200 mL×3). The organic phases were combined, washed with brine, dried over anhydrous sodium sulfate, concentrated, and purified by column chromatography to obtain compound 33a-1 (8 g, 81% yield).

MS (ESI), m/z, 222.2 [M+H]⁺.

### Synthesis of Compound 34a-2

To a solution of 34a-1 (8 g, 36 mmol, 1.0 eq) in DMF (50 mL) was added NBS (6.8 g, 36 mmol) at room temperature 25° C. under nitrogen atmosphere and stirred overnight. After the reaction was completed, the reaction solution was quenched with water (200 ml) and extracted with ethyl acetate (100 mL×3). The organic phases were combined, washed with brine, dried over anhydrous sodium sulfate, concentrated, and purified by column chromatography to obtain compound 34a-2 (700 mg, 6.4% yield).

MS (ESI), m/z, 300.2, 302.2 [M+H]⁺.

### Synthesis of Compound 34a-3

To a solution of 34a-2 (0.7 g, 2.3 mmol, 1.0 eq) in THF (100 mL) was added i-PrMgCl (2 M, 3 mL, 4 mmol, 2.0 eq) and 2b (600 mg, 4 mmol, 1.5 eq) at −25° C. under nitrogen atmosphere. The reaction solution was gradually warmed to room temperature and stirred for 5 h. After the reaction was completed, the reaction solution was quenched with ammonium chloride aqueous solution and extracted with ethyl acetate (50 mL×3). The organic phases were combined, washed with brine, dried over anhydrous sodium sulfate, filtered and concentrated, and purified by Prep-TLC to obtain 34a-3 (45 mg, purity 80%, yield 4.5%). MS (ESI), m/z, 427.2 [M+H]⁺.

### Synthesis of Compound 34a-4

To a solution of 34a-3 (42 mg, 0.098 mmol) in THF/MeOH (10/10 mL) was added hydrochloric acid (6M, 1 mL) at room temperature 25° C. under nitrogen atmosphere and stirred overnight. After the reaction was completed, the mixture was concentrated and purified by Prep-HPLC (CH₃CN/H₂O, 0.05% TFA) to obtain the target product 34a-4 (20 mg, purity 80%, yield 45%).

MS (ESI), m/z, 385.2 [M+H]⁺.

### Synthesis of Compound 34a-5

To a solution of 34a-4 (20 mg, 0.05 mmol) in toluene (10 mL) was added ketone (CAS is 110351-94-5) (10 mg, 0.05 mmol) and p-toluenesulfonic acid monohydrate (10 mg, 1.0 mmol) under nitrogen atmosphere. The reaction solution was heated to 120° C. and stirred for 5 h. The reaction solution was cooled to room temperature, concentrated and purified by column chromatography to obtain product 34a-5 (10 mg, 32% yield).

MS (ESI), m/z, 612.2 [M+H]⁺.

### Synthesis of Example 34a

To a suspension solution of 34a-5 (10 mg, 0.16 mmol) in MeOH/THF (2/2 mL) was added 10% Pd/C (3 mg) at room temperature 25° C., backfilled with H₂ (3×), and stirred overnight under H₂ atmosphere (H₂ balloon). After the reaction was completed, the reaction was filtered with celite, concentrated, and purified by Prep-HPLC (CH₃CN/H₂O, 0.05% TFA) to obtain product 34a (2.3 mg).

MS (ESI), m/z, 478.2 [M+H]⁺.

### Example 34b

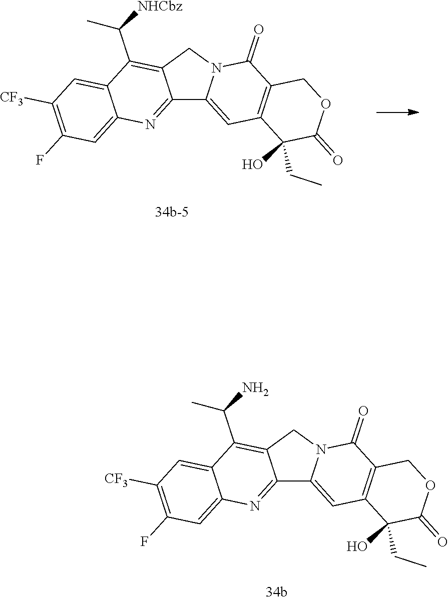 [SMILES: CC[C@@]1(O)C(=O)OCc2c1cc1n(c2=O)Cc2c-1nc1cc(F)c(C(F)(F)F)cc1c2[C@@H](C)NC(=O)OCc1ccccc1 ; CC[C@@]1(O)C(=O)OCc2c1cc1n(c2=O)Cc2c-1nc1cc(F)c(C(F)(F)F)cc1c2[C@@H](C)N]

### Synthesis of Compounds 34b-1˜5

According to the synthesis method of compounds 34a-1˜5.

### Synthesis of Example 34b

To a suspension solution of 34b-5 (30 mg, 0.05 mmol) in MeOH/THF (2/2 mL) was added 10% Pd/C (10 mg) at room temperature 25° C., backfilled with H₂ (3×), and stirred for 5 h under H₂ atmosphere (H₂ balloon). After the reaction was completed, the reaction was filtered with celite, concentrated, and purified by Prep-HPLC (CH₃CN/H₂O, 0.05% TFA) to obtain product 34b (9.3 mg).

MS (ESI), m/z, 478.2 [M+H]⁺.

### Example 35a

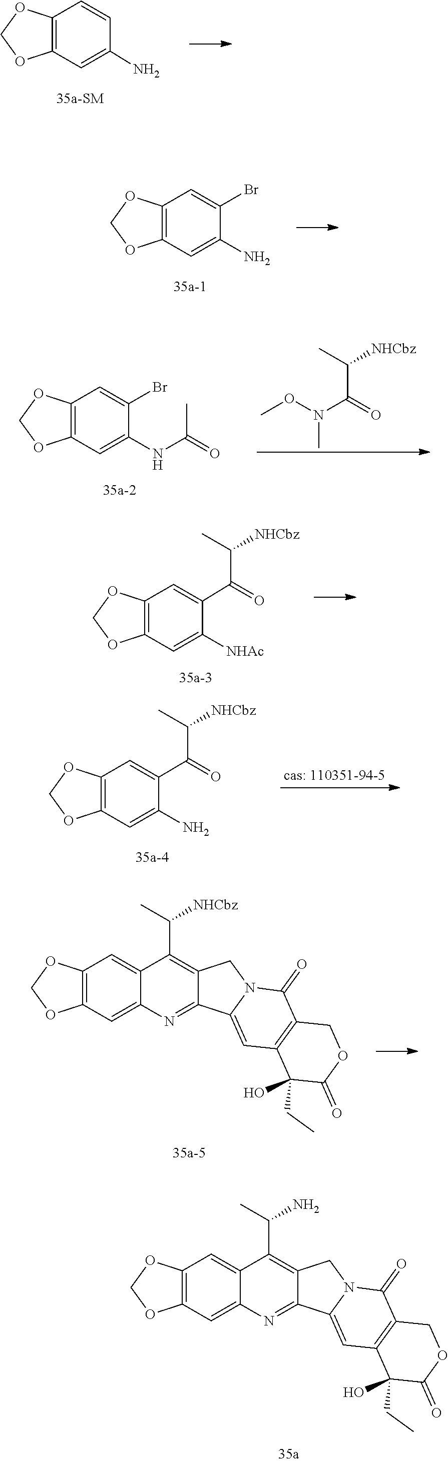

### Synthesis of Compound 35a-1

To a solution of 3,4-methylenedioxyaniline (4.5 g, 32.8 mmol, 1.0 eq) in DMF (50 mL) (50 mL) was added NBS (7.0 g, 39.7 mmol) at room temperature 25° C. under nitrogen atmosphere, and then stirred overnight. After the reaction was completed, the reaction solution was quenched with water (200 ml) and extracted with ethyl acetate (100 mL×3). The organic phases were combined, washed with brine, dried over anhydrous sodium sulfate, concentrated, and purified by column chromatography to obtain compound 35a-1 (1.1 g, 15.7% yield). MS (ESI), m/z, 216.2 [M+H]⁺.

### Synthesis of Compound 35a-2

To a solution of 35a-1 (1 g, 4.6 mmol, 1.0 eq) in CH₂Cl₂ (150 mL) was added triethylamine (0.6 g, 6 mmol) and acetyl chloride (0.45 g, 6 mmol) at room temperature 25° C. under nitrogen atmosphere, and then stirred for 3 h. After the reaction was completed, the reaction solution was quenched with water (100 ml) and extracted with dichloromethane (100 mL×3). The organic phases were combined, washed with brine, dried over anhydrous sodium sulfate, concentrated, and purified by column chromatography to obtain compound 35a-2 (1.1 g, 87% yield).

MS (ESI), m/z, 257.2 [M+H]⁺.

### Synthesis of Compound 35a-3

To a solution of 35a-2 (1.0 g, 3.8 mmol, 1.0 eq) in THF (10 0 mL) was added i-PrMgCl (2 M, 3.8 mL, 9.6 mmol, 2.0 eq) and 2b (1.5 g, 5.7 mmol, 1.5 eq) at −25° C. under nitrogen atmosphere. The reaction solution was gradually warmed to room temperature and stirred for 12 h. After the reaction was completed, the reaction solution was quenched with ammonium chloride aqueous solution, and extracted with ethyl acetate (50 mL×3). The organic phases were combined, washed with brine, dried over anhydrous sodium sulfate, filtered, concentrated and purified by Prep-TLC to obtain 90 mg (yield 6%).

MS (ESI), m/z, 385.2 [M+H]⁺.

### Synthesis of Compound 35a-4

To a solution of 35a-3 (90 mg, 0.53 mmol) in THF/MeOH (5/5 mL) was added hydrochloric acid (6 M, 10 mL) at room temperature 25° C. under a nitrogen atmosphere and then stirred overnight. After the reaction was completed, the reaction solution was concentrated, neutralized with sodium bicarbonate, and extracted with ethyl acetate (20 ml×3). The organic phases were combined, washed with brine, dried over anhydrous sodium sulfate, filtered, concentrated and purified by Prep-TLC to obtain product 35a-4 as a light yellow liquid (40 mg, 51% yield).

MS (ESI), m/z, 343.2 [M+H]⁺.

### Synthesis of Compound 35a-5

To a solution of 35a-4 (40 mg, 0.12 mmol) in toluene (5 mL) was added ketone (CAS is 110351-94-5) (46 mg, 0.17 mmol) and p-toluenesulfonic acid monohydrate (7 mg, 0.02 mmol) under nitrogen atmosphere. The reaction solution was heated to 120° C. and stirred for 5 h. The reaction solution was cooled to room temperature, concentrated, and purified by Prep-HPLC (CH₃CN/H₂O, 0.05% TFA) to obtain product 35a-5 (50 mg, 75% yield).

MS (ESI), m/z, 570.1 [M+H]⁺.

### Synthesis of Example 35a

To a suspension solution of 35a-5 (50 mg, 0.09 mmol) in MeOH/THF (5/5 mL) was added 10% Pd/C (10 mg) at room temperature 25° C., backfilled with H₂ (3×), and stirred for 8 h under H₂ atmosphere (H₂ balloon). After the reaction was completed, the reaction was filtered with celite, concentrated, and purified by Prep-HPLC (CH₃CN/H₂O, 0.05% TFA) to obtain product 35a (8 mg).

MS (ESI), m/z, 436.2 [M+H]⁺.

### Example 35b

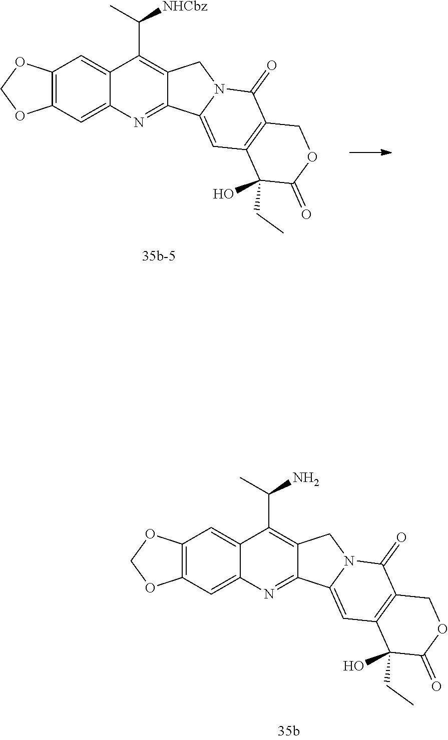 [SMILES: CC[C@@]1(O)C(=O)OCc2c1cc1n(c2=O)Cc2c-1nc1cc3c(cc1c2[C@@H](C)NC(=O)OCc1ccccc1)OCO3 ; CC[C@@]1(O)C(=O)OCc2c1cc1n(c2=O)Cc2c-1nc1cc3c(cc1c2[C@@H](C)N)OCO3]

### Synthesis of Compounds 35b-1˜5

According to the synthesis method of compounds 35a-1˜5.

### Synthesis of Example 35b

To a suspension solution of 35b-5 (40 mg, 0.07 mmol) in MeOH/THF (5/5 mL) was added 10% Pd/C (10 mg) at room temperature 25° C., backfilled with H₂ (3×), and stirred for 8 h under H₂ atmosphere (H₂ balloon). After the reaction was completed, the reaction was filtered with celite, concentrated, and purified by Prep-HPLC (CH₃CN/H₂O, 0.05% TFA) to obtain product 35b (11.6 mg).

MS (ESI), m/z, 436.2 [M+H]⁺.

### Example 36a

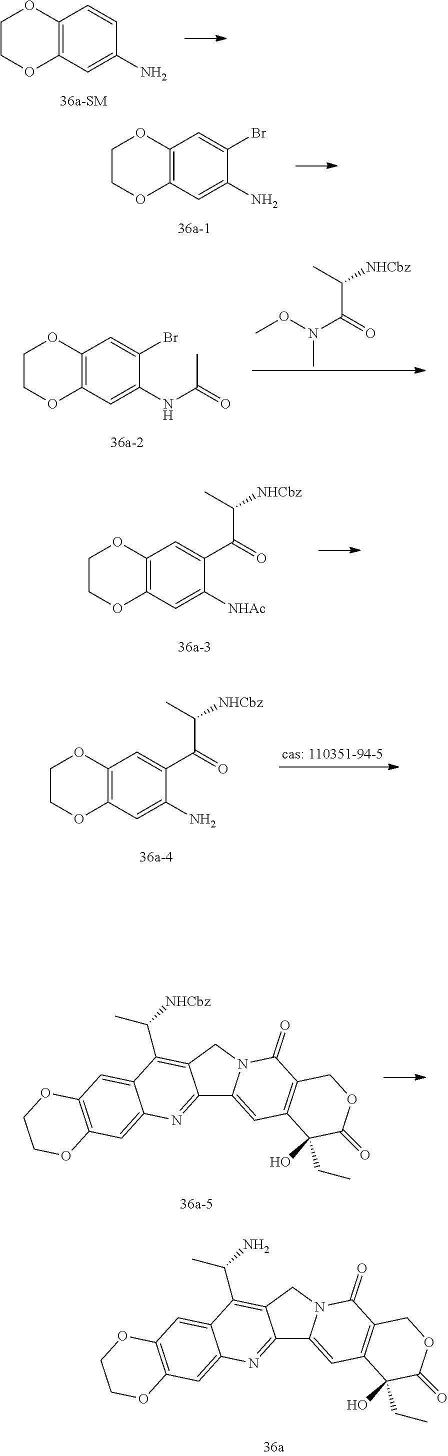

### Synthesis of Compound 36a-1

To a solution of 6-amino-1,4-benzodioxeterocycle (5 g, 33 mmol, 1.0 eq) in DMF (50 mL) was added NBS (7.0 g, 39.7 mmol) under nitrogen atmosphere at room temperature 25° C., and then stirred overnight. After the reaction was completed, the reaction was quenched with water (200 ml) and extracted with ethyl acetate (150 mL×3). The organic phases were combined, washed with brine, dried over anhydrous sodium sulfate, concentrated, and purified by column chromatography to obtain compound 36a-1 (2.3 g, 30.6% yield).

MS (ESI), m/z, 230.2 [M+H]⁺.

¹H NMR (400 MHZ, CDCl₃) δ 6.94 (s, 1H), 6.32 (s, 1H), 4.22-4.18 (m, 2H), 4.17-4.14 (m, 2H).

### Synthesis of Compound 36a-2

To a solution of 36a-1 (2.3 g, 10 mmol, 1.0 eq) in CH₂Cl₂ (150 mL) was added triethylamine (2.4 g, 24 mmol) and acetyl chloride (1.9 g, 24 mmol) under nitrogen atmosphere at room temperature 25° C., and then stirred for 2 h. After the reaction was completed, the reaction solution was quenched with water (100 ml) and extracted with dichloromethane (50 mL×3). The organic phases were combined, washed with brine, dried over anhydrous sodium sulfate, concentrated, and purified by column chromatography to obtain compound 36a-2 (2.6 g, 87% yield).

MS (ESI), m/z, 272.2 [M+H]⁺.

### Synthesis of Compound 36a-3

To a solution of 36a-2 (2.2 g, 8.1 mmol, 1.0 eq) in THF (100 mL) was added i-PrMgCl (2 M, 8 mL, 16 mmol, 2.0 eq) and 2b (2.1 g, 8 mmol, 1.5 eq) at −25° C. under nitrogen atmosphere. The reaction solution was gradually warmed to room temperature and stirred for 5 h. The reaction solution was quenched with ammonium chloride aqueous solution and extracted with ethyl acetate (50 mL×3). The organic phases were combined, washed with brine, dried over anhydrous sodium sulfate, filtered, concentrated and purified by Prep-TLC to obtain 80 mg (purity 60%, yield 1%).

MS (ESI), m/z, 399.2 [M+H]⁺.

### Synthesis of Compound 36a-4

To a solution of 36a-3 (80 mg, purity 60%, 0.53 mmol) in THF/MeOH (5/5 mL) was added hydrochloric acid (6 M, 10 mL) at room temperature 25° C. under nitrogen atmosphere and stirred overnight. After the reaction was completed, the reaction solution was concentrated, neutralized with sodium bicarbonate, and extracted with ethyl acetate (20 mL×3). The organic phases were combined, washed with brine, dried over anhydrous sodium sulfate, filtered, concentrated and purified by Prep-TLC to give product 36a-4 (180 mg, 53% yield).

MS (ESI), m/z, 335.2 [M+H]⁺.

### Synthesis of Compound 36a-5

To a solution of 36a-4 (180 mg, 0.53 mmol) in toluene (5 mL) was added ketone (CAS is 110351-94-5) (70 mg, 0.26 mmol) and p-toluenesulfonic acid monohydrate (50 mg, 4.12 mmol, 1.5 eq) under nitrogen atmosphere. The reaction solution was heated to 120° C. and stirred for 5 h. The reaction solution was cooled to room temperature, concentrated, and purified by Prep-TLC to obtain product 36a-5 (95 mg, 65% yield).

MS (ESI), m/z, 562.2 [M+H]⁺.

### Synthesis of Example 36a

To a suspension solution of 36b-5 (90 mg, 0.15 mmol) in MeOH/THF (5/5 mL) was added 10% Pd/C (10 mg) at room temperature 25° C., backfilled with H₂ (3×), and stirred for 8 h under H₂ atmosphere (H₂ balloon). After the reaction was completed, the reaction was filtered with celite, concentrated, and purified by Prep-HPLC (CH₃CN/H₂O, 0.05% TFA) to obtain product 36a (16 mg).

MS (ESI), m/z, 450.4 [M+H]⁺.

### Example 36b

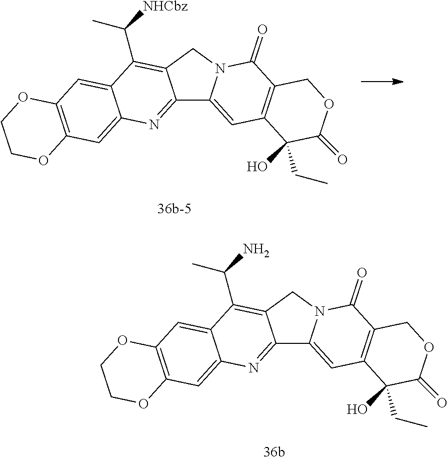 [SMILES: CC[C@@]1(O)C(=O)OCc2c1cc1n(c2=O)Cc2c-1nc1cc3c(cc1c2[C@@H](C)NC(=O)OCc1ccccc1)OCCO3 ; CC[C@@]1(O)C(=O)OCc2c1cc1n(c2=O)Cc2c-1nc1cc3c(cc1c2[C@@H](C)N)OCCO3]

### Synthesis of Compounds 36b-1˜5

According to the synthesis method of compounds 36a-1˜5.

### Synthesis of Example 36b

To a suspension solution of 36b-5 (120 mg, 0.21 mmol) in MeOH/THF (5/5 mL) was added 10% Pd/C (30 mg) at room temperature 25° C., backfilled with H₂ (3×), and stirred for 8 h under H₂ atmosphere (H₂ balloon). After the reaction was completed, the reaction was filtered with celite, concentrated, and purified by Prep-HPLC (CH₃CN/H₂O, 0.05% TFA) to obtain product 36b (32 mg).

MS (ESI), m/z, 450.4 [M+H]⁺.

### Example 37

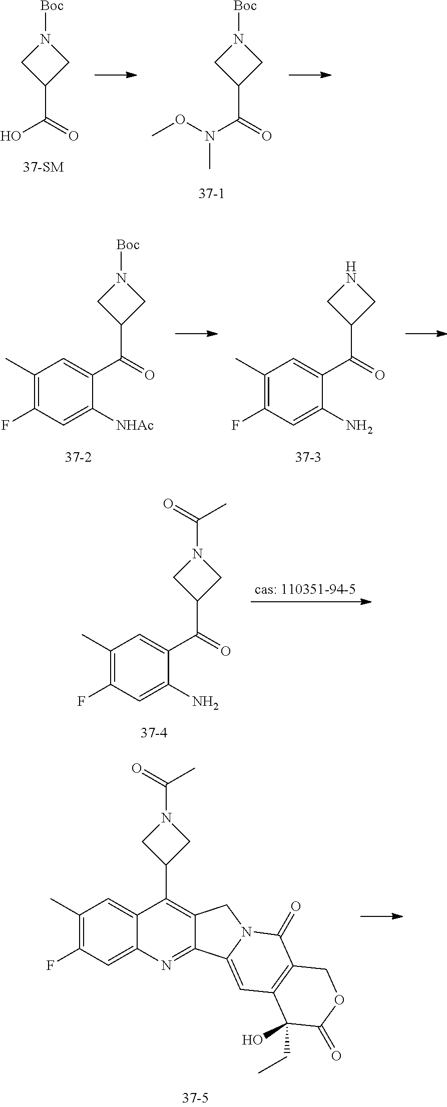 [SMILES: CC(C)(C)OC(=O)N1CC(C(=O)O)C1 ; CON(C)C(=O)C1CN(C(=O)OC(C)(C)C)C1 ; CC(=O)Nc1cc(F)c(C)cc1C(=O)C1CN(C(=O)OC(C)(C)C)C1 ; Cc1cc(C(=O)C2CNC2)c(N)cc1F ; CC(=O)N1CC(C(=O)c2cc(C)c(F)cc2N)C1 ; CC[C@@]1(O)C(=O)OCc2c1cc1n(c2=O)Cc2c-1nc1cc(F)c(C)cc1c2C1CN(C(C)=O)C1]

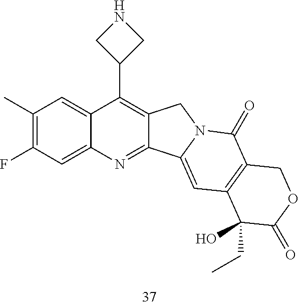 [SMILES: CC[C@@]1(O)C(=O)OCc2c1cc1n(c2=O)Cc2c-1nc1cc(F)c(C)cc1c2C1CNC1]

### Synthesis of Compound 37-1

To a solution of 1-N-Boc-3-azetidinecarboxylic acid (10 g, 49.7 mmol, 1.0 eq) in DMF (50 mL) was added N, O-Dimethylhydroxylamine hydrochloride (4.8 g, 79.7 mmol), DIPEA (15 mL) and EDCI (9.55 g, 50 mmol) at room temperature 25° C. under nitrogen atmosphere, and then stirred overnight. After the reaction was completed, the reaction solution was quenched with water (200 ml), and the organic phase was dried over anhydrous sodium sulfate and concentrated in vacuum to obtain a colorless liquid (9.0 g, 80.6% yield).

MS (ESI), m/z, 245.2 [M+H]⁺.

### Synthesis of Compound 37-2

To a solution of 2a (3.0 g, 12.2 mmol, 1.0 eq) in THF (100 mL) was added i-PrMgCl (2 M, 10 mL, 18 mmol, 2.0 eq) and 37-1 (3.1 g, 12 mmol, 1.0 eq) at −25° C. under nitrogen atmosphere. The reaction solution was gradually warmed to room temperature and stirred for 5 h. After the reaction was completed, the mixture was quenched with ammonium chloride aqueous solution and extracted with ethyl acetate (100 mL×3). The organic phases were combined, washed with brine, dried over anhydrous sodium sulfate, and purified by column chromatography to obtain compound 37-2 (100 mg, purity 70%, yield 3%).

MS (ESI), m/z, 351.2 [M+H]⁺.

### Synthesis of Compound 37-3

To a solution of compound 37-2 (120 mg, 0.53 mmol) in THF/MeOH (10/10 mL) was added hydrochloric acid (6 M, 10 mL) at 5° C. of ice-water bath under nitrogen atmosphere, and stirred for 1 h. After the reaction was completed, the reaction solution was directly concentrated to obtain a crude product, which was used directly in the next step.

MS (ESI), m/z, 209.2 [M+H]⁺.

### Synthesis of Compound 37-4

To the above crude product 37-3 (70 mg, 0.34 mmol, 1.0 eq) in THE solution (10 mL) was added dropwise acetic anhydride (38 mg, 0.37 mmol, 1.1 eq) in THE solution (1 mL) and triethylamine (68 mg, 0.67 mmol, 2.0 eq) at room temperature 25° C. under nitrogen atmosphere and stirred for 1 h. After the reaction was completed, the mixture was quenched with water and extracted with ethyl acetate (20 mL×3). The organic phases were combined, washed with brine, dried over anhydrous sodium sulfate, filtered, concentrated and purified by Prep-TLC to obtain compound 37-4 (30 mg).

MS (ESI), m/z, 251.2 [M+H]⁺.

### Synthesis of Compound 37-5

To a solution of 37-4 (30 mg, 0.12 mmol) in toluene (5 mL) was added ketone (CAS is 110351-94-5) (46 mg, 0.17 mmol) and p-toluenesulfonic acid monohydrate (7 mg, 0.02 mmol) under nitrogen atmosphere. The reaction solution was heated to 120° C. and stirred for 5 h. The reaction solution was cooled to room temperature, concentrated, and purified by Prep-HPLC (CH₃CN/H₂O, 0.05% TFA) to obtain product 37-5 (50 mg, 75% yield).

MS (ESI), m/z, 570.1 [M+H]⁺.

### Synthesis of Example 37

37-5 (50 mg, 0.088 mmol) was dissolved in 6 N aqueous hydrochloric acid (1.0 mL) at 25° C. The reaction solution was heated to 100-105° C. and stirred for 16 h. LCMS showed that the product was formed. After the reaction was completed, it was concentrated and purified by Prep-HPLC (CH₃CN/H₂O, 0.05% TFA) to obtain Example 37 (5 mg).

MS (ESI), m/z, 436.4 [M+H]⁺.

### Example 38a

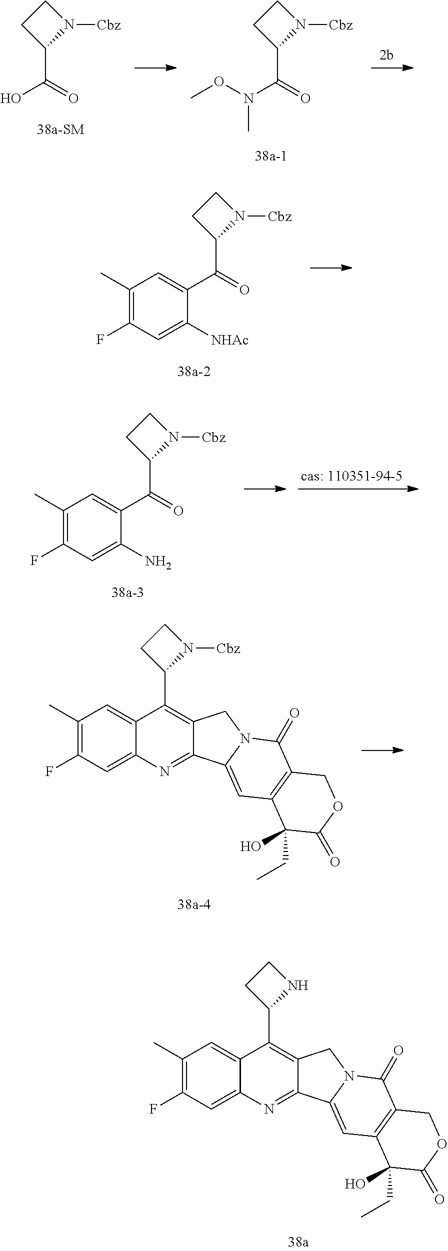 [SMILES: O=C(O)[C@@H]1CCN1C(=O)OCc1ccccc1 ; CON(C)C(=O)[C@@H]1CCN1C(=O)OCc1ccccc1 ; CC(=O)Nc1cc(F)c(C)cc1C(=O)[C@@H]1CCN1C(=O)OCc1ccccc1 ; Cc1cc(C(=O)[C@@H]2CCN2C(=O)OCc2ccccc2)c(N)cc1F ; CC[C@@]1(O)C(=O)OCc2c1cc1n(c2=O)Cc2c-1nc1cc(F)c(C)cc1c2[C@@H]1CCN1C(=O)OCc1ccccc1 ; CC[C@@]1(O)C(=O)OCc2c1cc1n(c2=O)Cc2c-1nc1cc(F)c(C)cc1c2[C@@H]1CCN1]

### Synthesis of Compound 38a-1

To a solution of N-CBZ-S-2-azetidine-1-carboxylic acid (1.0 g, 0.043 mol) in dichloromethane (20 mL) was added CDI (0.9 g, 0.055 mol, 1.3 eq) at 5° C. and stirred for 1 h, then N, O-Dimethylhydroxylamine hydrochloride (0.6 g, 0.064 mol, 1.5 eq) and triethylamine (0.8 g, 0.085 mol, 2.0 eq) were added to the above mixture and continued to stir for 1 h. The reaction solution was quenched with water, and the aqueous phase was extracted with dichloromethane (50 mL×3). The organic phases were combined, washed with 1 N HCl, saturated sodium bicarbonate, brine, dried over anhydrous sodium sulfate, filtered and concentrated to obtain crude product 38a-1 (1 g, yield 85%).

MS (ESI), m/z, 278.90 [M+H]⁺.

### Synthesis of Compound 38a-2

To a solution of 2a (0.6 g, 2.44 mmol) in dry tetrahydrofuran (30 mL) was added dropwise n-butyllithium solution (2.5 M in hexane, 2.2 mL, 5.37 mmol, 2.2 eq) at −60° C., and stirred for 1 h. A solution of 38a-1 (1.0 g, 3.66 mmol, 1.5 eq) in tetrahydrofuran solution (5 mL) was added dropwise to the above solution and stirred for 1 h (−60-−20° C.). The reaction solution was quenched with aqueous ammonium chloride solution, and the aqueous phase was extracted with ethyl acetate (50 mL×3). The organic phases were combined, washed with brine, dried over anhydrous sodium sulfate, filtered, concentrated, and purified by column chromatography to obtain compound 38a-2 (100 mg).

MS (ESI), m/z, 385.0 [M+H]⁺.

### Synthesis of Compound 38a-3

To a solution of 38a-2 (100 mg, 0.26 mmol) in methanol and tetrahydrofuran (5/5 mL) was added 3M hydrochloric acid (5 mL) at 5° C. in an ice-water bath. The reaction solution was then warmed to room temperature 25° C. and stirred for 6 h, diluted with water and extracted with ethyl acetate (20 mL×3). The organic phases were combined, washed with brine, dried over anhydrous sodium sulfate, filtered, and purified by Prep-HPLC (CH₃CN/H₂O, 0.05% TFA) to obtain compound 38a-3 (60 mg).

MS (ESI), m/z, 343.1 [M+H]⁺.

### Synthesis of Compound 38a-4

To a solution of 38a-3 (60 mg, 0.17 mmol, 1.0 eq) in toluene (5 mL) was added ketone (CAS is 110351-94-5) (55 mg, 0.21 mmol, 1.2 eq) and p-toluenesulfonic acid monohydrate (17 mg, 0.09 mmol, 0.5 eq). The reaction was heated to 110° C. and stirred for 3 h. After the reaction was completed, the mixture was concentrated under vacuum and purified directly by column chromatography to obtain compound 38a-4 (50 mg).

MS (ESI), m/z, 570.2 [M+H]⁺.

### Synthesis of Example 38a

To a mixed suspension solution of 38a-4 (50 mg, 0.09 mmol) in ethyl acetate (5 mL) and methanol (2 mL) was added 10% Pd/C (55% wet, 10 mg), and backfilled with hydrogen. The mixture was stirred for 1 h under hydrogen atmosphere at 25° C. The reaction solution was filtered with celite, and the crude product was purified by Prep-HPLC (CH₃CN/H₂O, 0.05% TFA) to obtain Example 38a (8.5 mg).

MS (ESI), m/z, 436.0 [M+1]⁺.

### Example 38b

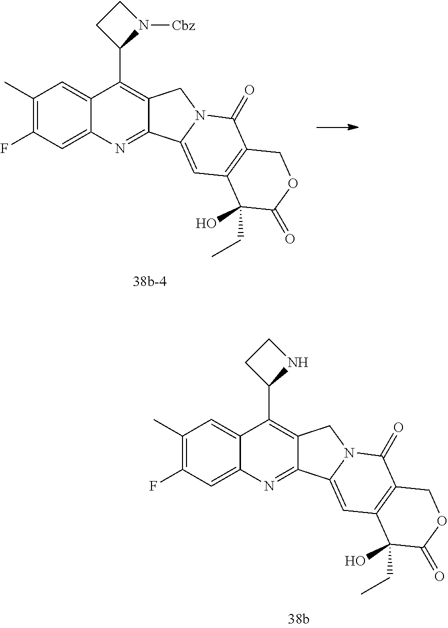 [SMILES: CC[C@@]1(O)C(=O)OCc2c1cc1n(c2=O)Cc2c-1nc1cc(F)c(C)cc1c2[C@H]1CCN1C(=O)OCc1ccccc1 ; CC[C@@]1(O)C(=O)OCc2c1cc1n(c2=O)Cc2c-1nc1cc(F)c(C)cc1c2[C@H]1CCN1]

### Synthesis of Compounds 38b-1˜4

According to the synthesis method of compounds 38a-1˜4.

### Synthesis of Example 38b

To a suspension solution of 38b-5 (100 mg, 0.2 mmol) in EtOAc/MeOH (5/2 mL) was added 10% Pd/C (55% wet, 10 mg) at room temperature 25° C., backfilled with H₂ (3×), and stirred for 1 h under H₂ atmosphere. The reaction was filtered with celite, concentrated, and purified by Prep-HPLC (CH₃CN/H₂O, 0.05% TFA) to obtain product 38b (21 mg).

MS (ESI), m/z, 436.0 [M+1]⁺.

### Example 39a

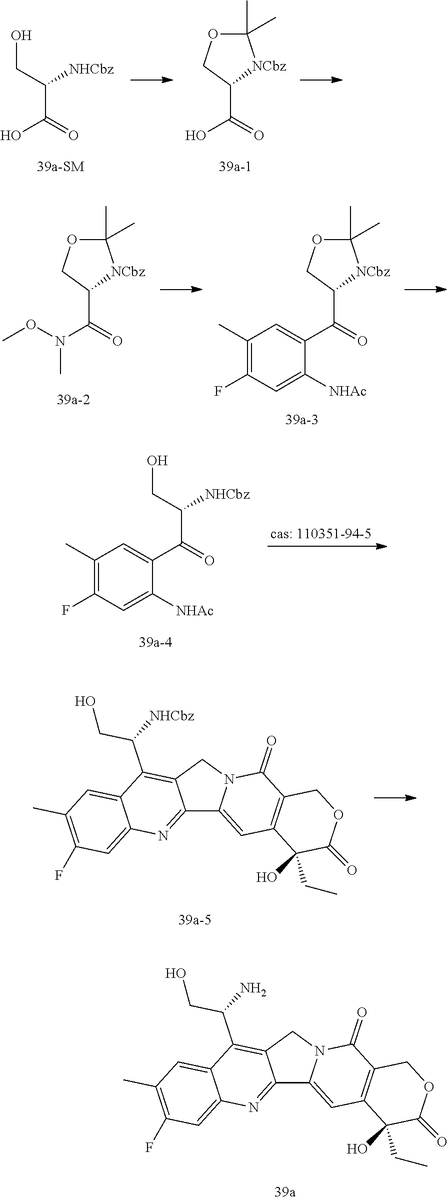

### Synthesis of Compound 39a-1

To a solution of N-benzyloxycarbonyl-L-serine (10 g, 41.8 mmol, 1.0 eq) in toluene (50 mL) was added 2, 2-methoxypropane (5.2 g, 50 mmol) and a catalytic amount of p-toluenesulfonic acid monohydrate at room temperature of 25° C. under nitrogen atmosphere and stirred overnight. After the reaction was completed, the reaction solution was directly concentrated to obtain crude product 39a-1 (8.5 g, 85.6% yield).

MS (ESI), m/z, 280.2 [M+H]⁺.

### Synthesis of Compound 39a-2

To a solution of 39a-1 (8.5 g, 30 mmol) in DMF (50 mL) was added dimethylhydroxylamine hydrochloride (2.1 g, 30 mmol), DIPEA (15 mL) and EDCI (8.4 g, 30 mmol) at 25° C. under nitrogen atmosphere and stirred overnight. After the reaction was completed, the reaction solution was quenched with water (200 ml) and extracted with ethyl acetate (150 mL×3). The organic phases were combined, washed with brine, dried over anhydrous sodium sulfate, and concentrated to obtain product 39a-2 as a colorless oil (9.5 g, 84.6% yield).

MS (ESI), m/z, 323.2 [M+H]⁺.

### Synthesis of Compound 39a-3

To a solution of 2a (3.0 g, 9.2 mmol, 1.0 eq) in THF (100 mL) was added i-PrMgCl (2 M, 8 mL, 16 mmol, 2.0 eq) and 39a-2 (2.6 g, 9.2 mmol, 1.5 eq) at −25° C. under nitrogen atmosphere. The reaction solution was slowly warmed to room temperature and stirred for 5 h. After the reaction was completed, the mixture was quenched with aqueous ammonium chloride solution and extracted with ethyl acetate (50 mL×3). The organic phases were combined, washed with brine, dried over anhydrous sodium sulfate, and purified by column chromatography to obtain 110 mg, purity 60%.

MS (ESI), m/z, 429.2 [M+H]⁺.

### Synthesis of Compound 39a-4

To a solution of 39a-3 (110 mg, 0.53 mmol) in THE/MeOH (5/5 mL) was added hydrochloric acid (6 M, 5 mL) at 25° C. under nitrogen atmosphere, and stirred overnight. After the reaction was completed, the mixture was concentrated and purified by Prep-HPLC (CH₃CN/H₂O, 0.05% TFA) to obtain compound 39a-4 (30 mg).

MS (ESI), m/z, 347.2 [M+H]⁺.

### Synthesis of Compound 39a-5

To a solution of 39a-4 (30 mg, 0.09 mmol) in toluene (2 mL) was added ketone (CAS is 110351-94-5) (27 mg, 0.10 mmol) and p-toluenesulfonic acid monohydrate (8 mg, 0.04 mmol, 0.5 eq) under nitrogen atmosphere. The reaction solution was heated to 120° C. and stirred for 5 h. The reaction solution was cooled to room temperature, concentrated, and purified by column chromatography to obtain compound 39a-5 (25 mg).

MS (ESI), m/z, 574.2 [M+H]⁺.

### Synthesis of Example 39a

To a suspension solution of 39a-5 (25 mg, 0.04 mmol) in MeOH/THF (5/5 mL) was added 10% Pd/C (10 mg) at room temperature 25° C., backfilled with H₂ (3×), and stirred for 1 h under H₂ atmosphere. After the reaction was completed, the mixture was filtered with celite, concentrated, and purified by Prep-HPLC (CH₃CN/H₂O, 0.05% TFA) to obtain product 39a (3.5 mg).

MS (ESI), m/z, 440.4 [M+H]⁺.

### Example 39b

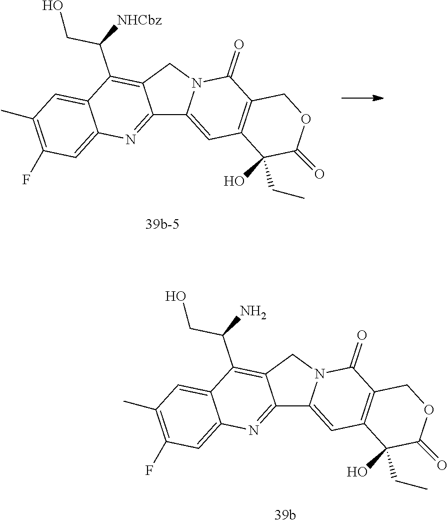 [SMILES: CC[C@@]1(O)C(=O)OCc2c1cc1n(c2=O)Cc2c-1nc1cc(F)c(C)cc1c2[C@@H](CO)NC(=O)OCc1ccccc1 ; CC[C@@]1(O)C(=O)OCc2c1cc1n(c2=O)Cc2c-1nc1cc(F)c(C)cc1c2[C@H](N)CO]

### Synthesis of Compounds 39b-1˜5

According to the synthesis method of compounds 39a-1˜5.

### Synthesis of Example 39b

To a suspension solution of 39b-5 (50 mg, 0.08 mmol) in MeOH/THF (5/5 mL) was added 10% Pd/C (55% wet, 10 mg) at room temperature 25° C., backfilled with H₂ (3×), and stirred for 1 h under H₂ atmosphere (H₂ ballnoon). The reaction was filtered with celite, concentrated, and purified by Prep-HPLC (CH₃CN/H₂O, 0.05% TFA) to obtain product 39b (6.9 mg).

MS (ESI), m/z, 440.4 [M+H]⁺.

### Example 40a

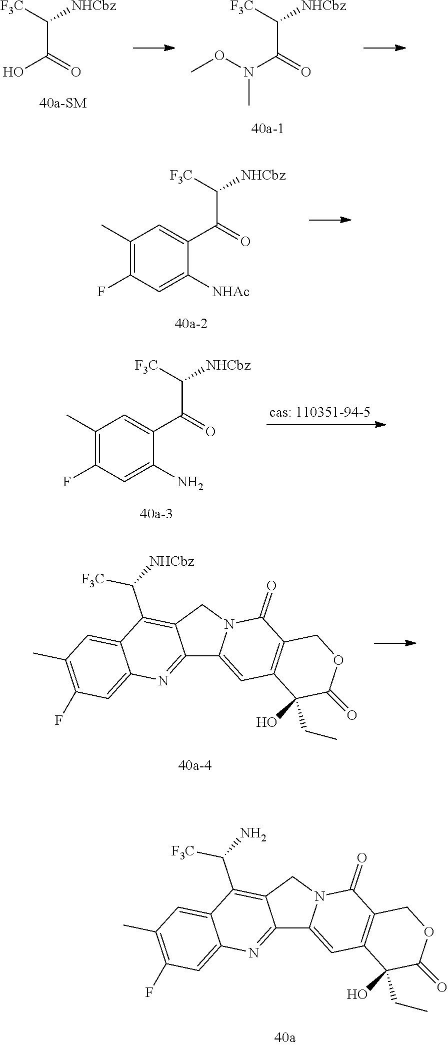 [SMILES: O=C(N[C@H](C(=O)O)C(F)(F)F)OCc1ccccc1 ; CON(C)C(=O)[C@@H](NC(=O)OCc1ccccc1)C(F)(F)F ; CC(=O)Nc1cc(F)c(C)cc1C(=O)[C@@H](NC(=O)OCc1ccccc1)C(F)(F)F ; Cc1cc(C(=O)[C@@H](NC(=O)OCc2ccccc2)C(F)(F)F)c(N)cc1F ; CC[C@@]1(O)C(=O)OCc2c1cc1n(c2=O)Cc2c-1nc1cc(F)c(C)cc1c2[C@@H](NC(=O)OCc1ccccc1)C(F)(F)F ; CC[C@@]1(O)C(=O)OCc2c1cc1n(c2=O)Cc2c-1nc1cc(F)c(C)cc1c2[C@@H](N)C(F)(F)F]

### Synthesis of Compound 40a-1

To a solution of 40a-SM (synthesized according to Org. Lett. 2001, 3, 17, 2621-2624.) (2.0 g, 7.2 mmol) in dichloromethane (40 mL) was added CDI (1.7 g, 10.8 mmol, 1.5 eq) at 5° C. and stirred for 1 h, then N, O-Dimethylhydroxylamine hydrochloride (1.4 g, 14.4 mmol, 2.0 eq) and triethylamine (1.5 g, 14.4 mmol, 2.0 eq) were added to the above mixture and stirred for 1 h. The reaction solution was quenched with water, and the aqueous phase was extracted with dichloromethane (100 mL×3). The organic phases were combined, washed with 1 N HCl, saturated sodium bicarbonate, brine, dried over anhydrous sodium sulfate, filtered and concentrated to obtain crude product 40a-1 (2.0 g, yield 87%).

MS (ESI), m/z, 321.10 [M+H]⁺.

### Synthesis of Compound 40a-2

To a solution of 2a (1.0 g, 0.041 mol) in dry tetrahydrofuran (20 mL) was added dropwise n-butyllithium solution (2.5 M in hexane, 3.6 mL, 0.09 mol) at −60° C., and stirred for 1 h. A solution of 40a-1 (1.9 g, 0.061 mol) in tetrahydrofuran solution (10 mL) was added dropwise to the above solution, and stirred for 2 h (−60-−20° C.). The reaction solution was quenched with aqueous ammonium chloride solution, and the aqueous phase was extracted with ethyl acetate (30 mL×3). The organic phases were combined, washed with brine, dried over anhydrous sodium sulfate, filtered, concentrated, and purified by column chromatography to obtain compound 40a-2 (0.2 g).

MS (ESI), m/z, 427.2 [M+H]⁺.

### Synthesis of Compound 40a-3

To a solution of 40a-2 (180 mg, 0.42 mmol) in methanol and tetrahydrofuran (3/5 mL) was added concentrated hydrochloric acid (3 mL) and stirred for 12 h. The reaction solution was concentrated, basified with sodium bicarbonate to pH>7, diluted with water and extracted with dichloromethane (50 mL×3). The organic phases were combined, washed with brine, dried over anhydrous sodium sulfate, filtered, and purified by column chromatography to obtain compound 40a-3 (110 mg).

MS (ESI), m/z, 385.1 [M+H]⁺.

### Synthesis of Compound 40a-4

To a solution of 40a-3 (100 mg, 0.26 mmol) in toluene (5 mL) was added ketone (CAS is 110351-94-5) (82 mg, 0.31 mmol) and p-toluenesulfonic acid monohydrate (25 mg, 0.13 mmol). The reaction solution was heated to 110° C. and stirred for 3 h. After the reaction was completed, the reaction solution was concentrated, filtered by adding water, and the solid was purified by column chromatography to obtain compound 40a-4 (85 mg).

MS (ESI), m/z, 612.2 [M+H]⁺.

### Synthesis of Example 40a

To a solution of 40a-4 (80 mg, 0.13 mmol) in methanol and ethyl acetate solution (5 mL/10 mL) was added 10% Pd/C (water content 55%, 30 mg) at 25° C., and backfilled with nitrogen, and then backfilled with hydrogen, and stirred for 2 h under H₂ atmosphere. The reaction solution was filtered with celite, washed with dichloromethane/methanol, and concentrated. The residue was purified by Prep-HPLC (CH₃CN/H₂O, 0.05% TFA) to obtain Example 40a (30 mg).

MS (ESI), m/z, 478.4 [M+H]⁺.

### Example 40b

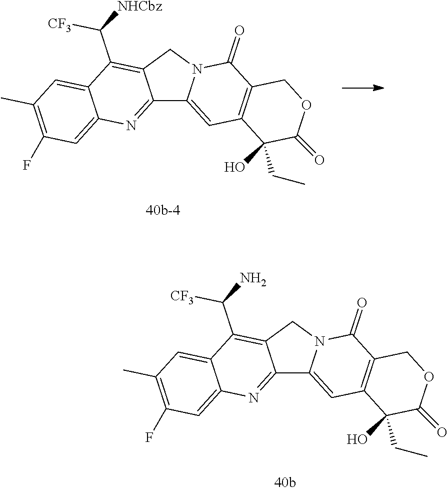 [SMILES: CC[C@@]1(O)C(=O)OCc2c1cc1n(c2=O)Cc2c-1nc1cc(F)c(C)cc1c2[C@H](NC(=O)OCc1ccccc1)C(F)(F)F ; CC[C@@]1(O)C(=O)OCc2c1cc1n(c2=O)Cc2c-1nc1cc(F)c(C)cc1c2[C@H](N)C(F)(F)F]

### Synthesis of Compounds 40b-1˜4

According to the synthesis method of compounds 40a-1˜4.

### Synthesis of Example 40b

To a solution of 40b-4 (40 mg, 0.07 mmol) in MeOH/EtOAc (5/10 mL) was added 10% Pd/C (55% wet, 10 mg) at room temperature 25° C., backfilled with N₂, and then backfilled with H₂ (3×), and stirred for 1 h under H₂ atmosphere. The reaction was filtered with celite, concentrated, and purified by Prep-HPLC (CH₃CN/H₂O, 0.05% TFA) to obtain product 40b (5.8 mg).

MS (ESI), m/z, 478.4 [M+H]⁺.

### Examples 41a and 41b

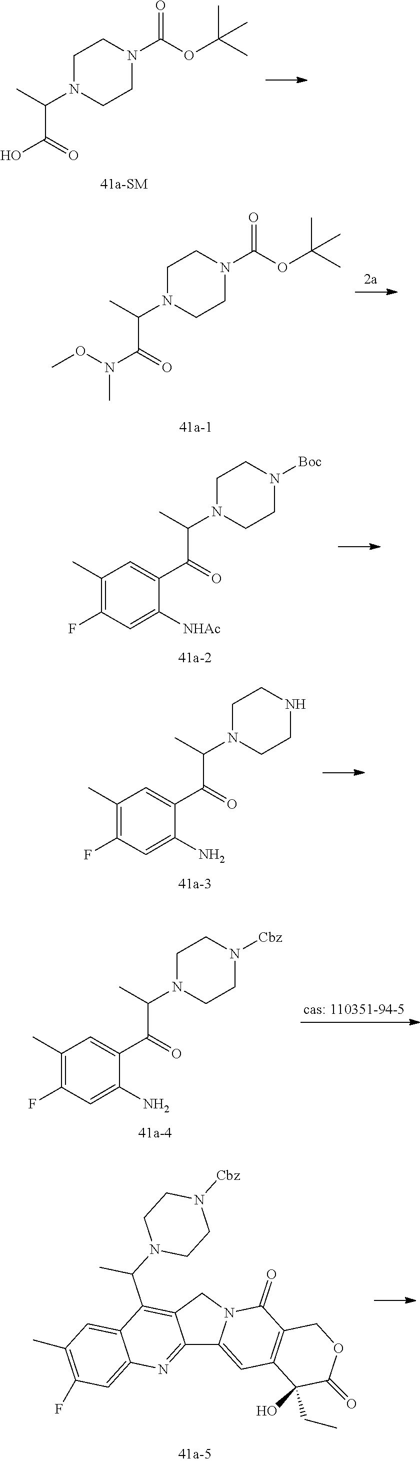

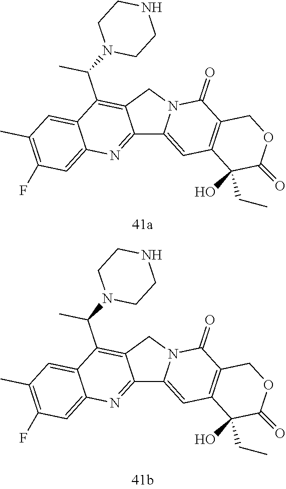 [SMILES: CC[C@@]1(O)C(=O)OCc2c1cc1n(c2=O)Cc2c-1nc1cc(F)c(C)cc1c2[C@H](C)N1CCNCC1 ; CC[C@@]1(O)C(=O)OCc2c1cc1n(c2=O)Cc2c-1nc1cc(F)c(C)cc1c2[C@@H](C)N1CCNCC1]

### Synthesis of Compound 41a-1

To a solution (40 mL) of 41a-SM (synthesized according to patent WO2018134213A1) (2.0 g, 7.8 mmol) in dichloromethane (40 mL) was added CDI (3.7 g, 11.6 mmol, 1.5 eq) and stirred for 2 h. Then dimethylhydroxylamine hydrochloride (1.5 g, 15.5 mmol, 2.0 eq) and triethylamine (1.6 g, 15.5 mmol, 2.0 eq) were added to the reaction solution and stirring for 2 h. The reaction solution was quenched with water, and the aqueous phase was extracted with dichloromethane (100 mL×3). The organic phases were combined, washed with 1 N HCl, saturated sodium bicarbonate, brine, dried over anhydrous sodium sulfate, filtered and concentrated to obtain crude product 41a-1 (2.1 g, yield 90%).

MS (ESI), m/z, 302.10 [M+H]⁺.

### Synthesis of Compound 41a-2

To a solution of 2a (1.0 g, 3.8 mmol) in dry tetrahydrofuran (20 mL) was added dropwise n-butyllithium solution (2.5 M in hexane, 3.4 mL, 8.4 mmol) at −60° C. and stirred for 1 h. A solution of 41a-1 (1.7 g, 5.7 mmol) in tetrahydrofuran (10 mL) was then added dropwise to the above solution and stirred for 12 h (−60-25° C.). The reaction solution was quenched with aqueous ammonium chloride solution, and the aqueous phase was extracted with ethyl acetate (50 mL×3). The organic phases were combined, washed with brine, dried over anhydrous sodium sulfate, filtered, concentrated, and purified by column chromatography to obtain compound 41a-2 (0.1 g).

MS (ESI), m/z, 408.2 [M+H]⁺.

### Synthesis of Compound 41a-3

To a solution of compound 41a-2 (100 mg, 0.25 mmol) in THE/MeOH (5/5 mL) was added hydrochloric acid (6 M, 5 mL) at room temperature 25° C. under nitrogen atmosphere and stirred for 12 h. After the reaction was completed, the mixture was concentrated to give crude product, which was used directly in the next step.

MS (ESI), m/z, 266.1 [M+H]⁺.

### Synthesis of Compound 41a-4

To the solution of the above crude product 41a-3 in THF (10 mL) was added a solution of benzyl chloroformate (50 mg, 0.29 mmol, 1.2 eq) in THF (1 mL) and triethylamine (50 mg, 0.50 mmol, 2.0 eq) under nitrogen atmosphere at room temperature 25° C. and stirred for 1 h. After the reaction was completed, the reaction was quenched with water, extracted with ethyl acetate, dried, concentrated, and purified by Prep-TLC to obtain compound 41a-4 (42 mg).

MS (ESI), m/z, 400.2 [M+H]⁺.

### Synthesis of Compound 41a-5

To a solution of 41a-4 (40 mg, 0.1 mmol) in toluene (5 mL) was added ketone (CAS is 110351-94-5) (32 mg, 0.12 mmol) and p-toluenesulfonic acid monohydrate (4 mg, 0.02 mmol) under nitrogen atmosphere. The reaction solution was heated to 120° C. and stirred for 12 h. The reaction solution was cooled to room temperature, concentrated, and purified by column chromatography to obtain product 41a-5 as a light yellow solid (35 mg).

MS (ESI), m/z, 627.2 [M+H]⁺.

### Synthesis of Examples 41a and 41b

To a solution of 41a-5 (35 mg, 0.06 mmol) in 3 M hydrochloric acid/methanol/ethyl acetate (1/5/10 mL) was added 10% Pd/C (Wet 55%, 10 mg) at room temperature 25°, backfilled with nitrogen, and then backfilled with hydrogen, and stirred for 2 h under H₂ atmosphere. The reaction solution was filtered with celite, washed with dichloromethane/methanol, and concentrated. A portion of the residue was purified by Prep-HPLC to obtain Example 41a and 41b (2.5 mg and 1.7 mg respectively).

MS (ESI), m/z, 492.1 [M+H]⁺

### Examples 42a and 42b

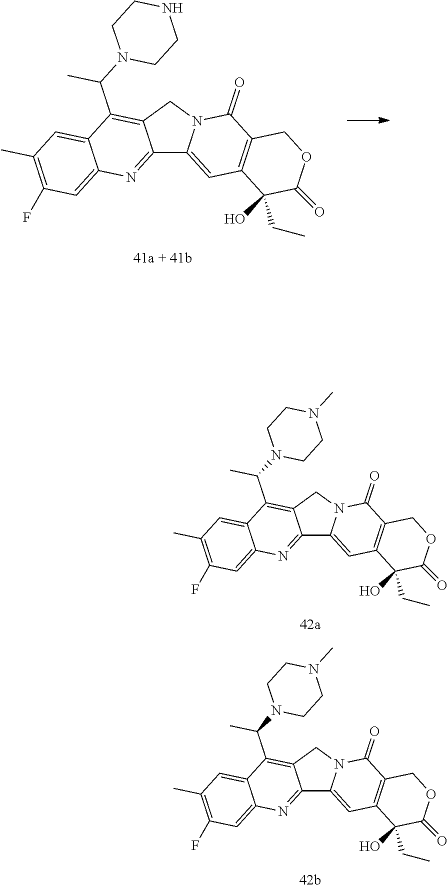 [SMILES: CC[C@@]1(O)C(=O)OCc2c1cc1n(c2=O)Cc2c-1nc1cc(F)c(C)cc1c2C(C)N1CCNCC1 ; CC[C@@]1(O)C(=O)OCc2c1cc1n(c2=O)Cc2c-1nc1cc(F)c(C)cc1c2[C@H](C)N1CCN(C)CC1 ; CC[C@@]1(O)C(=O)OCc2c1cc1n(c2=O)Cc2c-1nc1cc(F)c(C)cc1c2[C@@H](C)N1CCN(C)CC1]

To a solution (10 mL) of the crude mixture of 41a and 41b (15 mg) in methylene chloride was added one drop of triethylamine and one drop of 30% formaldehyde solution at 0-5° C. under nitrogen atmosphere. The reaction solution was stirred for 10 minutes, then sodium cyanoborohydride (5 mg) was added, the temperature was slowly warmed to room temperature, and the reaction was stirred for 16 h. LCMS showed that the product was formed. The reaction solution was concentrated and purified by Prep-HPLC (CH₃CN/H₂O, 0.05% TFA) to obtain Examples 42a and 42b, 2.5 mg and 1.6 mg respectively.

MS (ESI), m/z, 507.1 [M+H]⁺.

### Synthesis of Example 43a

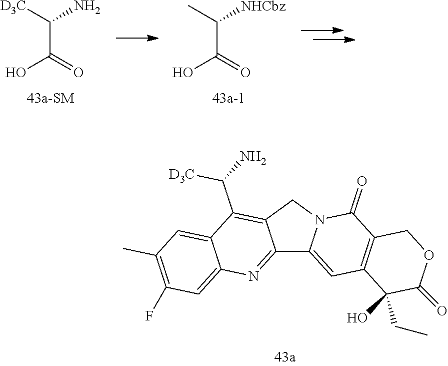 [SMILES: [2H]C([2H])([2H])[C@H](N)C(=O)O ; C[C@H](NC(=O)OCc1ccccc1)C(=O)O ; [2H]C([2H])([2H])[C@H](N)c1c2c(nc3cc(F)c(C)cc13)-c1cc3c(c(=O)n1C2)COC(=O)[C@]3(O)CC]

### Synthesis of Compound 43a-1

To a solution of L-alanine-3,3,3-D3 (1.0 g, 10.9 mmol, 1.0 eq) in sodium hydroxide (2N, 10 mL) was added benzyl chloroformate (1.8 g, 10.9 mmol, 1.0 eq) at 5° C. in an ice-water bath under nitrogen protection. After addition, the mixture was slowly warmed to room temperature and stirred for 3 h. The reaction solution was washed with MTBE (50 mL), the aqueous phase was adjusted to pH=4 with 2N dilute hydrochloric acid and extracted with ethyl acetate (100 mL×3). The organic phases were combined, washed with brine, dried over anhydrous sodium sulfate, filtered and concentrated to obtain compound 43a-1 as a white solid (2.0 g, yield 83%).

MS (ESI), m/z, 224.0 [M+H]⁺.

### Synthesis of Example 43a

Based on compound 43a-1 as the starting material, according to the synthesis method of Example 2 (synthesis method II), the Example 43a (36 mg) was finally obtained.

MS (ESI), m/z, 427.1 [M+H]⁺.

### Synthesis of Example 43b

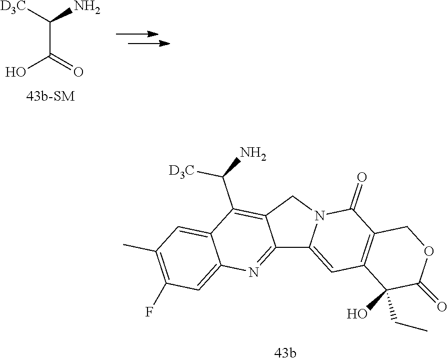 [SMILES: [2H]C([2H])([2H])[C@@H](N)C(=O)O ; [2H]C([2H])([2H])[C@@H](N)c1c2c(nc3cc(F)c(C)cc13)-c1cc3c(c(=O)n1C2)COC(=O)[C@]3(O)CC]

### Synthesis of Example 43b

Based on the commercially available compound 43b-SM as the starting material, according to the synthesis methods of Example 1 (synthesis method II) and Example 43a, Example 43b was finally obtained (15.3 mg).

MS (ESI), m/z, 427.1 [M+H]⁺.

### Biological Evaluation Example

### Biological Example 1: MCF-7 Cell Proliferation Inhibitory Assay

### 1. Experimental Materials

1). Cell line: MCF-7 cells (human breast cancer cells), ATCC, Catalog #HTB-22, culture medium was 90% DMEM+10% FBS, plated cell number (Cell/well) 4000.
2). Reagents

| Reagent | Source | Cat. No. |
|---|---|---|
| DMEM | Hyclone | SH30243.01 |
| Fetal Bovine Serum | Biological Industries | 04-001-1ACS |
| DPBS | Corning | 21-031-CVR |
| CellTiter-Glo ® | Promega | G7573 |
| DMSO | Sigma | D2650 |

3). Instruments

| Instruments | Source | Cat.No. |
|---|---|---|
| 96-well plate, black | Corning | 3904 |
| Cell Counter | Count star | IC1000 |
| Microplate Shaker | Hangzhou Allsheng | MX100-4A |
| | Instruments Co., Ltd. | |
| ELISA Analyzer | PerkinElmer | VICTOR NivoTM |
| CO₂ incubator | Thermo | HERACELL 240i |
| Biosafety Cabinet | Thermo | 1300 SERIES A2 |
| Ultrasonic Cleaner | Skymen Technology | JP-010T |
| | Corporation Limited | |

### 2. Experimental Steps

1). Day 0 plated: After digesting the target cells in the logarithmic growth phase, make a single cell suspension and count.
2). Resuspend an appropriate amount of cell suspension to 4000 cells/100 μL according to the count, mixed well and then put the corresponding number of cells into a 96-well plate by pipette.
3). Each hole of the edge was sealed with 100 μL DPBS and put into an incubator for culture.
4). Day 1 drug addition: Prepare the storage solution of the compound to be tested 1 hour in advance. 24 h after plating, the culture medium was gradiently diluted to a concentration twice the final concentration of each group. Take out the plated cells from the incubator and add 100 μL of the corresponding 2-fold concentration medium to each well by pipette.

Drug concentration grouping (nM): 0, 0.229, 0.686, 2.06, 6.17, 18.5, 55.6, 167, 500, 1500.
5). Day 1 detected: Take out the plate to be tested and an appropriate amount of CTG in advance and rewarm it at room temperature in the dark. 50 μL of CTG was added to each well at 24 h. Shake with the oscillator for 5 minutes, and after balancing in the dark for 5 minutes, detect the luminescence value with a microplate reader; The mixture was shaken for 5 min, and after equilibration in the dark for 5 min, the luminescence value was detected with ELISA.
6). Day 4 detection: Take an appropriate amount of CTG 1 h in advance and rewarm it at room temperature in the dark. At the same time, take out the plate to be tested from the incubator and leave it to equilibrate at room temperature for 30 minutes; 72 h after adding the drug, 100 μL of culture medium was sucked out with pipette carefully from each cell well, and 50 μL of CTG was added. The mixture was shaken for 5 min, and after equilibration in the dark for 5 min, the luminescence value was detected with ELISA analyzer.
7). Data analysis and processed.

### Proliferation Inhibition Curve Fitting:

Collect data and convert it into survival rate: Inhibition rate (%)=(100−(OD value of compound-OD value on the first day)/(OD value of control well-OD value on the first day))×100%

Input the compound into graphpad prism to obtain the corresponding IC₅₀ value.

### 3. Experimental Results

Table 1 proliferation inhibition assays on MCF-7 cells

| | Proliferation |
|---|---|
| | inhibition |
| | IC₅₀ |
| Examples | (nM) |
| Example 1 | 5.2 |
| Compound 1f-1 | 15.2 |
| Compound 1f-2 | 11.6 |
| Example 2 | 2.6 |
| Example 3 | 15.5 |
| Example 4 | 10.2 |
| Example 5 | 12.3 |
| Example 6 | 8.4 |
| Example 7 | 20.2 |
| Example 8 | 10.3 |
| Example 9 | 25.2 |
| Example 9R | 55.3 |
| Example 10 | 8.1 |
| Example 11 | 16.3 |
| Example 12 | 50.3 |
| Example 12-1 | 32.2 |
| Example 12-2 | 80.2 |
| Example 12b | 16.5 |
| Example 12c | 3.5 |
| Example 12d | 6.8 |
| Example 13 | 8.3 |
| Example 13b | 25.6 |
| Example 14 | 8.3 |
| Example 15 | 10.6 |
| Example 16 | 12.3 |
| Example 17 | 7.8 |
| Example 18 | 11.3 |
| Example 19 | 15.2 |
| Example 20 | 7.8 |
| Example 21 | 4.2 |
| Example 22 | 9.3 |
| Example 23 | 2.5 |
| Example 24 | 10.2 |
| Example 25 | 30.2 |
| Example 25a | 25.6 |
| Example 25b | 35.1 |
| Example 25c | 15.3 |
| Example 25d | 22.3 |
| Example 26 | 35.2 |
| Example 26a | 13.3 |
| Example 26b | 9.6 |
| Example 29a | 4.5 |
| Example 29b | 2.1 |
| Example 30a | 6.3 |
| Example 30b | 3.8 |
| Example 30c | 8.1 |
| Example 30d | 4.1 |
| Example 31a | 8.2 |
| Example 31b | 4.1 |
| Example 31c | 10.2 |
| Example 31d | 8.1 |
| Example 32a | 7.5 |
| Example 32b | 5.5 |
| Example 33a | 6.5 |
| Example 33b | 8.5 |
| Example 34a | 9.6 |
| Example 34b | 4.2 |
| Example 35a | 10.3 |
| Example 35b | 6.2 |
| Example 36a | 15.2 |
| Example 36b | 8.3 |
| Example 37 | 5.1 |
| Example 38a | 5.6 |
| Example 38b | 2.0 |
| Example 39a | 9.8 |
| Example 39b | 4.6 |
| Example 40a | 6.6 |
| Example 40b | 3.5 |
| Example 41a | 28.6 |
| Example 41b | 19.6 |
| Example 42a | 14.6 |
| Example 42b | 6.6 |
| Example 43a | 5.1 |
| Example 43b | 2.7 |
| Compound A | 15.3 |
| Compound B | 5.5 |
| (exatecan) | |
| Camptothecin | 42.2 |
| SN38 | 22.5 |
| Dxd | 20.3 |

The results of the data in Table 1 showed that the compounds of the present invention exhibited strong proliferation inhibitory activity against MCF-7 cells, and the activities of some compounds were better than camptothecin, SN-38, exatecan and Dxd.

### Biological Example 2: MDA-MB-231 Cell Proliferation Inhibitory Assay

### 1. Experimental Materials

1). Cell line: MDA-MB-231 cells (human breast cancer cells), ATCC, culture medium was 1640+10% FBS, plated cell number (Cell/well) 3000.
2). Reagents

| Reagents | Source | Cat. No. |
|---|---|---|
| 1640 | KeyGEN BioTECH | KGM31800-500 |
| Fetal Bovine Serum | Corning | 35-081-CV |
| DPBS | Corning | 21-031-CVC |
| CellTiter-Glo ® | Promega | G7573 |
| DMSO | Sigma | D2650 |

3). Instruments

| Instruments | Source | Cat.No. |
|---|---|---|
| 96-well plate, black | Corning | 3904 |
| Cell Counter | Count star | IC1000 |
| Microplate Shaker | SUZHOU JIMINUO | JN400-1 |
| | INSTRUMENT | |
| | CO . . . LTD | |
| ELISA Analyzer | BioTek | SYNERGY-H1 |
| CO₂ incubator | Panasonic | MCO- |
| | | 230AICUVHL-PC |
| Biosafety Cabinet | Thermo | 1300 SERIES A2 |
| Ultrasonic Cleaner | KUNSHAN ULTRASONSC | KQ-50DE |
| | INSTRUMENTS CO. LTD. | |

### 2. Experimental Steps

1). Day 0 plated: After digesting the target cells in the logarithmic growth phase, make a single cell suspension and count.
2). Resuspend an appropriate amount of cell suspension to 3000 cells/100 μL according to the count, mixed well and then put the corresponding number of cells into a 96-well plate by pipette.
3). Each hole of the edge was sealed with 100 μL DPBS and put into an incubator for culture.
4). Day 1 drug addition: Prepare the storage solution of the compound to be tested 1 hour in advance. 24 h after plating, the culture medium was gradiently diluted to a concentration twice the final concentration of each group. Take out the plated cells from the incubator and add 100 μL of the corresponding 2-fold concentration medium to each well by pipette.
5). Day 1 detected: Take out an appropriate amount of CTG 1 h in advance and the plate was allowed to equilibrate at room temperature for 30 min. 100 μL of CTG was added to each well and shaken for 5 mins, and then standed in the dark for 5 mins, the luminescence value was detected by ELISA.
6). Day 4 detection: Take an appropriate amount of CTG 1 h in advance and rewarm it at room temperature in the dark. At the same time, take out the plate to be tested from the incubator and leave it to equilibrate at room temperature for 30 minutes; 72 h after adding the drug, 100 μL of culture medium was sucked out with pipette carefully from each cell well, and 100 μL of CTG was added. The mixture was shaken for 5 min, and after equilibration in the dark for 5 min, the luminescence value was detected with ELISA.
7). Analyze data and write detailed experimental records.

### Proliferation Inhibition Curve Fitting:

Collect data and convert it into survival rate: Inhibition rate (%)=(100−(OD value of compound-OD value on the first day)/(OD value of control well-OD value on the first day))×100%

Input the compound into graphpad prism to obtain the corresponding IC₅₀ value.

### 3. Experimental Results

Table 2 proliferation inhibition assays on MDA-MB-231 cells

| | Proliferation |
|---|---|
| | inhibition |
| | IC₅₀ |
| Examples | (nM) |
| Example 1 | 8.2 |
| Compound 1f-1 | 110.3 |
| Compound 1f-2 | 74.2 |
| Example 2 | 15.7 |
| Example 3 | 120.4 |
| Example 4 | 280.2 |
| Example 5 | 600 |
| Example 6 | 1100 |
| Example 7 | 320 |
| Example 8 | 210 |
| Example 9 | 310 |
| Example 9R | 180 |
| Example 10 | 260.3 |
| Example 11 | 120.3 |
| Example 12 | 1200 |
| Example 12-1 | 500 |
| Example 12-2 | 1800 |
| Example 12b | 20.2 |
| Example 12c | 6.6 |
| Example 12d | 12.3 |
| Example 13 | 80.2 |
| Example 13b | 130.2 |
| Example 14 | 88.6 |
| Example 15 | 140.9 |
| Example 16 | 100.5 |
| Example 17 | 96.7 |
| Example 18 | 150.2 |
| Example 19 | 260.6 |
| Example 20 | 120.0 |
| Example 21 | 20.2 |
| Example 22 | 60.8 |
| Example 23 | 7.7 |
| Example 24 | 80.1 |
| Example 25 | 260.8 |
| Example 25a | 40.2 |
| Example 25b | 60.9 |
| Example 25c | 35.6 |
| Example 25d | 50.3 |
| Example 26 | 150.2 |
| Example 26a | 89.2 |
| Example 26b | 55.2 |
| Example 29a | 10 |
| Example 29b | 6.6 |
| Example 30a | 11.6 |
| Example 30b | 25.0 |
| Example 30c | 12.2 |
| Example 30d | 28.1 |
| Example 31a | 15.1 |
| Example 31b | 9.2 |
| Example 31c | 18.3 |
| Example 31d | 8.3 |
| Example 32a | 16.7 |
| Example 32b | 20.2 |
| Example 33a | 10.9 |
| Example 33b | 24.3 |
| Example 34a | 11.6 |
| Example 34b | 8.8 |
| Example 35a | 20.6 |
| Example 35b | 7.1 |
| Example 36a | 26.3 |
| Example 36b | 15.4 |
| Example 37 | 90.8 |
| Example 38a | 16.6 |
| Example 38b | 6.2 |
| Example 39a | 25.9 |
| Example 39b | 9.1 |
| Example 40a | 50 |
| Example 40b | 25.7 |
| Example 41a | 100.5 |
| Example 41b | 30.5 |
| Example 42a | 124.9 |
| Example 42b | 75.6 |
| Example 43a | 16.8 |
| Example 43b | 8.0 |
| Compound A | 260.2 |
| Compound B | 30.9 |
| (exatecan) | |
| Camptothecin | 250.2 |
| SN38 | 124.1 |
| Dxd | 101.2 |

The results of the data in Table 1 showed that the compounds of the present invention exhibited strong proliferation inhibitory activity against MDA-MB-231 cells, and the activities of some compounds were better than camptothecin, SN-38, exatecan and Dxd.

### Biological Example 3: MDA-MB-435s Cell Proliferation Inhibitory Assay

### 1. Experimental Materials

1). Cell line: MDA-MB-435s cells (human melanoma cells), CAS, culture medium was 1640+10% FBS, plated cell number (Cell/well) 3000.
2). Reagents

| Reagents | Source | Cat. No. |
|---|---|---|
| 1640 | KeyGEN BioTECH | KGM31800-500 |
| Fetal Bovine Serum | Corning | 35-081-CV |
| DPBS | Corning | 21-031-CVC |
| CellTiter-Glo ® | Promega | G7573 |
| DMSO | Sigma | D2650 |

2). Instruments

| Instruments | Source | Cat.No. |
|---|---|---|
| 96-well plate, black | Corning | 3904 |
| Cell Counter | Count star | IC1000 |
| Microplate Shaker | SUZHOU JIMINUO | JN400-1 |
| | INSTRUMENT | |
| | CO . . . LTD | |
| ELISA Analyzer | BioTek | SYNERGY-H1 |
| CO₂ incubator | Panasonic | MCO- |
| | | 230AICUVHL-PC |
| Biosafety Cabinet | Thermo | 1300 SERIES A2 |
| Ultrasonic Cleaner | KUNSHAN ULTRASONSC | KQ-50DE |
| | INSTRUMENTS CO. LTD. | |

### 2. Experimental Steps

1). Day 0 plated: After digesting the target cells in the logarithmic growth phase, make a single cell suspension and count.
2). Resuspend an appropriate amount of cell suspension to 3000 cells/100 μL according to the count, mixed well and then put the corresponding number of cells into a 96-well plate by pipette.
3). Each hole of the edge was sealed with 100 μL DPBS and put into an incubator for culture.
4). Day 1 drug addition: Prepare the storage solution of the compound to be tested 1 hour in advance. 24 h after plating, the culture medium was gradiently diluted to a concentration twice the final concentration of each group. Take out the plated cells from the incubator and add 100 μL of the corresponding 2-fold concentration medium to each well by pipette.
5). Day 1 detected: Take out an appropriate amount of CTG 1 h in advance and the plate was allowed to equilibrate at room temperature for 30 min. 100 μL of CTG was added to each well and shaken for 5 mins, and then standed in the dark for 5 mins, the luminescence value was detected by ELISA.
6). Day 4 detection: Take an appropriate amount of CTG 1 h in advance and rewarm it at room temperature in the dark. At the same time, take out the plate to be tested from the incubator and leave it to equilibrate at room temperature for 30 minutes; 72 h after adding the drug, 100 μL of culture medium was sucked out with pipette carefully from each cell well, and 100 μL of CTG was added. The mixture was shaken for 5 min, and after equilibration in the dark for 5 min, the luminescence value was detected with ELISA.
7). Analyze data and write detailed experimental records.

### Proliferation Inhibition Curve Fitting:

Collect data and convert it into survival rate: Inhibition rate (%)=(100−(OD value of compound−OD value on the first day)/(OD value of control well-OD value on the first day))×100%

Input the compound into graphpad prism to obtain the corresponding IC₅₀ value.

### 3. Experimental Results

Table 3 proliferation inhibition assays on MDA-MB-435s cells

| | Proliferation |
|---|---|
| | inhibition |
| | IC₅₀ |
| Examples | (nM) |
| Example 1 | 0.75 |
| Compound 1f-1 | 5.8 |
| Compound 1f-2 | 3.5 |
| Example 2 | 2.3 |
| Example 3 | 11.2 |
| Example 4 | 16.3 |
| Example 5 | 20.3 |
| Example 6 | 36.8 |
| Example 7 | 162.3 |
| Example 8 | 35.2 |
| Example 9 | 25.3 |
| Example 9R | 15.3 |
| Example 10 | 42.8 |
| Example 11 | 5.6 |
| Example 12 | 59.3 |
| Example 12-1 | 38.2 |
| Example 12-2 | 75.2 |
| Example 12b | 3.2 |
| Example 12c | 0.7 |
| Example 12d | 1.6 |
| Example 13 | 2.8 |
| Example 13b | 3.9 |
| Example 14 | 3.8 |
| Example 15 | 4.2 |
| Example 16 | 8.2 |
| Example 17 | 6.3 |
| Example 18 | 10.3 |
| Example 19 | 15.3 |
| Example 20 | 2.7 |
| Example 21 | 1.2 |
| Example 22 | 5.6 |
| Example 23 | 0.76 |
| Example 24 | 6.6 |
| Example 25 | 10.2 |
| Example 25a | 6.3 |
| Example 25b | 9.6 |
| Example 25c | 3.3 |
| Example 25d | 5.2 |
| Example 26 | 5.6 |
| Example 26a | 2.8 |
| Example 26b | 1.6 |
| Example 29a | 2.1 |
| Example 29b | 0.72 |
| Example 30a | 3.3 |
| Example 30b | 0.9 |
| Example 30c | 3.8 |
| Example 30d | 1.2 |
| Example 31a | 3.8 |
| Example 31b | 1.0 |
| Example 31c | 5.8 |
| Example 31d | 2.3 |
| Example 32a | 3.37 |
| Example 32b | 1.85 |
| Example 33a | 2.36 |
| Example 33b | 0.85 |
| Example 34a | 4.37 |
| Example 34b | 2.85 |
| Example 35a | 3.33 |
| Example 35b | 1.15 |
| Example 36a | 6.33 |
| Example 36b | 3.15 |
| Example 37 | 2.33 |
| Example 38a | 1.12 |
| Example 38b | 0.65 |
| Example 39a | 2.12 |
| Example 39b | 1.65 |
| Example 40a | 4.12 |
| Example 40b | 2.65 |
| Example 41a | 5.12 |
| Example 41b | 3.86 |
| Example 42a | 3.21 |
| Example 42b | 2.56 |
| Example 43a | 3.31 |
| Example 43b | 0.65 |
| Compound A | 21.35 |
| Compound B | 2.52 |
| (exatecan) | |
| Camptothecin | 10.22 |
| SN38 | 6.89 |
| Dxd | 5.12 |

The results of the data in Table 1 showed that the compounds of the present invention exhibited strong proliferation inhibitory activity against MDA-MB-435s cells, and the activities of some compounds were better than camptothecin, SN-38, exatecan and Dxd.

SAR analysis of substituent R⁵ of the compound in the present invention:

TABLE 4
Comparative table of cell proliferation activities
of some compounds in the present invention

| | | MDA- | MDA- |
|---|---|---|---|
| | MCF-7 | MB-231 | MB-435s |
| | Proliferation | Proliferation | Proliferation |
| | inhibition | inhibition | inhibition |
| | IC₅₀ | IC₅₀ | IC₅₀ |
| Compounds | (nM) | (nM) | (nM) |
| Example 1 | 5.2 | 8.2 | 0.75 |
| Compound A | 15.3 | 260.2 | 21.35 |
| Example 12d | 6.8 | 12.3 | 1.6 |
| Example 12b | 16.5 | 20.2 | 3.2 |

The cell data activity in Table 4 shows that when the methylene group in R⁵ has a substituent and is a methyl group, the activity was significantly better than that without a substituent

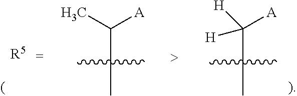

### Biological Example 4: Caco-2 Cell Membrane Permeability Assay

### 1. Experimental Materials

1). Cell line: Caco-2 cells (human colorectal adenocarcinoma cells), CAS, culture medium was 1640+10% FBS, plated cell number (Cell/well) 3000.
2). Reagents

| Reagents | Source | Cat. No. |
|---|---|---|
| MEM | BasalMedia | L550KJ |
| Fetal Bovine Serum | Biological Industries | 04-001-1ACS |
| DPBS | Corning | 21-031-CVC |
| DMSO | Sigma | D2650 |
| MEM | BasalMedia | L550KJ |

3). Instruments

| Instruments | Source | Cat.No. |
|---|---|---|
| Cell Counter | Count star | IC1000 |
| CO₂ incubator | Thermo | HERACELL 240i |
| Biosafety Cabinet | Thermo | 1300 SERIES A2 |
| transwell | Guangzhou Jet Bio- | TCS016024 |
| | Filtration Co., Ltd. | |

### 2. Experimental Steps

1). Day 0 plated: After digesting the target cells in the logarithmic growth phase, make a single cell suspension and count.
2). Took out the corresponding number of transwell chambers and placed them in a 24-well plate, added 200 μL PBS to the upper chamber, incubated for 10 mins, and removed the PBS from the upper chamber by pipette.
3). According to the count, took an appropriate amount of cell suspension and resuspended it to 5000 cells/200 μL, mixed well and added it to the upper chamber of the transwell, and added 600 μL of complete culture medium to the lower chamber.
4). medium exchanged on DAY1: The upper chamber and the lower chamber were exchanged with complete culture medium containing 0.1 mg/mL ascorbic acid for 9 days continuously, and the resistance value of membrane expansion was measured. When the transmembrane resistance value in transwell chamber was ≥220 (2 cm2, it could be used for drug permeability transport.
5). Added drug on DAY10: Washed the transwell plate cultured for 9 days with HBSS buffer for 2-3 times, added 200 μL HBSS buffer to the upper chamber, added 600 μL HBSS buffer to the lower chamber, and incubate for 15 mins. Afterwards, the HBSS buffer was sucked out, and 200 μL of the drug solution prepared with HBSS buffer was added to the upper chamber (the concentration was 100 μg/mL), and 600 μL of HBSS buffer was added to the lower chamber as the receiving solution, and two duplicate wells were set in each chamber and the experimental process is 180 min.
6). Collected the liquid in the upper chamber and the lower chamber for detection.
7). Calculated Papp according to the formula “Papp=dQ/(dt×A×C₀)”. dQ/dt was the transport amount of drugs per unit time, which was the slope of the linear regression of cumulative drug release amount Q versus time t. A was the bottom area of the Transwell orifice plate membrane, and the bottom area was 0.33 cm² of 24-well plate in this experiment. Co was the initial concentration of drugs in the upper chamber (μg mL-1).

### 3. Experimental Results

TABLE 5
Cell membrane permeability results of compounds

| | Permeability | | Permeability |
|---|---|---|---|
| | Coefficient | | Coefficient |
| | (Pₐₚₚ 10⁻⁶ cm/s) | | (Pₐₚₚ 10⁻⁶ cm/s) |
| Compounds | A→B | Compounds | A→B |
| Metoprolol | 192.9 | Dxd | 42.0 |
| Tartrate | | | |
| Atenolol | 34.9 | Example 13b | 56.1 |
| SN38 | 46.8 | Example 12b | 49.3 |
| Compound B | 68.6 | Example 12c | 96.0 |
| (exatecan) | | | |
| Compound A | 68.8 | | |
| Example 1 | 100.0 | | |

NOTE:
A→B represent the permeability of test compound from apical (AP) of the Caco-2 monolayer across the single cell layer or the intercellular space to basolateral (BL).

Table 5 show that the compounds of the present invention have high permeability. For example, Examples 1 and 12c have obvious advantages over SN38 and exatecan, and Example 12c has obvious advantages over Dxd. In particular, when the substituent R⁵ (methylene) has a substituent, especially methyl, the permeability was obviously better than that of the compound without substituent (compound 1 versus compound A, compound 12c versus compound 12b). Highly permeable compounds will help increase the concentration of drugs in targeted tissues and especially enhance the bystander effect of conjugated drugs.

### Biological Example 5: In Vivo Pharmacodynamic Study of Polymer Conjugate Example 27 and Example 28 on Human Pancreatic Cancer BxPC-3 Subcutaneous Xenograft Tumor BALB/C NUDE Mouse Model

### 1. Experimental Animals

BALB/c nude mice, 4-5 weeks old, weighing 18-24 g, female, provided by Hangzhou Ziyuan Experimental Animal Technology Co., Ltd., animal certificate number: 20210718Aabbb0105000741.

### 2. Feeding Conditions

After the animals arrived, they were kept in the experimental environment for 3-7 days before starting the experiment. Animals were kept in IVC (independent ventilation system) cages (5 animals per cage) in SPF-grade animal rooms. The animal information card for each cage indicates the number of animals in the cage, gender, strain, date of receipt, dosage regimen, experiment number, group and experiment start date. All cages, bedding and drinking water are sterilized before use. Cages, feed and drinking water should be refreshed twice a week. The feeding environment and lighting conditions were as follows: temperature: 20˜26° C., humidity: 40˜70%, lighting period: 12 h of light, 12 h of no light (lights on at 8 a.m.-lights off at 8 p.m.).

Cage: Made of polycarbonate, volume 325 mm×210 mm×180 mm. The bedding material was corn cobs and is refreshed weekly.

Food: Experimental animals could eat by themselves during the entire experimental period (irradiation sterilization, dry granular food).

Drinking water: Experimental animals could drink sterilized water by themselves.

Cage identification: The animal information card of each cage should indicate the number of animals in the cage, gender, strain, date of receipt, dosage regimen, experiment number, group and experiment start date.

Animal identification: Experimental animals were identified by toe clipping.

### 3. Tumor Cell Inoculation Methods

Human pancreatic cancer BxPC-3 cells (Cell Bank, Chinese Academy of Sciences) were cultured in monolayer in vitro. The culture conditions were RPMI 1640 medium plus 10% fetal bovine serum and cultured in a 37° C. 5% CO₂ incubator. Trypsin-EDTA was used twice a week for routine digestion and passage. When the cell saturation was 80%-90% and the number reached the requirements, cells were collected, counted, and inoculated. BxPC-3 was subcutaneously inoculated into the right back of mice, and administration was started in groups when the tumor growth reached 128 mm³.

### 4. Preparation of Test Samples

Appropriate amount of Examples 27 and 28 were weighed, and the corresponding volume of physiological saline was added, and heated with ultrasound until dissolved.

### 5. Drug Administration.

Dosage and administration schedule were showed in Table 3. The subcutaneous tumor volume of nude mice was measured 2-3 times a week, and the mice were weighed, and the data were recorded.

TABLE 6

| | | dose | | Numer of | Dosing |
|---|---|---|---|---|---|
| No. | Group | (mg/kg) | Route | animals | cycle |
| 1 | Vehicle | — | i.v | 6 | qw x3 |
| 2 | Example 27 | 15 | i.v | 6 | qw x3 |
| 3 | Example 28 | 15 | i.v | 6 | qw x3 |

Note:
Dose volume was 10 mg/mL.

### 6. Tumor Measurement and Analysis Index

The experimental index was to examine whether tumor growth was inhibited, delayed or cured. Tumor diameter was measured using vernier calipers twice weekly. The calculation formula of tumor volume was V=0.5a×b², where a and b represent the long and short diameters of the tumor, respectively.

The antitumor efficacy of the compound was evaluated by the relative tumor proliferation rate T/C (%). The evaluation criteria are: T/C (%)>40% was considered invalid; T/C (%)≤40%, and P<0.05 was considered effective after statistical processing.

Relative tumor proliferation rate T/C (%): The calculation formula was as follows: T/C %=T_{RTV}/C_{RTV}×100% (T_{RTV}: the average value of RTV in treatment group; C_{RTV}: the average value RTV in negative control group). According to the results of tumor measurement, the relative tumor volume (RTV) was calculated, and the calculation formula was RTV=Vₜ/V₀, where V₀ was the tumor volume measured at the time of drug administration in groups (d₀), Vₜ was the tumor volume measured at a certain time, and T_{RTV} and C_{RTV} were collected on the same day.

Calculation of the relative tumor proliferation rate T/C (%): If T>T₀, then T/C (%)=(T−T₀)/(C−C₀)×100%, if T<T₀, then T/C (%)=(T−T₀)/T₀×100%, wherein T and C were tumor volumes at the end of the experiment; T₀ and C₀ were tumor volumes at the beginning of the experiment.

Calculation of percent tumor growth inhibition rate TGI (%): TGI (%)=(1−T/C)×100%.

Evaluation criteria: T/C (%)>40 (i.e. TGI (%)<60%) means invalid; T/C (%)≤40 (i.e. TGI (%)≥60%) means valid and statistical significance value P<0.05 was effective.

### 7. Pharmacodynamic Experimental Results

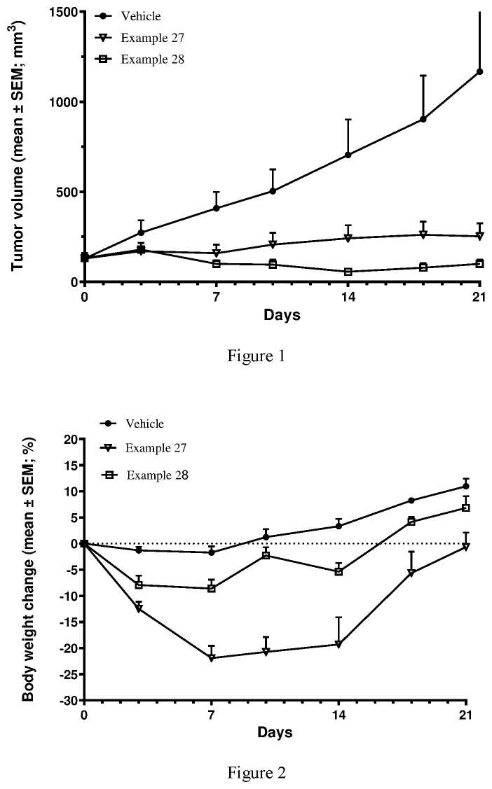

The inhibitory effects of control group, Example 27 and Example 28 on the tumor volume of BxPc-3 cells were shown in Table 7 and FIG. 1.

TABLE 7
Inhibitory effects of Example 27 and Example
28 on BxPc-3 cell tumor volume

| | | Tumor | Tumor | | |
|---|---|---|---|---|---|
| | | volume (mm³) | volume (mm³) | T/C | P |
| Group | Route | (day 0) | (day 21) | (%) | value |
| Vehicle | i.v | 129 ± 31 | 1167 ± 382 | — | — |
| Example 27 | i.v | 131 ± 31 | 253 ± 72 | 21.3 | 0.007 |
| Example 28 | i.v | 132 ± 31 | 100 ± 23 | 8.4 | 0.015 |

The results show that polymer conjugate Example 28 had a very strong inhibitory effect on the tumor growth of the BxPc-3 nude mouse model when administered once a week for three consecutive weeks via i.v route. Among them, Example 28 (polymer conjugate Example 13) had better anti-tumor effect than the positive control Example 27 (polymer conjugate exatecan).

### 8. Weight Change Evaluation

The effects of the control group, Example 27 and Example 28 on the body weight of the human pancreatic cancer BxPc-3 subcutaneous xenograft tumor female BALB/c nude mouse model were shown in FIG. 2.

The results showed that the polymer-conjugate Example 28 of Example 13 had less toxic and side effects than the positive control Example 27 (polymer-conjugate exatecan) in the nude mouse experimental model of BxPC-3.

1. A camptothecin compound of formula (I), or a pharmaceutically acceptable salt thereof: 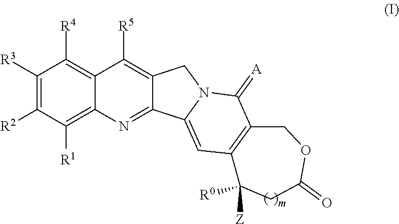
wherein:
R⁰ is C1-C4 alkyl, C1-C4 haloalkyl, C1-C4 cycloalkyl, C1-C4 deuterated alkyl, C2-C4 alkynyl, or C2-C4 alkenyl;
R¹, R², R³ and R⁴ are each independently hydrogen, deuterium, halogen, cyano, hydroxyl, thiol, sulfone, sulfoxide, substituted or unsubstituted amino, nitro, alkynyl, alkenyl, alkyl, haloalkyl, cycloalkyl, alkoxy, alkylmercapto, heterocycloalkyl, deuterated alkyl, aryl, or heteroaryl;
alternatively, R¹ and R², R² and R³, R³ and R⁴ each form a 5-7 membered cycloalkyl or heterocycloalkyl group together with their co-connected carbon atom;
R⁵ is 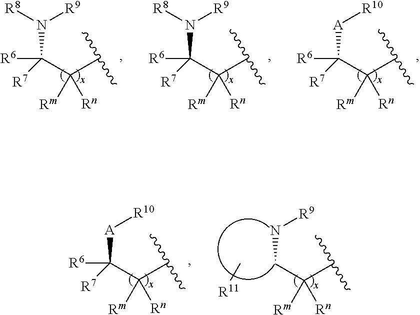
(the ring 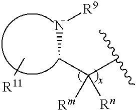
in is 3-7 membered heterocycle), 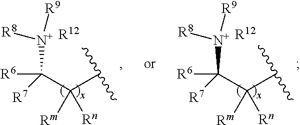
R⁶ is hydrogen, deuterium, cyano, alkynyl, alkenyl, alkyl, haloalkyl, cycloalkyl, heterocycloalkyl, deuterated alkyl, aryl or heteroaryl;
R⁷ is cyano, alkynyl, alkenyl, alkyl, haloalkyl, cycloalkyl, heterocycloalkyl, deuterated alkyl, aryl or heteroaryl;
alternatively, R⁶ and R⁷ each form a 3-7 membered cycloalkyl or heterocycloalkyl group together with their co-connected carbon atom;
R⁸ and R⁹ are each independently hydrogen, deuterium, hydroxyl, substituted or unsubstituted amino, alkyl, haloalkyl, cycloalkyl, heterocycloalkyl, deuterated alkyl, aryl, heteroaryl, 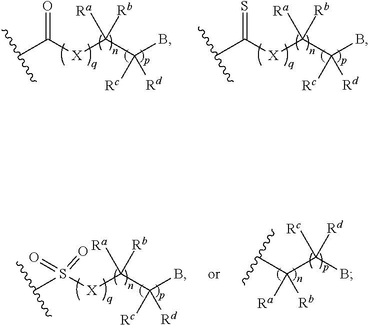
alternatively, R⁸ and R⁹ form a 3-7 membered heterocycloalkyl group together with their co-connected nitrogen atoms;
R¹⁰ is hydrogen, alkynyl, alkenyl, alkyl, haloalkyl, cycloalkyl, alkylmercapto, heterocycloalkyl, deuterated alkyl, aryl, heteroaryl, 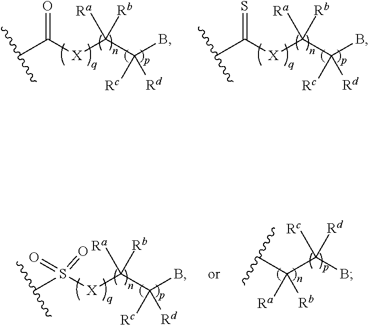
R¹¹ is one or more substituents on a 3-7 membered heterocyclic ring, which is selected from hydrogen, deuterium, halogen, cyano, hydroxyl, substituted or unsubstituted amino, alkynyl, alkenyl, alkyl, haloalkyl, ring Alkyl, alkoxy, alkylmercapto, heterocycloalkyl, deuterated alkyl, aryl, or heteroaryl;
R¹² is selected from alkyl;
R^{a} and R^{b} are each independently hydrogen, deuterium, halogen, cyano, hydroxyl, mercapto, substituted or unsubstituted amino, alkynyl, alkenyl, alkyl, haloalkyl, cycloalkyl, alkoxy, alkylmercapto, heterocycloalkyl, deuterated alkyl, aryl, or heteroaryl;
alternatively, R^{a} and R^{b} form a 3-7 membered cycloalkyl or heterocycloalkyl group together with their co-connected carbon atom;
R^{c} and R^{d} are each independently hydrogen, deuterium, halogen, cyano, hydroxyl, mercapto, substituted or unsubstituted amino, alkynyl, alkenyl, alkyl, haloalkyl, cycloalkyl, alkoxy, alkylmercapto, heterocycloalkyl, deuterated alkyl, aryl, or heteroaryl;
alternatively, R^{c} and R^{d} each form a 3-7 membered cycloalkyl or heterocycloalkyl group together with their co-connected carbon atom;
alternatively, R^{a} and R^{b}, R^{b} and R^{c}, R^{c} and R^{d} each form a cycloalkyl or heterocycloalkyl group together with their co-connected carbon atom;
B is hydrogen, deuterium, halogen, hydroxyl, mercapto, substituted or unsubstituted amino, alkynyl, alkenyl, alkoxy, alkylmercapto, aryl, or heteroaryl;
R^{m} and Rⁿ are each independently hydrogen, deuterium, halogen, cyano, hydroxyl, substituted or unsubstituted amino, alkynyl, alkenyl, alkyl, haloalkyl, cycloalkyl, alkoxy, alkylmercapto, heterocycloalkyl, deuterated alkyl, aryl, or heteroaryl;
alternatively, R^{m} and Rⁿ form a cycloalkyl or heterocycloalkyl group together with co-connected carbon atom;
x, n and p are 0, 1, 2, 3 or 4;
q is 0 or 1;
m is 0 or 1;
X is O, S or NH;
A is O or S, and when A-R¹⁰ is a hydroxyl group, not all R¹, R², R³ and R⁴ are hydrogen (H); and,
Z is OH, SH or F.

2. The camptothecin compound or the pharmaceutically acceptable salt thereof according to claim 1, characterized in that it satisfies one or more of the following conditions:
(1) R⁰ is C₁-C₄ alkyl;
(2) R¹, R², R³ and R⁴ are independently hydrogen, halogen, hydroxyl, alkyl, haloalkyl, alkoxy or deuterated alkyl; alternatively, R² and R³ form a heterocyclic alkyl group together with their co-connected carbon atom;
(3) R⁶ is hydrogen or alkyl;
(4) R⁷ is alkyl, haloalkyl or deuterated alkyl;
(5) R⁸ and R⁹ are each independently hydrogen, alkyl, 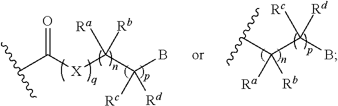
alternatively, R⁸ and R⁹ form a 3-7 membered heterocycloalkyl group together with their co-connected nitrogen atom;
(6) R¹⁰ is hydrogen;
(7) R¹¹ is hydrogen;
(8) R¹² is alkyl;
(9) R^{a} and R^{b} are each independently hydrogen, deuterium, alkyl, cycloalkyl or haloalkyl; alternatively, each of R^{a} and R^{b} forms a cycloalkyl group together with their co-connected carbon atom; alternatively, each of R^{b} and R^{c} forms a cycloalkyl group together with their co-connected carbon atom;
(10) R^{c} and R^{d} are each independently hydrogen, deuterium, alkyl, cycloalkyl or haloalkyl; alternatively, each of R^{c} and R^{d} form a cycloalkyl group together with their co-connected carbon atom; alternatively, R^{b} and R^{c} form a cycloalkyl group together with their co-connected carbon atom;
(11) B is hydrogen, hydroxyl or substituted or unsubstituted amino group; and,
(12) R^{m} and Rⁿ are each independently hydrogen or a substituted or unsubstituted amino group.

3. The camptothecin compound or the pharmaceutically acceptable salt thereof according to claim 1, characterized in that it satisfies one or more of the following conditions:
(1) R² is hydrogen or halogen; R³ is hydrogen, halogen, hydroxyl, alkyl, haloalkyl, alkoxy or deuterated alkyl; alternatively, R² and R³ form a heterocycloalkyl group together with their co-connected carbon atom; and,
(2) R⁴ is hydrogen, halogen or alkyl.

4. The camptothecin compound or the pharmaceutically acceptable salt thereof according to claim 1, characterized in that R⁵ is 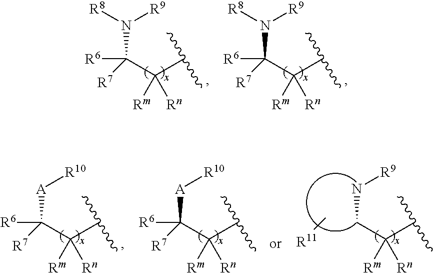
(the ring in 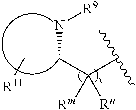
is a 3-7 membered heterocycle).

5. The camptothecin compound or the pharmaceutically acceptable salt thereof according to claim 1, characterized in that it satisfies one or more of the following conditions:
(1) R⁰ is ethyl;
(2) R¹ is hydrogen;
(3) R² is hydrogen or fluorine; R³ is hydrogen, fluorine, hydroxyl, methyl, difluoromethyl, trifluoromethyl, methoxy or methyl-d3; alternatively, R² and R³ form 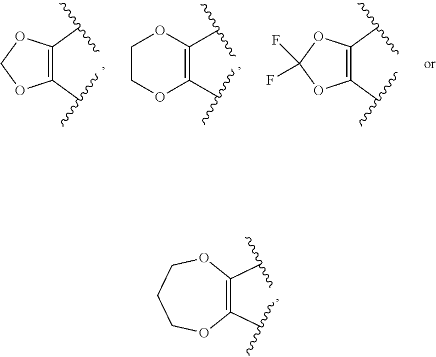 [SMILES: *C1=C(*)OCO1 ; *C1=C(*)OCCO1 ; *C1=C(*)OC(F)(F)O1 ; *C1=C(*)OCCCO1]
together with their co-connected carbon atom;
(4) R⁴ is hydrogen or methyl;
(5) R⁶ is hydrogen or methyl;
(6) R⁷ is methyl, ethyl, trifluoromethyl or methyl-d3;
(7) R⁸ and R⁹ are each independently hydrogen, methyl, 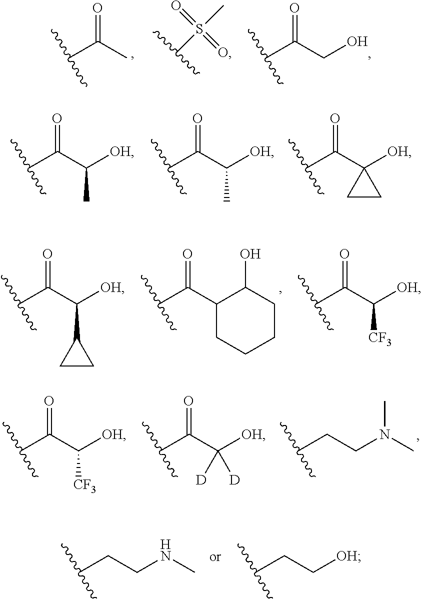
alternatively, R⁸ and R⁹ form 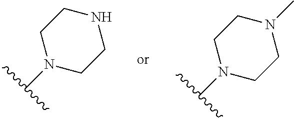
together with their co-connected nitrogen atom;
(8) R⁵ is 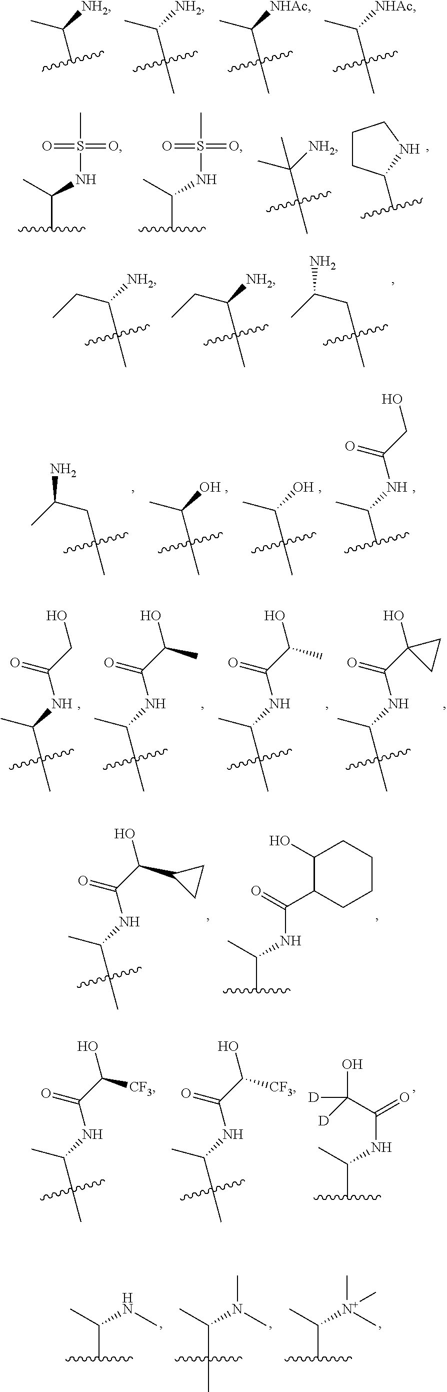 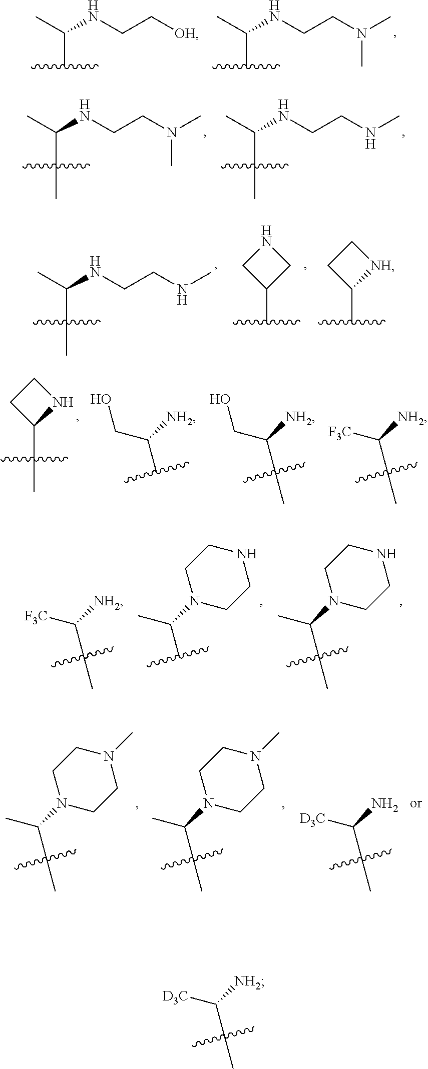
(9) A is oxygen;
(10) m is 0; and,
(11) Z is hydroxyl group.

6. The camptothecin compound or the pharmaceutically acceptable salt thereof according to claim 1, characterized in that it is a camptothecin compound as shown in general formula (II) or a pharmaceutically acceptable salt thereof:

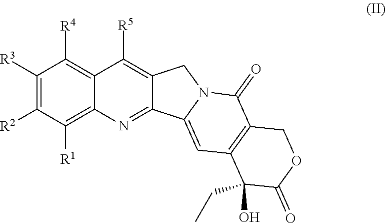

7. The camptothecin compound or the pharmaceutically acceptable salt thereof according to claim 1, characterized in that it is a camptothecin compound as shown in general formula (III) or a pharmaceutically acceptable salt thereof: 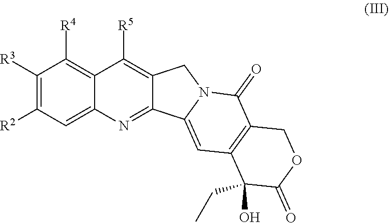
wherein:
R², R³ and R⁴ are each independently hydrogen, deuterium, halogen, cyano, hydroxyl, mercapto, sulfonyl, sulfoxide, substituted or unsubstituted amino, alkenyl, alkynyl, alkyl, haloalkyl, cycloalkyl base, alkoxy, heterocycloalkyl or deuterated alkyl;
alternatively, R² and R³, R³ and R⁴ each form a 5-7 membered cycloalkyl or heterocycloalkyl group together with their co-connected carbon atom;
R⁵ is 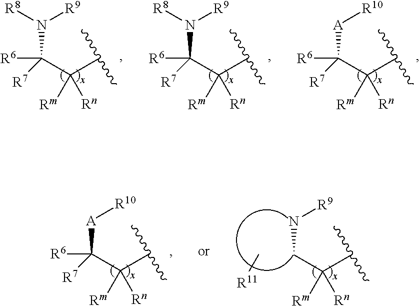
(the ring in 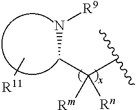
is a 3-7 membered heterocycle);
R⁶ is hydrogen, deuterium, alkyl, haloalkyl, cycloalkyl, heterocycloalkyl, deuterated alkyl, aryl or heteroaryl;
R⁷ is alkyl, haloalkyl, cycloalkyl, heterocycloalkyl, deuterated alkyl, aryl or heteroaryl;
alternatively, R⁶ and R⁷ form a 3-7 membered cycloalkyl or heterocycloalkyl group together with their co-connected carbon atom;
R⁸ and R⁹ are each independently hydrogen, deuterium, alkyl, haloalkyl, cycloalkyl, heterocycloalkyl, deuterated alkyl, aryl, heteroaryl, 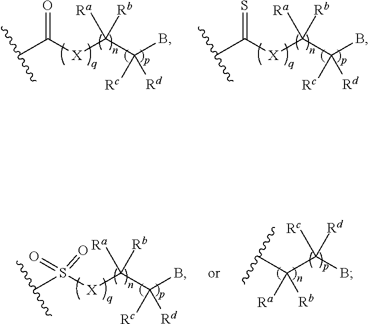
alternatively, R⁸ and R⁹ form a 3-7 membered cycloalkyl or heterocycloalkyl group together with their co-connected nitrogen atom;
R¹⁰ is hydrogen, alkyl, haloalkyl, cycloalkyl, heterocycloalkyl, deuterated alkyl, aryl or heteroaryl, 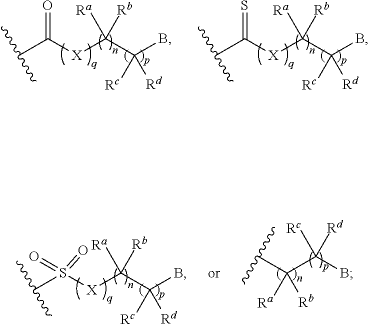
R¹¹ is one or more substituents of a 3-7 membered heterocycle, which is selected from hydrogen, deuterium, halogen, cyano, hydroxyl, substituted or unsubstituted amino, alkynyl, alkenyl, alkyl, haloalkyl, cycloalkyl, alkoxy, alkylmercapto, heterocycloalkyl, deuterated alkyl, aryl or heteroaryl;
R^{a} and R^{b} are each independently hydrogen, deuterium, halogen, alkyl, haloalkyl, cycloalkyl, heterocycloalkyl or deuterated alkyl;
alternatively, R^{a} and R^{b} form a 3-7 membered cycloalkyl or heterocycloalkyl group together with their co-connected carbon atom;
R^{c} and R^{d} are each independently hydrogen, deuterium, halogen, cyano, hydroxyl, substituted or unsubstituted amino, alkynyl, alkenyl, alkyl, haloalkyl, cycloalkyl, alkoxy, alkylmercapto, heterocycle alkyl, deuterated alkyl, aryl or heteroaryl;
alternatively, R^{c} and R^{d} form a 3-7 membered cycloalkyl or heterocycloalkyl group together with their co-connected carbon atom;
alternatively, R^{a} and R^{b}, R^{b} and R^{c}, R^{c} and R^{d} each form a cycloalkyl or heterocycloalkyl group together with their co-connected carbon atom;
B is hydrogen, deuterium, hydroxyl, mercapto, substituted or unsubstituted amino group;
R^{m} and Rⁿ are each independently hydrogen, deuterium, halogen, hydroxyl, alkyl, haloalkyl, cycloalkyl or deuterated alkyl;
alternatively, R^{m} and Rⁿ form a cycloalkyl or heterocycloalkyl group together with their co-connected carbon atom;
x, n and p are 0, 1, 2, 3 or 4;
q is 0 or 1;
X is O, S or NH; and,
A is O or S, and when A-R¹⁰ is OH, R², not all R³ and R⁴ are hydrogen (H).

8. The camptothecin compound or the pharmaceutically acceptable salt thereof according to claim 1, characterized in that it is a camptothecin compound as shown in general formula (IV) or a pharmaceutically acceptable salt thereof: 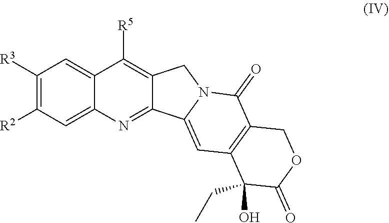
wherein:
R² and R³ are each independently hydrogen, deuterium, halogen, hydroxyl, mercapto, C1-C4 alkyl sulfoxide, C1-C4 alkyl, C1-C4 haloalkyl, C1-C4 alkoxy, C1-C4 deuterium Alkyl, C2-C4 alkynyl or C2-C4 alkenyl;
alternatively, R² and R³ together with their co-attached carbon atoms form the following cyclic structure: 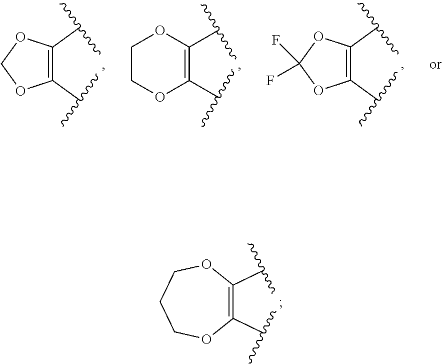 [SMILES: *C1=C(*)OCO1 ; *C1=C(*)OCCO1 ; *C1=C(*)OC(F)(F)O1 ; *C1=C(*)OCCCO1]
R⁵ is 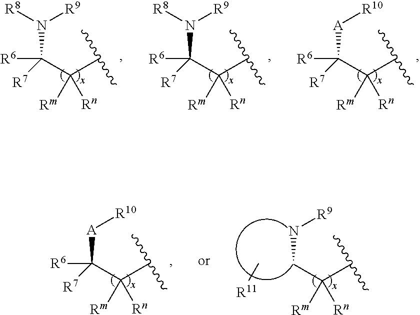
(the ring in 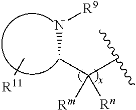
is a 3-7 membered heterocycle);
R⁶ is hydrogen, deuterium, alkyl, haloalkyl, cycloalkyl, heterocycloalkyl or deuterated alkyl;
R⁷ is alkyl, haloalkyl, cycloalkyl, heterocycloalkyl or deuterated alkyl;
alternatively, R⁶ and R⁷ form a 3-7-membered cycloalkyl or heterocycloalkyl group together with their co-connected carbon atoms;
R⁸ and R⁹ are each independently hydrogen, deuterium, alkyl, haloalkyl, cycloalkyl, heterocycloalkyl, deuterated alkyl, aryl, heteroaryl, 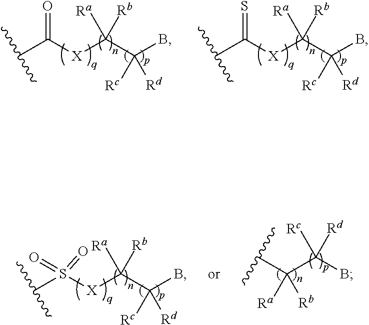
alternatively, R⁸ and R⁹ form a 3-7-membered cycloalkyl or heterocycloalkyl group together with their co-connected nitrogen atoms;
R¹⁰ is hydrogen, alkyl, haloalkyl, cycloalkyl, heterocycloalkyl or deuterated alkyl, 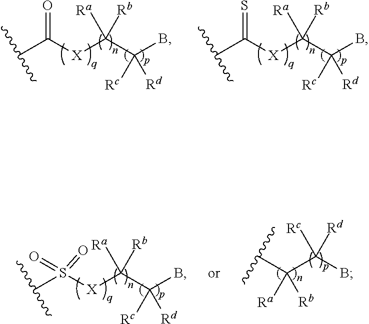
R¹¹ is one or more substituents of a 3-7 membered heterocycle, which is selected from hydrogen, deuterium, halogen, cyano, hydroxyl, substituted or unsubstituted amino, alkyl, haloalkyl, cycloalkyl or heterocycloalkyl;
R^{a} and R^{b} are each independently hydrogen, deuterium, halogen, alkyl, haloalkyl or cycloalkyl;
alternatively, R^{a} and R^{b} form a 3-7 membered cycloalkyl or heterocycloalkyl group together with their co-connected carbon atoms;
R^{c} and R^{d} are each independently hydrogen, deuterium, halogen, cyano, hydroxyl, substituted or unsubstituted amino, alkyl, haloalkyl, cycloalkyl, alkoxy, alkylmercapto, heterocycloalkyl or deuterated alkyl base;
alternatively, R^{c} and R^{d} form a 3-7 membered cycloalkyl or heterocycloalkyl group together with their co-connected carbon atom;
alternatively, R^{a} and R^{b}, R^{b} and R^{c}, R^{c} and R^{d} each form a 3-7 membered cycloalkyl or heterocycloalkyl group together with their carbon atom;
B is hydroxyl, substituted or unsubstituted amino group;
R^{m} and Rⁿ are each independently hydrogen, deuterium, halogen, hydroxyl, alkyl, haloalkyl, cycloalkyl or deuterated alkyl;
alternatively, R^{m} and Rⁿ form a 3-7 membered cycloalkyl or heterocycloalkyl group together with their co-connected carbon atom;
x is 0 or 1;
n and p are 0, 1 or 2;
q is 0 or 1;
X is O, S or NH; and,
A is O or S, and when A-R¹⁰ is OH, not all R² and R³ both are hydrogen (H).

9. The camptothecin compound or the pharmaceutically acceptable salt thereof according to claim 1, characterized in that it is a camptothecin compound as shown in the general formula (I-1) or a pharmaceutically acceptable salt thereof: 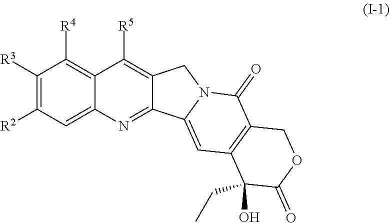
wherein:
R² and R³ are each independently H, D, F, Cl, CH₃, CD₃, CF₂H, CF₃, OH, SH, S(O)CH₃, S(O₂)CH₃, OCH₃ or SCH₃;
R⁴ is each independently H, D, F, Cl, CH₃;
alternatively, R² and R³ together with their co-connected carbon atom form a ring structure as follows: 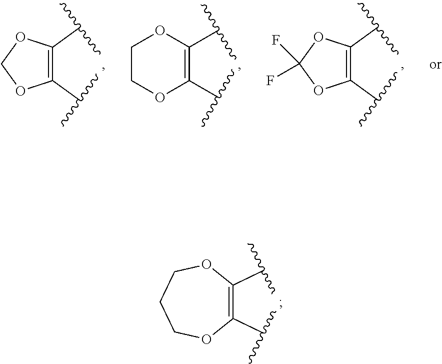 [SMILES: *C1=C(*)OCO1 ; *C1=C(*)OCCO1 ; *C1=C(*)OC(F)(F)O1 ; *C1=C(*)OCCCO1]
and,
R⁵ is

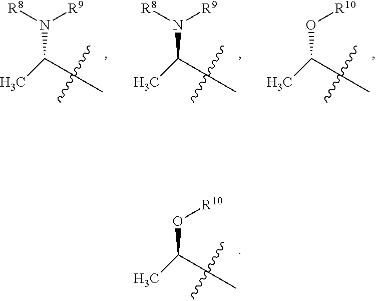

10. The camptothecin compound or the pharmaceutically acceptable salt thereof according to claim 1, characterized in that it is a camptothecin compound as shown in general formula (V) or the pharmaceutically acceptable salt therefore: 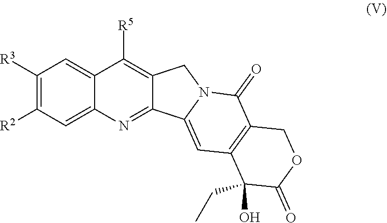
wherein:
R² and R³ are each independently H, D, F, Cl, CH₃, CD₃, CF₂H, CF₃, OH, SH, S(O)CH₃, S(O₂)CH₃, OCH₃ or SCH₃;
alternatively, R² and R³ together with their co-connected carbon atom form a ring structure as follows: 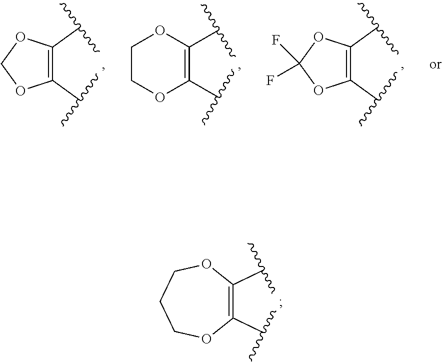 [SMILES: *C1=C(*)OCO1 ; *C1=C(*)OCCO1 ; *C1=C(*)OC(F)(F)O1 ; *C1=C(*)OCCCO1]
R⁵ is any one of the following structures: 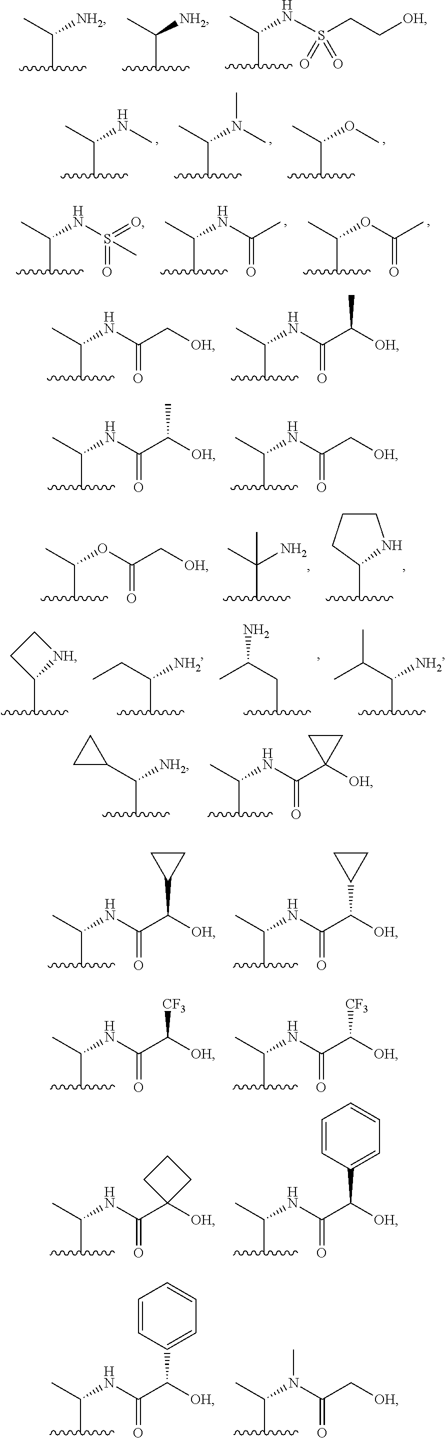 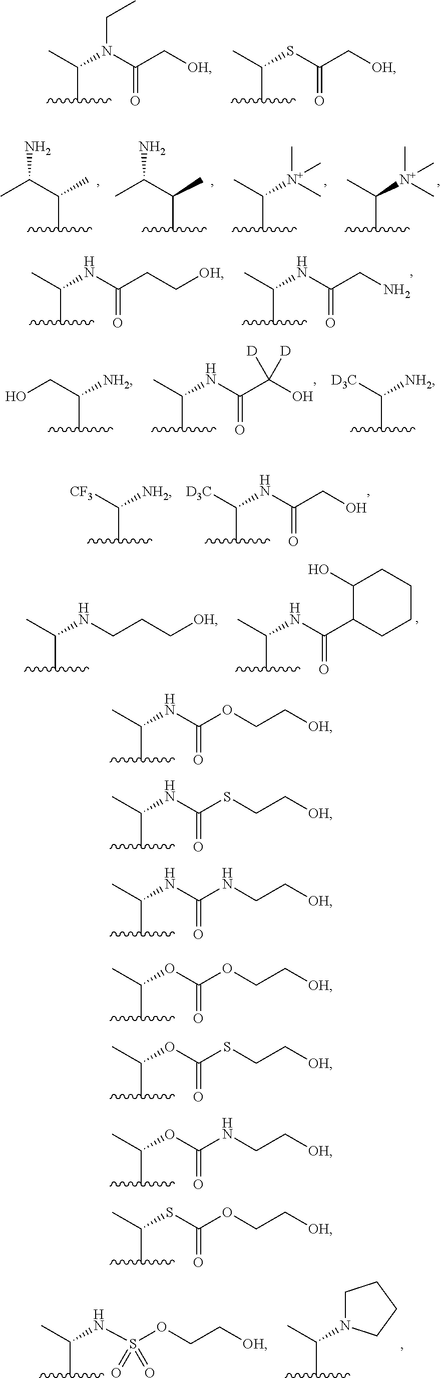 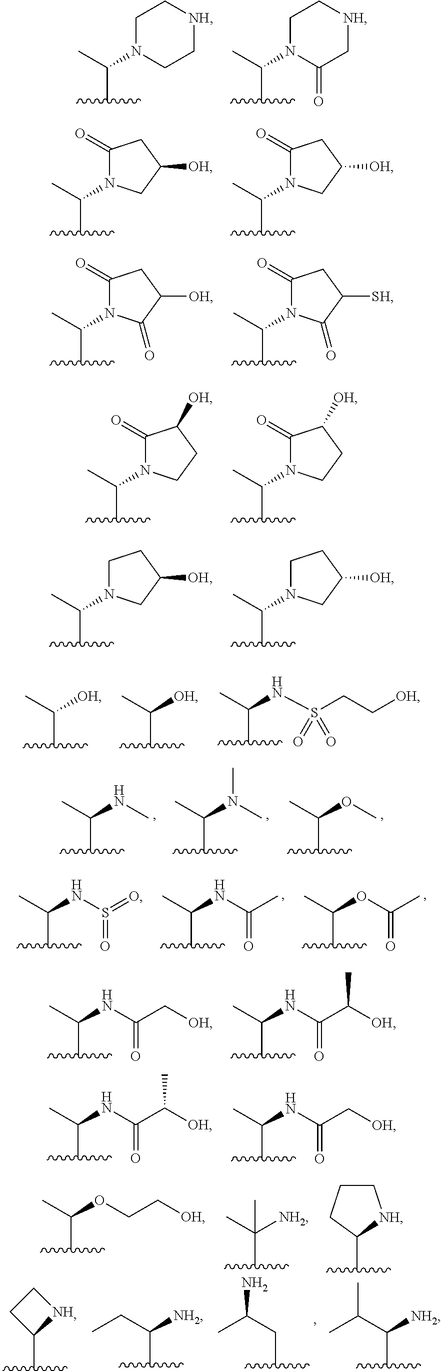 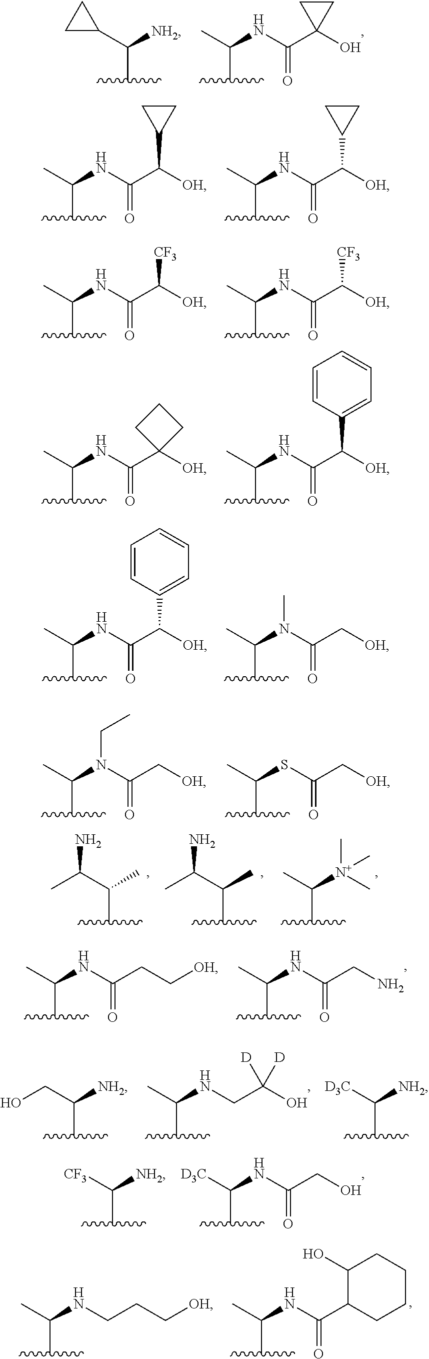 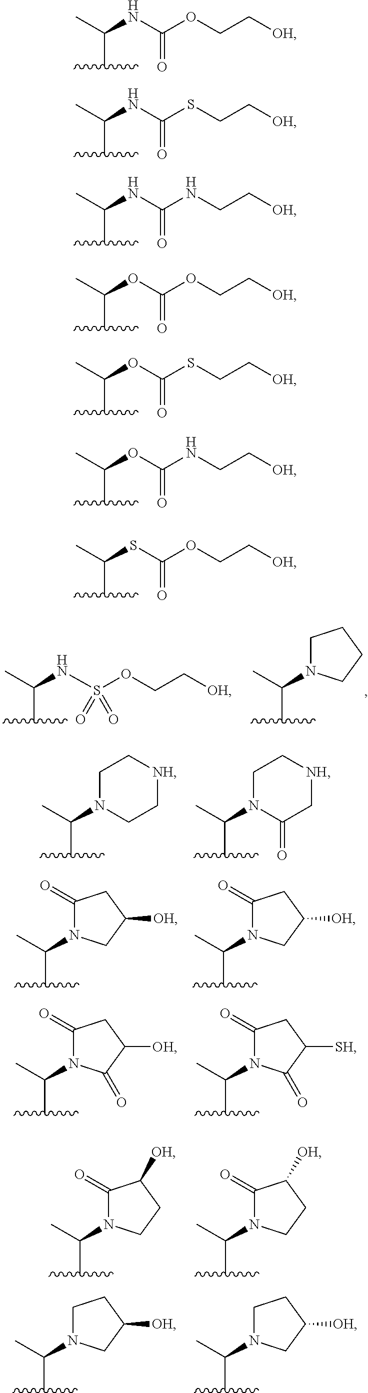 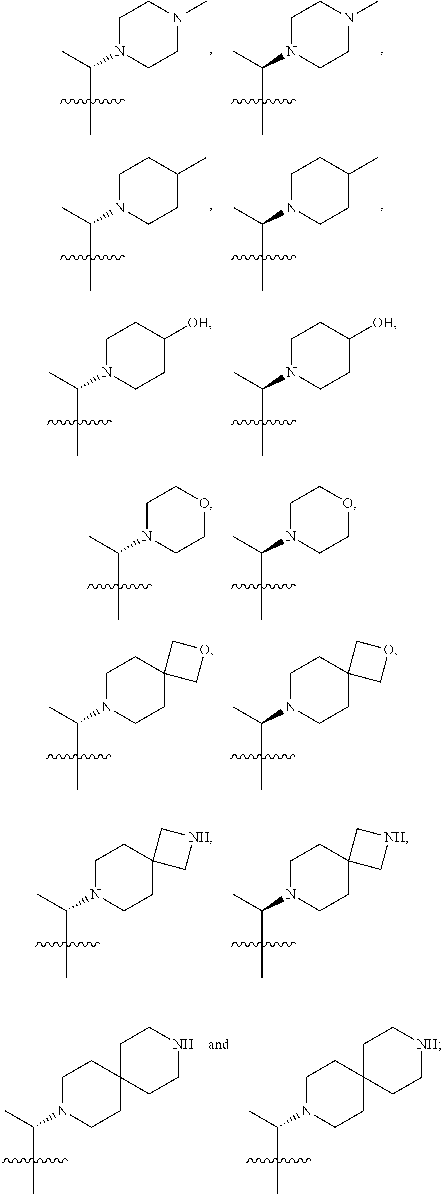
when R⁵ is selected from 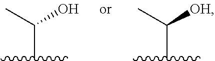
not all R² and R³ are hydrogen (H).

11. The camptothecin compound or the pharmaceutically acceptable salt thereof according to claim 1, characterized in that the compound is any one of the following compounds:

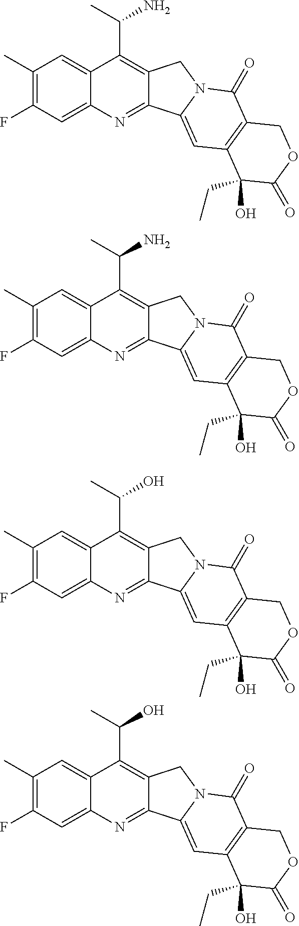 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@H](C)O ; CC[C@@]1(O)C(=O)OCc2c1cc1n(c2=O)Cc2c-1nc1cc(F)c(C)cc1c2[C@@H](C)O]

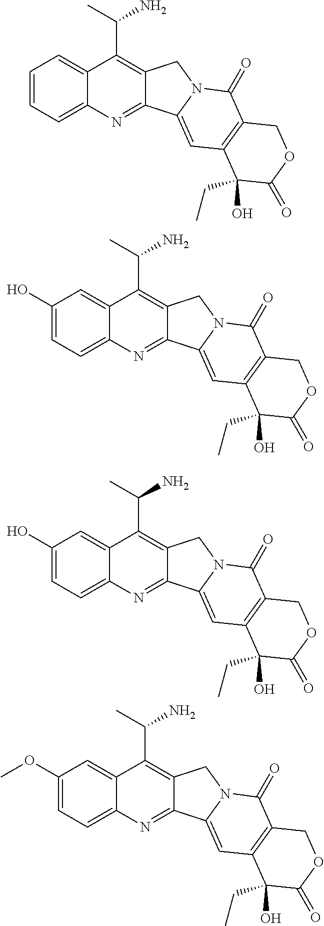

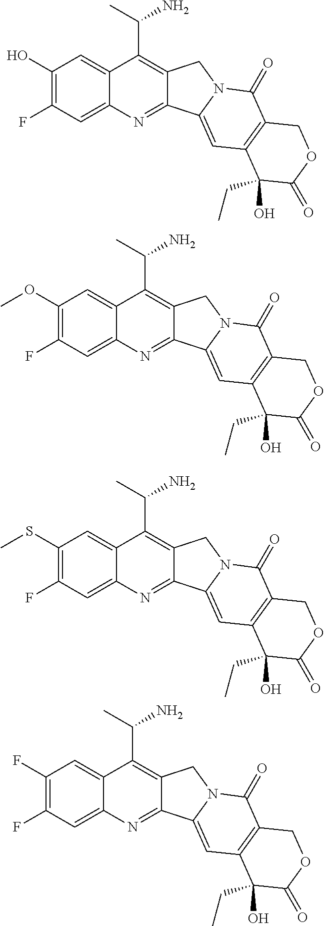 [SMILES: CC[C@@]1(O)C(=O)OCc2c1cc1n(c2=O)Cc2c-1nc1cc(F)c(O)cc1c2[C@H](C)N ; CC[C@@]1(O)C(=O)OCc2c1cc1n(c2=O)Cc2c-1nc1cc(F)c(OC)cc1c2[C@H](C)N ; CC[C@@]1(O)C(=O)OCc2c1cc1n(c2=O)Cc2c-1nc1cc(F)c(SC)cc1c2[C@H](C)N ; CC[C@@]1(O)C(=O)OCc2c1cc1n(c2=O)Cc2c-1nc1cc(F)c(F)cc1c2[C@H](C)N]

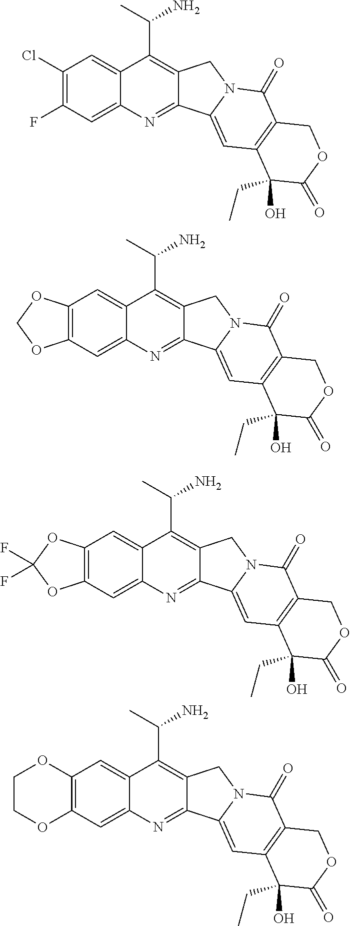

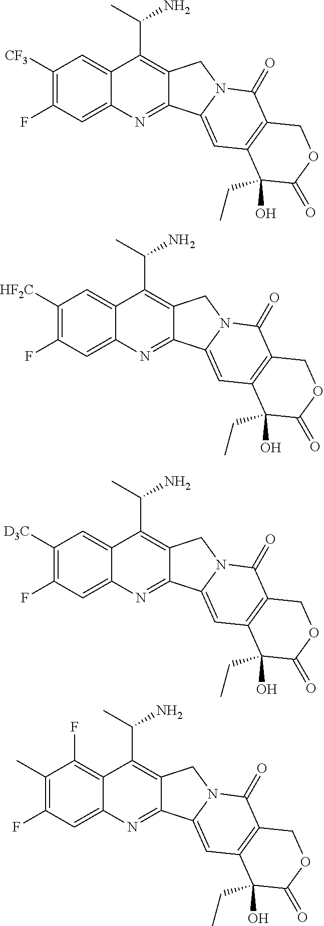

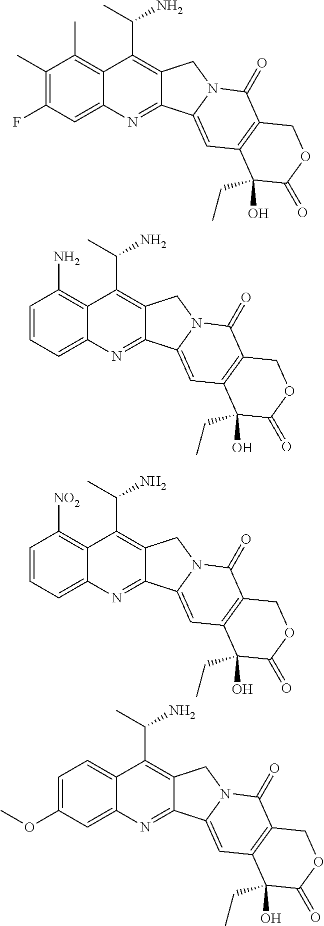

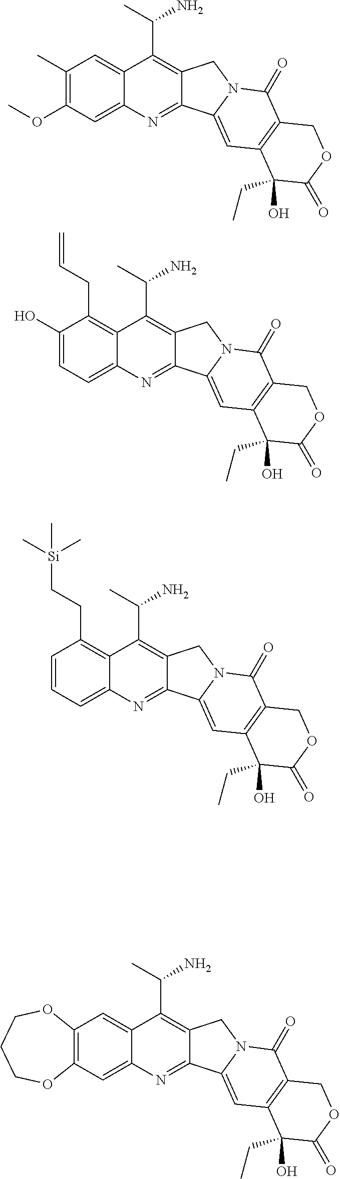 [SMILES: CC[C@@]1(O)C(=O)OCc2c1cc1n(c2=O)Cc2c-1nc1cc(OC)c(C)cc1c2[C@H](C)N ; C=CCc1c(O)ccc2nc3c(c([C@H](C)N)c12)Cn1c-3cc2c(c1=O)COC(=O)[C@]2(O)CC ; CC[C@@]1(O)C(=O)OCc2c1cc1n(c2=O)Cc2c-1nc1cccc(CC[Si](C)(C)C)c1c2[C@H](C)N ; CC[C@@]1(O)C(=O)OCc2c1cc1n(c2=O)Cc2c-1nc1cc3c(cc1c2[C@H](C)N)OCCCO3]

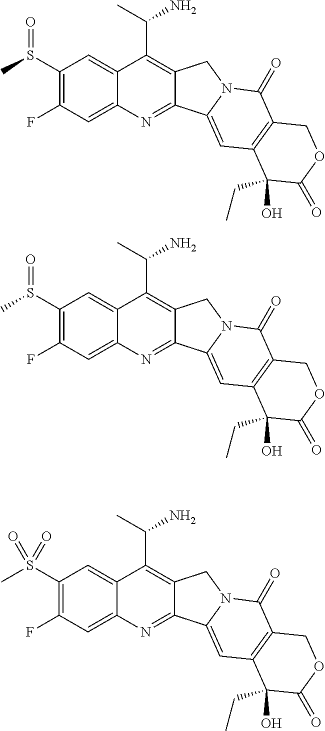 [SMILES: CC[C@@]1(O)C(=O)OCc2c1cc1n(c2=O)Cc2c-1nc1cc(F)c([S@@](C)=O)cc1c2[C@H](C)N ; CC[C@@]1(O)C(=O)OCc2c1cc1n(c2=O)Cc2c-1nc1cc(F)c([S@](C)=O)cc1c2[C@H](C)N ; CC[C@@]1(O)C(=O)OCc2c1cc1n(c2=O)Cc2c-1nc1cc(F)c(S(C)(=O)=O)cc1c2[C@H](C)N]

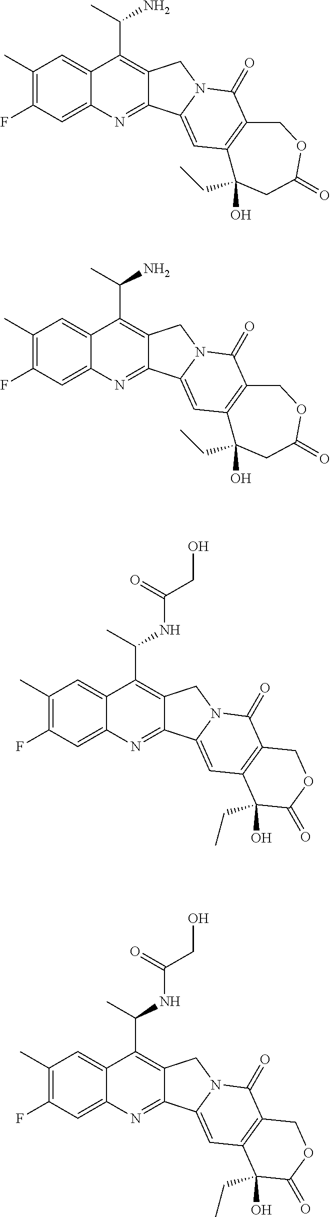 [SMILES: CC[C@@]1(O)CC(=O)OCc2c1cc1n(c2=O)Cc2c-1nc1cc(F)c(C)cc1c2[C@H](C)N ; CC[C@@]1(O)CC(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@H](C)NC(=O)CO ; CC[C@@]1(O)C(=O)OCc2c1cc1n(c2=O)Cc2c-1nc1cc(F)c(C)cc1c2[C@@H](C)NC(=O)CO]

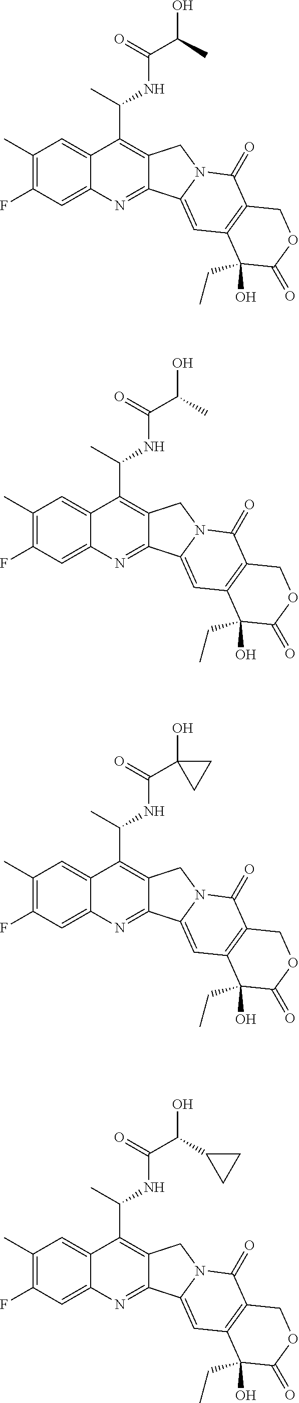 [SMILES: CC[C@@]1(O)C(=O)OCc2c1cc1n(c2=O)Cc2c-1nc1cc(F)c(C)cc1c2[C@H](C)NC(=O)[C@H](C)O ; CC[C@@]1(O)C(=O)OCc2c1cc1n(c2=O)Cc2c-1nc1cc(F)c(C)cc1c2[C@H](C)NC(=O)[C@@H](C)O ; CC[C@@]1(O)C(=O)OCc2c1cc1n(c2=O)Cc2c-1nc1cc(F)c(C)cc1c2[C@H](C)NC(=O)C1(O)CC1 ; CC[C@@]1(O)C(=O)OCc2c1cc1n(c2=O)Cc2c-1nc1cc(F)c(C)cc1c2[C@H](C)NC(=O)[C@H](O)C1CC1]

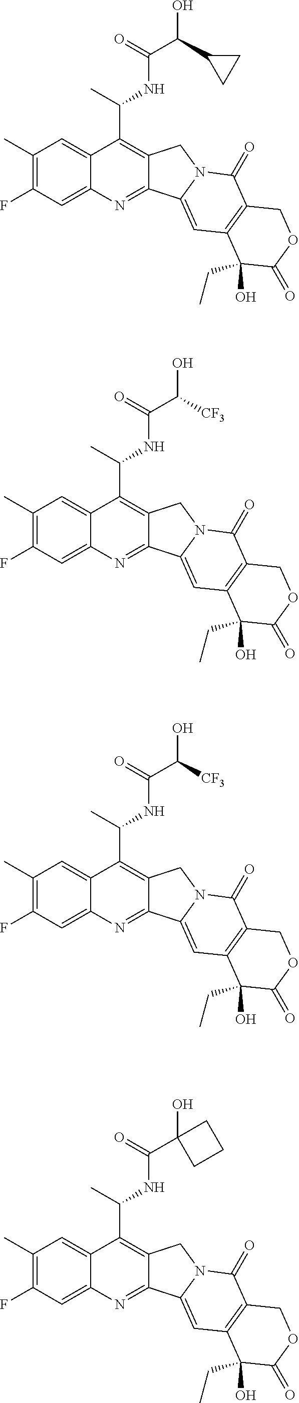 [SMILES: CC[C@@]1(O)C(=O)OCc2c1cc1n(c2=O)Cc2c-1nc1cc(F)c(C)cc1c2[C@H](C)NC(=O)[C@@H](O)C1CC1 ; CC[C@@]1(O)C(=O)OCc2c1cc1n(c2=O)Cc2c-1nc1cc(F)c(C)cc1c2[C@H](C)NC(=O)[C@H](O)C(F)(F)F ; CC[C@@]1(O)C(=O)OCc2c1cc1n(c2=O)Cc2c-1nc1cc(F)c(C)cc1c2[C@H](C)NC(=O)[C@@H](O)C(F)(F)F ; CC[C@@]1(O)C(=O)OCc2c1cc1n(c2=O)Cc2c-1nc1cc(F)c(C)cc1c2[C@H](C)NC(=O)C1(O)CCC1]

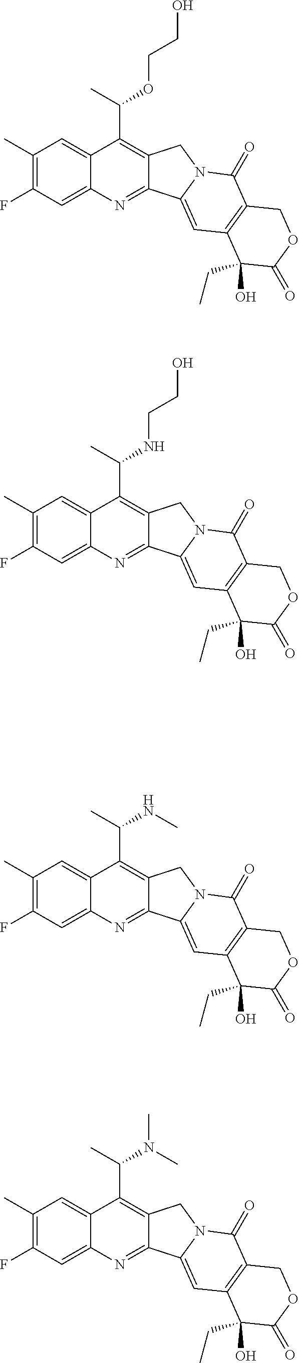 [SMILES: CC[C@@]1(O)C(=O)OCc2c1cc1n(c2=O)Cc2c-1nc1cc(F)c(C)cc1c2[C@H](C)OCCO ; CC[C@@]1(O)C(=O)OCc2c1cc1n(c2=O)Cc2c-1nc1cc(F)c(C)cc1c2[C@H](C)NCCO ; CC[C@@]1(O)C(=O)OCc2c1cc1n(c2=O)Cc2c-1nc1cc(F)c(C)cc1c2[C@H](C)NC ; CC[C@@]1(O)C(=O)OCc2c1cc1n(c2=O)Cc2c-1nc1cc(F)c(C)cc1c2[C@H](C)N(C)C]

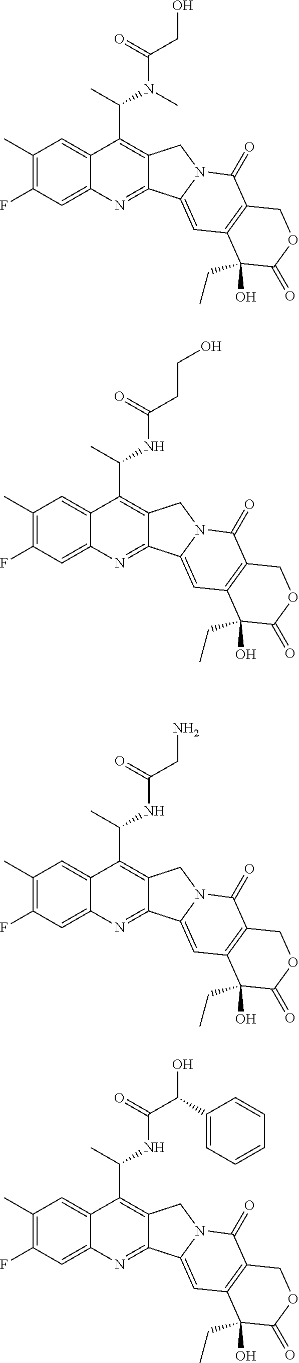

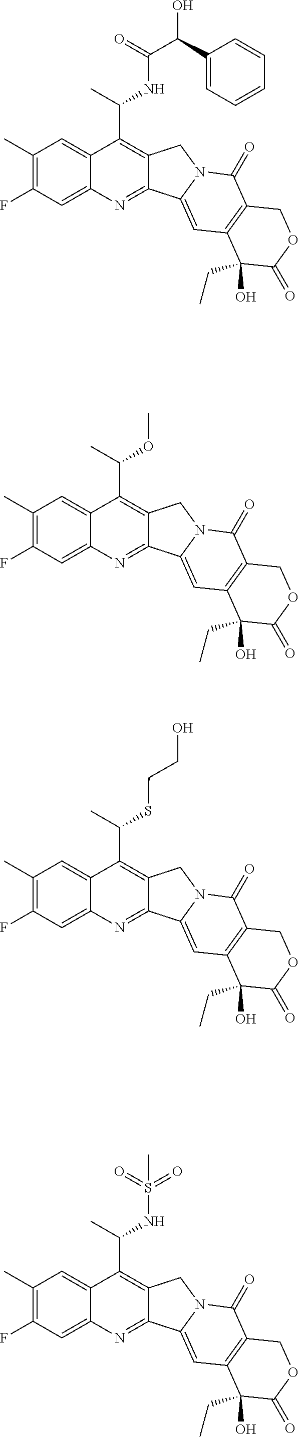 [SMILES: CC[C@@]1(O)C(=O)OCc2c1cc1n(c2=O)Cc2c-1nc1cc(F)c(C)cc1c2[C@H](C)NC(=O)[C@@H](O)c1ccccc1 ; CC[C@@]1(O)C(=O)OCc2c1cc1n(c2=O)Cc2c-1nc1cc(F)c(C)cc1c2[C@H](C)OC ; CC[C@@]1(O)C(=O)OCc2c1cc1n(c2=O)Cc2c-1nc1cc(F)c(C)cc1c2[C@H](C)SCCO ; CC[C@@]1(O)C(=O)OCc2c1cc1n(c2=O)Cc2c-1nc1cc(F)c(C)cc1c2[C@H](C)NS(C)(=O)=O]

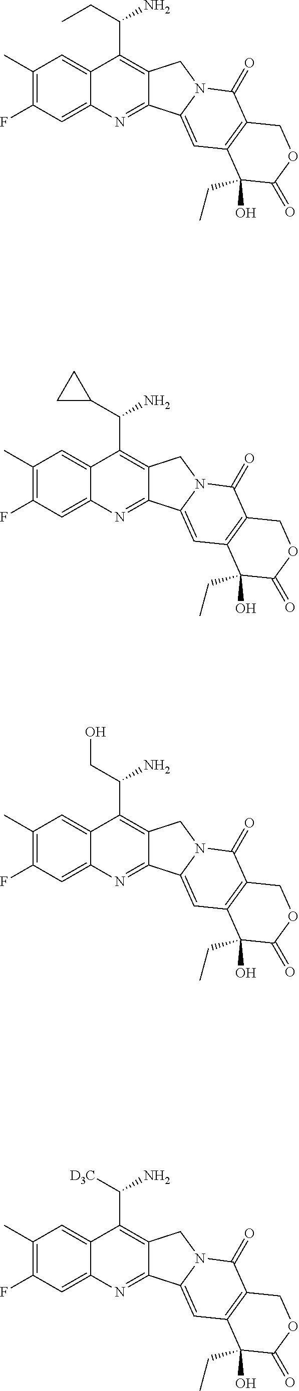 [SMILES: CC[C@H](N)c1c2c(nc3cc(F)c(C)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2[C@@H](N)C1CC1 ; CC[C@@]1(O)C(=O)OCc2c1cc1n(c2=O)Cc2c-1nc1cc(F)c(C)cc1c2[C@@H](N)CO ; [2H]C([2H])([2H])[C@H](N)c1c2c(nc3cc(F)c(C)cc13)-c1cc3c(c(=O)n1C2)COC(=O)[C@]3(O)CC]

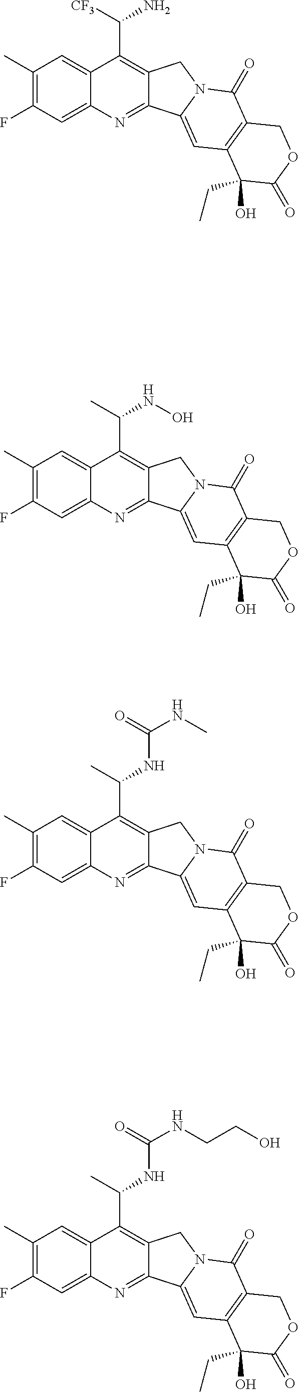 [SMILES: CC[C@@]1(O)C(=O)OCc2c1cc1n(c2=O)Cc2c-1nc1cc(F)c(C)cc1c2[C@@H](N)C(F)(F)F ; CC[C@@]1(O)C(=O)OCc2c1cc1n(c2=O)Cc2c-1nc1cc(F)c(C)cc1c2[C@H](C)NO ; CC[C@@]1(O)C(=O)OCc2c1cc1n(c2=O)Cc2c-1nc1cc(F)c(C)cc1c2[C@H](C)NC(=O)NC ; CC[C@@]1(O)C(=O)OCc2c1cc1n(c2=O)Cc2c-1nc1cc(F)c(C)cc1c2[C@H](C)NC(=O)NCCO]

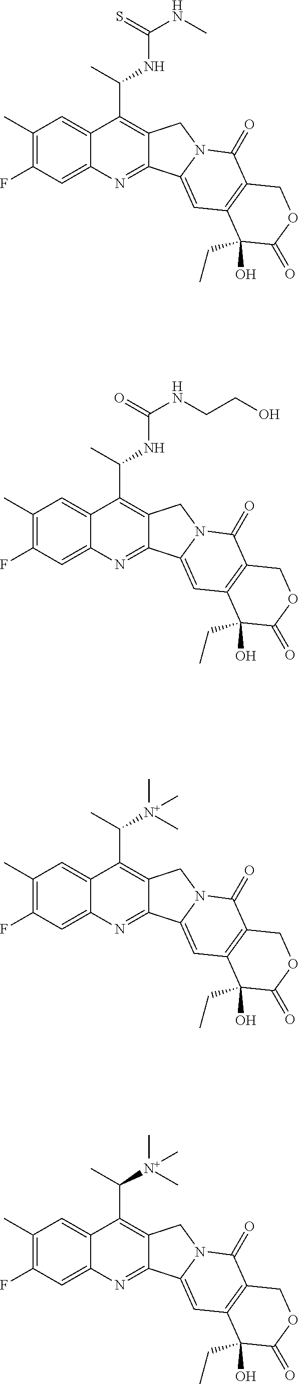 [SMILES: CC[C@@]1(O)C(=O)OCc2c1cc1n(c2=O)Cc2c-1nc1cc(F)c(C)cc1c2[C@H](C)NC(=S)NC ; CC[C@@]1(O)C(=O)OCc2c1cc1n(c2=O)Cc2c-1nc1cc(F)c(C)cc1c2[C@H](C)NC(=O)NCCO ; CC[C@@]1(O)C(=O)OCc2c1cc1n(c2=O)Cc2c-1nc1cc(F)c(C)cc1c2[C@H](C)[N+](C)(C)C ; CC[C@@]1(O)C(=O)OCc2c1cc1n(c2=O)Cc2c-1nc1cc(F)c(C)cc1c2[C@@H](C)[N+](C)(C)C]

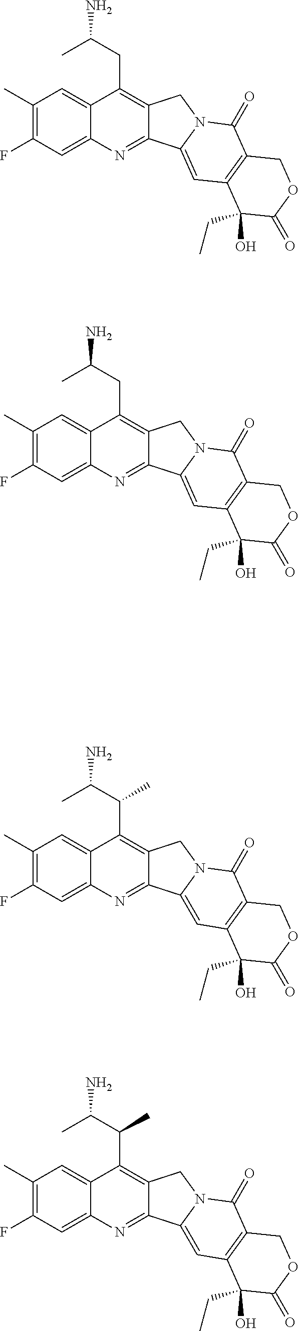 [SMILES: CC[C@@]1(O)C(=O)OCc2c1cc1n(c2=O)Cc2c-1nc1cc(F)c(C)cc1c2C[C@H](C)N ; CC[C@@]1(O)C(=O)OCc2c1cc1n(c2=O)Cc2c-1nc1cc(F)c(C)cc1c2C[C@@H](C)N ; CC[C@@]1(O)C(=O)OCc2c1cc1n(c2=O)Cc2c-1nc1cc(F)c(C)cc1c2[C@@H](C)[C@H](C)N ; CC[C@@]1(O)C(=O)OCc2c1cc1n(c2=O)Cc2c-1nc1cc(F)c(C)cc1c2[C@H](C)[C@H](C)N]

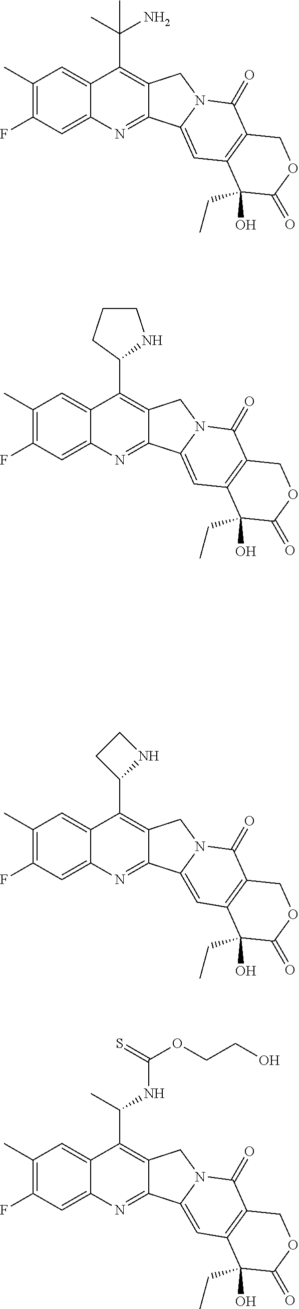 [SMILES: CC[C@@]1(O)C(=O)OCc2c1cc1n(c2=O)Cc2c-1nc1cc(F)c(C)cc1c2C(C)(C)N ; CC[C@@]1(O)C(=O)OCc2c1cc1n(c2=O)Cc2c-1nc1cc(F)c(C)cc1c2[C@@H]1CCCN1 ; CC[C@@]1(O)C(=O)OCc2c1cc1n(c2=O)Cc2c-1nc1cc(F)c(C)cc1c2[C@@H]1CCN1 ; CC[C@@]1(O)C(=O)OCc2c1cc1n(c2=O)Cc2c-1nc1cc(F)c(C)cc1c2[C@H](C)NC(=S)OCCO]

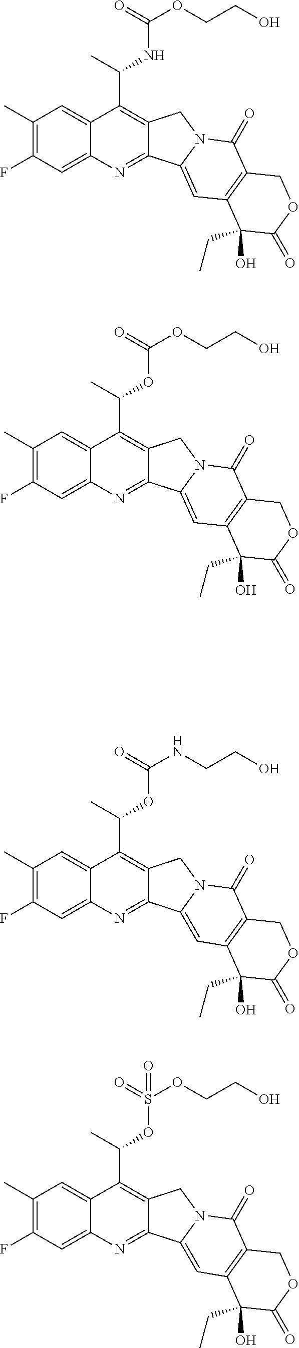 [SMILES: CC[C@@]1(O)C(=O)OCc2c1cc1n(c2=O)Cc2c-1nc1cc(F)c(C)cc1c2[C@H](C)NC(=O)OCCO ; CC[C@@]1(O)C(=O)OCc2c1cc1n(c2=O)Cc2c-1nc1cc(F)c(C)cc1c2[C@H](C)OC(=O)OCCO ; CC[C@@]1(O)C(=O)OCc2c1cc1n(c2=O)Cc2c-1nc1cc(F)c(C)cc1c2[C@H](C)OC(=O)NCCO ; CC[C@@]1(O)C(=O)OCc2c1cc1n(c2=O)Cc2c-1nc1cc(F)c(C)cc1c2[C@H](C)OS(=O)(=O)OCCO]

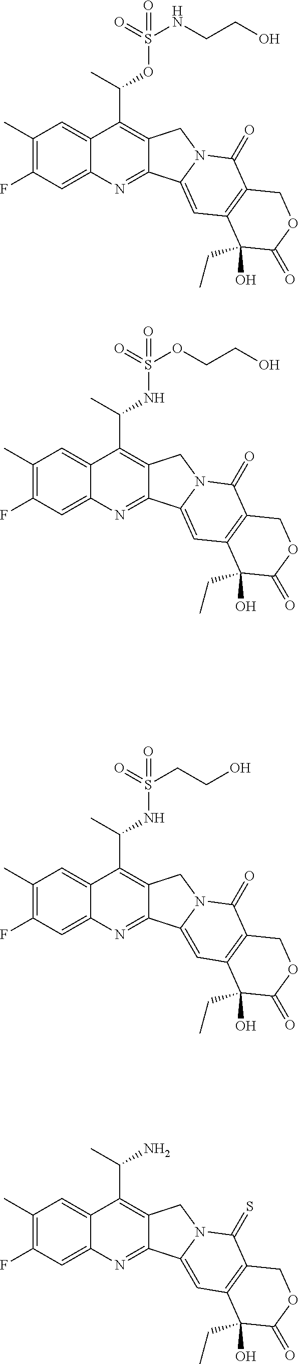 [SMILES: CC[C@@]1(O)C(=O)OCc2c1cc1n(c2=O)Cc2c-1nc1cc(F)c(C)cc1c2[C@H](C)OS(=O)(=O)NCCO ; CC[C@@]1(O)C(=O)OCc2c1cc1n(c2=O)Cc2c-1nc1cc(F)c(C)cc1c2[C@H](C)NS(=O)(=O)OCCO ; CC[C@@]1(O)C(=O)OCc2c1cc1n(c2=O)Cc2c-1nc1cc(F)c(C)cc1c2[C@H](C)NS(=O)(=O)CCO ; CC[C@@]1(O)C(=O)OCc2c1cc1n(c2=S)Cc2c-1nc1cc(F)c(C)cc1c2[C@H](C)N]

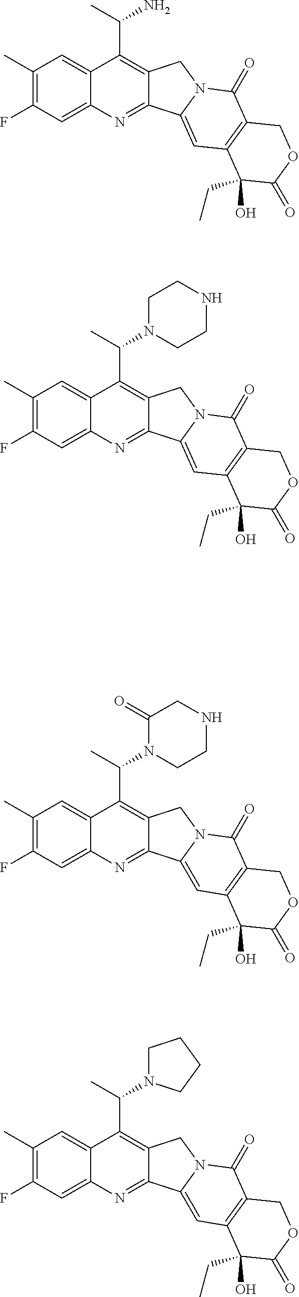 [SMILES: CC[C@@]1(O)C(=O)OCc2c1cc1n(c2=O)Cc2c-1nc1cc(F)c(C)cc1c2[C@H](C)N ; CC[C@@]1(O)C(=O)OCc2c1cc1n(c2=O)Cc2c-1nc1cc(F)c(C)cc1c2[C@H](C)N1CCNCC1 ; CC[C@@]1(O)C(=O)OCc2c1cc1n(c2=O)Cc2c-1nc1cc(F)c(C)cc1c2[C@H](C)N1CCNCC1=O ; CC[C@@]1(O)C(=O)OCc2c1cc1n(c2=O)Cc2c-1nc1cc(F)c(C)cc1c2[C@H](C)N1CCCC1]

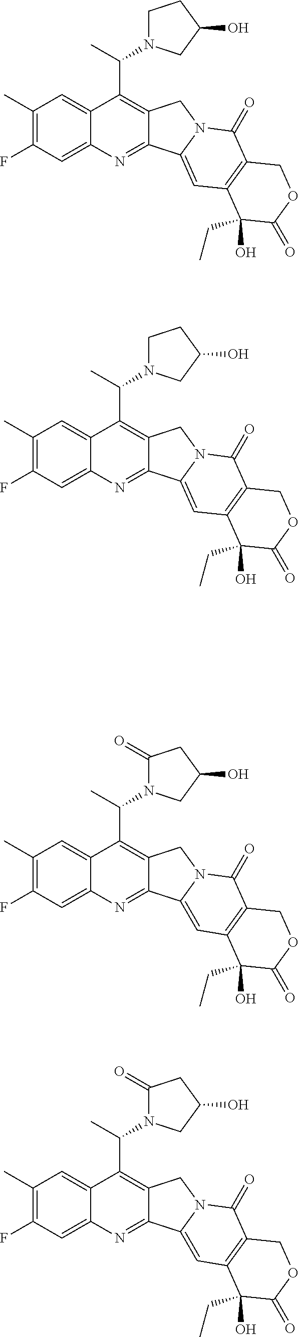 [SMILES: CC[C@@]1(O)C(=O)OCc2c1cc1n(c2=O)Cc2c-1nc1cc(F)c(C)cc1c2[C@H](C)N1CC[C@@H](O)C1 ; CC[C@@]1(O)C(=O)OCc2c1cc1n(c2=O)Cc2c-1nc1cc(F)c(C)cc1c2[C@H](C)N1CC[C@H](O)C1 ; CC[C@@]1(O)C(=O)OCc2c1cc1n(c2=O)Cc2c-1nc1cc(F)c(C)cc1c2[C@H](C)N1C[C@H](O)CC1=O ; CC[C@@]1(O)C(=O)OCc2c1cc1n(c2=O)Cc2c-1nc1cc(F)c(C)cc1c2[C@H](C)N1C[C@@H](O)CC1=O]

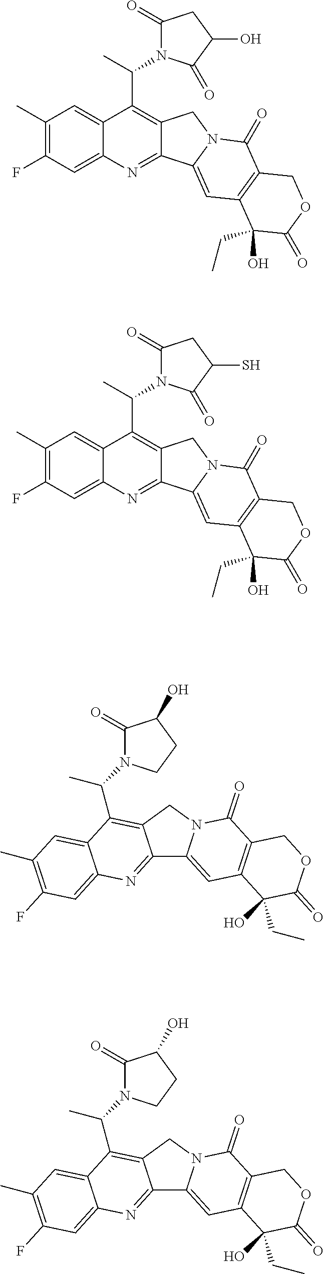 [SMILES: CC[C@@]1(O)C(=O)OCc2c1cc1n(c2=O)Cc2c-1nc1cc(F)c(C)cc1c2[C@H](C)N1C(=O)CC(O)C1=O ; CC[C@@]1(O)C(=O)OCc2c1cc1n(c2=O)Cc2c-1nc1cc(F)c(C)cc1c2[C@H](C)N1C(=O)CC(S)C1=O ; CC[C@@]1(O)C(=O)OCc2c1cc1n(c2=O)Cc2c-1nc1cc(F)c(C)cc1c2[C@H](C)N1CC[C@H](O)C1=O ; CC[C@@]1(O)C(=O)OCc2c1cc1n(c2=O)Cc2c-1nc1cc(F)c(C)cc1c2[C@H](C)N1CC[C@@H](O)C1=O]

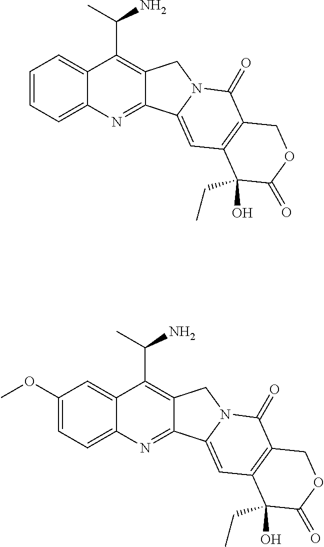 [SMILES: CC[C@@]1(O)C(=O)OCc2c1cc1n(c2=O)Cc2c-1nc1ccccc1c2[C@@H](C)N ; CC[C@@]1(O)C(=O)OCc2c1cc1n(c2=O)Cc2c-1nc1ccc(OC)cc1c2[C@@H](C)N]

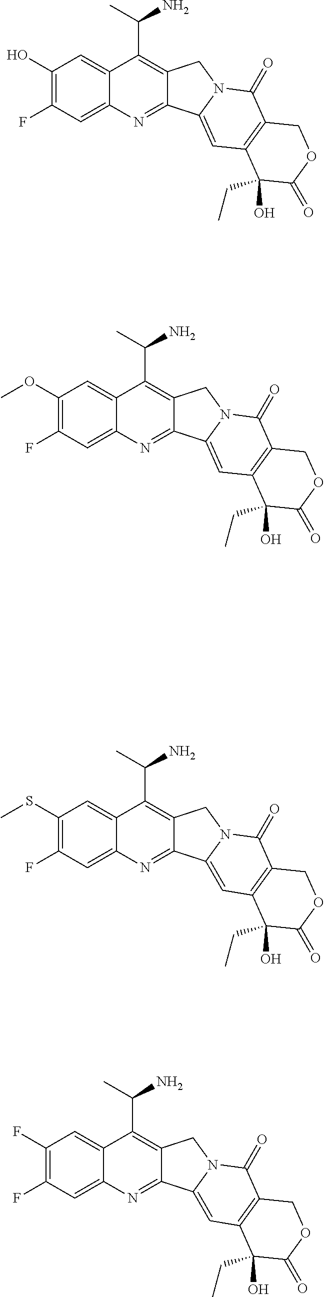 [SMILES: CC[C@@]1(O)C(=O)OCc2c1cc1n(c2=O)Cc2c-1nc1cc(F)c(O)cc1c2[C@@H](C)N ; CC[C@@]1(O)C(=O)OCc2c1cc1n(c2=O)Cc2c-1nc1cc(F)c(OC)cc1c2[C@@H](C)N ; CC[C@@]1(O)C(=O)OCc2c1cc1n(c2=O)Cc2c-1nc1cc(F)c(SC)cc1c2[C@@H](C)N ; CC[C@@]1(O)C(=O)OCc2c1cc1n(c2=O)Cc2c-1nc1cc(F)c(F)cc1c2[C@@H](C)N]

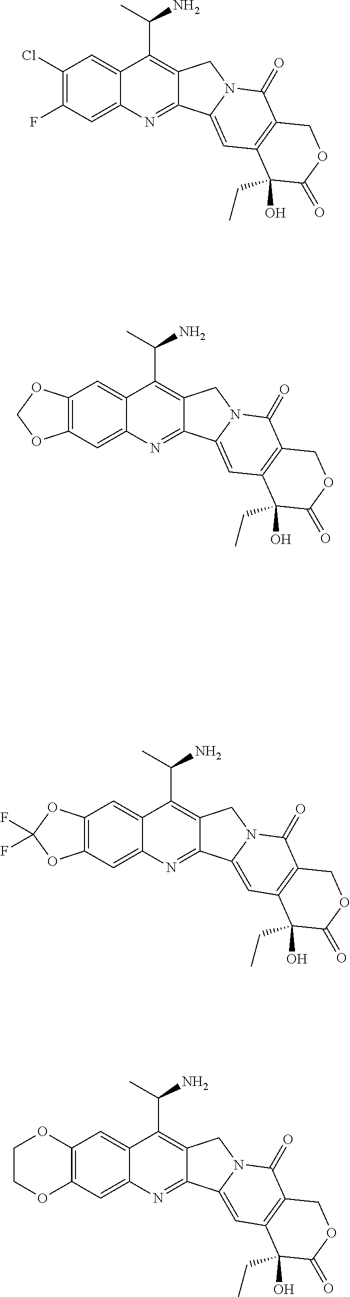 [SMILES: CC[C@@]1(O)C(=O)OCc2c1cc1n(c2=O)Cc2c-1nc1cc(F)c(Cl)cc1c2[C@@H](C)N ; CC[C@@]1(O)C(=O)OCc2c1cc1n(c2=O)Cc2c-1nc1cc3c(cc1c2[C@@H](C)N)OCO3 ; CC[C@@]1(O)C(=O)OCc2c1cc1n(c2=O)Cc2c-1nc1cc3c(cc1c2[C@@H](C)N)OC(F)(F)O3 ; CC[C@@]1(O)C(=O)OCc2c1cc1n(c2=O)Cc2c-1nc1cc3c(cc1c2[C@@H](C)N)OCCO3]

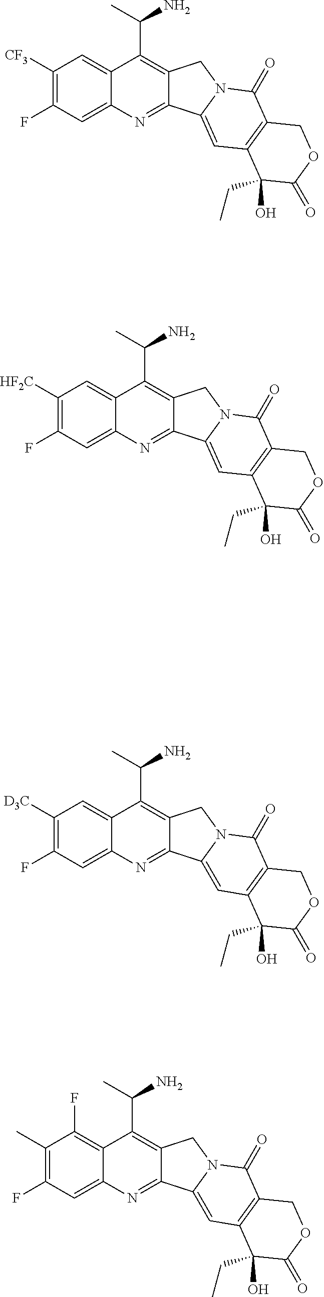

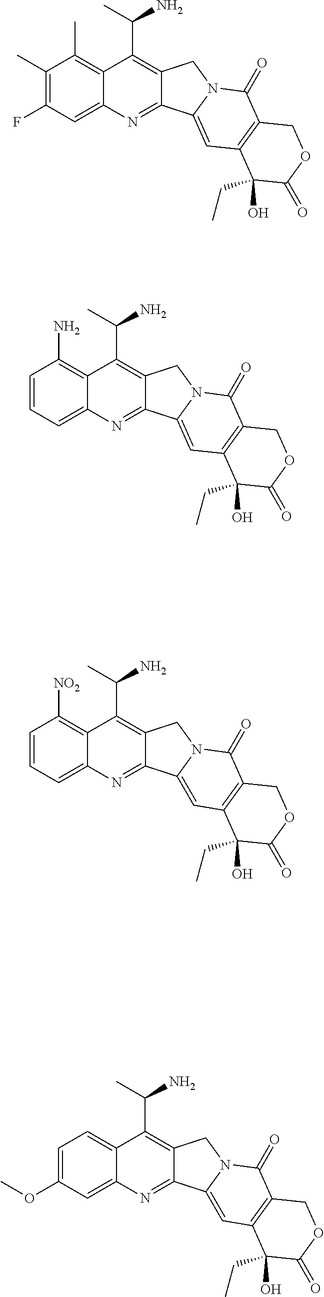 [SMILES: CC[C@@]1(O)C(=O)OCc2c1cc1n(c2=O)Cc2c-1nc1cc(F)c(C)c(C)c1c2[C@@H](C)N ; CC[C@@]1(O)C(=O)OCc2c1cc1n(c2=O)Cc2c-1nc1cccc(N)c1c2[C@@H](C)N ; CC[C@@]1(O)C(=O)OCc2c1cc1n(c2=O)Cc2c-1nc1cccc([N+](=O)[O-])c1c2[C@@H](C)N ; CC[C@@]1(O)C(=O)OCc2c1cc1n(c2=O)Cc2c-1nc1cc(OC)ccc1c2[C@@H](C)N]

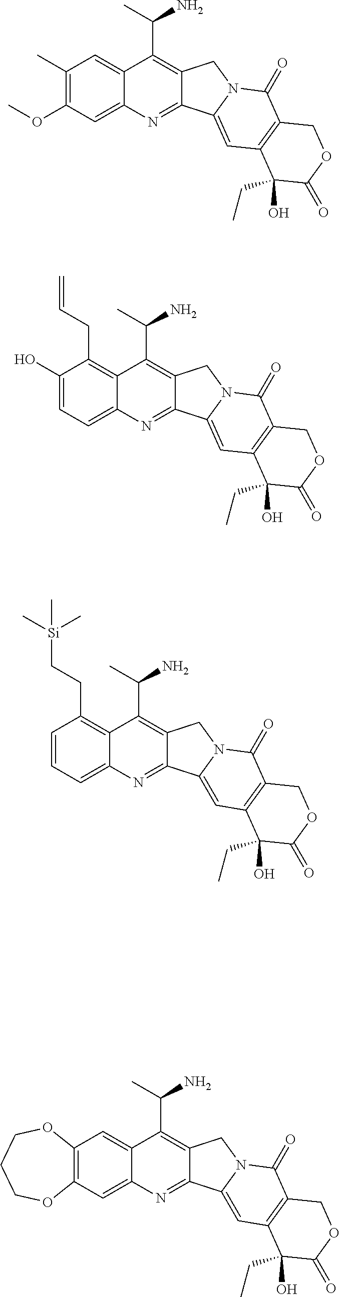 [SMILES: CC[C@@]1(O)C(=O)OCc2c1cc1n(c2=O)Cc2c-1nc1cc(OC)c(C)cc1c2[C@@H](C)N ; C=CCc1c(O)ccc2nc3c(c([C@@H](C)N)c12)Cn1c-3cc2c(c1=O)COC(=O)[C@]2(O)CC ; CC[C@@]1(O)C(=O)OCc2c1cc1n(c2=O)Cc2c-1nc1cccc(CC[Si](C)(C)C)c1c2[C@@H](C)N ; CC[C@@]1(O)C(=O)OCc2c1cc1n(c2=O)Cc2c-1nc1cc3c(cc1c2[C@@H](C)N)OCCCO3]

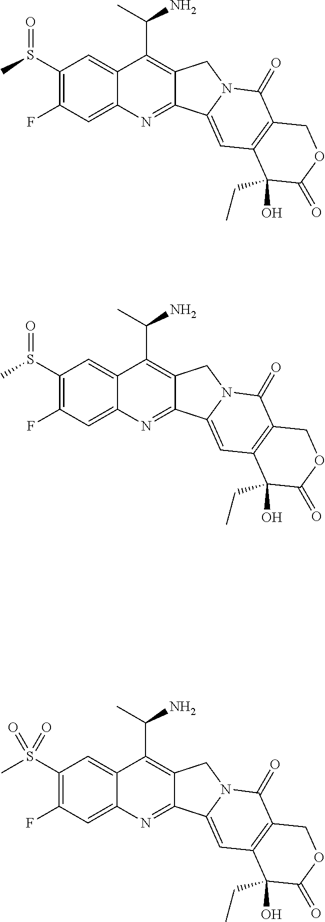 [SMILES: CC[C@@]1(O)C(=O)OCc2c1cc1n(c2=O)Cc2c-1nc1cc(F)c([S@@](C)=O)cc1c2[C@@H](C)N ; CC[C@@]1(O)C(=O)OCc2c1cc1n(c2=O)Cc2c-1nc1cc(F)c([S@](C)=O)cc1c2[C@@H](C)N ; CC[C@@]1(O)C(=O)OCc2c1cc1n(c2=O)Cc2c-1nc1cc(F)c(S(C)(=O)=O)cc1c2[C@@H](C)N]

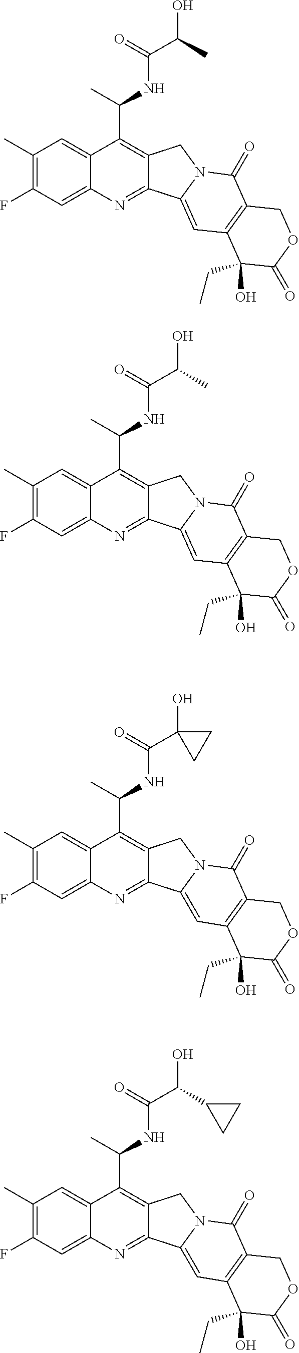

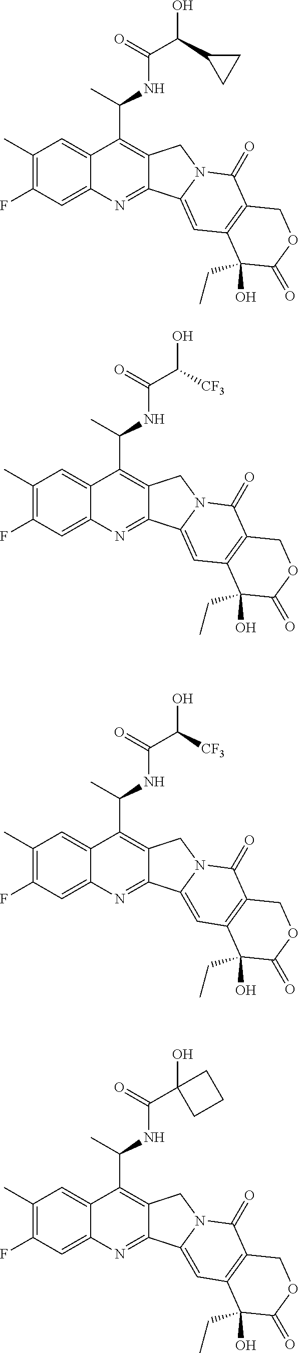

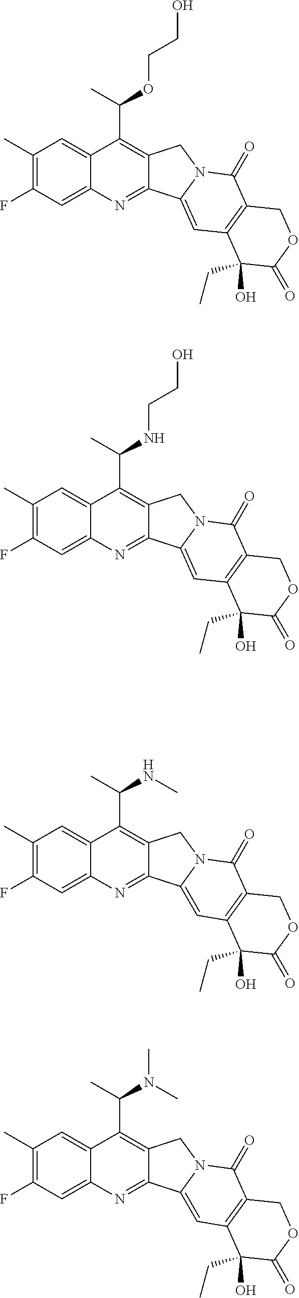 [SMILES: CC[C@@]1(O)C(=O)OCc2c1cc1n(c2=O)Cc2c-1nc1cc(F)c(C)cc1c2[C@@H](C)OCCO ; CC[C@@]1(O)C(=O)OCc2c1cc1n(c2=O)Cc2c-1nc1cc(F)c(C)cc1c2[C@@H](C)NCCO ; CC[C@@]1(O)C(=O)OCc2c1cc1n(c2=O)Cc2c-1nc1cc(F)c(C)cc1c2[C@@H](C)NC ; CC[C@@]1(O)C(=O)OCc2c1cc1n(c2=O)Cc2c-1nc1cc(F)c(C)cc1c2[C@@H](C)N(C)C]

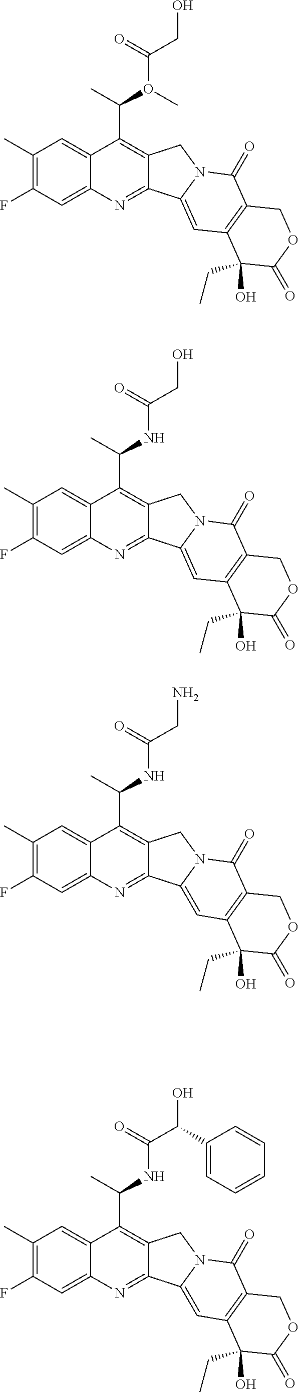

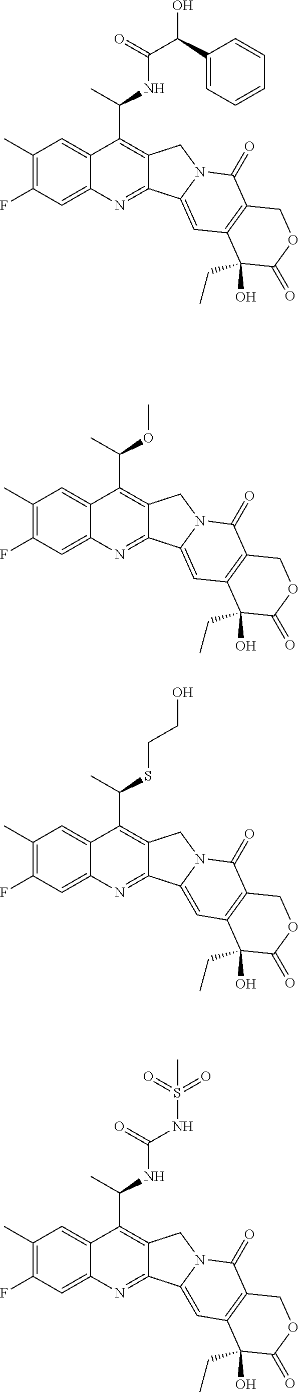

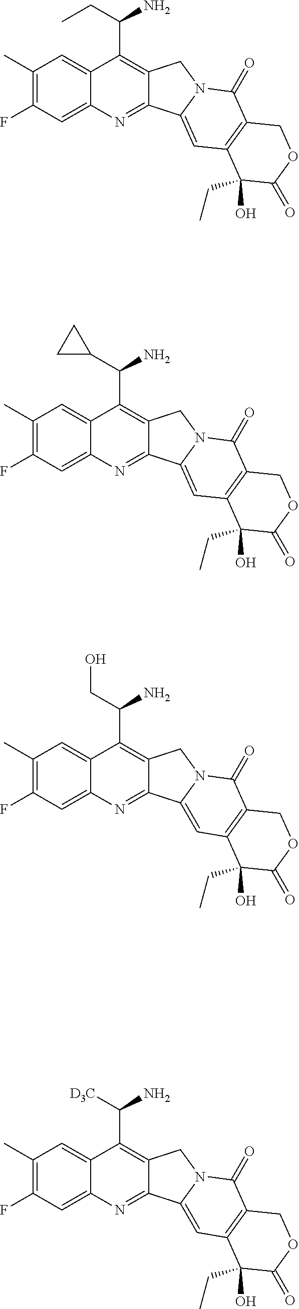 [SMILES: CC[C@@H](N)c1c2c(nc3cc(F)c(C)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2[C@H](N)C1CC1 ; CC[C@@]1(O)C(=O)OCc2c1cc1n(c2=O)Cc2c-1nc1cc(F)c(C)cc1c2[C@H](N)CO ; [2H]C([2H])([2H])[C@@H](N)c1c2c(nc3cc(F)c(C)cc13)-c1cc3c(c(=O)n1C2)COC(=O)[C@]3(O)CC]

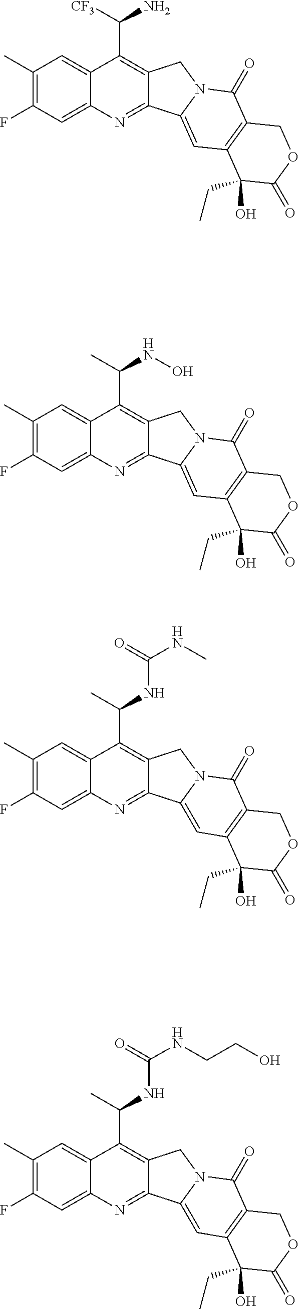 [SMILES: CC[C@@]1(O)C(=O)OCc2c1cc1n(c2=O)Cc2c-1nc1cc(F)c(C)cc1c2[C@H](N)C(F)(F)F ; CC[C@@]1(O)C(=O)OCc2c1cc1n(c2=O)Cc2c-1nc1cc(F)c(C)cc1c2[C@@H](C)NO ; CC[C@@]1(O)C(=O)OCc2c1cc1n(c2=O)Cc2c-1nc1cc(F)c(C)cc1c2[C@@H](C)NC(=O)NC ; CC[C@@]1(O)C(=O)OCc2c1cc1n(c2=O)Cc2c-1nc1cc(F)c(C)cc1c2[C@@H](C)NC(=O)NCCO]

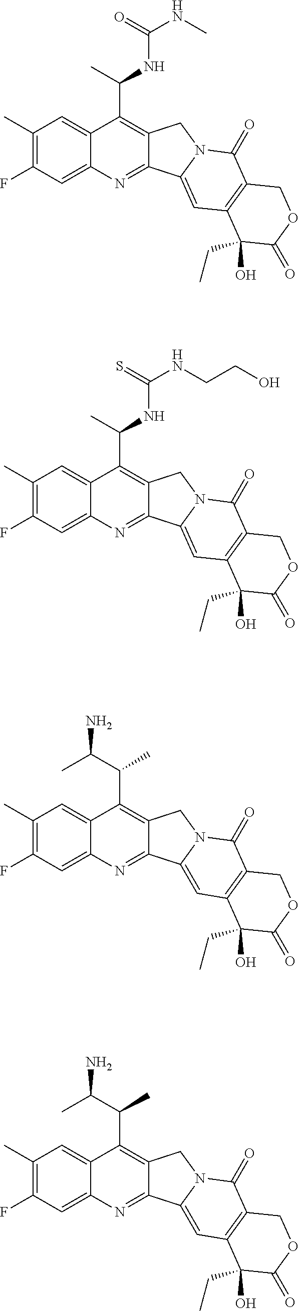 [SMILES: CC[C@@]1(O)C(=O)OCc2c1cc1n(c2=O)Cc2c-1nc1cc(F)c(C)cc1c2[C@@H](C)NC(=O)NC ; CC[C@@]1(O)C(=O)OCc2c1cc1n(c2=O)Cc2c-1nc1cc(F)c(C)cc1c2[C@@H](C)NC(=S)NCCO ; CC[C@@]1(O)C(=O)OCc2c1cc1n(c2=O)Cc2c-1nc1cc(F)c(C)cc1c2[C@@H](C)[C@@H](C)N ; CC[C@@]1(O)C(=O)OCc2c1cc1n(c2=O)Cc2c-1nc1cc(F)c(C)cc1c2[C@H](C)[C@@H](C)N]

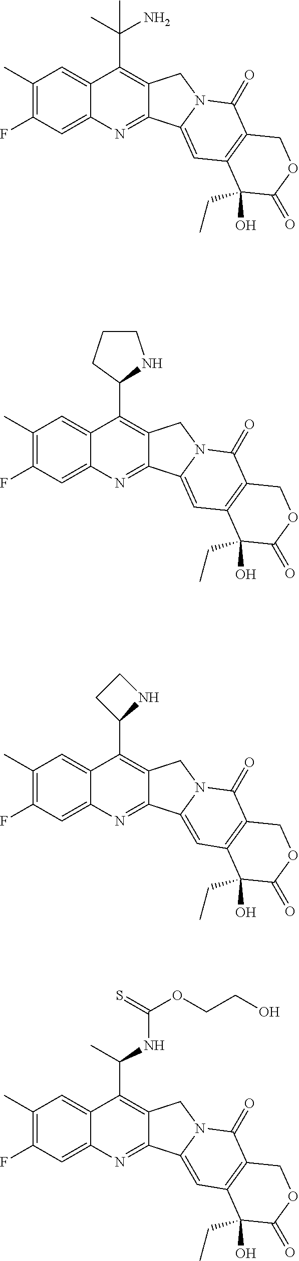 [SMILES: CC[C@@]1(O)C(=O)OCc2c1cc1n(c2=O)Cc2c-1nc1cc(F)c(C)cc1c2C(C)(C)N ; CC[C@@]1(O)C(=O)OCc2c1cc1n(c2=O)Cc2c-1nc1cc(F)c(C)cc1c2[C@H]1CCCN1 ; CC[C@@]1(O)C(=O)OCc2c1cc1n(c2=O)Cc2c-1nc1cc(F)c(C)cc1c2[C@H]1CCN1 ; CC[C@@]1(O)C(=O)OCc2c1cc1n(c2=O)Cc2c-1nc1cc(F)c(C)cc1c2[C@@H](C)NC(=S)OCCO]

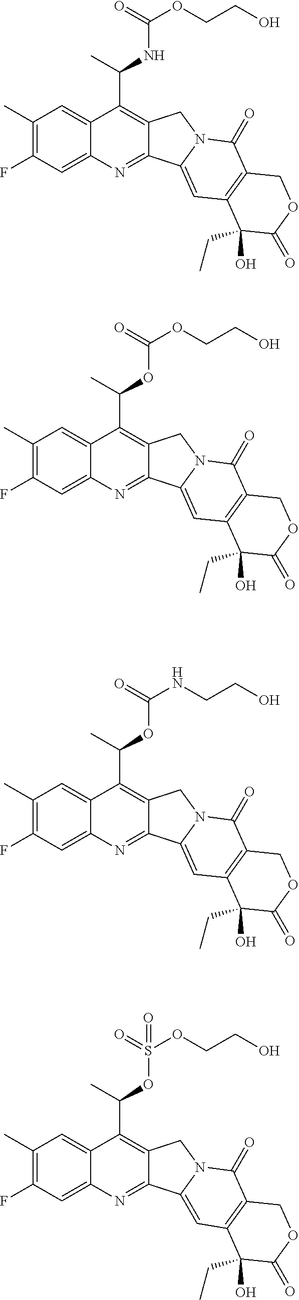 [SMILES: CC[C@@]1(O)C(=O)OCc2c1cc1n(c2=O)Cc2c-1nc1cc(F)c(C)cc1c2[C@@H](C)NC(=O)OCCO ; CC[C@@]1(O)C(=O)OCc2c1cc1n(c2=O)Cc2c-1nc1cc(F)c(C)cc1c2[C@@H](C)OC(=O)OCCO ; CC[C@@]1(O)C(=O)OCc2c1cc1n(c2=O)Cc2c-1nc1cc(F)c(C)cc1c2[C@@H](C)OC(=O)NCCO ; CC[C@@]1(O)C(=O)OCc2c1cc1n(c2=O)Cc2c-1nc1cc(F)c(C)cc1c2[C@@H](C)OS(=O)(=O)OCCO]

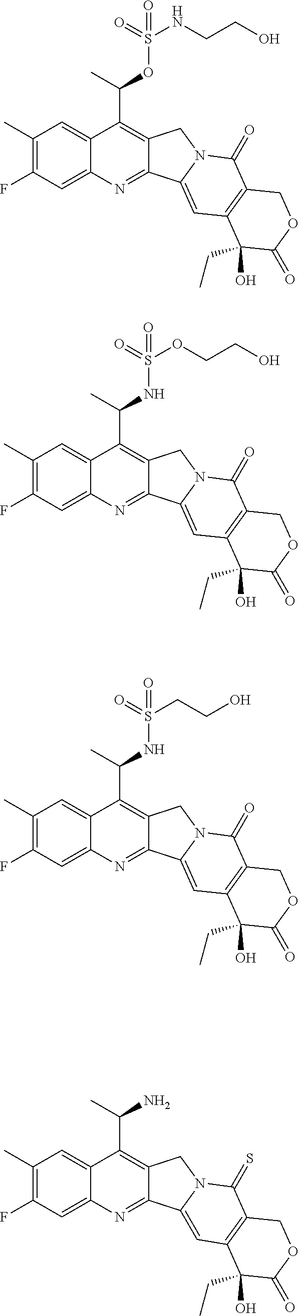 [SMILES: CC[C@@]1(O)C(=O)OCc2c1cc1n(c2=O)Cc2c-1nc1cc(F)c(C)cc1c2[C@@H](C)OS(=O)(=O)NCCO ; CC[C@@]1(O)C(=O)OCc2c1cc1n(c2=O)Cc2c-1nc1cc(F)c(C)cc1c2[C@@H](C)NS(=O)(=O)OCCO ; CC[C@@]1(O)C(=O)OCc2c1cc1n(c2=O)Cc2c-1nc1cc(F)c(C)cc1c2[C@@H](C)NS(=O)(=O)CCO ; CC[C@@]1(O)C(=O)OCc2c1cc1n(c2=S)Cc2c-1nc1cc(F)c(C)cc1c2[C@@H](C)N]

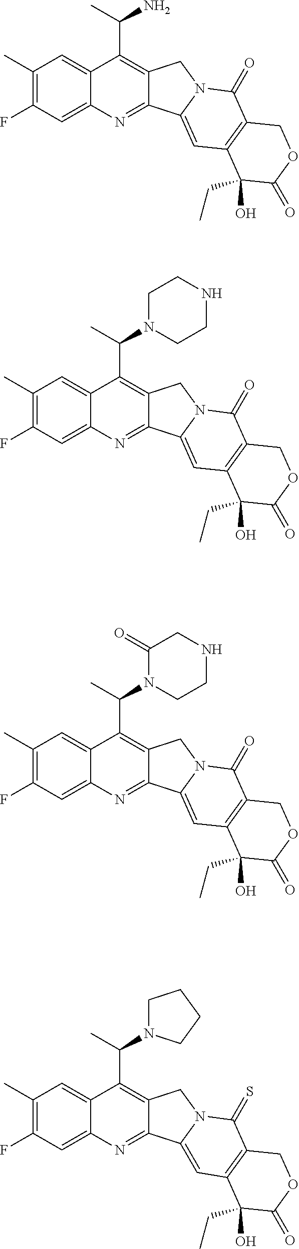 [SMILES: CC[C@@]1(O)C(=O)OCc2c1cc1n(c2=O)Cc2c-1nc1cc(F)c(C)cc1c2[C@@H](C)N ; CC[C@@]1(O)C(=O)OCc2c1cc1n(c2=O)Cc2c-1nc1cc(F)c(C)cc1c2[C@@H](C)N1CCNCC1 ; CC[C@@]1(O)C(=O)OCc2c1cc1n(c2=O)Cc2c-1nc1cc(F)c(C)cc1c2[C@@H](C)N1CCNCC1=O ; CC[C@@]1(O)C(=O)OCc2c1cc1n(c2=S)Cc2c-1nc1cc(F)c(C)cc1c2[C@@H](C)N1CCCC1]

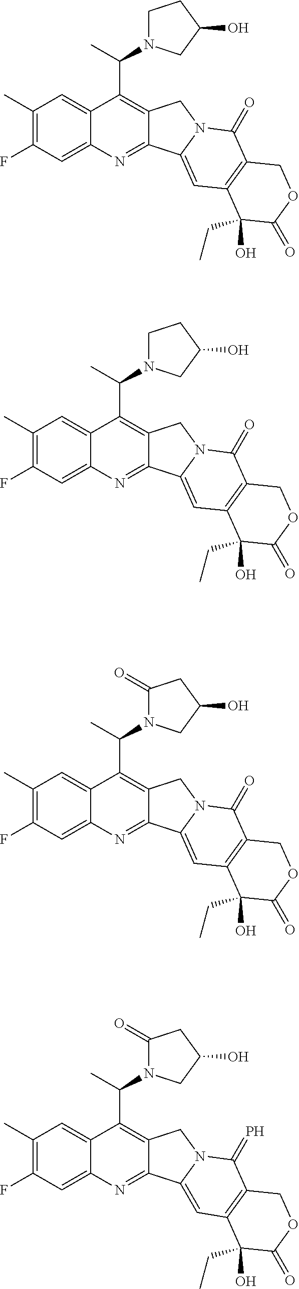

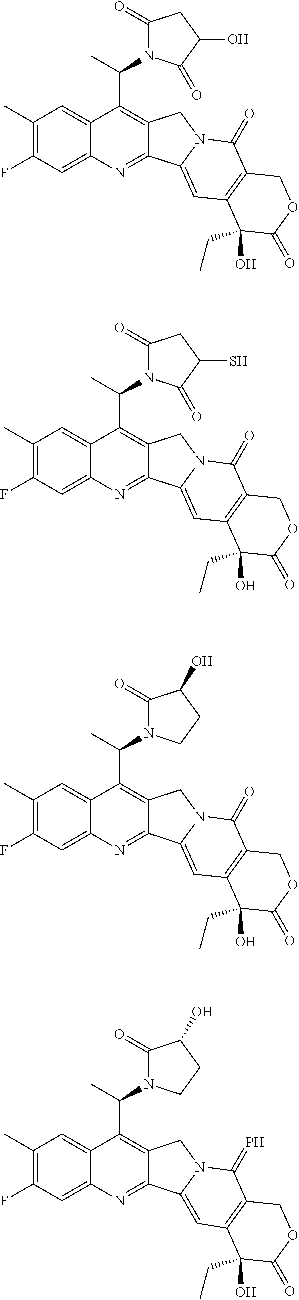

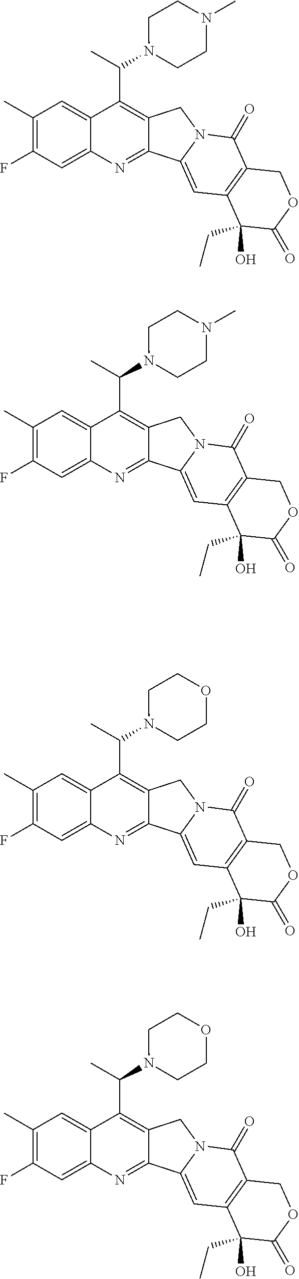

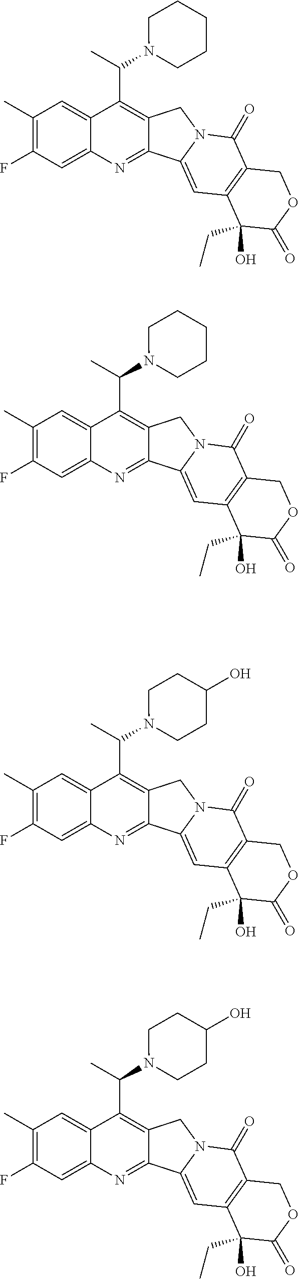

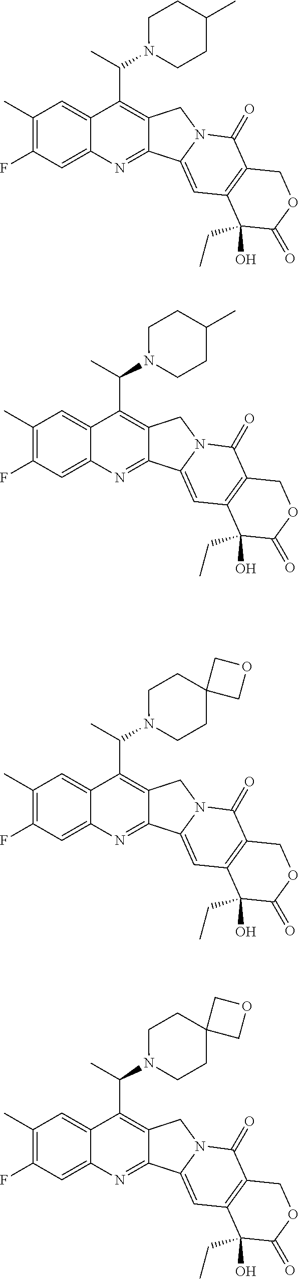

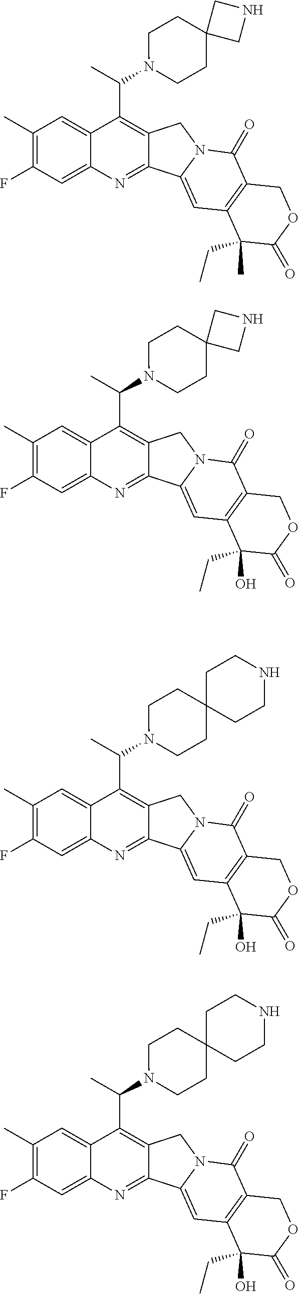

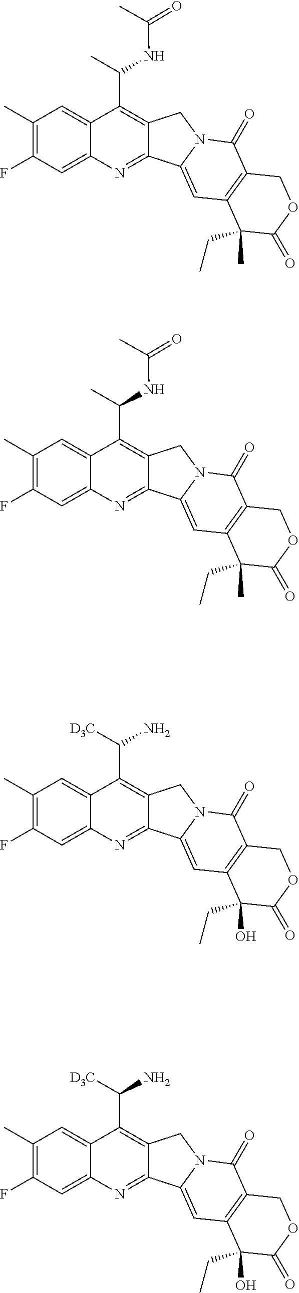

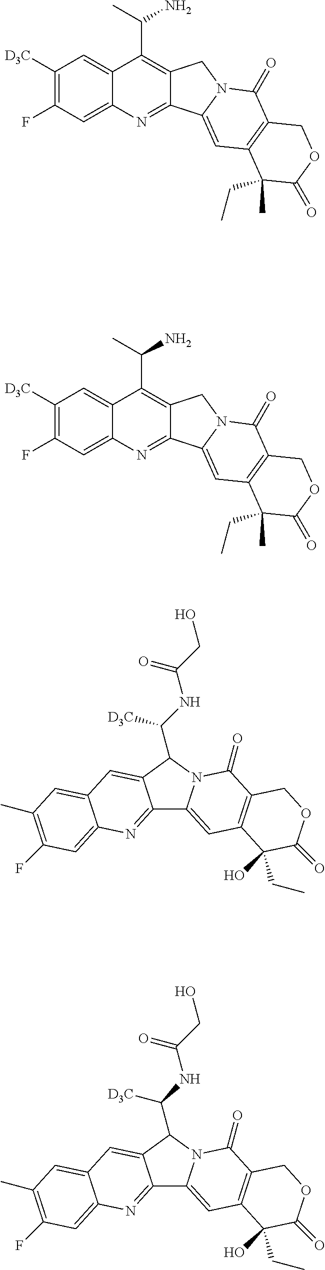

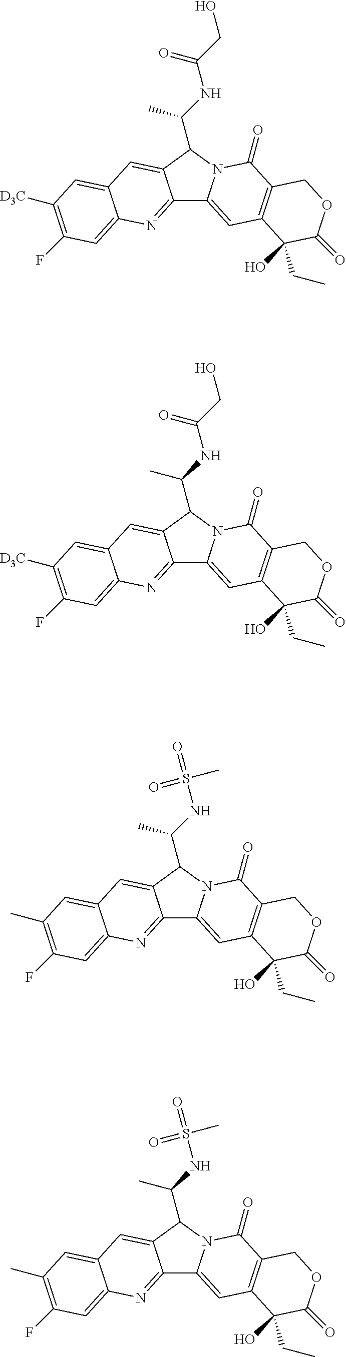 [SMILES: [2H]C([2H])([2H])c1cc2cc3c(nc2cc1F)-c1cc2c(c(=O)n1C3[C@H](C)NC(=O)CO)COC(=O)[C@]2(O)CC ; [2H]C([2H])([2H])c1cc2cc3c(nc2cc1F)-c1cc2c(c(=O)n1C3[C@@H](C)NC(=O)CO)COC(=O)[C@]2(O)CC ; CC[C@@]1(O)C(=O)OCc2c1cc1n(c2=O)C([C@H](C)NS(C)(=O)=O)c2cc3cc(C)c(F)cc3nc2-1 ; CC[C@@]1(O)C(=O)OCc2c1cc1n(c2=O)C([C@@H](C)NS(C)(=O)=O)c2cc3cc(C)c(F)cc3nc2-1]

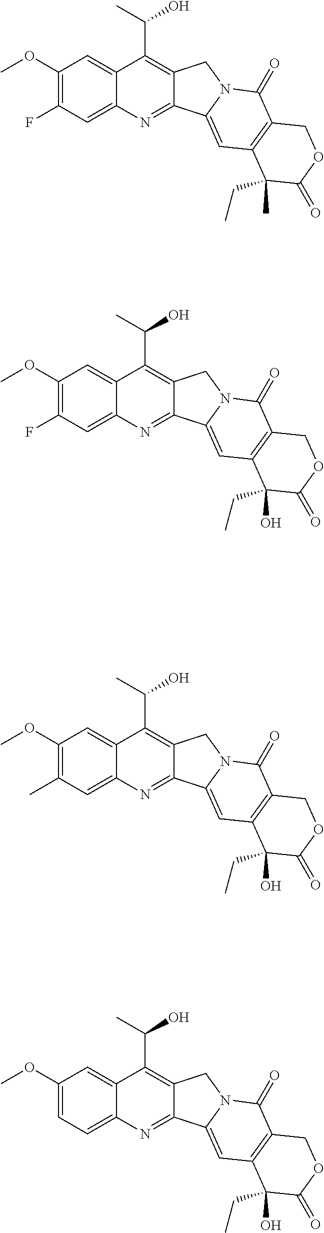 [SMILES: CC[C@@]1(C)C(=O)OCc2c1cc1n(c2=O)Cc2c-1nc1cc(F)c(OC)cc1c2[C@H](C)O ; CC[C@@]1(O)C(=O)OCc2c1cc1n(c2=O)Cc2c-1nc1cc(F)c(OC)cc1c2[C@@H](C)O ; CC[C@@]1(O)C(=O)OCc2c1cc1n(c2=O)Cc2c-1nc1cc(C)c(OC)cc1c2[C@H](C)O ; CC[C@@]1(O)C(=O)OCc2c1cc1n(c2=O)Cc2c-1nc1ccc(OC)cc1c2[C@@H](C)O]

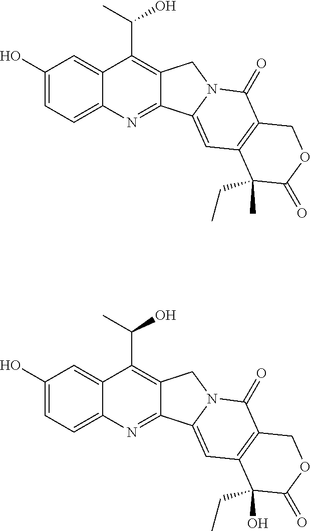 [SMILES: CC[C@@]1(C)C(=O)OCc2c1cc1n(c2=O)Cc2c-1nc1ccc(O)cc1c2[C@H](C)O ; CC[C@@]1(O)C(=O)OCc2c1cc1n(c2=O)Cc2c-1nc1ccc(O)cc1c2[C@@H](C)O]

12. A method for preparing the compound or the pharmaceutically acceptable salt thereof according to claim 1, comprising: 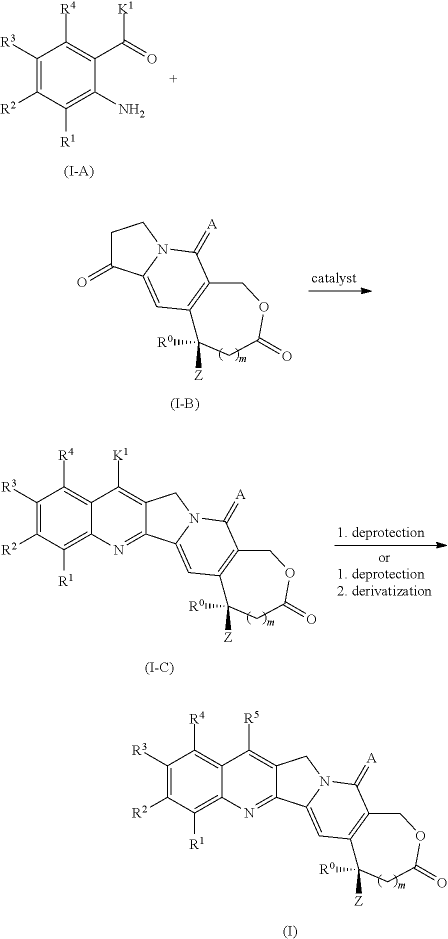
intermediate (I-A) and intermediate (I-B) were heated for ring-closing under catalyst conditions to obtain intermediate formula (I-C), and then deprotected or derivatized after deprotecting to obtain compound formula (I);
wherein the catalyst is p-toluenesulfonic acid monohydrate, pyridine p-toluenesulfonate complex (PPTS) or camphorsulfonic acid (CSA), the reaction temperature is 50-200° C., and the molar ratio of intermediate (I-A) to intermediate (I-B) is 5:1-1:5;
K1 is 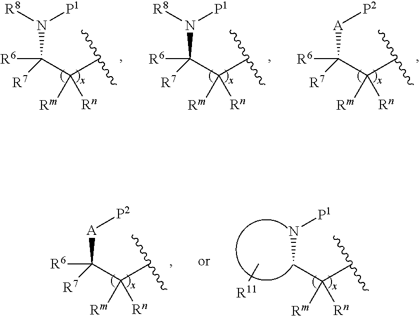
(the ring in 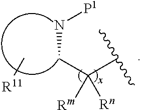
is 3-7 membered heterocycle); and,
P¹ and P² are protective groups, where P¹ is selected from benzyloxycarbonyl, benzyl, p-methoxybenzyl, 9-fluorenylmethoxycarbonyl, allyloxy carbonyl, 2-(trimethylsilyl) ethoxycarbonylation, phthalyl, p-toluenesulfonyl, trifluoroacetyl, trityl, 2, 4-dimethoxybenzyl, acetyl or other ester protecting groups; where P² is selected from benzyloxycarbonyl, benzyl, p-methoxy benzyl, tert-butyldiphenylsilyl, tert-butyldimethylsilyl, triisopropylsilyl, allyloxycarbonyl, 2-(trimethylsilyl) ethoxycarbonylation, trityl, 2, 4-dimethoxybenzyl, methoxymethyl ether or acetyl.

13. A pharmaceutical composition comprising an effective amount of the compound or the pharmaceutically acceptable salt thereof according to claim 1 and a pharmaceutically acceptable carrier, diluent or excipient.

14. A method for treating or preventing tumors in a subject in need thereof, comprising: administering the compound or the pharmaceutically acceptable salt thereof according to claim 1 to the subject.

15. A method for treating cancer in a subject in need thereof, comprising: administering the compound or the pharmaceutically acceptable salt thereof according to claim 1 to the subject, wherein the cancer is selected from one or more of breast cancer, ovarian cancer, prostate cancer, melanoma cancer, brain cancer, nasopharyngeal cancer, esophageal cancer, gastric cancer, liver cancer, pancreatic cancer, colorectal cancer, lung cancer, renal cancer, skin cancer, glioblastoma, neuroblastoma, sarcoma, osteochondroma, bone cancer, seminomas, testicular tumors, uterine tumors, head and neck tumors, multiple myeloma, malignant lymphoma, polycythemia vera, leukemia, thyroid tumors, ureteral tumors, bladder tumors, gallbladder carcinoma, cholangiocarcinoma, or choriocarcinoma.

16. A drug conjugate prepared from the compound or the pharmaceutically acceptable salt thereof according to claim 1; and the drug conjugate is antibody drug conjugate, peptide drug conjugate, small molecule drug conjugate, polymer drug conjugate, lipid drug conjugate or protein drug conjugate.

17. A drug delivery system comprising the compound or the pharmaceutically acceptable salt thereof according to claim 1, wherein the drug delivery system includes microspheres, micelles, liposomes, polymer nanoparticles, liposome nanoparticles or small molecule nanoparticles.

18. The camptothecin compound or the pharmaceutically acceptable salt thereof according to claim 4, characterized in that R⁵ is

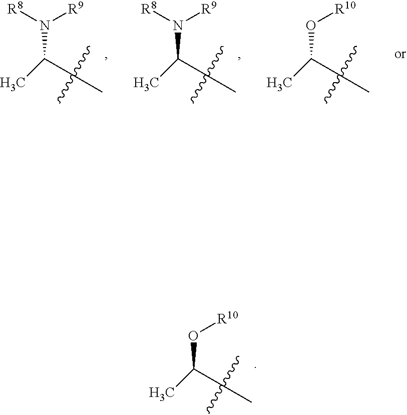

19. A method for treating or preventing tumors in a subject in need thereof, comprising: administering the pharmaceutical composition according to claim 11 to the subject.

20. A method for treating cancer in a subject in need thereof, comprising: administering the pharmaceutical composition according to claim 11 to the subject, wherein the cancer is selected from one or more of breast cancer, ovarian cancer, prostate cancer, melanoma cancer, brain cancer, nasopharyngeal cancer, esophageal cancer, gastric cancer, liver cancer, pancreatic cancer, colorectal cancer, lung cancer, renal cancer, skin cancer, glioblastoma, neuroblastoma, sarcoma, osteochondroma, bone cancer, seminomas, testicular tumors, uterine tumors, head and neck tumors, multiple myeloma, malignant lymphoma, polycythemia vera, leukemia, thyroid tumors, ureteral tumors, bladder tumors, gallbladder carcinoma, cholangiocarcinoma, or choriocarcinoma.
